SEQUENCE LISTING (11 sequences)

SEQ ID NO: 1 - protein, 1449 aa, Homo sapiens
LTELYIENQQHLQHLELRDLRGLGELRNLTIVKSGLRFVAPDAFHFTPRLSRLNLSFNALLYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEKKDETPFGVSVAVGLAVFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAWYQLSNTEAIDCITQGRELERPRACPPEVYAIMRGCWQREPQQRHSIKDVHARLQALAQAPPVYLDVLGPPVYLDVLG

SEQ ID NO: 2 - DNA, 2663 bp, Homo sapiens
tgcagctgggagcgcacagacggctgccccgcctgagcgaggcgggcgccgccgcgatgctgcgaggcggacggcgcgggcagcttggctggcacagctgggctgcggggccgggcagcctgctggcttggctgatactggcatctgcgggcgccgcaccctgccccgatgcctgctgcccccacggctcctcgggactgcgatgcacccgggatggggccctggatagcctccaccacctgcccggcgcagagaacctgactgagctctacatcgagaaccagcagcatctgcagcatctggagctccgtgatctgaggggcctgggggagctgagaaacctcaccatcgtgaagagtggtctccgtttcgtggcgccagatgccttccatttcactcctcggctcagtcgcctgaatctctccttcaacgctctggagtctctctcctggaaaactgtgcagggcctctccttacaggaactggtcctgtcggggaaccctctgcactgttcttgtgccctgcgctggctacagcgctgggaggaggagggactgggcggagtgcctgaacagaagctgcagtgtcatgggcaagggcccctggcccacatgcccaatgccagctgtggtgtgcccacgctgaaggtccaggtgcccaatgcctcggtggatgtgggggacgacgtgctgctgcggtgccaggtggaggggcggggcctggagcaggccggctggatcctcacagagctggagcagtcagccacggtgatgaaatctgggggtctgccatccctggggctgaccctggccaatgtcaccagtgacctcaacaggaagaacgtgacgtgctgggcagagaacgatgtgggccgggcagaggtctctgttcaggtcaacgtctccttcccggccagtgtgcagctgcacacggcggtggagatgcaccactggtgcatccccttctctgtggatgggcagccggcaccgtctctgcgctggctcttcaatggctccgtgctcaatgagaccagcttcatcttcactgagttcctggagccggcagccaatgagaccgtgcggcacgggtgtctgcgcctcaaccagcccacccacgtcaacaacggcaactacacgctgctggctgccaaccccttcggccaggcctccgcctccatcatggctgccttcatggacaaccctttcgagttcaaccccgaggaccccatccctgtctccttctcgccggtggacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctaggcaaggtcccgtcatagcaattatatttattatcccttgaaaaaaaa

SEQ ID NO: 3 - protein, 723 aa, Homo sapiens
LAASYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 4 - DNA, 2581 bp, Homo sapiens
gcaccctggtcatctgcggactcagcctgagcttccagagggcctaggagcagtaagggagtgagtgggcaactcggcgcatgaaggaggccgccctcatctgcctggcaccctctgtacccccgatcttgacggtgaagtcctgggacaccatgcagttgcgggctgctagatctcggtgcacaaacttgttggcagcaagctacatcgagaaccagcagcatctgcagcatctggagctccgtgatctgaggggcctgggggagctgagaaacctcaccatcgtgaagagtggtctccgtttcgtggcgccagatgccttccatttcactcctcggctcagtcgcctgaatctctccttcaacgctctggagtctctctcctggaaaactgtgcagggcctctccttacaggaactggtcctgtcggggaaccctctgcactgttcttgtgccctgcgctggctacagcgctgggaggaggagggactgggcggagtgcctgaacagaagctgcagtgtcatgggcaagggcccctggcccacatgcccaatgccagctgtggtgtgcccacgctgaaggtccaggtgcccaatgcctcggtggatgtgggggacgacgtgctgctgcggtgccaggtggaggggcggggcctggagcaggccggctggatcctcacagagctggagcagtcagccacggtgatgaaatctgggggtctgccatccctggggctgaccctggccaatgtcaccagtgacctcaacaggaagaacgtgacgtgctgggcagagaacgatgtgggccgggcagaggtctctgttcaggtcaacgtctccttcccggccagtgtgcagctgcacacggcggtggagatgcaccactggtgcatccccttctctgtggatgggcagccggcaccgtctctgcgctggctcttcaatggctccgtgctcaatgagaccagcttcatcttcactgagttcctggagccggcagccaatgagaccgtgcggcacgggtgtctgcgcctcaaccagcccacccacgtcaacaacggcaactacacgctgctggctgccaaccccttcggccaggcctccgcctccatcatggctgccttcatggacaaccctttcgagttcaaccccgaggaccccatccctgacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctaggcaaggtcccgtcatagcaattatatttattatcccttgaaaaaaaaaa

SEQ ID NO: 5 - protein, 822 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 6 - DNA, 3194 bp, Homo sapiens
ggaaggtttaaagaagaagccgcaaagcgcagggaaggcctcccggcacgggtgggggaaagcggccggtgcagcgcggggacaggcactcgggctggcactggctgctagggatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaacccaaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggctagggcccttttccccagaccgatccttcccaacgtactcctcagacgggctgagaggatgaacatcttttaactgccgctggaggccaccaagctgctctccttcactctgacagtattaacatcaaagactccgagaagctctcgagggaagcagtgtgtacttcttcatccatagacacagtattgacttctttttggcattatctctttctctctttccatctcccttggttgttcctttttctttttttaaattttctttttcttcttttttttcgtcttccctgcttcacgattcttaccctttcttttgaatcaatctggcttctgcattactattaactctgcatagacaaaggccttaacaaacgtaatttgttatatcagcagacactccagtttgcccaccacaactaacaatgccttgttgtattcctgcctttgatgtggatgaaaaaaagggaaaacaaatatttcacttaaactttgtcacttctgctgtacagatatcgagagtttctatggattcacttctatttatttattattattactgttcttattgtttttggatggcttaagcctgtgtataaaaaaaaaaaaaaatctaga

SEQ ID NO: 7 - protein, 839 aa, Homo sapiens [sequence omitted]

SEQ ID NO: 8 - DNA, 2715 bp, Homo sapiens
ggatccgcgtcggagatggatgtctctctttgcccagccaagtgtagtttctggcggattttcttgctgggaagcgtctggctggactatgtgggctccgtgctggcttgccctgcaaattgtgtctgcagcaagactgagatcaattgccggcggccggacgatgggaacctcttccccctcctggaagggcaggattcagggaacagcaatgggaacgccaatatcaacatcacggacatctcaaggaatatcacttccatacacatagagaactggcgcagtcttcacacgctcaacgccgtggacatggagctctacaccggacttcaaaagctgaccatcaagaactcaggacttcggagcattcagcccagagcctttgccaagaacccccatttgcgttatataaacctgtcaagtaaccggctcaccacactctcgtggcagctcttccagacgctgagtcttcgggaattgcagttggagcagaactttttcaactgcagctgtgacatccgctggatgcagctctggcaggagcagggggaggccaagctcaacagccagaacctctactgcatcaatgctgatggctcccagcttcctctcttccgcatgaacatcagtcagtgtgaccttcctgagatcagcgtgagccacgtcaacctgaccgtacgagagggtgacaatgctgttatcacttgcaatggctctggatcaccccttcctgatgtggactggatagtcactgggctgcagtccatcaacactcaccagaccaatctgaactggaccaatgttcatgccatcaacttgacgctggtgaatgtgacgagtgaggacaatggcttcaccctgacgtgcattgcagagaacgtggtgggcatgagcaatgccagtgttgccctcactgtctactatcccccacgtgtggtgagcctggaggagcctgagctgcgcctggagcactgcatcgagtttgtggtgcgtggcaaccccccaccaacgctgcactggctgcacaatgggcagcctctgcgggagtccaagatcatccatgtggaatactaccaagagggagagatttccgagggctgcctgctcttcaacaagcccacccactacaacaatggcaactataccctcattgccaaaaacccactgggcacagccaaccagaccatcaatggccacttcctcaaggagccctttccagagagcacggataactttatcttgtttgacgaagtgagtcccacacctcctatcactgtgacccacaaaccagaagaagacacttttggggtatccatagcagttggacttgctgcttttgcctgtgtcctgttggtggttctcttcgtcatgatcaacaaatatggtcgacggtccaaatttggaatgaagggtcccgtggctgtcatcagtggtgaggaggactcagccagcccactgcaccacatcaaccacggcatcaccacgccctcgtcactggatgccgggcccgacactgtggtcattggcatgactcgcatccctgtcattgagaacccccagtacttccgtcagggacacaactgccacaagccggacacgtatgtgcagcacattaagaggagagacatcgtgctgaagcgagaactgggtgagggagcctttggaaaggtcttcctggccgagtgctacaacctcagcccgaccaaggacaagatgcttgtggctgtgaaggccctgaaggatcccaccctggctgcccggaaggatttccagagggaggccgagctgctcaccaacctgcagcatgagcacattgtcaagttctatggagtgtgcggcgatggggaccccctcatcatggtctttgaatacatgaagcatggagacctgaataagttcctcagggcccatgggccagatgcaatgatccttgtggatggacagccacgccaggccaagggtgagctggggctctcccaaatgctccacattgccagtcagatcgcctcgggtatggtgtacctggcctcccagcactttgtgcaccgagacctggccaccaggaactgcctggttggagcgaatctgctagtgaagattggggacttcggcatgtccagagatgtctacagcacggattattacaggctctttaatccatctggaaatgatttttgtatatggtgtgaggtgggaggacacaccatgctccccattcgctggatgcctcctgaaagcatcatgtaccggaagttcactacagagagtgatgtatggagcttcggggtgatcctctgggagatcttcacctatggaaagcagccatggttccaactctcaaacacggaggtcattgagtgcattacccaaggtcgtgttttggagcggccccgagtctgccccaaagaggtgtacgatgtcatgctggggtgctggcagagggaaccacagcagcggttgaacatcaaggagatctacaaaatcctccatgctttggggaaggccaccccaatctacctggacattcttggctagtggtggctggtggtcatgaattcatactctgttgcctcctctctccctgcctcacatctcccttccacctcacaactccttccatccttgactgaagcgaacatcttcatataaactcaagtgcctgctacacatacaacactgaaaaaaggaaaaaaaaagaaaaaaaaaaaaaaccgc

SEQ ID NO: 9 - protein, 796 aa, Homo sapiens
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 10 - protein, 822 aa, Homo sapiens; identical to SEQ ID NO: 5

SEQ ID NO: 11 - protein, 839 aa, Homo sapiens
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRLFNPSGNDFCIWCEVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

(19) United States
(12) Patent Application Publication
(10) Pub. No.: US 2021/0023086 A1
(43) Pub. Date: Jan. 28, 2021

(54) TREATMENT OF TRK-ASSOCIATED CANCERS

(71) Applicant: LOXO ONCOLOGY, INC., Stamford, CT (US)

(72) Inventors: Joshua H. BILENKER, Stamford, CT (US); Jacob VAN NAARDEN, Stamford, CT (US); Nisha NANDA, South San Francisco, CA (US)

(21) Appl. No.: 17/043,134

(22) PCT Filed: Mar. 29, 2019
(86) PCT No.: PCT/US2019/024961

§ 371 Date: Sep. 29, 2020

Related U.S. Application Data
(60) Provisional application No. 62/650,255, filed on Mar. 29, 2018.
(60) Provisional application No. 62/679,426, filed on Jun. 1, 2018.
(60) Provisional application No. 62/683,821, filed on Jun. 12, 2018.
(60) Provisional application No. 62/686,524, filed on Jun. 18, 2018.
(60) Provisional application No. 62/721,269, filed on Aug. 22, 2018.
(60) Provisional application No. 62/795,422, filed on Jan. 22, 2019.
(60) Provisional application No. 62/804,617, filed on Feb. 12, 2019.

(51) Int. Cl. A61K 31/519; A61P 35/00

(52) U.S. Cl. CPC A61K 31/519; A61K 45/06; A61P 35/00

(57) ABSTRACT

Provided herein are compounds and pharmaceutical compositions comprising the compounds and the use of the compounds in the treatment of cancer. More particularly, provided herein are method of treating cancer (e.g., a Trk-associated cancer) by administration of one or more Trk inhibitors and optionally an immunotherapy agent.

### TECHNICAL FIELD

Provided herein are compounds and pharmaceutical compositions comprising the compounds and the use of the compounds in the treatment of cancer. More particularly, provided herein are method of treating cancer (e.g., a Trk-associated cancer) by administration of one or more Trk inhibitors and optionally an immunotherapy agent.

### BACKGROUND

Tropomyosin-related kinase (TRK) is a receptor tyrosine kinase family of neurotrophin receptors that are found in multiple tissues types. Three members of the TRK proto-oncogene family have been described: TrkA, TrkB, and TrkC, encoded by the NTRK1, NTRK2, and NTRK3 genes, respectively. The TRK receptor family is involved in neuronal development, including the growth and function of neuronal synapses, memory development, and maintenance, and the protection of neurons after ischemia or other types of injury (Nakagawara, Cancer Lett. 169:107-114, 2001).

TRK was originally identified from a colorectal cancer cell line as an oncogene fusion containing 5′ sequences from tropomyosin-3 (TPM3) gene and the kinase domain encoded by the 3′ region of the neurotrophic tyrosine kinase, receptor, type 1 gene (NTRK1) (Pulciani et al., Nature 300:539-542, 1982; Martin-Zanca et al., Nature 319:743-748, 1986). TRK gene fusions follow the well-established paradigm of other oncogenic fusions, such as those involving ALK and ROS1, which have been shown to drive the growth of tumors and can be successfully inhibited in the clinic by targeted drugs (Shaw et al., New Engl. J. Med. 371:1963-1971, 2014; Shaw et al., New Engl. J. Med. 370:1189-1197, 2014). Oncogenic TRK fusions induce cancer cell proliferation and engage critical cancer-related downstream signaling pathways such as mitogen activated protein kinase (MAPK) and AKT (Vaishnavi et al., Cancer Discov. 5:25-34, 2015). Numerous oncogenic rearrangements involving NTRK1 and its related TRK family members NTRK2 and NTRK3 have been described (Vaishnavi et al., Cancer Disc. 5:25-34, 2015; Vaishnavi et al., Nature Med. 19:1469-1472, 2013). Although there are numerous different 5′ gene fusion partners identified, all share an in-frame, intact TRK kinase domain. A variety of different Trk inhibitors have been developed to treat cancer (see, e.g., U.S. Patent Application Publication No. 62/080,374, International Application Publication Nos. WO 11/006074, WO 11/146336, WO 10/033941, and WO 10/048314, and U.S. Pat. Nos. 8,933,084, 8,791,123, 8,637,516, 8,513,263, 8,450,322, 7,615,383, 7,384,632, 6,153,189, 6,027,927, 6,025,166, 5,910,574, 5,877,016, and 5,844,092).

### SUMMARY

Provided herein are methods of treating cancer by administration of one or more Trk inhibitors and optionally an immunotherapy agent. In some embodiments, the methods provided herein comprise administering a therapeutically effective amount of a first Trk inhibitor or a pharmaceutically acceptable salt thereof, a second Trk inhibitor or a pharmaceutically acceptable salt thereof, and an immunotherapy agent. Also provided herein are methods of treating cancer comprising administering a Trk inhibitor or a pharmaceutically acceptable salt thereof and an immunotherapy agent. In some embodiments the Trk inhibitor is selected from (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate or (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one.

Also provided herein is a method for treating cancer, the method comprising administering to the patient a therapeutically effective amount of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, a second Trk inhibitor or a pharmaceutically acceptable salt thereof, and optionally an immunotherapy agent.

Further provided herein is a method for treating cancer, the method comprising administering to the patient a therapeutically effective amount of (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one, a second Trk inhibitor, and optionally an immunotherapy agent.

This disclosure also provides a method for treating cancer, the method comprising administering to the patient a therapeutically effective amount of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one, and optionally an immunotherapy agent.

Further provided herein is a method for treating cancer, the method comprising administering to the patient a therapeutically effective amount of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate and optionally an immunotherapy agent.

This disclosure also provides a method for treating cancer, the method comprising administering to the patient a therapeutically effective amount of (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one and optionally an immunotherapy agent. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DETAILED DESCRIPTION

Provided herein are methods of treating cancer by administration of one or more Trk inhibitors and optionally an immunotherapy agent. In some embodiments, the methods provided herein comprise administering a therapeutically effective amount of a first Trk inhibitor or a pharmaceutically acceptable salt thereof, a second Trk inhibitor or a pharmaceutically acceptable salt thereof, and an immunotherapy agent. Also provided herein are methods of treating cancer comprising administering a Trk inhibitor or a pharmaceutically acceptable salt thereof and an immunotherapy agent. In some embodiments the Trk inhibitor is selected from (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate or (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one.

As can be appreciated in the art, the various aspects described below can be used in any combination without limitation.

### Tropomyosin Receptor Kinases (Trks)

Three different NTRK genes have been implicated as having a role in cancer (e.g., through discovery of chromosome translocations resulting in constitutively active Trk fusion proteins): NTRK1, NTRK2, and NTRK3. The NTRK1, NTRK2, and NTRK3 genes encode TrkA, TrkB, and TrkC, respectively.

Non-limiting exemplary amino acid and cDNA sequences for wild-type TrkA are provided below. The exemplary wild-type protein and cDNA sequences provided below can be used to identify a point mutation in a NTRK1 gene or can be used to determine mutation in a TrkA protein caused by a point mutation in a NTRK1 gene, respectively. Additional wild-type protein and cDNA sequences for TrkA are known in the art.

The amino acid positions used to describe the TrkA substitutions herein are based on the wild-type sequence of TrkA of SEQ ID NO: 1. The corresponding amino acid position in the wild-type sequence of another isoform of TrkA (SEQ ID NO: 3) can be identified by performing a sequence alignment between SEQ ID NO: 1 and SEQ ID NO: 3. A similar method (e.g., alignment of SEQ ID NO: 1 to the amino acid sequence of any other isoform of TrkA) can be used to match the amino acid positions of the substitutions in TrkA described herein to the corresponding amino acid position in other isoforms of TrkA known in the art.

### Wildtype Human TrkA Protein Isoform A (NP_002520) (SEQ ID NO: 1)

Wildtype Human TrkA cDNA Isoform A (NM_002529) (SEQ ID NO: 2)

### Wildtype Human TrkA Protein Isoform B (NP_001007793) (SEQ ID NO: 3)

Wildtype Human TrkA cDNA Isoform B (NM_001007792) (SEQ ID NO: 4)

### Alignment of TrkA Isoforms (SEQ ID NO: 1 and SEQ ID NO: 3)

| | | | |
|---|---|---|---|
| S1 | 68 | LTELYIENQQHLQHLELRDLRGLGELRNLTIVKSGLRFVAPDAFHFTPRLSRLNLSFNAL | 127 |
| | | L YIENQQHLQHLELRDLRGLGELRNLTIVKSGLRFVAPDAFHFTPRLSRLNLSFNAL | |
| S3 | 38 | LAASYIENQQHLQHLELRDLRGLGELRNLTIVKSGLRFVAPDAFHFTPRLSRLNLSFNAL | 97 |
| S1 | 128 | ESLSWKTVQGLSLQELVLSGNPLHCSCALRWLQRWEEEGLGGVPEQKLQCHGQGPLAHMP | 187 |
| | | ESLSWKTVQGLSLQELVLSGNPLHCSCALRWLQRWEEEGLGGVPEQKLQCHGQGPLAHMP | |
| S3 | 98 | ESLSWKTVQGLSLQELVLSGNPLHCSCALRWLQRWEEEGLGGVPEQKLQCHGQGPLAHMP | 157 |
| S1 | 188 | NASCGVPTLKVQVPNASVDVGDDVLLRCQVEGRGLEQAGWILTELEQSATVMKSGGLPSL | 247 |
| | | NASCGVPTLKVQVPNASVDVGDDVLLRCQVEGRGLEQAGWILTELEQSATVMKSGGLPSL | |
| S3 | 158 | NASCGVPTLKVQVPNASVDVGDDVLLRCQVEGRGLEQAGWILTELEQSATVMKSGGLPSL | 217 |
| S1 | 248 | GLTLANVTSDLNRKNVTCWAENDVGRAEVSVQVNVSFPASVQLHTAVEMHHWCIPFSVDG | 307 |
| | | GLTLANVTSDLNRKNVTCWAENDVGRAEVSVQVNVSFPASVQLHTAVEMHHWCIPFSVDG | |
| S3 | 218 | GLTLANVTSDLNRKNVTCWAENDVGRAEVSVQVNVSFPASVQLHTAVEMHHWCIPFSVDG | 277 |
| S1 | 308 | QPAPSLRWLFNGSVLNETSFIFTEFLEPAANETVRHGCLRLNQPTHVNNGNYTLLAANPF | 367 |
| | | QPAPSLRWLFNGSVLNETSFIFTEFLEPAANETVRHGCLRLNQPTHVNNGNYTLLAANPF | |
| S3 | 278 | QPAPSLRWLFNGSVLNETSFIFTEFLEPAANETVRHGCLRLNQPTHVNNGNYTLLAANPF | 337 |
| S1 | 368 | GQASASIMAAFMDNPFEFNPEDPIPVSFSPVDTNSTSGDPVEKKDETPFGVSVAVGLAVF | 427 |
| | | GQASASIMAAFMDNPFEFNPEDPIP DTNSTSGDPVEKKDETPFGVSVAVGLAVF | |
| S3 | 338 | GQASASIMAAFMDNPFEFNPEDPIP------DTNSTSGDPVEKKDETPFGVSVAVGLAVF | 391 |
| S1 | 428 | ACLFLSTLLLVLNKCGRRNKFGINRPAVLAPEDGLAMSLHFMTLGGSSLSPTEGKGSGLQ | 487 |
| | | ACLFLSTLLLVLNKCGRRNKFGINRPAVLAPEDGLAMSLHFMTLGGSSLSPTEGKGSGLQ | |
| S3 | 392 | ACLFLSTLLLVLNKCGRRNKFGINRPAVLAPEDGLAMSLHFMTLGGSSLSPTEGKGSGLQ | 451 |
| S1 | 488 | GHIIENPQYFSDACVHHIKRRDIVLKWELGEGAFGKVFLAECHNLLPEQDKMLVAVKALK | 547 |
| | | GHIIENPQYFSDACVHHIKRRDIVLKWELGEGAFGKVFLAECHNLLPEQDKMLVAVKALK | |
| S3 | 452 | GHIIENPQYFSDACVHHIKRRDIVLKWELGEGAFGKVFLAECHNLLPEQDKMLVAVKALK | 511 |
| S1 | 548 | EASESARQDFQREAELLTMLQHQHIVRFFGVCTEGRPLLMVFEYMRHGDLNRFLRSHGPD | 607 |
| | | EASESARQDFQREAELLTMLQHQHIVRFFGVCTEGRPLLMVFEYMRHGDLNRFLRSHGPD | |
| S3 | 512 | EASESARQDFQREAELLTMLQHQHIVRFFGVCTEGRPLLMVFEYMRHGDLNRFLRSHGPD | 571 |
| S1 | 608 | AKLLAGGEDVAPGPLGLGQLLAVASQVAAGMVYLAGLHFVHRDLATRNCLVGQGLVVKIG | 667 |
| | | AKLLAGGEDVAPGPLGLGQLLAVASQVAAGMVYLAGLHFVHRDLATRNCLVGQGLVVKIG | |
| S3 | 572 | AKLLAGGEDVAPGPLGLGQLLAVASQVAAGMVYLAGLHFVHRDLATRNCLVGQGLVVKIG | 631 |
| S1 | 668 | DFGMSRDIYSTDYYRVGGRTMLPIRWMPPESILYRKFTTESDVWSFGVVLWEIFTYGKQP | 727 |
| | | DFGMSRDIYSTDYYRVGGRTMLPIRWMPPESILYRKFTTESDVWSFGVVLWEIFTYGKQP | |
| S3 | 632 | DFGMSRDIYSTDYYRVGGRTMLPIRWMPPESILYRKFTTESDVWSFGVVLWEIFTYGKQP | 691 |
| S1 | 728 | WYQLSNTEAIDCITQGRELERPRACPPEVYAIMRGCWQREPQQRHSIKDVHARLQALAQA | 787 |
| | | WYQLSNTEAIDCITQGRELERPRACPPEVYAIMRGCWQREPQQRHSIKDVHARLQALAQA | |
| S3 | 692 | WYQLSNTEAIDCITQGRELERPRACPPEVYAIMRGCWQREPQQRHSIKDVHARLQALAQA | 751 |
| S1 | 788 | PPVYLDVLG | 796 |
| | | PPVYLDVLG | |
| S3 | 752 | PPVYLDVLG | 760 |

Non-limiting exemplary amino acid and cDNA sequences for wildtype TrkB are provided below. The exemplary wildtype protein and cDNA sequences provided below can be used to identify a point mutation in a NTRK2 gene or can be used to determine mutation in a TrkB protein caused by a point mutation in a NTRK2 gene, respectively. Additional wildtype protein and cDNA sequences for TrkB are known in the art.

The amino acid positions used to describe the TrkB substitutions herein are based on the wildtype sequence of TrkB of SEQ ID NO: 5. The corresponding amino acid position in the wildtype sequence of another isoform of TrkB can be identified by performing a sequence alignment between SEQ ID NO: 5 and the amino acid sequence of the other isoform of TrkB.

### Wildtype Human TrkB Protein Isoform A (AAB33109.1) (SEQ ID NO: 5)

Wildtype Human TrkB cDNA Isoform A (S76473.1) (SEQ ID NO: 6)

Non-limiting exemplary amino acid and cDNA sequences for wildtype TrkC are provided below. The exemplary wildtype protein and cDNA sequences provided below can be used to identify a point mutation in a NTRK3 gene or can be used to determine mutation in a TrkC protein caused by a point mutation in a NTRK3 gene, respectively. Additional wildtype protein and cDNA sequences for TrkC are known in the art.

The amino acid positions used to describe the TrkC substitutions herein are based on the wildtype sequence of TrkC of SEQ ID NO: 7. The corresponding amino acid position in the wildtype sequence of another isoform of TrkC can be identified by performing a sequence alignment between SEQ ID NO: 7 and the amino acid sequence of the other isoform of TrkC.

### Wildtype Human TrkC Protein (AAB33111.1) (SEQ ID NO: 7)

Wildtype Human TrkC cDNA (S76475.1) (SEQ ID NO: 8)

### Trk Inhibitors

A variety of Trk inhibitors are known in the art. The ability of a Trk inhibitor to act as a Trk inhibitor may be tested using one or both of the assays described in Examples A and B in U.S. Pat. No. 8,513,263, which is incorporated herein by reference.

A Trk inhibitor can bind to one or more of the sites on TrkA: the extracellular cysteine-rich region (domain 1), the extracellular leucine rich region (domain 2), the extracellular cysteine-rich region (domain 3), the extracellular immunoglobulin-like region (domain 4), the extracellular immunoglobulin-like region (domain 5), the transmembrane region, the intracellular kinase domain, an amino acid in the active site, the ATP-binding pocket, the tyrosine substrate binding site, the activation loop (e.g., the DFG motif of the activation loop), the kinase insert domain (KID) region (e.g., amino acids 603 to 623), the hinge region of the kinase, the α-C helix in the catalytic domain, the N-lobe lysine responsible for the stabilization of the α phosphate of the ATP substrate, the C-terminus (see, e.g., Bertrand et al., J. Mol. Biol. 423:439-453, 2012), the α-D helix in the C-terminus, the α-E helix in the C-terminus, an amino acid in the kinase domain that interacts with a ligand in the ATP binding site (see, e.g., Cherry et al., Curr. Med. Chem. 11:663-673, 2004). For example, a Trk inhibitor can bind to domain 5 or the intracellular kinase domain of a TrkA.

As used herein, a “first Trk kinase inhibitor” or “first Trk inhibitor” is a Trk inhibitor as described herein. As used herein, a “second Trk kinase inhibitor” or a “second Trk inhibitor” is a Trk inhibitor as described herein. When both a first and a second Trk inhibitor are present in a method provided herein, the first and second Trk kinase inhibitors are different.

Non-limiting examples of Trk inhibitors include: entrectinib (N-[5-(3,5-difluoro-benzyl)-1H-indazol-3-yl]-4-(4-methylpiperazin-1-yl)-2-(tetrahydro-pyran-4-ylamino)-benzamide), (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, cabozantinib ((N-(4-((6,7-Dimethoxyquinolin-4-yl)oxy)phenyl)-N′-(4-fluorophenyl)cyclopropane-1,1-dicarboxamide)), dovitinib (4-amino-5-fluoro-3-[6-(4-methylpiperazin-1-yl)-1H-benzimidazol-2-yl]quinolin-2(1H)-one mono 2-hydroxypropanoate hydrate), belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide), sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide), PLX7486, altiratinib (N-(4-((2-(cyclopropanecarboxamido)pyridin-4-yl)oxy)-2,5-difluorophenyl)-N-(4-fluorophenyl)cyclopropane-1,1-dicarboxamide), AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine), (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one, a (R)-2-phenylpyrrolidine substituted imadazopyridazine, AZD6918, GNF-4256, GTx-186, GNF-5837, AZ623, AG-879, CT327, AR-772, AR-523, AR-786, AR-256, AR-618, AZ-23, CEP-701, CEP-751, PHA-739358, CH7057288, AK-1830 (ARRY-954), dovitinib, HL5101 (NOV 1601), PF-06273340, Go 6976, GW441756, MGCD516, ONO-5390556, PHA-848125AC, Regorafenib, Sorafenib, Sunitinib, TSR-011, VM-902A, K252a, SNA-120a (Pegcantratinib; CT 301/P; CT 327; K-252a-PEG2K; Pegylated K252A), SNA-125a (CT 340), a 4-aminopyrazolylpyrimidine, a substituted pyrazolo[1,5-a] pyrimidine compound, BMS-754807, ONO-7579, F17752, ANA-12, ONO-4474, GZ389988, or TPX-0005 (repotrectinib; (7S,13R)-11-fluoro-6,7,13,14-tetrahydro-7,13-dimethyl-1,15-etheno-1H-pyrazolo[4,3-f][1,4,8,10]benzoxatriazacyclotridecin-4(5H)-one).

Non-limiting examples of receptor tyrosine kinase (e.g., Trk) targeted therapeutic agents, include afatinib, cabozantinib, cetuximab, crizotinib, dabrafenib, entrectinib, erlotinib, gefitinib, imatinib, lapatinib, lestaurtinib, nilotinib, pazopanib, panitumumab, pertuzumab, sunitinib, trastuzumab, 1-((3 S,4R)-4-(3-fluorophenyl)-1-(2-methoxyethyl)pyrrolidin-3-yl)-3-(4-methyl-3-(2-methylpyrimidin-5-yl)-1-phenyl-1H-pyrazol-5-yl)urea, AG 879, AR-772, AR-786, AR-256, AR-618, AZ-23, AZ623, DS-6051, Go 6976, GNF-5837, GTx-186, GW 441756, LOXO-101, MGCD516, PLX7486, RXDX101, TPX-0005, CG′806, and TSR-011. Additional Trk targeted therapeutic agents include those described in U.S. Pat. Nos. 8,450,322; 8,513,263; 8,933,084; 8,791,123; 8,946,226; 8,450,322; 8,299,057; and 8,912,194; U.S. Publication No. 2016/0137654; 2015/0166564; 2015/0051222; 2015/0283132; and 2015/0306086; International Publication No. WO 2010/033941; WO 2010/048314; WO 2016/077841; WO 2011/146336; WO 2011/006074; WO 2010/033941; WO 2012/158413; WO 2014078454; WO 2014078417; WO 2014078408; WO 2014078378; WO 2014078372; WO 2014078331; WO 2014078328; WO 2014078325; WO 2014078323; WO 2014078322; WO 2015175788; WO 2009/013126; WO 2013/174876; WO 2015/124697; WO 2010/058006; WO 2015/017533; WO 2015/112806; WO 2013/183578; and WO 2013/074518, all of which are hereby incorporated by reference in their entireties.

Further examples of Trk inhibitors can be found in U.S. Pat. No. 8,637,516, International Publication No. WO 2012/034091, U.S. Pat. No. 9,102,671, International Publication No. WO 2012/116217, U.S. Publication No. 2010/0297115, International Publication No. WO 2009/053442, U.S. Pat. No. 8,642,035, International Publication No. WO 2009092049, U.S. Pat. No. 8,691,221, International Publication No. WO2006131952, all of which are incorporated by reference in their entireties herein. Exemplary Trk inhibitors include GNF-4256, described in Cancer Chemother. Pharmacol. 75(1):131-141, 2015; and GNF-5837 (N-[3-[[2,3-dihydro-2-oxo-3-(1H-pyrrol-2-ylmethylene)-1H-indol-6-yl]amino]-4-methylphenyl]-N′-[2-fluoro-5-(trifluoromethyl)phenyl]-urea), described in ACS Med. Chem. Lett. 3(2):140-145, 2012, each of which is incorporated by reference in its entirety herein.

Additional examples of Trk inhibitors include those disclosed in U.S. Publication No. 2010/0152219, U.S. Pat. No. 8,114,989, and International Publication No. WO 2006/123113, all of which are incorporated by reference in their entireties herein. Exemplary Trk inhibitors include AZ623, described in Cancer 117(6):1321-1391, 2011; AZD6918, described in Cancer Biol. Ther. 16(3):477-483, 2015; AZ64, described in Cancer Chemother. Pharmacol. 70:477-486, 2012; AZ-23 ((S)-5-Chloro-N2-(1-(5-fluoropyridin-2-yl)ethyl)-N4-(5-isopropoxy-1H-pyrazol-3-yl)pyrimidine-2,4-diamine), described in Mol. Cancer Ther. 8:1818-1827, 2009; and AZD7451; each of which is incorporated by reference in its entirety.

A Trk inhibitor can include those described in U.S. Pat. Nos. 7,615,383; 7,384,632; 6,153,189; 6,027,927; 6,025,166; 5,910,574; 5,877,016; and 5,844,092, each of which is incorporated by reference in its entirety.

Further examples of Trk inhibitors include CEP-751, described in Int. J Cancer 72:672-679, 1997; CT327, described in Acta Derm. Venereol. 95:542-548, 2015; compounds described in International Publication No. WO 2012/034095; compounds described in U.S. Pat. No. 8,673,347 and International Publication No. WO 2007/022999; compounds described in U.S. Pat. No. 8,338,417; compounds described in International Publication No. WO 2016/027754; compounds described in U.S. Pat. No. 9,242,977; compounds described in U.S. Publication No. 2016/0000783; sunitinib (N-(2-diethylaminoethyl)-5-[(Z)-(5-fluoro-2-oxo-1H-indol-3-ylidene)methyl]-2,4-dimethyl-1H-pyrrole-3-carboxamide), as described in PLoS One 9:e95628, 2014; compounds described in International Publication No. WO 2011/133637; compounds described in U.S. Pat. No. 8,637,256; compounds described in Expert. Opin. Ther. Pat. 24(7):731-744, 2014; compounds described in Expert Opin. Ther. Pat. 19(3):305-319, 2009; (R)-2-phenylpyrrolidine substituted imidazopyridazines, e.g., GNF-8625, (R)-1-(6-(6-(2-(3-fluorophenyl)pyrrolidin-1-yl)imidazo[1,2-b]pyridazin-3-yl)-[2,4′-bipyridin]-2′-yl)piperidin-4-ol as described in ACS Med. Chem. Lett. 6(5):562-567, 2015; GTx-186 and others, as described in PLoS One 8(12):e83380, 2013; K252a ((9S-(9α,10β,12α))-2,3,9,10,11,12-hexahydro-10-hydroxy-10-(methoxycarbonyl)-9-methyl-9,12-epoxy-1H-diindolo[1,2,3-fg:3′,2′,1′-kl]pyrrolo[3,4-i][1,6]benzodiazocin-1-one), as described in Mol. Cell Biochem. 339(1-2):201-213, 2010; 4-aminopyrazolylpyrimidines, e.g., AZ-23 (((S)-5-chloro-N2-(1-(5-fluoropyridin-2-yl)ethyl)-N4-(5-isopropoxy-1H-pyrazol-3-yl)pyrimidine-2,4-diamine)), as described in J. Med. Chem. 51(15):4672-4684, 2008; PHA-739358 (danusertib), as described in Mol. Cancer Ther. 6:3158, 2007; Gö 6976 (5,6,7,13-tetrahydro-13-methyl-5-oxo-12H-indolo[2,3-a]pyrrolo[3,4-c]carbazole-12-propanenitrile), as described in J. Neurochem. 72:919-924, 1999; GW441756 ((3Z)-3-[(1-methylindol-3-yl)methylidene]-1H-pyrrolo[3,2-b]pyridin-2-one), as described in IJAE 115:117, 2010; milciclib (PHA-848125AC), described in J. Carcinog. 12:22, 2013; AG-879 ((2E)-3-[3,5-Bis(1,1-dimethylethyl)-4-hydroxyphenyl]-2-cyano-2-propenethioamide); altiratinib (N-(4-((2-(cyclopropanecarboxamido)pyridin-4-yl)oxy)-2,5-difluorophenyl)-N-(4-fluorophenyl)cyclopropane-1,1-dicarboxamide); cabozantinib (N-(4-((6,7-Dimethoxyquinolin-4-yl)oxy)phenyl)-N′-(4-fluorophenyl)cyclopropane-1,1-dicarboxamide); lestaurtinib ((5S,6S,8R)-6-Hydroxy-6-(hydroxymethyl)-5-methyl-7,8,14,15-tetrahydro-5H-16-oxa-4b,8a,14-triaza-5,8-methanodibenzo[b,h]cycloocta[jkl]cyclopenta[e]-as-indacen-13(6H)-one); dovatinib (4-amino-5-fluoro-3-[6-(4-methylpiperazin-1-yl)-1H-benzimidazol-2-yl]quinolin-2(1H)-one mono 2-hydroxypropanoate hydrate); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); ONO-5390556; regorafenib (4-[4-({[4-Chloro-3-(trifluoromethyl)phenyl]carbamoyl}amino)-3-fluorophenoxy]-N-methylpyridine-2-carboxamide hydrate); and VSR-902A; all of the references above are incorporated by reference in their entireties herein.

Trk inhibitors are also described in U.S. Pat. Nos. 9,670,207, 9,701,681, and 9,346,788 and U.S. patent application Ser. No. 14/883,072 and are incorporated herein by reference in their entireties.

In some embodiments, the Trk inhibitor is selected from the group consisting of: (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate; (R)—N-cyclopropyl-5-(2-(5-fluoropyridin-3-yl)pyrrolidin-1-yl)pyrazolo[1,5-a]pyrimidine-3-carboxamide; (6R,13S)-9-fluoro-13-methyl-2,11,15,19,20,23-hexaazapentacyclo[15.5.2.17,11.2,6.20,24]pentacosa-1(23),7,9, 17(24), 18,21-hexaene-16,25-dione; and (6R)-9-fluoro-15-methyl-2,11,16,20,21,24-hexaazapentacyclo[16.5.2.2,6.7,12.21,25]pentacosa-1(24),7,9,11,18(25),19,22-heptaen-17-one.

Non-limiting examples of Trk inhibitors are described in U.S. Pat. No. 8,513,263 and International Publication No. WO 2010/048314 both of which are incorporated by reference in their entireties herein, and include a compound of Formula I:

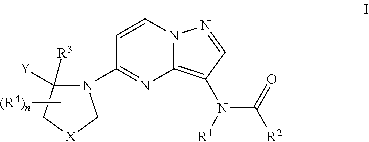

or a pharmaceutically acceptable salt thereof. For example, a Trk inhibitor can include one or more compounds selected from the group consisting of:
(R)—N-(5-(2-(2,5-difluorophenyl)pyrrolidin-1-yl)pyrazolo[1,5-a]pyrimidin-3-yl)-3-hydroxyazetidine-1-carboxamide;
N-(5-(2-(3-fluorophenyl)-2-methylpyrrolidin-1-yl)pyrazolo[1,5-a]pyrimidin-3-yl)-3-hydroxyazetidine-1-carboxamide;
(R)-1-(5-(2-(2,5-difluorophenyl)pyrrolidin-1-yl)pyrazolo[1,5-a]pyrimidin-3-yl)-3-phenylurea;
(R)—N-(5-(2-(2-(difluoromethyl)-5-fluorophenyl)pyrrolidin-1-yl)pyrazolo[1,5-a]pyrimidin-3-yl)-3-hydroxyazetidine-1-carboxamide;
(R)—N-(5-(2-(3-fluorophenyl)pyrrolidin-1-yl)pyrazolo[1,5-a]pyrimidin-3-yl)-1-methyl-6-oxo-1,6-dihydropyridazine-3-carboxamide;
(S)—N-(5-((R)-2-(2,5-difluorophenyl)pyrrolidin-1-yl)pyrazolo[1,5-a]pyrimidin-3-yl)-3-hydroxypyrrolidine-1-carboxamide;
(3R,4R)—N-(5-((R)-2-(2,5-difluorophenyl)pyrrolidin-1-yl)pyrazolo[1,5-a]pyrimidin-3-yl)-3,4-dihydroxypyrrolidine-1-carboxamide;
(S)—N-(5-((R)-2-(2-chloro-5-fluorophenyl)pyrrolidin-1-yl)pyrazolo[1,5-a]pyrimidin-3-yl)-3-methylpiperazine-1-carboxamide;
(R)—N-(5-(2-(2,5-difluorophenyl)pyrrolidin-1-yl)pyrazolo[1,5-a]pyrimidin-3-yl)-3-hydroxy-3-methylazetidine-1-carboxamide;
(R)—N-(5-(2-(3-fluorophenyl)pyrrolidin-1-yl)pyrazolo[1,5-a]pyrimidin-3-yl)-3-hydroxyazetidine-1-carboxamide; and
(R)-1-(4-chlorophenyl)-3-(5-(2-(2,5-difluorophenyl)pyrrolidin-1-yl)pyrazolo[1,5-a]pyrimidin-3-yl)urea,
   or a pharmaceutically acceptable salt thereof.

Additional examples of Trk inhibitors are the substituted pyrazolo[1,5-a] pyrimidine compounds described in U.S. Pat. No. 8,791,123 and International Publication No. WO 2011/006074, both of which are herein incorporated by reference in their entireties. For example, Trk inhibitors that are substituted pyrazolo[1,5-a]pyrimidine compounds can have the general formula II:

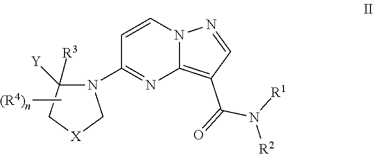

or a salt thereof. For example, a Trk inhibitor can include one or more compounds selected from the group consisting of:
(R)-5-(2-(2,5-difluorophenyl)pyrrolidin-1-yl)-N-(pyridin-2-yl)pyrazolo[1,5-a]pyrimidine-3-carboxamide;
(R)-5-(2-(2,5-difluorophenyl)pyrrolidin-1-yl)-N-(2-morpholinoethyl)pyrazolo[1,5-a]pyrimidine-3-carboxamide;
N-((2S)-bicyclo[2.2.1]heptan-2-yl)-5-((R)-2-(2,5-difluorophenyl)pyrrolidin-1-yl)pyrazolo[1,5-a]pyrimidine-3-carboxamide;
(R)-5-(2-(2,5-difluorophenyl)pyrrolidin-1-yl)-N-(2-(2-oxoimidazolidin-1-yl)ethyl)pyrazole[1,5-a]pyrimidine-3-carboxamide;
5-((R)-2-(2,5-difluorophenyl)pyrrolidin-1-yl)-N—((R)-2,3-dihydroxypropyl)pyrazolo[1,5-a]pyrimidine-3-carboxamide;
(R)—N-cyclopropyl-5-(2-(5-fluoropyridin-3-yl)pyrrolidin-1-yl)pyrazolo[1,5-a]pyrimidine-3-carboxamide;
(R)—N-tert-butyl-5-(2-(5-fluoropyridin-3-yl)pyrrolidin-1-yl)pyrazolo[1,5-a]pyrimidine-3-carboxamide;
(R)-5-(2-(2,5-difluorophenyl)pyrrolidin-1-yl)pyrazolo[1,5-a]pyrimidine-3-carboxamide;
(R)-5-(2-(5-fluoropyridin-3-yl)pyrrolidin-1-yl)-N-(1-methylcyclobutyl)pyrazolo[1,5-a]pyrimidine-3-carboxamide; and
5-((R)-2-(5-fluoropyridin-3-yl)pyrrolidin-1-yl)-N—((S)-1,1,1-trifluoropropan-2-yl)pyrazolo[1,5-a]pyrimidine-3-carboxamide;
   or a pharmaceutically acceptable salt thereof.

Additional examples of Trk inhibitors are the macrocyclic compounds described in U.S. Pat. No. 8,933,084 and International Publication No. WO 2011/146336, both of which are herein incorporated by reference in their entireties. For example, Trk inhibitors that are macrocyclic compounds can have the general formula III:

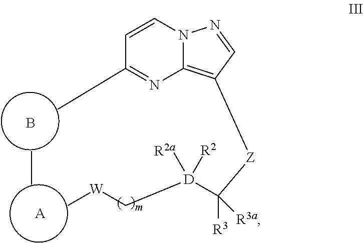

or a pharmaceutically acceptable salt thereof. For example, a Trk inhibitor can include one or more compounds selected from the group consisting of:
(6R)-9-fluoro-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one;
(6R)-9-fluoro-15-hydroxy-13-oxa-2,11,17,21,22,25-hexaazapentacyclo[17.5.2.0^{2,6}.0^{7,12}.0^{22,26}] hexacosa-1(25),7,9,11,19(26),20,23-heptaen-18-one;
(6R,15R)-9-fluoro-15-hydroxy-13-oxa-2,11,17,21,22,25-hexaazapentacyclo-[17.5.2.0^{2,6}.0^{7,12}.0^{22,26}] hexacosa-1(25),7,9,11,19(26),20,23-heptaen-18-one;
(6R)-9-fluoro-13-oxa-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R)-9-fluoro-13-oxa-2,11,18,22,23,26-hexaazapentacyclo[18.5.2.0^{2,6}.0^{7,12}.0^{23,27}]heptacosa-1(26),7,9,11,20(27),21,24-heptaen-19-one;
(6R,13S)-9-fluoro-13-methyl-2,11,15,19,20,23-hexaazapentacyclo [15.5.2.1^{7,11}.0^{2,6}.0^{20,24}]pentacosa-1(23),7,9,17(24),18,21-hexaene-16,25-dione;
(6R)-9-fluoro-2,11,13,16,20,21,24-heptaazapentacyclo[16.5.2.0^{2,6}.0^{7,12}.0^{21,25}]pentacosa-1(24),7,9,11,18(25),19,22-heptaen-17-one;
(6R)-9-fluoro-2,11,13,17,21,22,25-heptaazapentacyclo[17.5.2.0^{2,6}.0^{7,12}.0^{22,26}]hexacosa-1(25),7,9,11,19(26),20,23-heptaen-18-one;
(6R)-9-fluoro-17-methyl-13-oxa-2,11,17,21,22,25-hexaazapentacyclo[17.5.2.0^{2,6}.0^{7,12}.0^{22,26}] hexacosa-1(25),7,9,11,19(26),20,23-heptaen-18-one;
(6R)-9,15,15-trifluoro-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one;
(6R)-9-fluoro-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R)-9-fluoro-(15R)-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R)-9-fluoro-15,15-dimethyl-13-oxa-2,11,17,21,22,25-hexaazapentacyclo [17.5.2.0^{2,6}.0^{7,12}.0^{22,26}] hexacosa-1(25),7,9,11,19(26),20,23-heptaen-18-one; and
(6R)-9-fluoro-15,15-dimethyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
   or a pharmaceutically acceptable salt thereof.

Additional examples of Trk inhibitors are the substituted imidazo[1,2-b]pyridazine compounds described in U.S. Pat. No. 8,450,322 and International Publication No. WO 2010/033941, both of which are herein incorporated by reference in their entireties. For example, Trk inhibitors that are substituted imidazo[1,2B]pyridazine compounds can have the general formula IV:

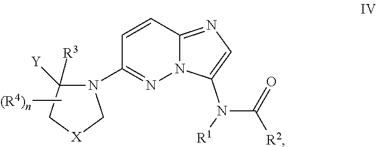

or a pharmaceutically acceptable salt thereof.

Additional examples of Trk inhibitors are the substituted pyrazolo[1,5-a]pyrimidine compounds described in WO 10/048314, herein incorporated by reference in its entirety. For example, Trk inhibitors that are substituted pyrazolo[1,5-a]pyrimidine compounds can have the general formula V:

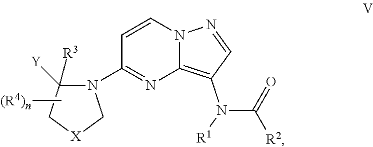

or a pharmaceutically acceptable salt thereof.

For example, a Trk inhibitor can include one or more compounds selected from the group consisting of:
(R)-5-(2-(2,5-difluorophenyl)pyrrolidin-1-yl)-N-(pyridin-2-yl)pyrazolo[1,5-a]pyrimidine-3-carboxamide;
(R)-5-(2-(2,5-difluorophenyl)pyrrolidin-1-yl)-N-(2-morpholinoethyl)pyrazolo[1,5-a]pyrimidine-3-carboxamide;
N-((2S)-bicyclo[2.2.1]heptan-2-yl)-5-((R)-2-(2,5-difluorophenyl)pyrrolidin-1-yl)pyrazolo[1,5-a]pyrimidine-3-carboxamide;
(R)-5-(2-(2,5-difluorophenyl)pyrrolidin-1-yl)-N-(2-(2-oxoimidazolidin-1-yl)ethyl)pyrazole[1,5-a]pyrimidine-3-carboxamide;
5-((R)-2-(2,5-difluorophenyl)pyrrolidin-1-yl)-N—((R)-2,3-dihydroxypropyl)pyrazolo[1,5-a]pyrimidine-3-carboxamide;
(R)—N-cyclopropyl-5-(2-(5-fluoropyridin-3-yl)pyrrolidin-1-yl)pyrazolo[1,5-a]pyrimidine-3-carboxamide;
(R)—N-tert-butyl-5-(2-(5-fluoropyridin-3-yl)pyrrolidin-1-yl)pyrazolo[1,5-a]pyrimidine-3-carboxamide;
(R)-5-(2-(2,5-difluorophenyl)pyrrolidin-1-yl)pyrazolo[1,5-a]pyrimidine-3-carboxamide;
(R)-5-(2-(5-fluoropyridin-3-yl)pyrrolidin-1-yl)-N-(1-methylcyclobutyl)pyrazolo[1,5-a]pyrimidine-3-carboxamide; and
5-((R)-2-(5-fluoropyridin-3-yl)pyrrolidin-1-yl)-N—((S)-1,1,1-trifluoropropan-2-yl)pyrazolo[1,5-a]pyrimidine-3-carboxamide;
   or a pharmaceutically acceptable salt thereof.

Additional Trk inhibitors can be found in U.S. Publication No. 2015/0166564 and WO 2012/158413, both of which are incorporated by reference in their entireties herein. For example, a Trk inhibitor can be a compound of Formula I:

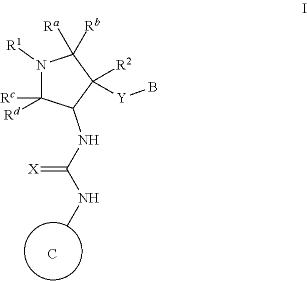

or stereoisomers, tautomers, or pharmaceutically acceptable salts, solvates or prodrugs thereof.

Further examples of Trk inhibitors can be found in International Publication No. WO 2014078454, which is incorporated by reference in its entirety herein. For example, a Trk inhibitor can be a compound of Formula I:

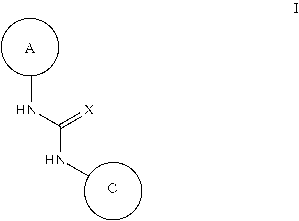

or stereoisomers, tautomers, or pharmaceutically acceptable salts, or solvates thereof.

Further examples of Trk inhibitors can be found in International Publication No. WO 2014078417, which is incorporated by reference in its entirety herein. For example, a Trk inhibitor can be a compound of Formula I:

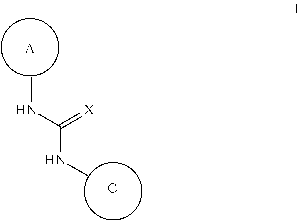

or stereoisomers, tautomers, or pharmaceutically acceptable salts, solvates or prodrugs thereof.

Further examples of Trk inhibitors can be found in International Publication No. WO 2014078378, which is incorporated by reference in its entirety herein. For example, a Trk inhibitor can be a compound of Formula I:

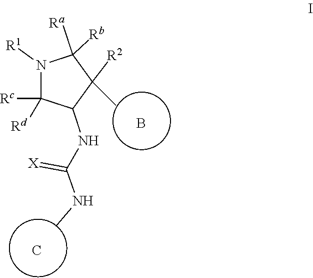

or stereoisomers, tautomers, or pharmaceutically acceptable salts, solvates or prodrugs thereof.

Additional examples of Trk inhibitors can be found in International Publication No. WO 2014078372, which is incorporated by reference in its entirety herein. For example, a Trk inhibitor can be a compound of Formula I:

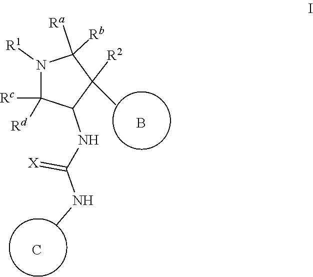

or stereoisomers, tautomers, or pharmaceutically acceptable salts, solvates or prodrugs thereof.

Additional examples of Trk inhibitors can be found in International Publication No. WO 2014078328, which is incorporated by reference in its entirety herein. For example, a Trk inhibitor can be a compound of Formula I-1:

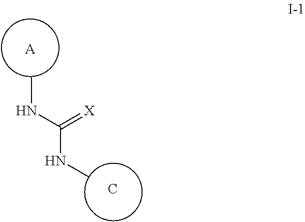

or stereoisomers, tautomers, or pharmaceutically acceptable salts, solvates or prodrugs thereof.

Further examples of Trk inhibitors can be found in International Publication No. WO 2014078325, which is incorporated by reference in its entirety herein. For example, a Trk inhibitor can be a compound of Formula I:

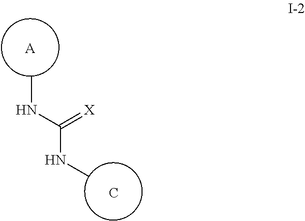

or a stereoisomer, tautomer, or pharmaceutically acceptable salt, solvate or prodrug thereof.

Additional examples of Trk inhibitors can be found in International Publication No. WO 2014078323, which is incorporated by reference in its entirety herein. For example, a Trk inhibitor can be a compound of Formula I:

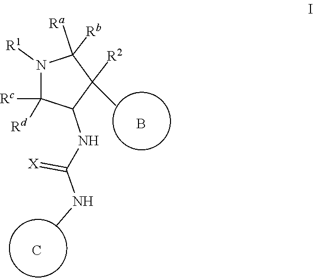

or stereoisomers, tautomers, or pharmaceutically acceptable salts, solvates or prodrugs.

Additional examples of Trk inhibitors can be found in International Publication No. WO 2014078322, which is incorporated by reference in its entirety herein. For example, a Trk inhibitor can be a compound of Formula I:

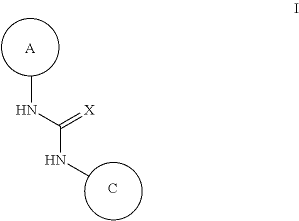

or stereoisomers, tautomers, or pharmaceutically acceptable salts, solvates or prodrugs thereof.

Exemplary Trk inhibitors include AR-772, AR-786, AR-256, and AR-618.

Non-limiting examples of Trk inhibitors can be found in U.S. Pat. No. 8,299,057 and International Publication No. WO 2009/013126 both of which are incorporated by reference in their entireties. For example, a Trk inhibitor can be a compound of Formula (I):

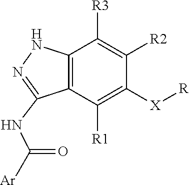

or optical isomers, tautomers or pharmaceutically acceptable salt thereof.

For example, a Trk inhibitor can be entrectinib (N-[5-(3,5-difluoro-benzyl)-1H-indazol-3-yl]-4-(4-methyl-piperazin-1-yl)-2-(tetrahydro-pyran-4-ylamino)-benzamide), or a pharmaceutically acceptable salt thereof. For example, a Trk inhibitor can be a polymorph such as those described in U.S. Publication No. 2015/0051222 or International Publication No. WO 2013/174876, both of which are incorporated by reference in their entireties herein. In some embodiments, a Trk inhibitor can be any disclosed in U.S. Publication No. 2015/0283132, International Publication No. WO 2015/124697, U.S. Pat. No. 8,946,226, International Publication No. WO 2010/012733, U.S. Pat. No. 8,912,194, and International Publication No. WO 2010/058006, all of which are incorporated by reference in their entireties herein.

Additional examples of Trk inhibitors can be found in U.S. Publication No. International Publication No. WO 2015/017533, which is incorporated by reference in its entirety herein.

Further examples of Trk inhibitors can be found in U.S. Publication No. 2016/0272725 and International Publication No. WO 2015/112806, both of which are incorporated by reference in their entirety herein. For example, a Trk inhibitor can be a compound of Formula (I-A):

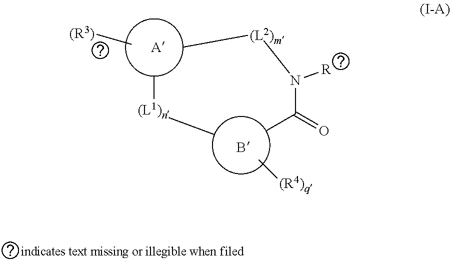

or a pharmaceutically acceptable salt thereof; or a pharmaceutically acceptable salt thereof. Exemplary Trk inhibitors include TPX-0005 (repotrectinib; (7S,13R)-11-fluoro-6,7,13,14-tetrahydro-7,13-dimethyl-1,15-etheno-1H-pyrazolo[4,3-f][1,4,8,10]benzoxatriazacyclotridecin-4(5H)-one).

A Trk inhibitor can be one found in U.S. Pat. No. 9,187,489 and International Publication No. WO 2013/183578, both of which are incorporated by reference in their entireties herein. Exemplary Trk inhibitors include PLX7486 and DS-6051.

Non-limiting examples of Trk inhibitors can be found in U.S. Publication No. 2015/0306086 and International Publication No. WO 2013/074518, both of which are incorporated by reference in their entireties herein. Exemplary Trk inhibitors include TSR-011.

Further examples of Trk inhibitors can be found in U.S. Pat. No. 8,637,516, International Publication No. WO 2012/034091, U.S. Pat. No. 9,102,671, International Publication No. WO 2012/116217, U.S. Publication No. 2010/0297115, International Publication No. WO 2009/053442, U.S. Pat. No. 8,642,035, International Publication No. WO 2009092049, U.S. Pat. No. 8,691,221, International Publication No. WO2006131952, all of which are incorporated by reference in their entireties herein. Exemplary Trk inhibitors include GNF-4256, described in Cancer Chemother. Pharmacol. 75(1):131-141, 2015; and GNF-5837 (N-[3-[[2,3-dihydro-2-oxo-3-(1H-pyrrol-2-ylmethylene)-1H-indol-6-yl]amino]-4-methylphenyl]-N′-[2-fluoro-5-(trifluoromethyl)phenyl]-urea), described in ACS Med. Chem. Lett. 3(2):140-145, 2012, each of which is incorporated by reference in its entirety herein.

Additional examples of Trk inhibitors include those disclosed in U.S. Publication No. 2010/0152219, U.S. Pat. No. 8,114,989, and International Publication No. WO 2006/123113, all of which are incorporated by reference in their entireties herein. Exemplary Trk inhibitors include AZ623, described in Cancer 117(6):1321-1391, 2011; AZD6918, described in Cancer Biol. Ther. 16(3):477-483, 2015; AZ64, described in Cancer Chemother. Pharmacol. 70:477-486, 2012; AZ-23 ((S)-5-Chloro-N2-(1-(5-fluoropyridin-2-yl)ethyl)-N4-(5-isopropoxy-1H-pyrazol-3-yl)pyrimidine-2,4-diamine), described in Mol. Cancer Ther. 8:1818-1827, 2009; and AZD7451; each of which is incorporated by reference in its entirety.

A Trk inhibitor can include those described in U.S. Pat. Nos. 7,615,383; 7,384,632; 6,153,189; 6,027,927; 6,025,166; 5,910,574; 5,877,016; and 5,844,092, each of which is incorporated by reference in its entirety.

Further examples of Trk inhibitors include CEP-751, described in Int. J. Cancer 72:672-679, 1997; CT327, described in Acta Derm. Venereol. 95:542-548, 2015; compounds described in International Publication No. WO 2012/034095; compounds described in U.S. Pat. No. 8,673,347 and International Publication No. WO 2007/022999; compounds described in U.S. Pat. No. 8,338,417; compounds described in International Publication No. WO 2016/027754; compounds described in U.S. Pat. No. 9,242,977; compounds described in U.S. Publication No. 2016/0000783; sunitinib (N-(2-diethylaminoethyl)-5-[(Z)-(5-fluoro-2-oxo-1H-indol-3-ylidene)methyl]-2,4-dimethyl-1H-pyrrole-3-carboxamide), as described in PLoS One 9:e95628, 2014; compounds described in International Publication No. WO 2011/133637; compounds described in U.S. Pat. No. 8,637,256; compounds described in Expert. Opin. Ther. Pat. 24(7):731-744, 2014; compounds described in Expert Opin. Ther. Pat. 19(3):305-319, 2009; (R)-2-phenylpyrrolidine substituted imadizopyridazines, e.g., (4-((5-chloro-4-(methylamino)-7H-pyrrolo[2,3-d]pyrimidin-2-yl)amino)-3-methoxyphenyl)(morpholino)methanone as described in ACS Med. Chem. Lett. 6(5):562-567, 2015; GTx-186 and others, as described in PLoS One 8(12):e83380, 2013; K252a ((9S-(9α,10β,12α))-2,3,9,10,11,12-hexahydro-10-hydroxy-10-(methoxycarbonyl)-9-methyl-9,12-epoxy-1H-diindolo[1,2,3-fg:3′,2′,1′-kl]pyrrolo[3,4-i][1,6]benzodiazocin-1-one), as described in Mol. Cell Biochem. 339(1-2):201-213, 2010; 4-aminopyrazolylpyrimidines, e.g., AZ-23 (((S)-5-chloro-N2-(1-(5-fluoropyridin-2-yl)ethyl)-N4-(5-isopropoxy-1H-pyrazol-3-yl)pyrimidine-2,4-diamine)), as described in J. Med. Chem. 51(15):4672-4684, 2008; PHA-739358 (danusertib), as described in Mol. Cancer Ther. 6:3158, 2007; Go 6976 (5,6,7,13-tetrahydro-13-methyl-5-oxo-12H-indolo[2,3-a]pyrrolo[3,4-c]carbazole-12-propanenitrile), as described in J. Neurochem. 72:919-924, 1999; GW441756 ((3Z)-3-[(1-methylindol-3-yl)methylidene]-1H-pyrrolo[3,2-b]pyridin-2-one), as described in IJAE 115:117, 2010; milciclib (PHA-848125AC), described in J. Carcinog. 12:22, 2013; AG-879 ((2E)-3-[3,5-Bis(1,1-dimethylethyl)-4-hydroxyphenyl]-2-cyano-2-propenethioamide); altiratinib (N-(4-((2-(cyclopropanecarboxamido)pyridin-4-yl)oxy)-2,5-difluorophenyl)-N-(4-fluorophenyl)cyclopropane-1,1-dicarboxamide); cabozantinib (N-(4-((6,7-Dimethoxyquinolin-4-yl)oxy)phenyl)-N′-(4-fluorophenyl)cyclopropane-1,1-dicarboxamide); lestaurtinib ((5S,6S,8R)-6-Hydroxy-6-(hydroxymethyl)-5-methyl-7,8,14,15-tetrahydro-5H-16-oxa-4b,8a,14-triaza-5,8-methanodibenzo[b,h]cycloocta[jkl]cyclopenta[e]-as-indacen-13(6H)-one); dovatinib (4-amino-5-fluoro-3-[6-(4-methylpiperazin-1-yl)-1H-benzimidazol-2-yl]quinolin-2(1H)-one mono 2-hydroxypropanoate hydrate); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); ONO-5390556; regorafenib (4-[4-({[4-Chloro-3-(trifluoromethyl)phenyl]carbamoyl}amino)-3-fluorophenoxy]-N-methylpyridine-2-carboxamide hydrate); VSR-902A; all of the references above are incorporated by reference in their entireties herein.

In some embodiments, a Trk inhibitor is one or more compounds of Table 1, or a pharmaceutically acceptable salt thereof.

TABLE 1
Exemplary Trk inhibitors

| Compound | Compound | Compound |
|---|---|---|
| No. | Structure | Name |
| 1 | 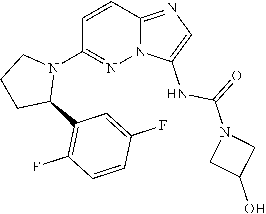 [SMILES: O=C(Nc1cnc2ccc(N3CCC[C@@H]3c3cc(F)ccc3F)nn12)N1CC(O)C1] | (R)-N-(6-(2-(2,5- difluorophenyl)pyrrolidin-1- yl)imidazo[1,2-b]pyridazin-3-yl)-3- hydroxyazetidine-1-carboxamide |
| 2 | 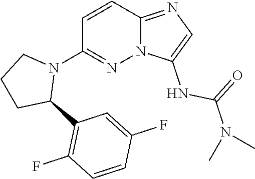 [SMILES: CN(C)C(=O)Nc1cnc2ccc(N3CCC[C@@H]3c3cc(F)ccc3F)nn12] | (R)-3-(6-(2-(2,5- difluorophenyl)pyrrolidin-1- yl)imidazo[1,2-b]pyridazin-3-yl)-1,1- dimethylurea |
| 3 | 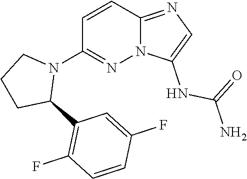 [SMILES: NC(=O)Nc1cnc2ccc(N3CCC[C@@H]3c3cc(F)ccc3F)nn12] | (R)-1-(6-(2-(2,5- difluorophenyl)pyrrolidin-1- yl)imidazo[1,2-b]pyridazin-3-yl)urea |
| 4 | 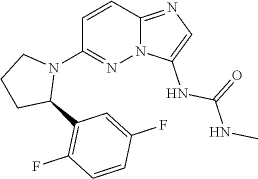 [SMILES: CNC(=O)Nc1cnc2ccc(N3CCC[C@@H]3c3cc(F)ccc3F)nn12] | (R)-1-(6-(2-(2,5- difluorophenyl)pyrrolidin-1- yl)imidazo[1,2-b]pyridazin-3-yl)-3- methylurea |
| 5 | 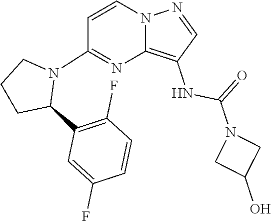 [SMILES: O=C(Nc1cnn2ccc(N3CCC[C@@H]3c3cc(F)ccc3F)nc12)N1CC(O)C1] | (R)-N-(5-(2-(2,5- difluorophenyl)pyrrolidin-1- yl)pyrazolo[1,5-a]pyrimidin-3-yl)-3- hydroxyazetidine-1-carboxamide |
| 6 | 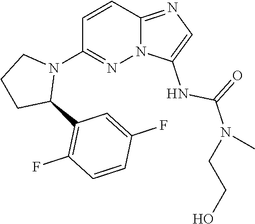 [SMILES: CN(CCO)C(=O)Nc1cnc2ccc(N3CCC[C@@H]3c3cc(F)ccc3F)nn12] | (R)-3-(6-(2-(2,5- difluorophenyl)pyrrolidin-1- yl)imidazo[1,2-b]pyridazin-3-yl)-1-(2- hydroxyethyl)-1-methylurea |
| 7 | 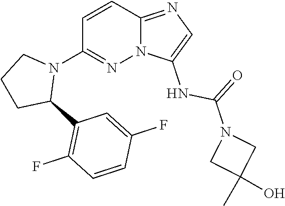 [SMILES: CC1(O)CN(C(=O)Nc2cnc3ccc(N4CCC[C@@H]4c4cc(F)ccc4F)nn23)C1] | (R)-N-(6-(2-(2,5- difluorophenyl)pyrrolidin-1- yl)imidazo[1,2-b]pyridazin-3-yl)-3- hydroxy-3-methylazetidine-1- carboxamide |
| 8 | 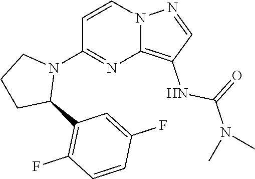 [SMILES: CN(C)C(=O)Nc1cnn2ccc(N3CCC[C@@H]3c3cc(F)ccc3F)nc12] | (R)-3-(5-(2-(2,5- difluorophenyl)pyrrolidin-1- yl)pyrazolo[1,5-a]pyrimidin-3-yl)-1,1- dimethylurea |
| 9 | 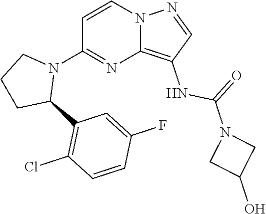 [SMILES: O=C(Nc1cnn2ccc(N3CCC[C@@H]3c3cc(F)ccc3Cl)nc12)N1CC(O)C1] | (R)-N-(5-(2-(2-chloro-5- fluorophenyl)pyrrolidin-1- yl)pyrazolo[1,5-a]pyrimidin-3-yl)-3- hydroxyazetidine-1-carboxamide |
| 10 | 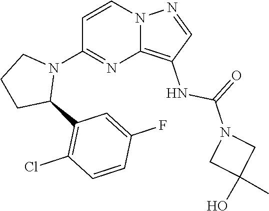 [SMILES: CC1(O)CN(C(=O)Nc2cnn3ccc(N4CCC[C@@H]4c4cc(F)ccc4Cl)nc23)C1] | (R)-N-(5-(2-(2-chloro-5- fluorophenyl)pyrrolidin-1- yl)pyrazolo[1,5-a]pyrimidin-3-yl)- 3-hydroxy-3-methylazetidine-1- carboxamide |
| 11 | 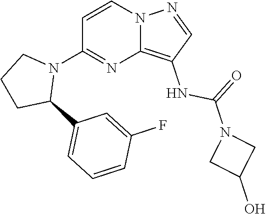 [SMILES: O=C(Nc1cnn2ccc(N3CCC[C@@H]3c3cccc(F)c3)nc12)N1CC(O)C1] | (R)-N-(5-(2-(3-fluorophenyl)pyrrolidin- 1-yl)pyrazolo[1,5-a]pyrimidin-3-yl)-3- hydroxyazetidine-1-carboxamide |
| 12 | 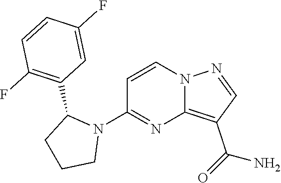 [SMILES: NC(=O)c1cnn2ccc(N3CCC[C@@H]3c3cc(F)ccc3F)nc12] | (R)-5-(2-(2,5-difluorophenyl)pyrrolidin- 1-yl)pyrazolo[1,5-a]pyrimidine-3- carboxamide |
| 13 | 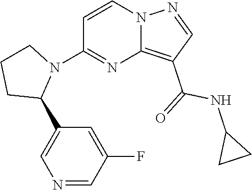 [SMILES: O=C(NC1CC1)c1cnn2ccc(N3CCC[C@@H]3c3cncc(F)c3)nc12] | (R)-N-cyclopropyl-5-(2-(5-fluoropyridin- 3-yl)pyrrolidin-1-yl)pyrazolo[1,5- a]pyrimidine-3-carboxamide |
| 14 | 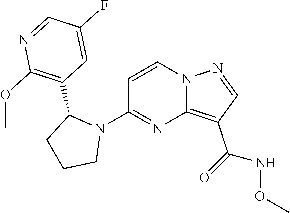 [SMILES: CONC(=O)c1cnn2ccc(N3CCC[C@@H]3c3cc(F)cnc3OC)nc12] | (R)-5-(2-(5-fluoro-2-methoxypyridin-3- yl)pyrrolidin-1-yl)-N- methoxypyrazolo[1,5- a]pyrimidine-3-carboxamide |
| 15 | 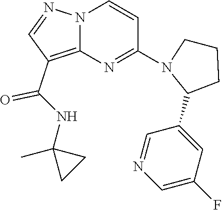 | (R)-5-(2-(5-fluoropyridin-3- yl)pyrrolidin-1-yl)-N-(1- methylcyclopropyl)pyrazolo[1,5- a]pyrimidine-3-carboxamide |
| 16 | 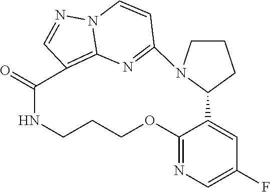 [SMILES: O=C1NCCCOc2ncc(F)cc2[C@H]2CCCN2c2ccn3ncc1c3n2] | (6R)-9-fluoro-13-oxa-2, 11, 17,21,22,25- hexaazapentacyclo[17.5.2.0^{2,6}.0^{7,12}.0^{22,26}]- hexacosa-1(25),7,9,11,19(26),20,23- heptaen-18-one |
| 17 | 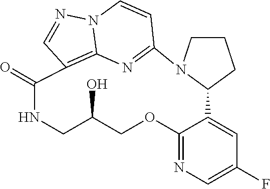 [SMILES: O=C1NC[C@@H](O)COc2ncc(F)cc2[C@H]2CCCN2c2ccn3ncc1c3n2] | (6R,15R)-9-fluoro-15-hydroxy-13-oxa-2, 11, 17,21,22,25-hexaazapentacyclo- [17.5.2.0^{2,6}.0^{7,12}.0^{22,26}]-hexacosa- 1(25),7,9,11, 19(26),20,23-heptaen-18- one |
| 18 | 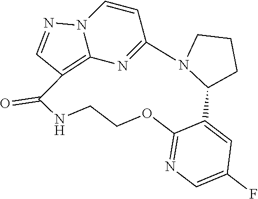 [SMILES: O=C1NCCOc2ncc(F)cc2[C@H]2CCCN2c2ccn3ncc1c3n2] | (6R)-9-fluoro-13-oxa-2,11,18,22,23,26- hexaazapentacyclo[18.5.2.0^{2,6}.0^{7,12}.0^{23,27}]- heptacosa-1(26),7,9,11,20(27),21,24- heptaen-19-one |
| 19 | 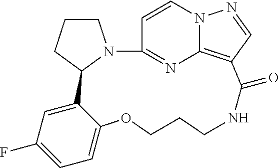 [SMILES: O=C1NCCCOc2ccc(F)cc2[C@H]2CCCN2c2ccn3ncc1c3n2] | (6R)-9-fluoro-13-oxa-2,17,21,22,25- pentaazapentacyclo[17.5.2.02,6.07,12.022,26]- hexacosa-1(25),7,9,11,19(26),20,23- heptaen-18-one |
| 20 | 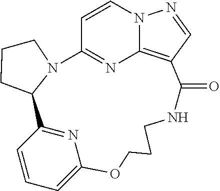 [SMILES: O=C1NCCCOc2cccc(n2)[C@H]2CCCN2c2ccn3ncc1c3n2] | (6R)-12-oxa-2,16,20,21,24,26- hexaazapentacyclo[16.5.2.^{17,11}.0^{2,6}.0^{21,25}]- hexacosa-1(24),7(26),8,10,18(25),19,22- heptaen-17-one |
| 21 | 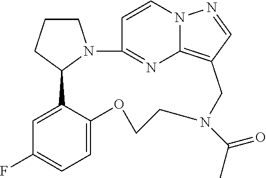 [SMILES: CC(=O)N1CCOc2ccc(F)cc2[C@H]2CCCN2c2ccn3ncc(c3n2)C1] | 1-[(6R)-9-fluoro-13-oxa-2,16,20,21,24- pentaazapentacyclo[16.5.2.0^{2,6}.0^{7,12}.0^{21,25}]- pentacosa-1(24),7,9,11,18(25),19,22- heptaen-16-yl]ethan-1-one |
| 22 | 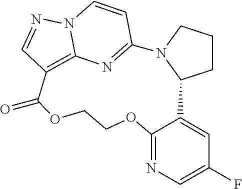 [SMILES: O=C1OCCOc2ncc(F)cc2[C@H]2CCCN2c2ccn3ncc1c3n2] | (6R)-9-fluoro-13,16-dioxa- 2,11,20,21,24- pentaazapentacyclo[16.5.2.0^{2,6}.0^{7,12}.0^{21,25}]- pentacosa-1(24),7,9,11,18(25),19,22- heptaen-17-one |
| 23 | 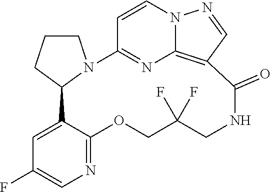 [SMILES: O=C1NCC(F)(F)COc2ncc(F)cc2[C@H]2CCCN2c2ccn3ncc1c3n2] | (6R)-9,15,15-trifluoro-13-oxa- 2,11,17,21,22,25- hexaazapentacyclo[17.5.2.0^{2,6}.0^{7,12}.0^{22,26}] hexacosa-1(25),7,9,11,19(26),20,23- heptaen-18-one |
| 24 | 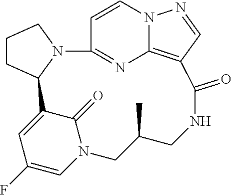 [SMILES: C[C@H]1CNC(=O)c2cnn3ccc(nc23)N2CCC[C@@H]2c2cc(F)cn(c2=O)C1] | (6R,13S)-9-fluoro-13-methyl- 2,11,15,19,20,23- hexaazapentacyclo[15.5.2.^{17,11}.0^{2,6}.0^{20,24}] pentacosa-1(23),7,9,17(24),18,21- hexaene-16,25-dione |
| 25 | 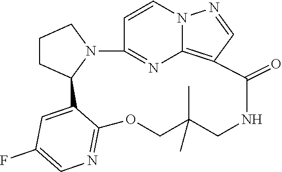 [SMILES: CC1(C)CNC(=O)c2cnn3ccc(nc23)N2CCC[C@@H]2c2cc(F)cnc2OC1] | (6R)-9-fluoro-15,15-dimethyl-13-oxa- 2,11,17,21,22,25-hexaazapentacyclo- [17.5.2.0^{2,6}.0^{7,12}.0^{22,26}]hexacosa- 1(25),7,9,11,19(26),20,23-heptaen-18- one |
| 26 | 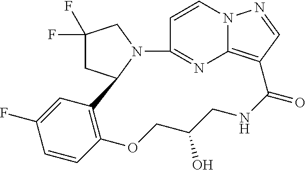 [SMILES: O=C1NC[C@H](O)COc2ccc(F)cc2[C@H]2CC(F)(F)CN2c2ccn3ncc1c3n2] | (15S)-4,4,9-trifluoro-15-hydroxy-13-oxa- 2,17,21,22.25- pentaazapentacyclo[17.5.2.0^{2,6}.0^{7,12}.0^{22,26}]- hexacosa-1(25),7(12),8,10,19(26),20,23- heptaen-18-one |
| 27 | 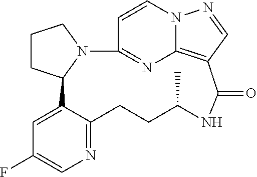 [SMILES: C[C@H]1CCc2ncc(F)cc2[C@H]2CCCN2c2ccn3ncc(c3n2)C(=O)N1] | (6R,15S)-9-fluoro-15-methyl- 2,11,16,20,21,24- hexaazapentacyclo[16.5.2.0^{2,6}.0^{7,12}.0^{21,25}] pentacosa-1(24),7,9,11,18(25),19,22- heptaen-17-one |
| 28 | 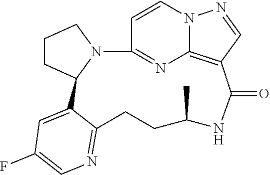 [SMILES: C[C@@H]1CCc2ncc(F)cc2[C@H]2CCCN2c2ccn3ncc(c3n2)C(=O)N1] | (6R,15R)-9-fluoro-15-methyl- 2,11,16,20,21,24- hexaazapentacyclo[16.5.2.0^{2,6}.0^{7,12}.0^{21,25}] pentacosa-1(24),7,9,11,18(25),19,22- heptaen-17-one |

Additional examples of Trk inhibitors are described in U.S. Patent Application Ser. No. 62/080,374, International Application Publication Nos. WO 11/006074, WO 11/146336, WO 10/033941, and WO 10/048314, and U.S. Pat. Nos. 8,933,084, 8,791,123, 8,637,516, 8,513,263, 8,450,322, 7,615,383, 7,384,632, 6,153,189, 6,027,927, 6,025,166, 5,910,574, 5,877,016, and 5,844,092, each of which is herein incorporated by reference in its entirety. Additional Trk inhibitors are known in the art.

In some embodiments, a Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide); (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate; cabozantinib ((N-(4-((6,7-Dimethoxyquinolin-4-yl)oxy)phenyl)-N′-(4-fluorophenyl)cyclopropane-1,1-dicarboxamide)); dovatinib (4-amino-5-fluoro-3-[6-(4-methylpiperazin-1-yl)-1H-benzimidazol-2-yl]quinolin-2(1H)-one mono 2-hydroxypropanoate hydrate); belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); PLX7486; altiratinib (N-(4-((2-(cyclopropanecarboxamido)pyridin-4-yl)oxy)-2,5-difluorophenyl)-N-(4-fluorophenyl)cyclopropane-1,1-dicarboxamide); and AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine)). For example, a first Trk inhibitor can be entrectinib or S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate (or a polymorph thereof).

### Immunotherapy

The term “immunotherapy” refers to an agent that modulates the immune system. In some embodiments, an immunotherapy can increase the expression and/or activity of a regulator of the immune system. In some embodiments, an immunotherapy can decrease the expression and/or activity of a regulator of the immune system. In some embodiments, an immunotherapy can recruit and/or enhance the activity of an immune cell.

In some embodiments, the immunotherapy is a cellular immunotherapy (e.g., adoptive T-cell therapy, dendritic cell therapy, natural killer cell therapy). In some embodiments, the cellular immunotherapy is sipuleucel-T (APC8015; Provenge™; Plosker (2011) Drugs 71(1): 101-108). In some embodiments, the cellular immunotherapy includes cells that express a chimeric antigen receptor (CAR). In some embodiments, the cellular immunotherapy is a CAR-T cell therapy. In some embodiments, the CAR-T cell therapy is tisagenlecleucel (Kymriah™).

In some embodiments, the immunotherapy is an antibody therapy (e.g., a monoclonal antibody, a conjugated antibody). In some embodiments, the antibody therapy is bevacizumab (Mvasti™, Avastin®), trastuzumab (Herceptin®), avelumab (Bavencio®), rituximab (MabThera™, Rituxan®), edrecolomab (Panorex), daratumuab (Darzalex®), olaratumab (Lartruvo™), ofatumumab (Arzerra®), alemtuzumab (Campath®), cetuximab (Erbitux®), oregovomab, pembrolizumab (Keytruda®), dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), avelumab (Bavencio®), necitumumab (Portrazza™) cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), ganitumab, urelumab, pidilizumab or amatuximab.

In some embodiments, the immunotherapy is an antibody-drug conjugate. In some embodiments, the antibody-drug conjugate is gemtuzumab ozogamicin (Mylotarg™), inotuzumab ozogamicin (Besponsa®), brentuximab vedotin (Adcetris®), ado-trastuzumab emtansine (TDM-1; Kadcyla®), mirvetuximab soravtansine (IMGN853) or anetumab ravtansine

In some embodiments, the immunotherapy includes blinatumomab (AMG103; Blincyto®) or midostaurin (Rydapt).

In some embodiments, the immunotherapy includes a toxin. In some embodiments, the immunotherapy is denileukin diftitox (Ontak®).

In some embodiments, the immunotherapy is a cytokine therapy. In some embodiments, the cytokine therapy is an interleukin 2 (IL-2) therapy, an interferon alpha (IFNα) therapy, a granulocyte colony stimulating factor (G-CSF) therapy, an interleukin 12 (IL-12) therapy, an interleukin 15 (IL-15) therapy, an interleukin 7 (IL-7) therapy or an erythropoietin-alpha (EPO) therapy. In some embodiments, the IL-2 therapy is aldesleukin (Proleukin®). In some embodiments, the IFNα therapy is interferon alfa-2b (e.g., IntronA®) or interferon alfa-2a (e.g., Roferon-A®). In some embodiments, the G-CSF therapy is filgrastim (Neupogen®).

In some embodiments, the immunotherapy is an immune checkpoint inhibitor. In some embodiments, the immunotherapy includes one or more immune checkpoint inhibitors. In some embodiments, the immune checkpoint inhibitor is a CTLA-4 inhibitor, a PD-1 inhibitor or a PD-L1 inhibitor. In some embodiments, the CTLA-4 inhibitor is ipilimumab (Yervoy®) or tremelimumab (CP-675,206). In some embodiments, the PD-1 inhibitor is pembrolizumab (Keytruda®) or nivolumab (Opdivo®). In some embodiments, the PD-L1 inhibitor is atezolizumab (Tecentriq®), avelumab (Bavencio®) or durvalumab (Imfinzi™).

In some embodiments, the immunotherapy is mRNA-based immunotherapy. In some embodiments, the mRNA-based immunotherapy is CV9104 (see, e.g., Rausch et al. (2014) Human Vaccin Immunother 10(11): 3146-52; and Kubler et al. (2015) J. Immunother Cancer 3:26).

In some embodiments, the immunotherapy is bacillus Calmette-Guerin (BCG) therapy.

In some embodiments, the immunotherapy is an oncolytic virus therapy. In some embodiments, the oncolytic virus therapy is talimogene alherparepvec (T-VEC; Imlygic®).

In some embodiments, the immunotherapy is a cancer vaccine. In some embodiments, the cancer vaccine is a human papillomavirus (HPV) vaccine. In some embodiments, the HPV vaccine is a recombinant human papillomavirus vaccine [types 6, 11, 16, and 18] (Gardasil®); a recombinant human papillomavirus vaccine [types 6, 11, 16, 18, 31, 33, 45, 52, and 58] (Gardasil9®); or a recombinant human papillomavirus vaccine [types 16 and 18] (Cervarix®). In some embodiments, the cancer vaccine is a hepatitis B virus (HBV) vaccine. In some embodiments, the HBV vaccine is Engerix-B®, Recombivax HB® or GS-4774 (GI-13020 or Tarmogen®). In some embodiments, the cancer vaccine is a combination Hepatitis A and Hepatitis B vaccine (e.g., Twinrix®) or a combination diphtheria, tetanus, pertussis, hepatitis B virus, and poliomyelitis vaccine (e.g., Pediarix®). In some embodiments, the cancer vaccine is dasiprotimut-T (BiovaxID®), an HSPPC-96 vaccine (e.g., Oncophage®), GVAX, ADXS11-001, ALVAC-CEA, rilimogene galvacirepvec/rilimogene glafolivec (PROSTVAC®), CDX-110 (Rindopepimut®), CimaVax-EGF, lapuleucel-T (APC8024; Neuvenge™) GRNVAC1, GRNVAC2, GRN-1201, hepcortespenlisimut-L (Hepko-V5), a dendritic cell vaccine (e.g., DCVax-L®, ICT-107), SCIB1, BMT CTN 1401, PrCa VBIR, PANVAC, a prostate cancer vaccine (e.g., ProstAtak®), DPX-Survivac, or viagenpumatucel-L (HS-110).

In some embodiments, the immunotherapy is a peptide vaccine. In some embodiments, the peptide vaccine is nelipepimut-S (E75) (NeuVax™), IMA901, or SurVaxM (SVN53-67). In some embodiments, the cancer vaccine is an immunogenic personal neoantigen vaccine (see, e.g., Ott et al. (2017) Nature 547: 217-221; Sahin et al. (2017) Nature 547: 222-226). In some embodiments, the cancer vaccine is RGSH4K, or NEO-PV-01.

In some embodiments, the cancer vaccine is a DNA-based vaccine. In some embodiments, the DNA-based vaccine is a mammaglobin-A DNA vaccine (see, e.g., Kim et al. (2016) OncoImmunology 5(2): e1069940).

### Methods for Treating Cancer

In some embodiments, provided herein is a method for treating a patient diagnosed with a TRK-associated cancer, comprising administering to the patient a therapeutically effective amount of one or more Trk inhibitors and optionally an immunotherapy agent. The Trk family of neurotrophin receptors, TrkA, TrkB, and TrkC (encoded by NTRK1, NTRK2, and NTRK3 genes, respectively) and their neurotrophin ligands regulate growth, differentiation and survival of neurons. Dysregulation in a NTRK gene, a Trk protein, or expression or activity, or level of the same, such as translocations involving the NTRK kinase domain, mutations involving the TRK ligand-binding site, amplifications of a NTRK gene, Trk mRNA splice variants, and Trk autocrine/paracrine signaling are described in a diverse number of tumor types and may contribute to tumorigenesis. Recently NTRK1 fusions were described in a subset of adenocarcinoma lung cancer patients. Translocations in NTRK1, NTRK2, and NTRK3 that lead to the production of constitutively-active TrkA, TrkB, and TrkC fusion proteins are oncogenic and prevalent in a wide array of tumor types, including lung adenocarcinoma, thyroid, head and neck cancer, glioblastoma, and others.

In some embodiments, the dysregulation in a NTRK gene, a Trk protein, or expression or activity, or level of the same, includes overexpression of wild-type TrkA, TrkB, or TrkC (e.g., leading to autocrine activation). In some embodiments, the dysregulation in a NTRK gene, a Trk protein, or expression or activity, or level of the same, includes overexpression, activation, amplification or mutation in a chromosomal segment comprising the NTRK1, NTRK2, or NTRK3 gene or a portion thereof. In some embodiments, the dysregulation of a NTRK gene, a Trk protein, or expression or activity, or level of the same, includes one or more chromosome translocations or inversions resulting in NTRK1, NTRK2, or NTRK3 gene fusions, respectively. In some embodiments, the dysregulation of a NTRK gene, a Trk protein, or expression or activity, or level of the same, is a result of genetic translocations in which the expressed protein is a fusion protein containing residues from a non-TrkA partner protein and TrkA, a non-TrkB partner protein and TrkB, or a non-TrkC partner protein and TrkC proteins, and include a minimum of a functional TrkA, TrkB, or TrkC kinase domain, respectively.

In some embodiments, a TrkA fusion protein is one of the TrkA fusion proteins shown in Table 2.

TABLE 2
Exemplary TrkA Fusion Proteins and Cancers

| | | Non-limiting Exemplary TrkA |
|---|---|---|
| | | Fusions and Synonyms of |
| Fusion Protein | Non-TrkA Fusion Partner | Associated Cancer(s) |
| TP53-TrkA^{1, 2} | Tumor Protein P53 | Spitzoid Neoplasms³, Spitz |
| | | Tumors¹, Pediatric High-Grade |
| | | Glioma² |
| LMNA-TrkA¹⁷ | Lamin A/C | Spitzoid Neoplasms¹, Spitz |
| | | Tumors ⁴, Sarcoma⁶³ (e.g., Adult |
| | | Soft Tissue Sarcoma¹², Spindle |
| | | Cell Sarcoma including Uterine |
| | | Spindle Cell Sarcoma⁶ and |
| | | Paediatric Haemangiopericytoma- |
| | | Like Sarcoma⁵), Congenital |
| | | Infantile Fibrosarcoma^{7, 65}, |
| | | Colorectal Cancer^{8,18}, Pediatric |
| | | Soft Tissue Tumor⁹, Soft Tissue |
| | | Primitive Neuroectodermal |
| | | Tumor⁶⁴, Lipofibromatosis-like |
| | | Neural Tumor (LPF-NT)^{10, 11}, |
| | | Histiocytic Neoplasms^{13, 14} (e.g., |
| | | Non-Langerhan Histocytosis¹⁵), |
| | | Melanoma¹⁷ (e.g., Skin Cutaneous |
| | | Melanoma⁵⁷) |
| CD74-TrkA¹⁹ | MHC class II invariant chain | Lung Adenocarcinoma²⁰ |
| TFG-TrkA (TRK- | TRK-Fused Gene | Papillary Thyroid Carcinoma |
| T3)²¹ | | (PTC)^{22, 54}, Histiocytic |
| | | Neoplasms¹⁴, Thyroid |
| | | Carcinoma⁵⁷ |
| TPM3-TrkA²¹ | Tropomyosin 3 | Non-Small Cell Lung Cancer⁶³, |
| | | Papillary Thyroid Carcinoma |
| | | (PTC)^{21, 53}, Sarcoma^{37, 57} (e.g., |
| | | Spindle Cell Uterine Sarcoma⁶, |
| | | Pediatric Spindle Cell Sarcoma²⁴, |
| | | Uterine Leiomyosarcoma |
| | | (LMS)²⁵, Spindle Cell Sarcoma |
| | | with a Prominent |
| | | Myopericytic/Haemangiopericytic |
| | | Pattern⁵), Glioblastoma⁶³, |
| | | Colorectal Cancer (CRC)^{8, 16, 51, 56}, |
| | | Soft Tissue Schwannoma¹², |
| | | Spitzoid Melanocytic Tumors²³, |
| | | Lipofibromatosis-Like Neural |
| | | Tumors(LPF-NT)¹¹, |
| | | Lipofibromatosis (LPF)²⁶, |
| | | Bladder Urothelial Carcinoma⁵⁷, |
| | | Gall Bladder Cancer⁶³, |
| | | Cholangiocarcinoma⁶³ |
| NFASC-TrkA³⁰ | Neurofascin | Gliobastoma multiforme |
| | | (GBM)^{27, 30, 60} |
| BCAN-TrkA²⁷ | Brevican | Glioma (e.g., Glioblastoma |
| | | Multiforme (GBM)²⁷, High-Grade |
| | | Glioma²⁸, Glioneuronal Tumor^{29, 61}, |
| | | Pilocytic Astrocytoma³¹) |
| MPRIP-TrkA^{19, 32} | Myosin Phosphatase Rho | Lung Adenocarcinoma^{15, 33} |
| | Interacting Protein or Rho | |
| | Interacting Protein 3 | |
| TPR-TrkA (e.g., | Translocated Promoter Region, | Papillary Thyroid Carcinoma |
| TRK-T1 or TRK- | Nuclear Basket Protein | (PTC)^{62, 67}, Post-Chernobyl |
| T2)²⁷ | | Radiation-Induced Thyroid |
| | | cancer⁴⁵, Colorectal Cancer |
| | | (CRC)³⁴, LPF-Like Neural |
| | | Tumors², Sporadic Pediatric |
| | | Differentiated Thyroid |
| | | Carcinomas (DTC)³⁵, Spindle |
| | | Cell Uterine Sarcoma⁶, |
| | | Myofibroma/Myofibromatosis²⁶, |
| | | Dendritic Cell Neoplasm¹⁴ |
| RFWD2-TrkA³⁶ | Ring Finger and WD Repeat | Large Cell Neuroendrocine |
| | Domain 2 | Cancer (LCNEC)³⁶ |
| IRF2BP2-TrkA⁴⁴ | Interferon Regulatory Factor 2 | Thyroid Gland Carcinoma^{44, 59}, |
| | Binding Protein 2 | Thyroid Carcinoma⁵⁷, Non-Small |
| | | Cell Lung Cancer⁶³, Prostate |
| | | Cancer⁷¹ |
| SQSTM1-TrkA⁴⁴ | Sequestosome 1 | Thyroid Cancer (e.g., Papillary |
| | | Thyroid Cancer⁶³, Thyroid Gland |
| | | Carcinoma⁵⁹), Soft Tissue |
| | | Fibrosarcoma¹², Non-Small Cell |
| | | Lung Cancer^{38, 39}, Lung |
| | | Adenocarcinoma⁵⁸ |
| SSBP2-TrkA⁴⁴ | Single-Stranded DNA Binding | Thyroid Cancer⁵⁷ (e.g., Papillary |
| | Protein 2 | Thyroid Cancer); Thyroid Gland |
| | | Carcinoma⁵⁹ |
| RABGAP1L- | RAB GTPase Activating Protein | Intrahepatic Cholangiocarcinoma |
| TrkA⁴¹ | 1-Like | (ICC)⁴¹ |
| C180RF8-TrkA⁴⁷ | Chromosome 18 Open Reading | Non-Small Cell Lung Cancer |
| | Frame 8 | (NSCLC)⁴⁷ |
| RNF213-TrkA⁴⁷ | Ring Finger Protein 213 | Non-Small Cell Lung Cancer |
| | | (NSCLC)⁴⁷ |
| TBC1D22A- | TBC1 Domain Family, Member | Non-Small Cell Lung Cancer |
| TrkA⁴⁷ | 22A | (NSCLC)⁴⁷ |
| C20ORF112- | Chromosome 20 Open Reading | Non-Small Cell Lung Cancer |
| TrkA⁴⁷ | Frame 112 | (NSCLC)⁴⁷ |
| DNER-TrkA⁴⁷ | Delta/Notch-Like EGF Repeat | Non-Small Cell Lung Cancer |
| | Containing | (NSCLC)⁴⁷ |
| ARHGEF2- | Rho Guanine Nucleotide | Glioblastoma^{42, 43}, Sarcoma⁵⁷ |
| TrkA^{42, 57} | Exchange Factor 2 | |
| CHTOP-TrkA⁴² | Chromatin Target of PRMT1 | Glioblastoma⁴² |
| PPL-TrkA⁴² | Periplakin | Thyroid Carcinoma⁴² |
| PLEKHA6-TrkA | Pleckstrin Homology Domain- | Colon cancer⁷⁴ |
| | Containing Family A Member 6 | |
| PEAR1-TrkA⁶³ | Platelet Endothelial Aggregation | Sarcoma⁶³, Breast Cancer⁶³ |
| | Receptor 1 | |
| MRPL24-TrkA⁶³ | 39S Ribosomal Protein L24, | Non-Small Cell Lung Cancer⁶³ |
| | Mitochondrial | |
| MDM4-TrkA⁶³ | Human Homolog of Mouse | Breast Cancer⁶³ |
| | Double Minute 4 | |
| LRRC71-TrkA⁶³ | Leucine Rich Repeat Containing | Uterus Carcinoma⁶³ |
| | 71 | |
| GRIPAP1-TrkA⁶³ | GRIP1 Associated Protein 1 | Non-Small Cell Lung Cancer⁶³ |
| TAF-TrkA⁶³ | | Papillary Thyroid Carcinoma⁶³ |
| EPS15-TrkA | Epidermal Growth Factor | Lung cancer⁷¹ |
| | Receptor Substrate 15 | |
| DYNC2H1- | Dynein, Cytoplasmic 2, Heavy | Sarcoma |
| TrkA⁴⁴ | Chain 1 | |
| CEL-TrkA⁵⁷ | Carboxyl Ester Lipase | Pancreatic adenocarcinoma |
| | | sample⁵⁷ |
| EPHB2-TrkA⁴⁴ | EPH Receptor B2 | Lower Grade Glioma^{44, 57} |
| TGF-TrkA⁴⁶ | Transforming Growth Factor | Papillary Thyroid Cancer (PTC) |
| NELL1 -TrkA⁴⁷ | Cytoplasmic Protein That | Non-Small Cell Lung Cancer |
| | Contains Epidermal Growth | (NSCLC)⁴⁷ |
| | Factor (Egf)-Like Repeats | |
| EPL4-TrkA⁴⁷ | EPH-Related Receptor Tyrosine | Non-Small Cell Lung Cancer |
| | Kinase Ligand 4/ Ephrin-A4 | (NSCLC)⁴⁷ |
| | Protein | |
| CTNND2-TrkA⁴⁷ | Catenin (Cadherin-Associated | Non-Small Cell Lung Cancer |
| | Protein), Delta 2 | (NSCLC)⁴⁷ |
| TCEANC2- | Transcription Elongation Factor A | Non-Small Cell Lung Cancer |
| TrkA⁴⁷ | (Sll) N-Terminal And Central | (NSCLC)⁴⁷ |
| | Domain | |
| SCYL3-TrkA⁴⁸ | SCY1 Like Pseudokinase 3 | Colorectal cancer |
| AMOTL2-TrkA⁴⁹ | | Non-small cell lung cancer |
| MEF2D-TrkA⁵⁰ | | Glioma⁷⁶ |
| L7a-TrkA⁵⁵ (Trk- | | Breast carcinoma (human cell |
| 2h) | | line)⁵⁵ |
| ZBTB7B-TrkA⁵⁷ | | Bladder Urothelial Carcinoma⁵⁷ |
| TRIM63-TrkA⁶⁶ | | Non-Spitzoid Metastasizing |
| | | Melanomas⁶⁶ |
| DDR2-TrkA⁶⁶ | | Non-Spitzoid Metastasizing |
| | | Melanomas⁶⁶ |
| GON4L-TrkA⁶⁶ | | Non-Spitzoid Metastasizing |
| | | Melanomas⁶⁶ |
| PDE4DIP-TrkA | | Soft Tissue Sarcoma |
| | | (Myopericytoma) |
| NTRK1- | | Lung cancer⁷⁸ |
| P2RY8⁵²* | | |
| VANGL2-TrkA⁶⁸ | | Non-Small Cell Lung Cancer⁶⁸ |
| CTRC-TrkA⁷⁴ | Chymotrypsin C | Pancreatic Cancer⁷⁴ |
| ETV6-TrkA⁶⁹ | ETS Variant 6 | |
| COP1-TrkA | | Large cell neuroendocrine |
| | | cancer⁷³ |
| GATAD2B-TrkA | | Breast cancer⁷² |
| CGN-TrkA⁷² | | Breast cancer⁷² |
| AFAP1-TrkA⁷⁰ | | Glioblastoma⁷⁰ |
| AMOTL2-TrkA⁷¹ | | Lung cancer⁷¹ |
| DIAPH1-TrkA | | Thyroid cancer⁷⁵ |
| GSN-TrkA⁷⁶ | | |
| MIR548F1-TrkA | | Pediatric mesenchymal tumor⁷⁷ |
| PIP5K1A-TrkA | | Neuroendocrine tumor⁷⁹ |
| PRDX1-TrkA | | Lung cancer⁷¹ |

*The transcript of this fusion was not detected.
¹Wiesner et al., Nature Comm. 5: 3116, 2014.
²Wu et al., Nat. Genet. 46: 444-450, 2014.
³U.S. Patent Application Publication No. 2016/0010068.
⁴P.C.T. Patent Application Publication No. WO 2013/059740.
⁵Haller et al., J. Path. 238(5): 700-10, 2016.
⁶Chiang et al., Am. J. Surg. Pathol. doi: 10.1097/PAS.0000000000001055, 2018.
⁷Wong et al., J. Natl. Cancer Inst. 108(1): doi: 10.1093/jnci/djv307, 2016.
⁸Park et al., Oncotarget. 7(7): 8399-412, 2016.
⁹Kohsaka et al., Hum. Pathol. 72: 167-173, 2017.
¹⁰Bartenstein et al., JAAD Case Reports. 4(2): 185-188, 2018.
¹¹Agaram et al., Am. J. Surg. Pathol. 40(10): 1407-1416, 2016.
¹²Doebele et al., Cancer Discov. 5(10): 1049-1057, 2015.
¹³Durham et al. Blood. 126(23): 481, 2015.
¹⁴Taylor et al., Abstract Number: 794. Meeting Info: 59th Annual Meeting of the American Society of Hematology, ASH 2017. Atlanta, GA, United States, 2017.
¹⁵U.S. Patent Application Publication No. 2016/0009785.
¹⁶Sartore-Bianchi et al., J. Natl. Cancer Inst. 108(1). doi: 10.1093/jnci/djv306, 2015.
¹⁷U.S . Patent Application Publication No. 2014/0336236.
¹⁸P.C.T Patent Application Publication No. WO 2015/064621.
¹⁹Vaishnavi et al., Nature Med. 19: 1469-1472, 2013.
²⁰Doebele et al., Abstract Number: 8023. Meeting Info: 2013 Annual Meeting of the American Society of Clinical Oncology, ASCO. Chicago, IL, United States, 2013.
²¹Greco et al., Mol. Cell. Endocrinol. 28: 321, 2010.
²²Greco et al., Mol. Cell. Biol. 15(11): 6118-6127, 1995.
²³Wu et al., Mod Pathol. 29(4): 359-69, 2016.
²⁴Kao et al., Am. J. Surg. Pathol. 42(1): 28-38, 2018.
²⁵Elvin et al., Abstract Number: 319. Meeting Info: 26 EORTC-NCI-AACR Symposium on Molecular Targets and Cancer Therapeutics. Barcelona, Spain, 2014.
²⁶Agaram, et al., Abstract Number: 33. Meeting Info: 105th Annual Meeting of the United States and Canadian Academy of Pathology, USCAP 2016. Seattle, WA, United States, 2016.
²⁷Kim et al., PloS ONE. 9(3): e91940, 2014.
²⁸Cook et al., Nat. Comm. 8(15987). DOI 10.1038/ncomms15987, 2017.
²⁹Alvarez-Breckenridge et al., NPJ Precision Oncology. 1(5) doi: 10.1038/s41698-017-0009-y, 2017.
³⁰U.S. Patent Application Publication No. 2016/0108380.
³¹Subramaniam et al., Meeting Info: 2017 Annual Meeting of the American Society of Clinical Oncology, ASCO. Chicago, IL, United States, 2017.
³²U.S. Patent Application Publication No. 2015/0073036.
³³U.S. Patent Application Publication No. 2015/0218652.
³⁴Créancier et al., Cancer Lett. 365(1): 107-111, 2015.
³⁵Picarsic et al., Pediatr. Dev. Pathol. 19(2): 115-22, 2016.
³⁶Fernandez-Cuesta et al., Abstract Number: 1531. Meeting Info: 105th Annual Meeting of the American Association for Cancer Research, AACR 2014. San Diego, CA, United States, 2014.
³⁷Stransky et al., Nature Comm. 5: 4846, 2014.
³⁸Drilon et al., Abstract Number: CT007; 107^{th} Annual Meeting of the American Association for Cancer Research, AACR 2016. New Orleans, LA, 2016.
³⁹Farago et al., Abstract Number: MINI30.09. Meeting Info: 16th World Conference on Lung Cancer. Denver, CO, United States, 2015.
⁴¹Ross et al., Oncologist 19: 235-242, 2014.
⁴²Zheng et al., Nat. Med. 20(12): 1479-84, 2014.
⁴³P.C.T. Patent Application Publication No. WO 2015/039006.
⁴⁴U.S. Patent Application Publication No. 2015/0315657.
⁴⁵Ricarte-Filho et al., J. Clin. Invest. 123(11): 4935-44, 2013.
⁴⁶U.S. Patent Application Publication No. 2015/0283132.
⁴⁷U.S. Patent Application Publication No. 2017/0114415.
⁴⁸Milione et al., Oncotarget, 8(33): 55353-55360, 2017.
⁴⁹Chen et al., Abstract Number: 40. Meeting Info: 3rd Molecular Analysis for Personalised Therapy Conference, MAP 2017. Zurich, Switzerland, 2017.
⁵⁰Gatalica et al., Abstract Number: A047. Meeting Info: AACR-NCI-EORTC International Conference: Molecular Targets and Cancer Therapeutics 2017. Philadelphia, PA, United States, 2017
⁵¹Martin-Zanca et al., Nature. 319(6056): 743-8, 1986.
⁵²Hechtman et al., Mol. Cancer Res. 14(3): 296-301, 2016.
⁵³Butti et al., Genomics. 28(1): 15-24, 1995.
⁵⁴Brzeziańska et al., Neuro. Endocrinol. Lett. 28(3): 221-9, 2007.
⁵⁵Ziemiecki et al., EMBO J. 9(1): 191-6, 1990.
⁵⁶Ardini et al., Mol. Oncol. 8(8): 1495-1507, 2014.
⁵⁷Gao et al., Cell Rep. 23(1): 227-238.e3, 2018.
⁵⁸Farago et al., J. Thorac Oncol. 10(12): 1670-1674, 2015.
⁵⁹U.S. Patent Application Publication No. 2014/0315199.
⁶⁰Frattini et al., Nat. Genet. 45(10): 1141-1149, 2013.
⁶¹Bastianos et al., Abstract Number: OS06.4. Meeting Info: 5th Quadrennial Meeting of the World Federation of Neuro-Oncology Societies, WFNOS. Zurich, Switzerland, 2017.
⁶²Greco et al., Oncogene. 7(2): 237-42, 1992.
⁶³Wei et al., Abstract Number: 78. Meeting Info: 28th EORTC-NCI-AACR Symposium on Molecular Targets and Cancer Therapeutics. Munich, Germany, 2016.
⁶⁴Pavlick et al., Pediatr. Blood Cancer. 64(8). 2017.
⁶⁵Wong et al., J. Natl. Cancer Inst. 108(1), 2015.
⁶⁶Lezcano et al., Am. J. Surg. Pathol. doi: 10.1097/PAS.0000000000001070, 2018.
⁶⁷Greco et al., Genes Chromosomes Cancer. 19(2): 112-23, 1997.
⁶⁸Zehir et al., Nat. Med. 23(6): 703-713, 2017.
⁶⁹Fagan et al., Abstract Number: 5158. Meeting Info: American Association for Cancer Research Annual Meeting, 2017.
⁷⁰Schram A, et al. Cancer Res. 77 (Suppl; abstract LB-302), 2017.
⁷¹Ling Q, et al. Annals of Oncology. 29: (suppl 8; viii22), 2018.
⁷²Ross J, et al. Cancer Res. 78: suppl; abstract P2-09-15, 2018.
⁷³George J, et al. Nat. Commun. 9: 1048, 2018.
⁷⁴Drilon A, et al. N. Engl. J. Med. 378: 731-739, 2018.
⁷⁵Okamura R, et al. JCO Precision Oncology. 2018: 1-20.
⁷⁷Gatalica Z, et al. Mod. Pathol. 32(1): 147-153, 2019.
⁷⁸Rudzinski ER, et al. Am. J. Surg. Pathol. 42: 927-935, 2018.
⁷⁹Hechtman JF, et al. Am. J. Surg. Pathol. 41: 1547-1551, 2017.
⁸⁰Sigal DS, et al. Oncotarget. 9: 35809-35812, 2018.

In some embodiments, the dysregulation of a NTRK gene, a Trk protein, or expression or activity or level of any of the same, includes at least one point mutation in a NTRK gene that results in the production of a TrkA protein that has one or more amino acid substitutions, insertions, or deletions as compared to the wildtype TrkA protein (see, for example, the point mutations listed in Table 3). An exemplary wildtype TrkA polypeptide is SEQ ID NO: 1, an exemplary wildtype TrkB polypeptide is SEQ ID NO: 5, and an exemplary TrkC polypeptide is SEQ ID NO: 7.

TABLE 3
TrkA Kinase Protein Amino Acid Substitutions/Insertions/Deletions^{A}
Amino acid position 6 (e.g., R6W³)
Amino acid position 33 (e.g., R33W⁴)
Amino acid position 336 (e.g., A336E)
Amino acid position 337 (e.g., A337T)
Amino acid position 324 (e.g., R324Q, R324W)
Amino acid position 420 (e.g., V420M)
Amino acid position 444 (e.g., R444Q, R444W)
Amino acid position 517 (e.g., G517R, G517V)
Amino acid position 538 (e.g., K538A)
Amino acid position 542 (e.g., A542V)
Amino acid position 564 (e.g., L564H²)
Amino acid position 568 (e.g., Q568x)
Amino acid position 573 (e.g., V573M⁵)
Amino acid position 583 (e.g., R583H³)
Amino acid position 589 (e.g., F589L⁵, F589C)
Amino acid position 595 (e.g., G595S, G595R¹, G595L²)
Amino acid position 597* (e.g., Q597X⁷)
Amino acid position 598 (e.g., F598L⁵)
Amino acid position 599 (e.g., D596V)
Amino acid position 600 (e.g., F600L)
Amino acid position 602 (e.g., R602x)
Amino acid position 627* (e.g., Q627X⁷)
Amino acid position 633* (e.g., Q633X⁷)
Amino acid position 646 (e.g., F646V, F646I²)
Amino acid position 649 (e.g., R649W, R649L)
Amino acid position 656 (e.g., C656Y, C656F)
Amino acid position 657 (e.g., L657V)
Amino acid position 667 (e.g., G667C¹, G667S)
Amino acid position 676 (e.g., Y676S)
Amino acid position 679 (e.g., D679G²)
Amino acid position 682 (e.g., R682S)
Amino acid position 683 (e.g., V683G)
Amino acid position 699 (e.g., I699V⁶)
Amino acid position 702 (e.g., R702C)
Amino acid position 744 (e.g., R744H³)
^{A}The TrkA kinase mutations shown above may be activating mutations and/or may confer increased resistance of the TrkA kinase to a TrkA inhibitor e.g., as compared to a wildtype TrkA kinase.
*Q627XC, Q597XC, and Q633XC are from NP_001012331.1G⁸, NP_001007793.1F⁹, and the Reference TrkA sequence¹⁰, respectively.
¹Russo et al., Acquired Resistance to the TRK Inhibitor Entrectinib in Colorectal Cancer, Cancer Discov. 6(1): 36-44, 2016.
²Fuse et al., Mechanisms of Resistance to NTRK Inhibitors and Therapeutic Strategies in NTRK1-Rearranged Cancers, Mol. Cancer Ther. 6(1): 36-44, 2016.
³Iniguez-Ariza et al., Journal of Clinical Oncology, (20 Jun. 2017) Vol. 35, No. 15, Supp.1, 2017 Annual Meeting of the American Society of Clinical Oncology, ASCO, 2017.
⁴Zhang et al., Blood 124(21): 1682, 2014. Mutation found in T-cell prolymphocytic leukemia.
⁵PCT Application No. WO2016196141A1.
⁶Deihimi et al., Oncotarget. 8(25): 39945-39962. doi: 10.18632/oncotarget.18098, 2017.
⁷Park et al., Proc. Natl. Acad. Sci. U.S.A. 112(40): 12492-12497, 2015. Mutation found in colorectal cancer.
⁸www.ncbi.nlm.nib.gov/protein/59889558
⁹www.ncbi.nlm.nib.gov/protein/56118210?report=genbank&log$=protalign&blast_rank=3&RID=0
¹⁰Reference TrkA sequence is UniProtKB/Swiss-Prot: P04629.4, and can be found at URL: www.ncbi.nlm.nib.gov/protein/94730402?report=genbank&log$=protalign&blast_rank=0&RID=0

In some embodiments, the dysregulation of a NTRK gene, a Trk protein, or expression or activity, or level of the same, includes one or more deletions, insertions, or point mutation(s) in a TrkA protein. In some embodiments, the dysregulation of a NTRK gene, a Trk protein, or expression or activity, or level of the same, includes a deletion of one or more residues from the TrkA protein, resulting in constitutive activity of the TrkA kinase domain. In some embodiments, the deletion includes a deletion of amino acids 303-377 in TrkA isoform 2.

In some embodiments, the dysregulation of a NTRK gene, a Trk protein, or expression or activity, or level of the same, includes at least one point mutation in a NTRK1 gene that results in the production of a TrkA protein that has one or more amino acid substitutions as compared to the wildtype TrkA protein. In some embodiments, the at least one or more amino acid substitutions are activating mutations (see, for example, the point mutations listed in Table 4).

TABLE 4
Activating TrkA Point Mutations¹

| | | Exemplary Isoform in |
|---|---|---|
| | | which Mutation is Present |
| Point Mutation | Rationale | (if known) |
| R6W¹ | | |
| R33W² | | NP_001007793.1⁶ |
| A336E | Near NGF Binding Site | Reference TrkA sequence |
| A337T | Near NGF Binding Site | Reference TrkA sequence |
| R324Q or R324W | Near NGF Binding Site | Unknown |
| V420M | Close to Membrane | Reference TrkA sequence |
| R444Q or R444W | Close to Membrane | Reference TrkA sequence |
| G517R or G517V | P-Loop | Reference TrkA sequence |
| K538A | Activating | Reference TrkA sequence |
| R583H⁹ | | |
| F598L⁵ | | Unknown |
| R649W or R649L | Arginine may stabilize | Reference TrkA sequence |
| | auto-inhibited | |
| | conformation. | |
| G667C⁴ | Catalytic Domain | Reference TrkA sequence |
| R682S | Activation Loop | Reference TrkA sequence |
| V683G | Activation Loop | Reference TrkA sequence |
| I699V⁸ | | |
| Q627X³, Q597X³, | | NP_001012331.1⁷, |
| Q633X³ | | NP_001007793.1⁶, and |
| | | Reference TrkA sequence, |
| | | respectively |
| R702C | Exposed, may form face- | Reference TrkA sequence |
| | to-face disulfide linked | |
| | dimer | |
| R744H⁹ | | |

¹Reference TrkA sequence is UniProtKB/Swiss-Prot: P04629.4, and can be found at URL: www.ncbi.nlm.nih.gov/protein/94730402?report=genbank&log$=protalign&blast_rank=0&RID=0
²Zhang et al., Blood 124(21): 1682, 2014. Mutation found in T-cell prolymphocytic leukemia.
³Park et al., Proc. Natl. Acad. Sci. U.S.A. 112(40): 12492-12497, 2015. Mutation found in colorectal cancer.
⁴Russo et al., Cancer Discov. January; 6(1): 36-44, 2016.
⁵PCT Application No. WO2016196141A1.
⁶www.ncbi.nlm.nih.gov/protein/56118210?report=genbank&log$=protalign&blast_rank=3&RID=0
⁷www.ncbi.nlm.nih.gov/protein/59889558
⁸Deihimi et al., Oncotarget. June 20; 8(25): 39945-39962. doi: 10.18632/oncotarget. 18098, 2017.
⁹Iniguez-Ariza et al., Journal of Clinical Oncology, (Jun. 20, 2017) Vol. 35, No. 15, Supp. 1, 2017 Annual Meeting of the American Society of Clinical Oncology, ASCO, 2017.

In some embodiments, the dysregulation of a NTRK gene, a Trk protein, or expression or activity, or level of the same, includes a splice variation in a TrkA mRNA which results in an expressed protein that is an alternatively spliced variant of TrkA having at least one residue deleted (as compared to a wild-type TrkA protein) resulting in constitutive activity of the TrkA kinase domain. In some embodiments, an alternatively spliced form of TrkA with constitutive activity has deletions of exons 8, 9, and 11 resulting in an expressed protein missing residues 192-284 and 393-398 relative to TrkA Isoform 2, has a deletion of exon 10 in TrkA, or has a deletion in a NTRK1 gene that encodes a TrkA protein with a 75 amino acid deletion in the transmembrane domain (Reuther et al., Mol. Cell Biol. 20:8655-8666, 2000).

Cancers identified as having dysregulation of a NTRK gene, a Trk protein, or expression or activity, or level of the same, (see references cited herein and also the www.cancer.gov and www.nccn.org websites) include:

(A) Cancers wherein the dysregulation of a NTRK gene, a Trk protein, or expression or activity, or level of the same, includes one or more chromosome translocations or inversions resulting in TrkA fusion proteins, e.g., including:

| Cancer | Standard of Care |
|---|---|
| Non-Small | radiotherapy (e.g., radioiodide therapy, external-beam |
| Cell Lung | radiation, or radium 223 therapy), chemotherapeutics as |
| Cancer² | single agents (e.g., afatinib dimaleate, bevacizumab, |
| | carboplatin, cetuximab, cisplatin, crizotinib, erlotinib, |
| | gefitinib, gemcitabine, methotrexate, paclitaxel, or |
| | pemetrexed) or combinations (e.g., carboplatin- |
| | paclitaxel, gemcitabine-paclitaxel, or chemoradiation) |
| Papillary | Radiotherapies (e.g., radioiodide therapy or external- |
| Thyroid | beam radiation) and chemotherapeutics (e.g., sorafenib, |
| Carcinoma¹⁴ | sunitinib, or pazopanib) |
| Glioblastoma | Chemotherapeutics (e.g., bevacizumab, everolimus, |
| Multiforme¹⁵ | lomustine, or temozolomide) |
| Colorectal | Chemotherapeutics as single agents (e.g., aflibercept, |
| Carcinoma¹⁶ | bevacizumab, capecitabine, cetuximab, fluorouracil, |
| | irinotecan, leucovorin, oxaliplatin, panitumumab, or |
| | regorafenib) or combinations (e.g., folfox, folfiri, capox, |
| | folfiri-bevacizumab, folfiri-cetuximab, or xelox) |
| Melanoma¹² | Chemotherapeutics (e.g., aldesleukin, dabrafenib, |
| | dacarbazine, interferon alfa-2b, ipilimumab, peginter- |
| | feron alfa-2b, trametinib, or vemurafenib) |

(B) Cancers wherein the dysregulation of a NTRK gene, a Trk protein, or expression or activity, or level of the same, includes one or more deletions, insertions, or mutations in the TrkA protein, e.g., including:

| Cancer | Standard of care |
|---|---|
| Acute Myeloid | Chemotherapeutics as single agents (e.g., arsenic |
| leukemia^{17, 18} | trioxide, cyclophosphamide, cytarabine, daunoru- |
| | bicin, doxorubicin, or vincristine) or combinations |
| | (e.g., ADE) |
| Large Cell | Radiotherapy (e.g., radioiodide therapy, external- |
| Neuroendocrine | beam radiation, or radium 223 therapy) and/or |
| Carcinoma¹⁹ | chemotherapeutics (e.g., cisplatin, carboplatin, or |
| | etoposide) |
| Neuroblastoma²⁰ | Chemotherapeutics (e.g., cyclophosphamide, |
| | doxorubicin, or vincristine) |

(C) Cancers wherein the dysregulation of a NTRK gene, a Trk protein, or expression or activity, or level of the same, includes overexpression of wildtype TrkA (autocrine activation), e.g., including:

| Cancer | Standard of care |
|---|---|
| Prostate | Radiotherapy (e.g., radium 223 therapy) or |
| Carcinoma^{21, 22} | chemotherapeutics (e.g. abiraterone, cabazitaxel, |
| | degarelix, denosumab, docetaxel, enzalutamide, |
| | leuprolide, prednisone, or sipuleucel-T) |
| Neuro- | Chemotherapeutics (e.g., cyclophosphamide, |
| blastoma²³ | doxorubicin, or vincristine) |
| Pancreatic | Chemotherapeutics as single agents (e.g., erlotinib, |
| Carcinoma²⁴ | fluorouracil, gemcitabine, or mitomycin C) or |
| | combinations (e.g., gemcitabine-oxaliplatin) |
| Melanoma²⁵ | Chemotherapeutics (e.g., aldesleukin, dabrafenib, |
| | dacarbazine, interferon alfa-2b, ipilimumab, |
| | peginterferon alfa-2b, trametinib, or vemurafenib) |
| Head and Neck | Radiotherapy and/or chemotherapeutics (e.g., |
| Squamous Cell | bleomycin, cetuximab, cisplatin, docetaxel, |
| Carcinoma²⁶ | fluorouracil, or methotrexate) |
| Gastric | Chemotherapeutics (e.g., docetaxel, doxorubucin, |
| Carcinoma²⁷ | fluorouracil, mitomycin C, or trastuzumab) |

In some embodiments, the dysregulation of a NTRK gene, a Trk protein, or expression or activity, or level of the same, includes a translocation that results in the expression of a TrkB fusion protein, e.g., one of the TrkB fusion proteins shown in Table 5.

TABLE 5
Exemplary TrkB Fusion Proteins and Cancers

| | | Non-limiting Exemplary TrkB |
|---|---|---|
| | | Fusions and Synonyms of |
| Fusion Protein | Non-TrkB Fusion Partner | Associated Cancer(s) |
| NACC2-TrkB¹ | NACC Family Member 2, | Pilocytic Astrocytoma¹ |
| | BEN and BTB (POZ) Domain | |
| | Containing | |
| QKI-TrkB^{1, 11} | QKI, KH Domain Containing, | Pilocytic Astrocytoma¹ |
| | RNA Binding | |
| AFAP1-TrkB² | Actin Filament Associated | Lower-Grade Glioma^{2, 5}, Pilocytic |
| | Protein 1 | Astrocytoma with Anaplasia |
| | | (PAA)⁴, In vitro (Murine Ba/F3 |
| | | cells)³ |
| PAN3-TrkB² | PAN3 Poly(A) Specific | Head and Neck Squamous Cell |
| | Ribonuclease Subunit | Carcinoma² |
| SQSTM1-TrkB² | Sequestosome 1 | Lower-Grade Glioma², |
| | | Glioblastoma¹² |
| TRIM24-TrkB² | Tripartite Motif Containing 24 | Lung adenocarcinoma², Non- |
| | | Small Cell Lung Cancer¹⁷ |
| VCL-TrkB⁶ | Vinculin | Pediatric gliomas (e.g., pediatric |
| | | high-grade glioma⁶) |
| AGBL4-TrkB⁶ | ATP/GTP Binding Protein- | Pediatric gliomas (e.g., pediatric |
| | Like 4 | high-grade glioma⁶) |
| DAB2IP-TrkB¹⁷ | Disabled Homolog 2- | Colorectal Cancer¹⁷ |
| | Interacting Protein | |
| TrkB-TERT⁷ | Telomerase Reverse | Thyroid Cancer^{7, 8} |
| | Transcriptase | |
| TEL-TrkB⁹ | ETS Variant 6 | In vitro (murine Ba/F3 cells)⁹, |
| (ETV6) | | Acute Myeloid Leukemia |
| | | (AML)¹⁰, Pediatric |
| | | Glioblastoma²¹ |
| NOS1AP-TrkB¹² | | Anaplastic Astrocytoma¹² |
| GKAP1-TrkB¹² | | Glioblastoma¹² |
| KCTD8-TrkB¹² | | Glioblastoma¹² |
| TBC1D2-TrkB¹² | | Glioblastoma¹² |
| VCAN-TrkB¹² | | Grade II Astrocytoma¹² |
| SLMAP-TrkB¹⁸ | | Ganglioma¹³ |
| TLE4-TrkB¹⁴ | | Ganglioma¹⁴ |
| STRN3-TrkB¹⁵ | Striatin | Ganglioglioma¹⁵ |
| WNK2-TrkB¹⁵ | | Complex Glioneuronal Tumor¹⁵ |
| TrkB- BEND5¹⁶ | | Malignant Epithelioid |
| | | Glioneuronal Tumor (MEGNT)¹⁶ |
| TrkB-TRAF2¹⁹ | | Melanoma¹⁹ |
| Nav1-TrkB²⁰ | | Oligoastrocytoma²⁰ |
| STRN-TrkB | | Salivary gland cancer; Soft tissue |
| | | sarcoma²⁵ |
| RIP13-TrkB²² | | |
| BCR-TrkB | | Glioma²³ |
| TLE-TrkB | | |
| GNAQ-TrkB | | Bone sarcoma²⁴ |
| PRKAR2A-TrkB | | |
| RBPMS-TrkB | | Soft tissue sarcoma |
| KANK-TrkB | | CNS |
| AGTPBP1-TrkB | | CNS |
| SPECC1L-TrkB | | CNS |

1Jones et al., Nature Genetics 45: 927-932, 2013.
²Stransky et al., Nature Comm. 5: 4846, 2014.
³Drilon et al., Ann Oncol. 27(5): 920-6, 2016.
⁴Lin et al., Abstract Number: HG-48. 17^{th} International Symposium on Pediatric Neuro-Oncology, ISPNO 2016. Liverpool, UK, 2016.
⁵U.S. Patent Application No. 2016/0272725.
⁶Wu et al., Nature Genetics 46: 444-450, 2014.
⁷P.C.T. Patent Application Publication No. WO 2015/183836.
⁸P.C.T. Patent Application Publication No. WO 2015/183837.
⁹Yuzugullu et al., Cell Discov. 2: 16030, 2016.
¹⁰Taylor et al. Abstract Number: 794. Meeting Info: 59th Annual Meeting of the American Society of Hematology, ASH 2017. Atlanta, GA, United States, 2017.
¹¹Ni et al., Neuro Oncol. 19(1): 22-30, 2017.
¹²Subramaniam et al., 2017 Annual Meeting of the American Society of Clinical Oncology, ASCO. Chicago, IL, United States, 2017.
¹³Ellison et al., Abstract Number: O13. 117th Meeting of the British Neuropathological Society, Royal College of Physicians. London, United Kingdom, 2017.
¹⁴Prabhakaran et al., Neuropathology. E-ISSN: 1440-1789. L-ISSN: 0919-6544, 2018.
¹⁵Alvarez-Breckenridge et al., NPJ Precision Oncology. 1(5) doi: 10.1038/s41698-017-0009-y, 2017.
¹⁶Bavle et al., Abstract Number: GENE-04. Meeting Info: 4th Biennial Conference on Pediatric Neuro-Oncology Basic and Translational Research. New York City, NY, United States, 2017.
¹⁷Wei et al., Abstract Number: 78. Meeting Info: 28th EORTC-NCI-AACR Symposium on Molecular Targets and Cancer Therapeutics. Munich, Germany, 2016.
¹⁸Qaddoumi et al., Acta Neuropathol. 131(6): 833-45, 2016.
¹⁹Lezcano et al., Am. J. Surg. Pathol. doi: 10.1097/PAS.0000000000001070, 2018.
²⁰Zhang et al., Nat. Genet. 45(6): 602-612, 2013.
²¹Bender et al., Abstract Number: HG-024. Meeting Info: 16th International Symposium on Pediatric Neuro-Oncology in Conjunction with the 8th St. Jude-VIVA Forum. Singapore, Singapore, 2014.
²²Fagan et al., Abstract Number: 5158. Meeting Info: American Association for Cancer Research Annual Meeting, 2017.
²³Hechtman JF, et al. Am. J. Surg. Pathol. 41: 1547-1551, 2017.
²⁴Gatalica Z, et al. Mod. Pathol. 32(1): 147-153, 2019.
²⁶Drilon A, et al. N. Engl. J. Med. 378: 731-739, 2018.

In some embodiments, the dysregulation of a NTRK gene, a Trk protein, or expression or activity or level of any of the same, includes at least one point mutation in a NTRK gene that results in the production of a TrkB protein that has one or more amino acid substitutions, insertions, or deletions as compared to the wildtype TrkB protein (see, for example, the point mutations listed in Table 6).

TABLE 6
TrkB Kinase Protein Amino Acid Substitutions/Insertions/Deletions^{A}

Amino acid position 13 (e.g., A13T²)
Amino acid position 142 (e.g., E142K²)
Amino acid position 136 (e.g., R136H²)
Amino acid position 167 (e.g., S167Y³)
Amino acid position 203 (e.g., A203T⁶)
Amino acid position 245 (e.g., H245Y⁶)
Amino acid position 458 (e.g., R458G⁶)
Amino acid position 545 (e.g., G545R)
Amino acid position 570 (e.g., A570V)
Amino acid position 596 (e.g., Q596E, Q596P)
Amino acid position 601 (e.g., V601G)
Amino acid position 617 (e.g., F617L, F617C, F617I)
Amino acid position 619 (e.g., V619M⁴)
Amino acid position 623 (e.g., G623S, G623R)
Amino acid position 624 (e.g., D624V)
Amino acid position 628 (e.g., F628x)
Amino acid position 630 (e.g., R630K)
Amino acid position 633 (e.g., F633L⁴)
Amino acid position 639 (e.g., G639R¹)
Amino acid position 672 (e.g., F672x)
Amino acid position 682 (e.g., C682Y, C682F)
Amino acid position 683 (e.g., L683V)
Amino acid position 693 (e.g., G693S)
Amino acid position 702 (e.g., Y702x)
Amino acid position 709 (e.g., G709C, G709A, G709S⁴)
Amino acid position 716 (e.g., P716S⁵)

^{A}The TrkB kinase mutations shown above may be activating mutations and/or may confer increased resistance of the TrkB kinase to a TrkB inhibitor e.g., as compared to a wildtype TrkB kinase.
¹PCT Application No. WO2017155018A1.
²Bonanno et al., Journal of Thoracic Oncology,Vol. 11, No. 4, Supp. Suppl. 1, pp S67. Abstract Number: 28P; 6th European Lung Cancer Conference, ELCC 2016, Geneva, Switzerland.
³Iniguez-Ariza et al., Journal of Clinical Oncology, (Jun. 20, 2017) Vol. 35, No. 15, Supp. 1, 2017 Annual Meeting of the American Society of Clinical Oncology, ASCO, 2017.
⁴PCT Application No. WO2016196141A1.
⁵Deihimi et al., Oncotarget. June 20; 8(25): 39945-39962. doi: 10.18632/oncotarget. 18098, 2017.
⁶Qian et al., Blood. 128: 1566, 2016.

In some embodiments, the dysregulation of a NTRK gene, a Trk protein, or expression or activity, or level of the same, includes at least one point mutation in a NTRK2 gene that results in the production of a TrkB protein that has one or more amino acid substitutions as compared to the wildtype TrkB protein. In some embodiments, the at least one or more amino acid substitutions are activating mutations (see, for example, the point mutations listed in Table 7).

TABLE 7
Activating TrkB Point Mutations¹

| | | Exemplary Isoform in which |
|---|---|---|
| Point Mutation | Rationale | Mutation is Present (if known) |
| A13T² | | Reference TrkB sequence |
| E142K² | | Reference TrkB sequence |
| R136H² | | Reference TrkB sequence |
| S167Y³ | | |
| A203T⁵ | | |
| A458G⁵ | | |
| P716S⁴ | | |

¹Reference TrkB sequence is UniProtKB/Swiss-Prot: Q16620.1, and can be found at URL: www.ncbi,nlm.nih.gov/protein/2497560?report=genbank&log$=protalign&blast_rank=0&RID=0
²Bonanno et al., Journal of Thoracic Oncology, Vol. 11, No. 4, Supp. Suppl. 1, pp S67. Abstract Number: 28P; 6^{th} European Lung Cancer Conference, ELCC 2016, Geneva, Switzerland.
³Iniguez-Ariza et al., Journal of Clinical Oncology, (Jun. 20, 2017) Vol. 35, No. 15, Supp. 1, 2017 Annual Meeting of the American Society of Clinical Oncology, ASCO, 2017.
⁴Deihimi et al., Oncotarget. June 20; 8(25): 39945-39962. doi: 10.18632/oncotarget. 18098, 2017.
⁵Qian et al., Blood. 128: 1566, 2016.

In some embodiments, the dysregulation of a NTRK gene, a Trk protein, or expression or activity, or level of the same, includes a translocation which results in the expression of a TrkC fusion protein, e.g., one of the TrkC fusion proteins shown in Table 8.

TABLE 8
Exemplary TrkC Fusion Proteins and Cancers

| | | Non-limiting Exemplary |
|---|---|---|
| | Non-TrkC Fusion | TrkC Fusions and Synonyms |
| Fusion Protein | Partner | of Associated Cancer(s) |
| ETV6-TrkC¹ | ETS Variant 6 | Fibrosarcoma (e.g., Infantile or |
| (TEL; e.g., | | Congenital Fibrosarcoma (IFS, |
| chromosomal | | CFS, or CIFS)^{6, 7, 29, 30}), |
| translocation | | Nephroma (e.g., Congenital |
| t(12; 15) (p13; q25)² | | Mesoblastic Nephroma^{3, 60}), |
| t(12; 15) (p13; q26), | | Melanoma (e.g., Skin |
| ins(12; 15) (p13; q22 | | Cutaneous Melanoma⁵⁶), |
| q26)³, or | | Colorectal Cancer (CRC)^{33, 58} |
| t(12; 15) (p13; q25)⁴) | | (colon adenocarcinoma⁵⁶), |
| | | Breast Cancer⁵⁶, |
| | | Gastrointestinal Stromal Tumor |
| | | (GIST)²⁸ (e.g., c-kit-Negative |
| | | GIST²⁸), Pediatric Gliomas |
| | | (e.g., Pediatric High-Grade |
| | | Gliomas^{1, 8}, Desmoplastic |
| | | Infantile Ganglioglioma¹¹), |
| | | Medulloblastoma¹, Thyroid |
| | | Cancer (e.g., Papillary Thyroid |
| | | Cancer^{12, 56, 59}, Sporadic |
| | | Pediatric Differentiated Thyroid |
| | | Carcinoma (DTC)¹³ Post- |
| | | Chernobyl PTCs³¹), Soft Tissue |
| | | Hemangioma³⁴, Mammary |
| | | Analogue Secretory Carcinoma |
| | | (MASC)^{14, 61}, Secretory Breast |
| | | Carcinoma (SBSC)^{10, 27, 57}), |
| | | Primary Thyroid Gland |
| | | Secretory Carcinoma¹⁵, Acinic |
| | | cell carcinoma (AcCC)¹⁶, |
| | | Polymorphous Low-Grade |
| | | Adenocarcinoma¹⁷, Sinonasal |
| | | Low-Grade Non-Intestinal- |
| | | Type Adenocarcinoma⁶², ALK- |
| | | Negative Inflammatory |
| | | Myofibroblastic Tumors |
| | | (IMT)^{18, 19}, Acute Myeloid (or |
| | | Myelogenous) Leukemia |
| | | (AML)³², Promyelocytic |
| | | Leukemia²⁶, Acute |
| | | Lymphoblastic Leukemia |
| | | (ALL) (e.g., Ph-like ALL^{5, 22}), |
| | | Chronic Eosinophilic |
| | | Leukemia²³, Relapsed Pediatric |
| | | B-ALL⁵³, Angiomatoid Fibrous |
| | | Histiocytoma²⁴, |
| | | Neuroendricrine Tumor²⁵ |
| BTBD1-TrkC¹ | BTB (POZ) Domain | Pediatric Gliomas (e.g., high- |
| | Containing 1 | grade gliomas¹) |
| LYN-TrkC³⁵ | V-Yes-1 Yamaguchi | Head and Neck Squamous Cell |
| | Sarcoma Viral Related | Carcinoma⁶³ |
| | Oncogene Homolog (also | |
| | known as Lck/Yes-Related | |
| | Novel Protein Tyrosine | |
| | Kinase) | |
| RBPMS-TrkC³⁵ | RNA Binding Protein with | Thyroid Cancer⁵⁶ (e.g., |
| | Multiple Splicing | Papillary Thyroid Cancer⁶³), |
| | | Uterine Spindle Cell Sarcoma³⁶ |
| EML4-TrkC³⁷ | Echinoderm Microtubule- | Fibrosarcoma (e.g., Pediatric |
| (e.g., | Associated Protein-Like 4 | Fibrosarcoma³⁹ or Infantile |
| t(2; 15) (2p21; 15q25))³⁸ | | Fibrosarcoma^{9, 37, 45, 64}), |
| | | Glioblastoma^{40, 20}, Colon |
| | | Cancer⁴¹, Mesenchymal |
| | | Tumor⁴², Thyroid Cancer⁴³, |
| | | Congenital Mesoblastic |
| | | Nephroma⁴⁴, Pancreatic |
| | | adenocarcinoma⁵⁶ |
| TrkC-HOMER2 | Homer Protein Homolog 2 | Soft Tissue Sarcoma³⁴ |
| TFG-TrkC | TRK-Fused Gene | Soft Tissue Solitary Fibrous |
| | | Tumor³⁴ |
| FAT1-TrkC⁴⁶ | FAT Atypical Cadherin 1 | Cervical Squamous Cell |
| | | Carcinoma^{46, 56} |
| MYO5A-TrkC⁴⁹ | Myosin VA | Melanocytic Tumor⁴⁹ (e.g., |
| | | Spitz tumor⁴⁷), Melanoma⁴⁸ |
| MYH9-TrkC⁴⁷ | Myosin Heavy Chain 9 | Spitz Tumor⁴⁷ |
| KANK1-TrkC²¹ | KANK1 | Renal Metanephric Adenoma |
| (e.g., | | (MA)²¹ |
| t(9; 15) (p24; q24))⁵⁰ | | |
| SQSTM1-TrkC⁵¹ | Sequestosome 1 | Papillary Thyroid Carcinoma, |
| | | thyroid carcinoma^{55, 56} |
| UBE2R2-TrkC | Ubiquitin Conjugating | Multiple Myeloma⁵² |
| | Enzyme E2 R2 | |
| HNRNPA2B1-TrkC | | Multiple Myeloma⁵² |
| VPS18-NTRK3⁵⁶ | | Colon Adenocarcinoma⁵⁶ |
| AKAP13-NTRK3⁵⁶ | | Lower Grade Glioma⁵⁶ |
| NTRK3-LOXL2⁵⁶ | | Lower Grade Glioma⁵⁶ |
| NTRK3-PEAK1⁵⁶ | | Lower Grade Glioma⁵⁶ |
| ZNF710-TrkC*^{54, 58} | | Glioblastoma⁶⁵ |
| TPM4-TrkC | | Soft Tissue Sarcoma⁶⁵ |
| LMNA-TrkC | | Soft Tissue Sarcoma |
| SPECC1L-TrkC | | Uterine sarcoma⁶⁶ |
| STRN-TrkC | | Adult fibrosarcoma⁶⁷ |
| STRN3-TrkC | | Adult fibrosarcoma⁶⁷ |
| VIM-TrkC | | Thyroid cancer⁶⁶ |
| AFAP1-TrkC | | Glioblastoma⁶⁵ |

*The transcript of this fusion was not detected.
¹Wu et al., Nature. Genet. 46: 444-450, 2014.
²Skalova et al., Mod. Pathol. 30:S27-S43, 2017.
³Watanbe et al., Cancer Genet. Cytogenet. 136(1): 10-16, 2002.
⁴Eguchi et al., Blood. 93: 1355-1363, 1999.
⁵Roberts et al., Abstract Number: 278, 58th Annual Meeting of the American Society of Hematology, ASH 2016. San Diego, CA, United States, 2016.
⁶Knezevich et al., Nat. Genet. 18(2): 184-7, 1998.
⁷Pavlick et al., Pediatr. Blood Cancer. doi: 10.1002/pbc.26433, 2017.
⁸Hover et al., Abstract Number: TMOD-07. Meeting Info: 4th Biennial Conference on Pediatric Neuro-Oncology Basic and Translational Research. New York City, NY, United States, 2017.
⁹Church et al., Mod. Pathol. 31(3), 463-473, 2018.
¹⁰Arce et al., World J. Surg. Oncol. 3: 35, 2005.
¹¹Carvalho et al., Abstract Number: HG-09. Meeting Info: 3rd Biennial Conference on Pediatric Neuro-Oncology Basic and Translational Research. San Diego, CA, United States, 2015.
¹²Otsubo et al., J. Pediatr., Endocrinol., Metab. 28; 31(4): 461-467, 2018.
¹³Picarsic et al., Pediatr., Dev. Pathol. 19(2): 115-22, 2016.
¹⁴Skalova et al., Am. J. Surg. Pathol. 42(2): 234-246, 2018.
¹⁵Farhat et al., Am. J. Clin. Pathol., 148(3): 251-258, 2017.
¹⁶Chintakuntlawar et al., Oral Surg. Oral Med. Oral Pathol. Oral Radiol. 121(5): 542-549.e1, 2016.
¹⁷Montalli et al., J. Oral Pathol. Med. doi: 10.1111/jop.12491, 2016.
¹⁸Alassiri et al., Am. J. Surg. Pathol. 40(8): 1051-61, 2016.
¹⁹Yamamoto et al., Histopathology. 69(1): 72-83, 2016.
²⁰Subramaniam et al., 2017 Annual Meeting of the American Society of Clinical Oncology, ASCO. Chicago, IL, United States, 2017.
²¹Catic et al., Cancer Genet. 214-215: 9-15, doi: 10.1016/j.cancergen.2017.03.001, 2017.
²²Reshmi et al., Abstract Number: 477. Meeting Info: 59th Annual Meeting of the American Society of Hematology, ASH 2017. Atlanta, GA, United States, 2017.
²³Forghieri et al., Abstract Number: P137. Meeting Info: 11th Congress of the Italian Society of Experimental Hematology. Turin, Italy, 2010.
²⁴Walther et al., Cancer Genet. 206(7-8), 299-303, 2013.
²⁵Sigal, et al., J. Natl. Compr. Canc. Netw. 15(11): 1317-1322, 2017.
²⁶Macleod, et al., Abstract Number: 0294. Meeting Info: 14th Congress of the European Hematology Association. Berlin, Germany, 2009.
²⁷Tognon et al., Cancer Cell. 2(5): 367-376, 2002.
²⁸Brenca et al., J. Pathol. 238(4): 543-549, 2016.
²⁹Rossi et al., Abstract Number: 84. Meeting Info: 105th Annual Meeting of the United States and Canadian Academy of Pathology, USCAP 2016. Seattle, WA, United States, 2016.
³⁰Sheng et al., Am. J. Clin. Pathol. 115(3): 348-355, 2001.
³¹Leeman-Neill et al., Cancer. 120(6): 799-807, 2014.
³²Kralik et al., Diagn. Pathol. 6: 19, 2011.
³³U.S. Patent Application No. 2016/0305943.
³⁴Doebele et al., Cancer Discov. 5(10): 1049-1057, 2015.
³⁵Stransky et al., Nature Comm. 5: 4846, 2014.
³⁶Chiang et al., Am. J. Surg. Pathol. doi: 10.1097/PAS.0000000000001055, 2018.
³⁷Tannenbaum et al., Cold Spring Harb. Mol. Case Stud. 1:a000471, 2015.
³⁸Tannenbaum, et al., Abstract Number: 749. Meeting Info: 2015 American Society of Pediatric Hematology/Oncology, ASPHO 2015. Phoenix, AZ, United States, 2015.
³⁹Sims et al., Abstract Number: P280; 31^{st} Annual Meeting and Associated Programs of the Society for Immunotherapy of Cancer, SITC 2016. National Harbor, MD, United States, 2016.
⁴⁰Schram et al., Cancer Research. Abstract Number: LB-302, American Association for Cancer Research Annual Meeting, Washington, DC, United States, 2017.
⁴¹Coebergh et al., Cancer Research. Abstract Number: 490, American Association for Cancer Research Annual Meeting, Washington, DC, United States, 2017.
⁴²Davis et al., Pediatr. Dev. Pathol. 21(1): 68-78, 2018.
⁴³Nikiforova et al., Abstract Number: 5. Meeting Info: 84th Annual Meeting of the American Thyroid Association. Coronado, CA, United States, 2014.
⁴⁴Church et al., Mod. Pathol. 31(3), 463-473, 2018.
⁴⁵Church et al., Abstract Number: ST16. Meeting Info: 2015 Annual Meeting of the Association for Molecular Pathology, AMP 2015. Austin, TX, United States, 2015.
⁴⁶U.S. Patent Application Publication No. 2015/0315657.
⁴⁷Yeh et al., J Pathol. 240(3): 282-90, 2016
⁴⁸Leyvraz et al., Abstract Number: 897. Meeting Info: 33. Deutscher Krebskongress, DKK. Berlin, Germany, 2018.
⁴⁹Wang et al., J. Mol. Diagn. 19(3): 387-396, 2017.
⁵⁰Catic et al., Meeting Info: 2017 Annual Meeting of the American Society of Clinical Oncology, ASCO. Chicago, IL, United States, 2017.
⁵¹Lu et al., Oncotarget. 8(28): 45784-45792, 2017.
⁵²Taylor et al., Abstract Number: 794. Meeting Info: 59th Annual Meeting of the American Society of Hematology, ASH 2017. Atlanta, GA, United States, 2017.
⁵³Baughn et al., Abstract Number: 5115. Meeting Info: 59th Annual Meeting of the American Society of Hematology, ASH 2017. Atlanta, GA, United States, 2017.
⁵⁴Hechtman et al., Abstract Number: 1837. Meeting Info: 106th Annual Meeting of the United States and Canadian Academy of Pathology, USCAP 2017. San Antonio, TX, United States, 2017.
⁵⁵Iyama et al., Thyroid. 27(6): 811-818, 2017.
⁵⁶Gao et al., Cell Rep. 23(1): 227-238.e3, 2018.
⁵⁷Zheng et al., Nat Med. 20(12): 1479-84, 2014.
⁵⁸Hechtman et al., Mol. Cancer Res. 14(3): 296-301, 2016.
⁵⁹Ricarte-Filho et al., J. Clin. Invest. 123(11): 4935-44, 2013.
⁶⁰Rubin et al., Am. J. Pathol. 153(5): 1451-8, 1998.
⁶¹Skálová et al., Am. J. Surg. Pathol. 2016 January; 40(1): 3-13.
⁶²Andreason et al., Am. J. Surg. Pathol. 41(11): 1552-1560, 2017.
⁶³Wei et al., Abstract Number: 78. Meeting Info: 28th EORTC-NCI-AACR Symposium on Molecular Targets and Cancer Therapeutics. Munich, Germany, 2016.
⁶⁴Kao et al., Am. J. Surg. Pathol. 42(1): 28-38, 2018.
⁶⁵Hechtman JF, et al. Am. J. Surg. Pathol. 41: 1547-1551, 2017.
⁶⁶Gatalica Z, et al. Mod. Pathol. 32(1): 147-153, 2019.
⁶⁷Yamazaki F, et al. Am J Surg Pathol 2018.

In some embodiments, the dysregulation of a NTRK gene, a Trk protein, or expression or activity or level of any of the same, includes at least one point mutation in a NTRK gene that results in the production of a TrkC protein that has one or more amino acid substitutions, insertions, or deletions as compared to the wildtype TrkC protein (see, for example, the point mutations listed in Table 9).

TABLE 9
TrkC Kinase Protein Amino Acid Substitutions/Insertions/Deletions^{A}

Amino acid position 176 (e.g., E176D⁵)
Amino acid position 261 (e.g., T261I⁵)
Amino acid position 378 (e.g., L378V⁵)
Amino acid position 449 (e.g., L449F⁵)
Amino acid position 545 (e.g., G545R)
Amino acid position 570 (e.g., A570V)
Amino acid position 596 (e.g., Q596x)
Amino acid position 601 (e.g., V601x)
Amino acid position 603 (e.g., V603M²)
Amino acid position 617 (e.g., F617x, F617L²)
Amino acid position 623 (e.g., G623R¹)
Amino acid position 624 (e.g., D624V)
Amino acid position 628 (e.g., F628x)
Amino acid position 630 (e.g., R630x)
Amino acid position 645 (e.g., R645C⁵)
Amino acid position 675 (e.g., F675x)
Amino acid position 685 (e.g., C685Y, C685F)
Amino acid position 686 (e.g., L686V)
Amino acid position 696 (e.g., G696x, G696C, G696A², G696S²)
Amino acid position 705 (e.g., Y705x)
Amino acid position 745 (e.g., R745L³)
Amino acid position 749 (e.g., I749M⁴)

^{A}The TrkC kinase mutations shown above may be activating mutations and/or may confer increased resistance of the TrkC kinase to a TrkC inhibitor e.g., as compared to a wildtype TrkC kinase.
¹Drilon et al., What hides behind the MASC: clinical response and acquired resistance to entrectinib after ETV6-NTRK3 identification in a mammary analogue secretory carcinoma (MASC), Ann Oncol. 2016 May; 27(5): 920-6. doi: 10.1093/annonc/mdw042. Epub 2016 Feb. 15.
²PCT Application No. WO2016196141A1.
³Deihimi et al., Oncotarget. June 20; 8(25): 39945-39962. doi: 10.18632/oncotarget.18098, 2017.
⁴Iniguez-Ariza et al., Journal of Clinical Oncology, (Jun. 20, 2017) Vol. 35, No. 15, Supp. 1, 2017 Annual Meeting of the American Society of Clinical Oncology, ASCO, 2017.
⁵Qian et al., Blood. 128: 1566, 2016.

In some embodiments, the dysregulation of a NTRK gene, a Trk protein, or expression or activity, or level of the same, includes at least one point mutation in a NTRK3 gene that results in the production of a TrkC protein that has one or more amino acid substitutions as compared to the wildtype TrkC protein. In some embodiments, the at least one or more amino acid substitutions are activating mutations (see, for example, the point mutations listed in Table 10).

TABLE 10
Activating TrkC Point Mutations¹

| | | Exemplary Isoform in |
|---|---|---|
| | | which Mutation is Present |
| Point Mutation | Rationale | (if known) |
| G176D⁵ | | |
| L449F⁵ | | |
| G623R² | Steric Hinderance | Reference TrkC sequence |
| R745L³ | | |
| I749M⁴ | | |

¹Reference TrkC sequence is UniProtKB/Swiss-Prot: Q16288.2, and can be found at URL: www.ncbi.nlm.nih.gov/protein/134035335?report=genbank&log$=protalign&blast_rank=0&RID=0
²Drilon et al., Ann Oncol. 2016 May; 27(5): 920-6. doi: 10.1093/annonc/mdw042. Epub 2016 Feb. 15.
³Deihimi et al., Oncotarget. June 20; 8(25): 39945-39962. doi: 10.18632/oncotarget.18098, 2017.
⁴Iniguez-Ariza et al., Journal of Clinical Oncology, (Jun. 20, 2017) Vol. 35, No. 15, Supp. 1, 2017 Annual Meeting of the American Society of Clinical Oncology, ASCO, 2017.
⁵Qian et al., Blood. 128: 1566, 2016.

In some embodiments, the dysregulation of a NTRK gene, a Trk protein, or expression or activity, or level of the same, includes at least one point mutation in a NTRK gene that results in the production of a Trk protein that has one or more amino acid substitutions as compared to the wildtype Trk protein. For example, a mutation can include one or more of a solvent front mutation (e.g., TrkA G595R), an xDFG mutation (e.g., TrkA G667S), or a gatekeeper mutation (e.g., TrkC F617L). In some embodiments, these mutations are associated with resistance (e.g., acquired resistance) to one or more Trk kinase inhibitors.

In some embodiments, a cancer with a dysregulation of a NTRK gene, a Trk protein, or expression or activity, or level of the same also has microsatellite instability (MSI). In some embodiments, an abnormal microsatellite marker is a microsatellite marker in a cancerous-tissue sample from a subject that is different from the corresponding microsatellite marker from an adjacent non-cancerous tissue sample from the subject. For example, the number of repeats of the microsatellite marker is different in a cancerous tissue sample from a subject compared to an adjacent non-cancerous tissue sample from the subject. In some embodiments, an abnormal microsatellite marker is a microsatellite marker in a cancerous-tissue sample from a subject that is different from the corresponding microsatellite marker from a control, e.g., the corresponding microsatellite from peripheral blood specimen(s). In some embodiments, a cancer is determined to have MSI if greater than about 20% of the microsatellite markers are abnormal (e.g., in a sample obtained from the patient). In some embodiments, the MSI cancer is a cancer that has high MSI (MSI-H) or low MSI (MSI-L). In some embodiments, a cancer is determined to have MSI-H if two or more microsatellite markers are abnormal (e.g., in a sample obtained from the patient). For example, if two microsatellite markers are abnormal out of five microsatellite markers analyzed. In some embodiments, a cancer is determined to have MSI-H if greater than about 30% of the microsatellite markers analyzed are abnormal (e.g., in a sample obtained from the patient). In some embodiments, a cancer is determined have low microsatellite instability (MSI-L) if only one microsatellite marker is abnormal (e.g., in a sample obtained from the patient). For example, if only one microsatellite marker is abnormal out of five microsatellite markers analyzed. In some embodiments, a cancer is determined to have MSI-L if about 3.5% to about 30% (e.g. about 3.5%, about 20%, or about 30%) of the microsatellite markers analyzed are abnormal (e.g., in a sample obtained from the patient). In some embodiments, a cancer is determined to have microsatellite stability (MSS) if the mononucleotide repeat markers are identical or substantially identical between samples from cancerous tissue and adjacent non-cancerous tissue. In some embodiments, a cancer is determined to have microsatellite stability (MSS) if less than about 3.5% of the microsatellite markers analyzed are abnormal.

In some embodiments, immunohistochemistry and/or sequencing is used to detect the microsatellite markers. In some embodiments, the microsatellite markers are selected from the group consisting of: MLH1, PMS2, MSH2, MSH6, BAT-25, BAT26, NR-21, NR24 and MONO-27. In some embodiments, immunohistochemistry is used to detect the microsatellite markers: MLH1, PMS2, MSH2, and MSH6. In some embodiments, sequencing is used to detect the microsatellite markers: BAT-25, BAT26, NR-21, NR24 and MONO-27. See, for example, Murphy et al., J Mol Diagn. 2006 July; 8(3): 305-311, which is incorporated by reference herein in its entirety.

In some embodiments, MSI status is determined by MSI-PCR, see, for example, Boland et al., Cancer Res. 1998 Nov. 15; 58(22):5248-57, which is incorporated by reference herein in its entirety.

In some embodiments, MSI status is determined by Microsatellite Instability Analysis.

In some embodiments, MSI status is determined by a smMIP assay, see, for example, Waalkes et al., Clin Chem. 2018 June; 64(6): 950-958, which is incorporated by reference herein in its entirety.

In some embodiments, MSI status is determined by MSIsensor, see, for example, Niu et al., Bioinformatics. 2014 Apr. 1; 30(7): 1015-1016, which is incorporated by reference herein in its entirety.

In some embodiments, MSI status is determined by mSINGS, see, for example, Salipante et al., Clin Chem. 2014 September; 60(9):1192-9, which is incorporated by reference herein in its entirety.

In some embodiments, MSI status is determined by MANTIS, see, for example, Kautto et al., Oncotarget. 2017 Jan. 31; 8(5): 7452-7463, which is incorporated by reference herein in its entirety.

In some embodiments, wherein the cancer has a dysregulation of a NTRK gene, a Trk protein, or expression or activity, or level of the same and has MSI, the dysregulation of a NTRK gene, a Trk protein, or expression or activity, or level of the same is one or more point mutations in a NTRK gene selected from the group consisting of NTRK1, NTRK2, and NTRK3. In some embodiments the one or more point mutations in an NTRK1 gene results in the expression of a TrkA protein comprising a mutation at one or more amino acid position(s) selected from the group consisting of: 613 and 699 (e.g., G613V and I699V). In some embodiments, the one or more point mutations in a NTRK2 gene results in the expression of a TrkB protein comprising a mutation at one or more amino acid position(s) selected from the group consisting of: 716, 675, and 662 (e.g., P716S, R675H, and A662T). In some embodiments, the one or more point mutations in a NTRK3 gene results in the expression of a TrkC protein comprising a mutation at one or more amino acid position(s) selected from the group consisting of: 678 and 745 (e.g., R678* and R745L). In some embodiments, the MSI is MSI-H. In some embodiments, the cancer that has a dysregulation of a NTRK gene, a Trk protein, or expression or activity, or level of the same and has MSI is colorectal cancer. Trk mutations in MSI-H cancers, such as MSI-H colorectal cancer, are described in Deihimi et al. Oncotarget, 2017, Vol. 8, (No. 25), pp: 39945-39962, and WO 2018/157032, both of which are hereby incorporated by reference in their entirety.

In some embodiments, a TRK-associated cancer has been identified as having one or more TRK inhibitor resistance mutations (that result in an increased resistance to a TRK inhibitor. Non-limiting examples of TRK inhibitor resistance mutations are listed in Tables 11-13.

TABLE 11
Exemplary TrkA Resistance Mutations

Amino acid position 517 (e.g., G517R)
Amino acid position 542 (e.g., A542V)
Amino acid position 564 (e.g., L564H²)
Amino acid position 568 (e.g., Q568x)
Amino acid position 573 (e.g., V573M)
Amino acid position 589 (e.g., F589L, F589C)
Amino acid position 595 (e.g., G595S, G595R¹, G595L²)
Amino acid position 599 (e.g., D596V)
Amino acid position 600 (e.g., F600L)
Amino acid position 602 (e.g., R602x)
Amino acid position 646 (e.g., F646V, F646I²)
Amino acid position 656 (e.g., C656Y, C656F)
Amino acid position 657 (e.g., L657V)
Amino acid position 667 (e.g., G667A³, G667C¹, G667S³)
Amino acid position 676 (e.g., Y676S)
Amino acid position 679 (e.g., D679G²)

¹Russo et al., Acquired Resistance to the TRK Inhibitor Entrectinib in Colorectal Cancer, Cancer Discov. 6(1): 36-44, 2016.
²Fuse et al., Mechanisms of Resistance to NTRK Inhibitors and Therapeutic Strategies in NTRK1-Rearranged Cancers, Mol. Cancer Ther. 6(1): 36-44, 2016.
³PCT Application No. WO2016196141A1.

The letter “x” when used to describe a mutation of an amino acid at a specific amino acid position means (i) a substitution of the amino acid present at the same amino acid position in the corresponding wild-type protein with a different naturally-occurring amino acid, or (ii) a deletion of the amino acid present at the same amino acid position in the corresponding wild-type protein.

Non-limiting examples of the specific amino acid positions discovered to have mutations (e.g., substitutions or deletions) in TrkA in Trk inhibitor-resistant cancer cells having a NTRK1 point mutation are listed below. Also listed below are the different specific amino acid mutations (e.g., substitutions) present in TrkA proteins present in Trk inhibitor resistant cancer cells having a NTRK1 point mutation.

Trk inhibitor-resistant cancer cells were discovered to have point mutations in a NTRK1 gene that result in a TrkA protein that includes one or more (e.g., two, three, four, five, six, seven, eight, nine, ten, eleven, twelve, thirteen, fourteen, or fifteen) amino acid substitutions or deletions at amino acid positions: 517, 542, 568, 573, 589, 595, 599, 600, 602, 646, 656, 657, 667, and 676 (e.g., amino acid positions corresponding to those in wild-type sequence NP_002520 (SEQ ID NO: 9)). Different specific amino acid substitutions present in a TrkA protein generated in a Trk inhibitor-resistant cancer cell include one or more (e.g., two, three, four, five, six, seven, eight, nine, ten, eleven, or twelve) of the following: G517R, A542V, V573M, F589L, F589C, G595S, G595R, D596V, F600L, F646V, C656Y, C656F, L657V, G667S, G667C, and Y676S (e.g., as compared to the wild-type sequence NP_002520 (SEQ ID NO: 9)).

TABLE 12
Exemplary TrkB Resistance Mutations

Amino acid position 545 (e.g., G545R)
Amino acid position 570 (e.g., A570V)
Amino acid position 596 (e.g., Q596E, Q596P)
Amino acid position 601 (e.g., V601G)
Amino acid position 617 (e.g., F617L, F617C, F617I)
Amino acid position 619 (e.g., V619M)²
Amino acid position 623 (e.g., G623S, G623R)
Amino acid position 624 (e.g., D624V)
Amino acid position 628 (e.g., F628x)
Amino acid position 630 (e.g., R630K)
Amino acid position 633 (e.g., F633L²)
Amino acid position 639 (e.g., G639R¹)
Amino acid position 672 (e.g., F672x)
Amino acid position 682 (e.g., C682Y, C682F)
Amino acid position 683 (e.g., L683V)
Amino acid position 693 (e.g., G693S)
Amino acid position 702 (e.g., Y702x)
Amino acid position 709 (e.g., G709C², G709A², G709S²)

¹PCT Application No. WO2017155018A1.
²PCT Application No. WO2016196141A1.

The letter “x” when used to describe a mutation of an amino acid at a specific amino acid position means (i) a substitution of the amino acid present at the same amino acid position in the corresponding wild-type protein with a different naturally-occurring amino acid, or (ii) a deletion of the amino acid present at the same amino acid position in the corresponding wild-type protein.

Trk inhibitor-resistant cancer cells were discovered to have point mutations in a NTRK2 gene that result in a TrkB protein that includes one or more (e.g., two, three, four, five, six, seven, eight, nine, ten, eleven, twelve, thirteen, fourteen, or fifteen) amino acid substitutions or deletions at amino acid positions: 545, 570, 596, 601, 617, 623, 624, 628, 630, 672, 682, 683, 693, and 702 (e.g., amino acid positions corresponding to those in wild-type sequence AAB33109.1 (SEQ ID NO: 10)). Different specific amino acid substitutions present in a TrkB protein generated in a Trk inhibitor-resistant cancer cell include one or more (e.g., two, three, four, five, six, seven, eight, nine, eleven, or twelve) of the following: G545R, A570V, Q596E, Q596P, V601G, F617L, F617C, F617I, G623S, G623R, D624V, R630K, C682Y, C682F, L683V, G693S, and G713S (e.g., as compared to the wild-type sequence AAB33109.1 (SEQ ID NO: 10)).

TABLE 13
Exemplary TrkC Resistance Mutations

Amino acid position 545 (e.g., G545R)
Amino acid position 570 (e.g., A570V)
Amino acid position 596 (e.g., Q596x)
Amino acid position 601 (e.g., V601x)
Amino acid position 603 (e.g., V603M²)
Amino acid position 617 (e.g., F617x, F617L²)
Amino acid position 623 (e.g., G623R¹)
Amino acid position 624 (e.g., D624V)
Amino acid position 628 (e.g., F628x)
Amino acid position 630 (e.g., R630x)
Amino acid position 675 (e.g., F675x)
Amino acid position 685 (e.g., C685Y, C685F)
Amino acid position 686 (e.g., L686V)
Amino acid position 696 (e.g., G696x, G696A², G696C², G696S²)
Amino acid position 705 (e.g., Y705x)

¹Drilon et al., What hides behind the MASC: clinical response and acquired resistance to entrectinib after ETV6-NTRK3 identification in a mammary analogue secretory carcinoma (MASC), Ann Oncol. 2016 May; 27(5): 920-6. doi: 10.1093/annonc/mdw042. Epub 2016 Feb. 15.
²PCT Application No. WO2016196141A1.

The letter “x” when used to describe a mutation of an amino acid at a specific amino acid position means (i) a substitution of the amino acid present at the same amino acid position in the corresponding wild-type protein with a different naturally-occurring amino acid, or (ii) a deletion of the amino acid present at the same amino acid position in the corresponding wild-type protein.

Trk inhibitor-resistant cancer cells were discovered to have point mutations in a NTRK3 gene that result in a TrkC protein that includes one or more (e.g., two, three, four, five, six, seven, eight, nine, ten, eleven, twelve, thirteen, fourteen, or fifteen) amino acid substitutions or deletions at amino acid positions: 545, 570, 596, 601, 617, 623, 624, 628, 630, 675, 685, 686, 696, and 705 (e.g., amino acid positions corresponding to those in a wild-type sequence (SEQ ID NO: 11)). Different specific amino acid substitutions present in a TrkC protein generated in a Trk inhibitor-resistant cancer cell include one or more (e.g., two, three, four, five, six, or seven, or eight) of the following: G545R, A570V, F617L, G623R, D624V, C685Y, C685F, L686V, and G696A (e.g., as compared to the wild-type sequence (SEQ ID NO: 11)).

As one skilled in the art can appreciate, the specific substitutions listed above are exemplary. For example, when a naturally-occurring amino acid at an amino acid position is substituted with a different amino acid, it is understood that an amino acid having a chemically-related amino acid side chain may also be substituted (and detected in a cancer cell). Amino acids that have chemically-related amino acid side chains are listed in Table 14.

TABLE 14
Chemically Related Amino Acid Side Chains

| | |
|---|---|
| Positively-Charged | Lysine, Arginine, Histidine |
| Side Chains | |
| Negatively-Charged | Glutamate and Aspartate |
| Side Chains | |
| Nonpolar and/or | Glycine, Alanine, Valine, Leucine, Isoleucine, and |
| Aliphatic Side | Proline |
| Groups | |
| Polar, Uncharged | Serine, Threonine, Cysteine, Methionine, Asparagine, |
| Side Groups | Glutamine |
| Aromatic Side | Phenylalanine, Tyrosine, and Tryptophan |
| Chains | |

In some embodiments, the dysregulation of a NTRK gene, a Trk protein, or expression or activity, or level of the same, includes a splice variation in a TrkA mRNA which results in an expressed protein that is an alternatively spliced variant of TrkA having at least one residue deleted (as compared to a wild-type TrkA protein) resulting in constitutive activity of the TrkA kinase domain. In some embodiments, an alternatively spliced form of TrkA with constitutive activity is the TrkAIII splice variant and, e.g., is associated with neuroectodermal-derived tumors including Wilm's tumor, neuroblastoma, and medulloblastoma (see, e.g., U.S. Patent Publication No. 2015/0218132).

Overexpression or increased expression of a Trk gene (e.g., as compared to a control non-cancerous cell of the same cell type) is another type of dysregulation of a NTRK gene that is associated with a variety of different pediatric cancers. For example, overexpression of a Trk receptor has been observed in neuroectodermal-derived tumors including Wilm's tumor, neuroblastoma, and medulloblastoma (see, e.g., U.S. Patent Application Publication No. 2015/0218132), overexpression of NTRK2 in pediatric colorectal cancer subjects indicates poor prognosis in subjects (see, e.g., Tanaka et al., PLoS One 9:E96410, 2014), overexpression of NTRK2 has been observed in medulloblastoma and neuroblastoma in pediatric subjects (see, e.g., Evans et al., Clin. Cancer Res. 5:3592-3602, 1999; Geiger et al., J. Cancer Res. 65:7033, 2005). Decreased NTRK1 expression has been detected in bilateral stage IV adrenal neuroblastoma with multiple skin metastases in a neonate (see, e.g., Yanai et al., J. Pediatr. Surg. 39:1782-1783, 2004).

In some embodiments, a Trk-associated cancer is advanced solid and primary central nervous system tumors (e.g., advanced solid and primary central nervous system tumors that are refractory to standard therapy). In some embodiments, the cancer is a solid or central nervous system tumors (e.g., advanced solid or primary central nervous system tumor) that is refractory to standard therapy.

Cancers identified as having dysregulation of a NTRK gene, a Trk protein, or expression or activity, or level of the same (see references cited herein and also the www.cancer.gov and www.nccn.org websites include:

(A) Cancers wherein the dysregulation of a NTRK gene, a Trk protein, or expression or activity, or level of the same, includes one or more chromosome translocations or inversions resulting in Trk fusion proteins;

(B) Cancers wherein the dysregulation of a NTRK gene, a Trk protein, or expression or activity, or level of the same, includes one or more deletions, insertions, or mutations in the Trk protein;

(C) Cancers wherein the dysregulation of a NTRK gene, a Trk protein, or expression or activity, or level of the same, includes overexpression of wildtype Trk (e.g., leading to autocrine activation of a Trk);

In some embodiments, the dysregulation of a NTRK gene, a Trk protein, or expression or activity, or level of the same, includes a translocation that results in the expression of a TrkA, TrkB, or TrkC fusion protein, e.g., one of the TrkA, TrkB, or TrkC fusion proteins shown in Table 2, 5, and 8.

In some embodiments, provided herein is a method for treating a patient diagnosed with a Trk-associated cancer, comprising administering to the patient a therapeutically effective amount of one or more Trk inhibitors as provided herein and optionally an immunotherapy agent. For example, the Trk-associated cancer can be selected from the group of: non-small cell lung cancer, papillary thyroid carcinoma (e.g., recurrent papillary thyroid cancer; younger papillary thyroid cancer), glioblastoma multiforme, acute myeloid leukemia, colorectal carcinoma, large cell neuroendocrine carcinoma, prostate cancer, neuroblastoma, pancreatic carcinoma, melanoma, head and neck squamous cell carcinoma, gastric carcinoma, Spitz cancer, papillary thyroid carcinoma, colon cancer, acute myeloid leukemia, gastrointestinal stromal tumor (GIST) (e.g., GIST testing wild type for KIT/PDGFR/BRAF/SDH), sarcoma, glioma (e.g., pediatric glioma), intrahepatic cholangiocarcinoma, pilocytic astrocytoma, lower grade glioma, lung adenocarcinoma, salivary gland cancer, secretory breast cancer, fibrosarcoma, nephroma, and breast cancer. In some embodiments, the Trk-associated cancer has MSI-H. In some embodiments, the MSI-H Trk-associated cancer is colorectal cancer. In some embodiments, the MSI-H Trk-associated cancer is selected from the group consisting of: esophageal carcinoma, rectum adenocarcinoma, stomach adenocarcinoma, and uterine or endometrial cancer (e.g., uterine corpus endometrial carcinoma).

In some embodiments, a Trk-associated cancer is selected from the group of: Spitzoid melanoma, Spitz tumors (e.g., metastatic Spitz tumors), non-small cell lung cancer (NSCLC), thyroid carcinoma (e.g., papillary thyroid carcinoma (PTC)), acute myeloid leukemia (AML), sarcoma (e.g., undifferentiated sarcoma, adult soft tissue sarcoma, peripheral nerve sheath sarcoma, sarcoma—NOS (not otherwise specified), stromal sarcoma, small round cell sarcoma, spindle cell sarcoma, and epithelioid sarcoma), hepatobiliary cancer, glioma (e.g., pediatric gliomas), colorectal cancer (CRC), gliobastoma multiforme (GBM), large cell neuroendocrine cancer (LCNEC), thyroid cancer, intrahepatic cholangicarcinoma (ICC), pilocytic astrocytoma, lower-grade glioma, head and neck squamous cell carcinoma, adenocarcinoma (e.g., lung adenocarcinoma), salivary gland cancer, secretory breast carcinoma, breast cancer, breast-invasive carcinoma (e.g., invasive ductal carcinoma, invasive ductal carcinoma (NOS), multifocal invasive ductal carcinoma with secretory features, and invasive ductal carcinoma with secretory features) acute myeloid leukemia, fibrosarcoma, nephroma, melanoma, bronchogenic carcinoma, B-cell cancer, Bronchus cancer, cancer of the oral cavity or pharynx, cancer of hematological tissues, cervical cancer, gastric cancer, kidney cancer, liver cancer, multiple myeloma, ovarian cancer, pancreatic cancer, salivary gland cancer, small bowel or appendix cancer, testicular cancer, urinary bladder cancer, uterine or endrometrial cancer, inflammatory myofibroblastic tumors (e.g., inflammatory myofibroblastic kidney tumors), infantile myofibromatosis, lipofibromatosis, sinonasal adenocarcinoma, gastrointestinal stromal tumor, non-Hodgkin's lymphoma, neuroblastoma, small cell lung cancer, squamous cell carcinoma, esophageal-gastric cancer, skin cancer, neoplasm (e.g., a melanocystic neoplasm), Spitz nevi, astrocytoma, medulloblastoma, glioma, large cell neuroendocrine tumors, mammary analogue secretory carconioma (e.g., MASC; mammary analogue secretory carcinoma of the salivary gland), nonparotid acinic cell carcinoma, bone cancer, dendritic cell neoplasms, and rectum carcinoma. In some embodiments, the Trk-associated cancer is MSI-H. In some embodiments, the MSI-H Trk-associated cancer is colorectal cancer.

In some embodiments, provided herein is a method for treating a patient (e.g., a pediatric patient) diagnosed with a Trk-associated cancer, comprising administering to the patient a therapeutically effective amount of the compound of one or more Trk inhibitors as provided herein and optionally an immunotherapy agent. For example, the Trk-associated cancer can be selected from the group consisting of: pediatric nephroma, congenital fibrosarcoma (CFS), pediatric high-grade glioma (HGG), mesenchymal cancers (infant fibrosarcoma (IF), congenital mesoblastic nephroma, congenital infantile fibrosarcoma (CIFS)); locally advanced infantile fibrosarcoma, pilocytic astrocytoma, brain tumors (e.g., glioglastomas), pediatic acute leukemia, Ph-like acute lymphoblastic leukemia, cellular congenital mesoblastic nephroma (CMN); mixed congenital mesoblastic nephroma; infantile fibrosarcoma, adult fibrosarcoma, pediatric high-grade glioma (HGG), diffuse intrinsic pontine gliomas (DIPGs), non-brainstem HGGs (NBS-HGGs), anaplastic large cell lymphoma (ALCL), non-Hodgkin's lymphoma (NHL), pediatric papillary thyroid carcinoma, secretory breast cancer, soft tissue sarcoma, fibrous tumors, hepatobiliary cancer, non-rhabdomyosarcoma soft tissue sarcomas (NRSTS), spitzoid melanoma, pediatric hemangiopericytoma-like sarcoma, spindle cell sarcoma, NOS with myo/haemangiopericytic growth pattern, advanced pediatric solid tumors, neuroectodermal-derived tumors (e.g., Wilm's tumor, neuroblastoma, and medulloblastoma), pediatric colorectal cancer, adrenal neuroblastoma, and central nervous system tumors (e.g., advanced solid and primary central nervous system tumors that are refractory to standard therapy). In some embodiments, the Trk-associated cancer is MSI-H. In some embodiments, the MSI-H Trk-associated cancer is colorectal cancer.

In some embodiments, the cancer can be a fibrosarcoma. For example, the cancer can be infantile fibrosarcoma. In some embodiments, the subject is an infant and the fibrosarcoma is infantile fibrosarcoma. In some embodiments, the cancer is locally advanced infantile fibrosarcoma that would necessitate disfiguring surgery or amputation to achieve complete surgical resection. In some embodiments, the cancer is a myofibroblastic/fibroblastic tumor. The cancer can be a solid tumor or a primary CNS tumor. The cancer can also be a congenital mesoblastic nephroma.

In some embodiments, one or more Trk inhibitors as provided herein and optionally an immunotherapy agent are useful for treating Trk-associated cancers in pediatric patients. For example, the one or more Trk inhibitors as provided herein and optionally an immunotherapy agent can be used to treat infantile sarcoma, glioma (e.g., pediatric gliomas), neuroblastoma, congenital mesoblastic nephroma, brain low-grade glioma, and pontine glioma.

In some embodiments, the Trk-associated cancer is a glioma. For example, the Trk-associated cancer is selected from the group consisting of: pediatric high-grade glioma (HGG), diffuse intrinsic pontine gliomas (DIPGs), and on-brainstem HGGs (NBS-HGGs). In some embodiments, the cancer is an extracranial solid tumor. For example, the pediatric cancer is selected from the group consisting of: neuroblastoma, nephroblastoma (e.g., Wilm's tumor), rhabdomyosarcoma and hepatoblastoma.

In some embodiments, the fibrosarcoma is infantile fibrosarcoma.

In some embodiments, the Trk-associated cancer is LMNA-NTRK1 fusion soft tissue sarcoma or EVT6-NTRK3 fusion papillary thyroid cancer.

In some embodiments, the cancer is a Trk inhibitor-resistant cancer. In some embodiments, a Trk inhibitor-resistant cancer can be resistant to treatment with (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate (or a polymorph thereof), but the Trk inhibitor-resistant cancer is still sensitive to a treatment including (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one or a pharmaceutically acceptable salt thereof. In some embodiments, a Trk inhibitor-resistant cancer can be resistant to treatment with entrectinib, but the Trk inhibitor-resistant cancer is still sensitive to a treatment including (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one or pharmaceutically acceptable salt thereof.

A Trk inhibitor-resistant cancer cell can have, e.g., an increased rate of growth in the presence of at least one Trk inhibitor (e.g., any of the Trk inhibitors described herein or known in the art) as compared to the rate of growth of a control cell from a control subject having the same type of cancer and not having one or more of the point mutations in a NTRK1 gene described herein or one or more of the point mutations in a NTRK2 gene described herein or a point mutation in a NTRK3 gene described herein, when it is contacted with the at least one Trk inhibitor (e.g., a first Trk inhibitor). One of skill in the overart will appreciate that the Trk inhibitor-resistant cancer cell and the control cell are contacted with the same concentration of the at least one Trk inhibitor.

A Trk inhibitor-resistant cancer in a subject can have, e.g., an increased rate of growth of a solid tumor when the subject is treated with at least one Trk inhibitor (e.g., a first Trk inhibitor) as compared to the rate of growth of a control solid tumor in a control subject treated with the at least one Trk inhibitor and having the same type of cancer and not having one or more of the point mutations in a NTRK1 gene described herein or one or more of the point mutations in a NTRK2 gene described herein or a point mutation in a NTRK3 gene described herein). One of skill in the art will appreciate that the subject and the control subject are administered the same concentration of the at least one Trk inhibitor.

Trk inhibitor-resistant cancer in a subject can have, e.g., a decreased rate of apoptosis in a solid tumor when the subject is treated with at least one Trk inhibitor (e.g., any of the Trk inhibitors described herein or known in the art) as compared to the rate of apoptosis of a control solid tumor in a control subject treated with the at least one Trk inhibitor and having the same type of cancer and not having one or more of the point mutations in a NTRK1 gene described herein or one or more of the point mutations in a NTRK2 gene described herein or one or more point mutations in a NTRK3 gene described herein). One of skill in the art will appreciate that the subject and the control subject are administered the same concentration of the at least one Trk inhibitor.

In some embodiments, the Trk inhibitor that inhibits a Trk kinase with one or more point mutations is entrectinib (N-[5-(3,5-difluoro-benzyl)-1H-indazol-3-yl]-4-(4-methylpiperazin-1-yl)-2-(tetrahydro-pyran-4-ylamino)-benzamide), DAST (4-{4-[3-(4-chloro-3-trifluoromethylphenyl)-ureido]-3-fluorophenoxy}-pyridine-2-carboxylic acid methylamide), or (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one. In some embodiments, the Trk inhibitor is administered with an immunotherapy agent.

In the field of medical oncology it is normal practice to use a combination of different forms of treatment to treat each patient with cancer. In medical oncology the other component(s) of such conjoint treatment or therapy in addition to compositions provided herein may be, for example, surgery, radiotherapy, and chemotherapeutic agents, such as other kinase inhibitors, signal transduction inhibitors and/or monoclonal antibodies. For example, a surgery may be open surgery or minimally invasive surgery. The one or more Trk inhibitors provided herein and optionally an immunotherapy agent may also be useful as adjuvants to cancer treatment, that is, they can be used in combination with one or more additional therapies or therapeutic agents, for example a chemotherapeutic agent that works by the same or by a different mechanism of action.

The term “combination therapy” as used herein refers to a dosing regimen of two different therapeutically active agents (i.e., the components or combination partners of the combination) during a period of time, wherein the therapeutically active agents are administered together or separately in a manner prescribed by a medical care taker or according to a regulatory agency as defined herein. As can be appreciated in the art, a combination therapy can be administered to a patient for a period of time. In some embodiments, the period of time occurs following the administration of a different cancer therapeutic treatment/agent or a different combination of cancer therapeutic treatments/agents to the patient. In some embodiments, the period of time occurs before the administration of a different cancer therapeutic treatment/agent or a different combination of cancer therapeutic treatments/agents to the patient.

In some embodiments, one or more Trk inhibitors provided herein and optionally an immunotherapy agent, can be used prior to administration of an additional therapeutic agent or additional therapy. For example, a patient in need thereof can be administered one or more doses of one or more Trk inhibitors provided herein and optionally an immunotherapy agent for a period of time and then under go at least partial resection of a tumor. In some embodiments, the treatment with one or more doses of one or more Trk inhibitors provided herein and optionally an immunotherapy agent reduce the size of the tumor (e.g., the tumor burden) prior to the at least partial resection of a tumor. For example, in some embodiments a patient is administered one or more doses of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate prior to at least partial resection of a tumor. In some embodiments, a patient is administered one or more doses of (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one prior to at least partial resection of a tumor. In some embodiments, a patient is administered one or more doses of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate and one or more doses of (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one prior to at least partial resection of a tumor. In any of such embodiments, the patient can be administered one or more doses of an immunotherapy agent.

In some embodiments, following at least partial resection of the tumor, the patient is administered one or more doses of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate. In some embodiments, following at least partial resection of the tumor, the patient is administered one or more doses of (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one. In some embodiments, following at least partial resection of the tumor, the patient is administered one or more doses of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate and one or more doses of (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one. In any of such embodiments, the patient can be administered one or more doses of an immunotherapy agent following at least partial resection of the tumor.

In some embodiments, one or more doses of one or more Trk inhibitors as provided herein and optionally an immunotherapy agent can be administered in combination with one or more additional therapeutic agents or therapies selected from the group consisting of surgery, radiotherapy, signal transduction inhibitors, monoclonal antibodies, anti-inflammatory compounds, steroids, mitotic inhibitors, alkylating agents, antimetabolites, antisense DNA or RNA, intercalating antibiotics, growth factor inhibitors, signal transduction inhibitors, cell cycle inhibitors, enzyme inhibitors, retinoid receptor modulators, proteasome inhibitors, topoisomerase inhibitors, biological response modifiers, antihormones, angiogenesis inhibitors, cytostatic agents anti-androgens, targeted antibodies, HMG-CoA reductase inhibitors, prenyl-protein transferase inhibitors, chemotherapeutic agents, hormone therapy drugs, targeted therapy drugs, and aromatase inhibitors.

For example, the aromatase inhibitor is selected from the group consisting of aminoglutethimide, testolactone, anastrozole, letrozole, exemestane, vorozole, formestane, fadrozole, and 1,4,6-androstatriene-3,17-dione (ATD). In some embodiments, the one or more additional therapeutic agents is selected from the group consisting of palbociclib, abemaciclib, fulvestrant, topotecan, gemcitabine, imatinib mesylate, herceptin, 5-fluorouracil, leucovorin, carboplatin, cisplatin, taxanes, decitabine, cyclophosphamide, vinca alkaloids, imatinib, lapatinib, anthracyclines, rituximab, tamoxifen, irinotecan (CPT 11), pertuzumab, trastuzumab, and ado-trastuzumab emtansine. In some such cases, the patient has breast cancer (e.g., a Trk-associated breast cancer). For example, the breast cancer can be selected from the group consisting of secretory breast carcinoma, ductal carcinoma, ductal carcinoma in situ, invasive ductal carcinoma, invasive ductal carcinoma with secretory features, lobular carcinoma, lobular carcinoma in situ, invasive lobular carcinoma, invasive ductal carcinoma (NOS), multifocal invasive ductal carcinoma with secretory features, medullary carcinoma, tubular carcinoma, mucinous (colloid) carcinoma, Paget's disease of the breast, inflammatory carcinoma, angiosarcoma, invasive comedocarcinoma, scirrhous carcinoma, metaplastic carcinoma, papillary carcinoma, papillary carcinoma in situ, micropapillary carcinoma, cribriform carcinoma, undifferentiated or anaplastic carcinoma, male breast cancer, phyllodes tumors, adenoid cystic carcinoma, onset breast cancer, relapse breast cancer, and refractory breast cancer. In some embodiments, the breast cancer is selected from the group consisting of metastatic, hormone resistant, hormone receptor positive, estrogen receptor positive, estrogen receptor negative, progesterone receptor negative, progesterone receptor positive, HER2 positive, HER2 negative, double positive, triple-negative, triple-positive, and combinations thereof.

In some embodiments, one or more Trk inhibitors as provided herein and optionally an immunotherapy agent is useful for treating a Trk-associated cancer in combination with one or more additional therapeutic agents or therapies that work by the same or a different mechanism of action.

In some embodiments, the additional therapeutic agent(s) is selected from the group of: receptor tyrosine kinase-targeted therapeutic agents, including cabozantinib, crizotinib, erlotinib, gefitinib, imatinib, lapatinib, nilotinib, pazopanib, pertuzumab, regorafenib, sunitinib, and trastuzumab.

In some embodiments, the additional therapeutic agent(s) is selected from signal transduction pathway inhibitors, including, e.g., Ras-Raf-MEK-ERK pathway inhibitors (e.g., sorafenib, trametinib, or vemurafenib), PI3K-Akt-mTOR-S6K pathway inhibitors (e.g., everolimus, rapamycin, perifosine, or temsirolimus) and modulators of the apoptosis pathway (e.g., obataclax).

In some embodiments, the additional therapeutic agent(s) is selected from the group of: cytotoxic chemotherapeutics, including, e.g., arsenic trioxide, bleomycin, cabazitaxel, capecitabine, carboplatin, cisplatin, cyclophosphamide, cytarabine, dacarbazine, daunorubicin, docetaxel, doxorubicin, etoposide, fluorouracil, gemcitabine, irinotecan, lomustine, methotrexate, mitomycin C, oxaliplatin, paclitaxel, pemetrexed, temozolomide, and vincristine.

In some embodiments, the additional therapeutic agent(s) is selected from the group of angiogenesis-targeted therapies, including e.g., aflibercept and bevacizumab.

In some embodiments, the additional therapeutic agent(s) is selected from the group of immune-targeted agents, e.g., including aldesleukin, ipilimumab, lambrolizumab, nivolumab, and sipuleucel-T.

In some embodiments, the additional therapeutic agent(s) is selected from agents active against the downstream Trk pathway, including, e.g., NGF-targeted biopharmaceuticals, such as NGF antibodies and panTrk inhibitors.

In some embodiments, the additional therapeutic agent or therapy is radiotherapy, including, e.g., radioiodide therapy, external-beam radiation, and radium 223 therapy.

In some embodiments, the additional therapeutic agent(s) includes any one of the above listed therapies or therapeutic agents which are standards of care in cancers wherein the cancer has a dysregulation of a NTRK gene, a Trk protein, or expression or activity, or level of the same.

In some embodiments, the additional therapeutic agent(s) or therapy are selected from surgery, radiotherapy, signal transduction inhibitors, monoclonal antibodies, anti-inflammatory compounds, steroids, mitotic inhibitors, alkylating agents, antimetabolites, antisense DNA or RNA, intercalating antibiotics, growth factor inhibitors, cell cycle inhibitors, enzyme inhibitors, retinoid receptor modulators, proteasome inhibitors, topoisomerase inhibitors, biological response modifiers, antihormones, angiogenesis inhibitors, cytostatic agents anti-androgens, targeted antibodies, HMG-CoA reductase inhibitors, prenyl-protein transferase inhibitors, chemotherapeutic agents, hormone therapy drugs, targeted therapy drugs, and aromatase inhibitors. Non-limiting examples of an aromatase inhibitor include aminoglutethimide, testolactone, anastrozole, letrozole, exemestane, vorozole, formestane, fadrozole, and 1,4,6-androstatriene-3,17-dione (ATD). In some embodiments the one or more additional therapeutic agents is selected from the group consisting of palbociclib, abemaciclib, fulvestrant, topotecan, gemcitabine, imatinib mesylate, herceptin, 5-fluorouracil, leucovorin, carboplatin, cisplatin, taxanes, decitabine, cyclophosphamide, vinca alkaloids, imatinib, lapatinib, anthracyclines, rituximab, tamoxifen, irinotecan (CPT 11), pertuzumab, trastuzumab, and ado-trastuzumab emtansine.

In some embodiments, the enzyme inhibitor is an EHMT2 inhibitor. Non-limiting examples of EHMT2 inhibitors include BIX-01294 (BIX), UNC0638, A-366, UNC0642, DCG066, UNC0321, BRD 4770, UNC 0224, UNC 0646, UNC0631, BIX-01338, EZM8266, N-(1-isopropylpiperidin-4-yl)-6-methoxy-2-(4-methyl-1,4-diazepan-1-yl)-7-(3-(piperidin-1-yl)propoxy)quinazolin-4-amine, 2-(4-isopropyl-1,4-diazepan-1-yl)-N-(1-isopropylpiperidin-4-yl)-6-methoxy-7-(3-(piperidin-1-yl)propoxy)quinazolin-4-amine, and 2-(4,4-difluoropiperidin-1-yl)-N-(1-isopropylpiperidin-4-yl)-6-methoxy-7-(3-(pyrrolidin-1-yl)propoxy)quinazolin-4-amine.

The phrase “dysregulation of a kinase gene, a kinase protein, or the expression or activity or level of any of the same” refers to a genetic mutation (e.g., a chromosomal translocation that results in the expression of a fusion protein including a kinase domain and a fusion partner, a mutation in a kinase gene that results in the expression of a protein that includes a deletion of at least one amino acid as compared to a wildtype kinase protein, a mutation in a kinase gene that results in the expression of a kinase protein with one or more point mutations as compared to a wildtype kinase protein, a mutation in a kinase gene that results in the expression of a kinase protein with at least one inserted amino acid as compared to a wildtype kinase protein, a gene duplication that results in an increased level of kinase protein in a cell, or a mutation in a regulatory sequence (e.g., a promoter and/or enhancer) that results in an increased level of kinase protein in a cell), an alternative spliced version of a mRNA that results in a kinase protein having a deletion of at least one amino acid in the protein as compared to the wild-type kinase protein), or increased expression (e.g., increased levels) of a wildtype kinase protein in a mammalian cell due to aberrant cell signaling and/or dysregulated autocrine/paracrine signaling (e.g., as compared to a control non-cancerous cell). As another example, a dysregulation of a kinase gene, a kinase protein, or expression or activity, or level of any of the same, can be a mutation in a kinase gene that encodes a kinase protein that is constitutively active or has increased activity as compared to a kinase protein encoded by a kinase gene that does not include the mutation. For example, a dysregulation of a kinase gene, a kinase protein, or expression or activity, or level of any of the same, can be the result of a gene or chromosome translocation which results in the expression of a fusion protein that contains a first portion of a kinase protein that includes a functional kinase domain, and a second portion of a partner protein (i.e., that is not the primary protein). In some examples, dysregulation of a kinase gene, a kinase protein, or expression or activity or level of any of the same can be a result of a gene translocation of one kinase gene with a different gene. In some such embodiments, a kinase is selected from the group consisting of BRAF, ERK, MEK, and MET.

Treatment of a patient having a Trk-associated cancer as provided herein with one or more Trk inhibitors as provided herein can result in dysregulation of another kinase gene, a kinase, or the expression or activity or level of the same in the cancer, and/or resistance to a Trk inhibitor. For example, treatment of a patient having a Trk-associated cancer as provided herein with one or more Trk inhibitors as provided herein can result in dysregulation of one or more of a KRAS, BRAF, ERK, MEK, or MET kinase gene, a KRAS, BRAF, ERK, MEK, or MET kinase, or the expression or activity or level of the same in the cancer.

Treatment of a patient having a Trk-associated cancer as provided herein with a Trk inhibitor as provided herein in combination with a multi-kinase inhibitor or a target-specific inhibitor (e.g., a KRAS inhibitor, a BRAF inhibitor, a ERK inhibitor, a MEK inhibitor, or a MET inhibitor) can have increased therapeutic efficacy as compared to treatment of the same patient or a similar patient with the Trk inhibitor as a monotherapy, or the multi-kinase inhibitor or the target-specific inhibitor as a monotherapy.

Provided herein are methods of treating a patient having a Trk-associated cancer (e.g., any of the cancers described herein) and previously administered one or more Trk inhibitors as provided herein (e.g., as a monotherapy) that include: administering to the patient (i) a multi-kinase inhibitor or a target-specific inhibitor (e.g., a KRAS inhibitor, a BRAF inhibitor, a ERK inhibitor, a MEK inhibitor, or a MET inhibitor) as a monotherapy, or (ii) a therapeutically effective dose of a multi-kinase inhibitor or a target-specific inhibitor (e.g., a KRAS inhibitor, a BRAF inhibitor, a ERK inhibitor, a MEK inhibitor, or a MET inhibitor), and a therapeutically effective dose of the previously administered Trk inhibitor.

Provided herein are methods of treating a patient having a Trk-associated cancer (e.g., any of the cancers described herein) previously administered one or more Trk inhibitors as provided herein (e.g., as a monotherapy) that include: identifying a patient having a cancer cell that has a dysregulation of a KRAS, BRAF, ERK, MEK, or MET gene, a KRAS, BRAF, ERK, MEK, or MET kinase, or the expression or activity or level of the same; and administering to the patient (i) a multi-kinase inhibitor or a target-specific inhibitor (e.g., a KRAS inhibitor, a BRAF inhibitor, a ERK inhibitor, a MEK inhibitor, or a MET inhibitor) as a monotherapy, or (ii) a therapeutically effective dose of a multi-kinase inhibitor or a target-specific inhibitor (e.g., a KRAS inhibitor, a BRAF inhibitor, a ERK inhibitor, a MEK inhibitor, or a MET inhibitor), and a therapeutically effective dose of the previously administered Trk inhibitor.

Provided herein are methods of treating a patient having a Trk-associated cancer (e.g., any of the cancers described herein) that include: administering to a patient a therapeutically effective amount of one or more Trk inhibitors as provided herein (e.g., as a monotherapy) for a first period of time; after the period of time, identifying the patient as having a dysregulation of a KRAS, BRAF, ERK, MEK, or MET gene, a BRAF, ERK, MEK, or MET kinase, or the expression or activity or level of the same; and administering to the identified patient (i) a multi-kinase inhibitor or a target-specific inhibitor (e.g., a KRAS inhibitor, a BRAF inhibitor, a ERK inhibitor, a MEK inhibitor, or a MET inhibitor) as a monotherapy, or (ii) a therapeutically effective dose of a multi-kinase inhibitor or a target-specific inhibitor (e.g., a KRAS inhibitor, a BRAF inhibitor, a ERK inhibitor, a MEK inhibitor, or a MET inhibitor), and a therapeutically effective dose of the previously administered Trk inhibitor.

Provided herein are methods of treating a patient having a Trk-associated cancer (e.g., any of the cancers described herein) that has dysregulation of a KRAS gene, a KRAS kinase, or the expression or activity or level of the same that include administering to the patient (i) a therapeutically effective amount of one or more Trk inhibitors as provided herein and (ii) a therapeutically effective amount of a KRAS inhibitor (e.g., any of the KRAS inhibitors described herein or known in the art).

Provided herein are methods of treating a patient having a Trk-associated cancer (e.g., any of the cancers described herein) that include: identifying a patient having a cancer cell that has dysregulation of a KRAS gene, a KRAS kinase, or the expression or activity or level of the same; and administering to the identified patient (i) a therapeutically effective amount of one or more Trk inhibitors as provided herein and (ii) a therapeutically effective amount of a KRAS inhibitor (e.g., any of the KRAS inhibitors described herein or known in the art).

Provided herein are methods of treating a patient having a Trk-associated cancer (e.g., any of the cancers described herein) that include: administering to a patient a therapeutically effective amount of one or more Trk inhibitors as provided herein (e.g., as a monotherapy)(e.g., (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one) for a first period of time; after the period of time, identifying the patient as having a dysregulation of a KRAS gene, a KRAS kinase, or the expression or activity or level of the same; and administering to the identified patient (i) a therapeutically effective amount of a KRAS inhibitor as a monotherapy, or (ii) a therapeutically effective amount of a KRAS inhibitor and a therapeutically effective dose of the previously administered Trk inhibitor.

Further provided herein are methods of treating a patient having a Trk-associated cancer (e.g., any of the cancers described herein) that include: administering to a patient a therapeutically effective amount of a first Trk inhibitor as provided herein (e.g., as a monotherapy) (e.g., (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate) for a first period of time; after the period of time, identifying the patient as having one or more TRK inhibitor resistance mutations (e.g., that result in an increased resistance to the first TRK inhibitor) (e.g., a solvent front mutation) (e.g., one or more of the Trk-inhibitor resistance mutations listed in Tables 11-13); administering to the identified patient (i) a therapeutically effective amount of a second Trk inhibitor as provided herein (e.g., as a monotherapy) (e.g., (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one) for a second period of time, or (ii) a therapeutically effective amount of the second Trk inhibitor and a therapeutically effective amount of the previously administered first Trk inhibitor; after the period of time, identifying the patient as having a dysregulation of a KRAS gene, a KRAS kinase, or the expression or activity or level of the same; and administering to the identified patient (i) a therapeutically effective amount of a KRAS inhibitor as a monotherapy, or (ii) a therapeutically effective amount of a KRAS inhibitor and a therapeutically effective dose of the previously administered first Trk inhibitor and/or a therapeutically effective amount of the previously administered second Trk inhibitor.

The phrase “dysregulation of a KRAS gene, a KRAS protein, or the expression or activity or level of any of the same” refers to a genetic mutation (e.g., a chromosomal translocation that results in the expression of a fusion protein including a KRAS kinase domain and a fusion partner, a mutation in a KRAS gene that results in the expression of a KRAS protein that includes a deletion of at least one amino acid as compared to a wildtype KRAS protein, a mutation in a KRAS gene that results in the expression of a KRAS protein with one or more point mutations as compared to a wildtype KRAS protein, a mutation in a KRAS gene that results in the expression of a KRAS protein with at least one inserted amino acid as compared to a wildtype KRAS protein, a gene duplication that results in an increased level of KRAS protein in a cell, or a mutation in a regulatory sequence (e.g., a promoter and/or enhancer) that results in an increased level of KRAS protein in a cell), an alternative spliced version of a KRAS mRNA that results in a KRAS protein having a deletion of at least one amino acid in the KRAS protein as compared to the wild-type KRAS protein), or increased expression (e.g., increased levels) of a wildtype KRAS protein in a mammalian cell due to aberrant cell signaling and/or dysregulated autocrine/paracrine signaling (e.g., as compared to a control non-cancerous cell). As another example, a dysregulation of a KRAS gene, a KRAS protein, or expression or activity, or level of any of the same, can be a mutation in a KRAS gene that encodes a KRAS protein that is constitutively active or has increased activity as compared to a protein encoded by a KRAS gene that does not include the mutation. For example, a dysregulation of a KRAS gene, a KRAS protein, or expression or activity, or level of any of the same, can be the result of a gene or chromosome translocation which results in the expression of a fusion protein that contains a first portion of a KRAS protein that includes a functional kinase domain, and a second portion of a partner protein (i.e., that is not KRAS). In some examples, dysregulation of a KRAS gene, a KRAS protein, or expression or activity or level of any of the same can be a result of a gene translocation of one KRAS gene with another non-KRAS gene.

In some embodiments, the KRAS dysregulation is a mutation in a KRAS protein. For example, the mutation results in an oncogenic activation of the KRAS pathway. In some embodiments, the mutation is a mutation at amino acid position 12. For example, the mutation is a G12V mutation in the KRAS protein.

Non-limiting examples of KRAS inhibitors include one or more of a RAS-targeted therapeutic, a receptor tyrosine kinase inhibitor, a Ras-Raf-MEK-ERK pathway inhibitor, a PI3K-Akt-mTOR pathway inhibitor, and a farnesyl transferase inhibitor. In some embodiments, the RAS-targeted therapeutic is one or more of SML-10-70-4 and AA12. In some embodiments, the Ras-Raf-MEK-ERK pathway inhibitor is one or more of a BRAF inhibitor, a MEK inhibitor, and an ERK inhibitor. In some embodiments, the BRAF inhibitor is one or more of vemurafenib (ZELBORAF®), dabrafenib (TAFINLAR®), and encorafenib (BRAFTOVI®), BMS-908662 (XL281), sorafenib, LGX818, PLX3603, RAF265, RO5185426, GSK2118436, ARQ 736, GDC-0879, PLX-4720, AZ304, PLX-8394, HM95573, RO5126766, and LXH254. In some embodiments, the MEK inhibitor is one or more of trametinib (MEKINIST®, GSK1120212), cobimetinib (COTELLIC®), binimetinib (MEKTOVI®, MEK162), selumetinib (AZD6244), PD0325901, MSC1936369B, SHR7390, TAK-733, RO5126766, CS3006, WX-554, PD98059, CI1040 (PD184352), and hypothemycin. In some embodiments, the ERK inhibitor is one or more of FRI-20 (ON-01060), VTX-11e, 25-OH-D3-3-BE (B3CD, bromoacetoxycalcidiol), FR-180204, AEZ-131 (AEZS-131), AEZS-136, AZ-13767370, BL-EI-001, LY-3214996, LTT-462, KO-947, KO-947, MK-8353 (SCH900353), SCH772984, ulixertinib (BVD-523), CC-90003, GDC-0994 (RG-7482), ASN007, FR148083, 5-7-Oxozeaenol, 5-iodotubercidin, GDC0994, and ONC201. In some embodiments, the PI3K-Akt-mTOR pathway inhibitor is one or more of a PI3K inhibitor, an AKT inhibitor, and a mTOR inhibitor. In some embodiments, the PI3K inhibitor is one or more of buparlisib (BKM120), alpelisib (BYL719), WX-037, copanlisib (ALIQOPA™, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, and GSK2636771. In some embodiments, the AKT inhibitor is one or more of miltefosine (IMPADIVO®), wortmannin, NL-71-101, H-89, GSK690693, CCT128930, AZD5363, ipatasertib (GDC-0068, RG7440), A-674563, A-443654, AT7867, AT13148, uprosertib, afuresertib, DC120, 2-[4-(2-aminoprop-2-yl)phenyl]-3-phenylquinoxaline, MK-2206, edelfosine, miltefosine, perifosine, erucylphophocholine, erufosine, SR13668, OSU-A9, PH-316, PHT-427, PIT-1, DM-PIT-1, triciribine (Triciribine Phosphate Monohydrate), API-1, N-(4-(5-(3-acetamidophenyl)-2-(2-aminopyridin-3-yl)-3H-imidazo[4,5-b] pyridin-3-yl)benzyl)-3-fluorobenzamide, ARQ092, BAY 1125976, 3-oxo-tirucallic acid, lactoquinomycin, boc-Phe-vinyl ketone, Perifosine (D-21266), TCN, TCN-P, GSK2141795, and ONC201. In some embodiments, the mTOR inhibitor is one or more of MLN0128, AZD-2014, CC-223, AZD2014, CC-115, everolimus (RAD001), temsirolimus (CCI-779), ridaforolimus (AP-23573), and sirolimus (rapamycin). In some embodiments, the farnesyl transferase inhibitor is one or more of lonafarnib, tipifarnib, BMS-214662, L778123, L744832 and FTI-277. In some embodiments, the KRAS inhibitor is a MEK inhibitor and a PI3K inhibitor. In some embodiments, the KRAS inhibitor is a MEK inhibitor and an ERK inhibitor. Provided herein are methods of treating a patient having a Trk-associated cancer (e.g., any of the cancers described herein) that has dysregulation of a BRAF gene, a BRAF kinase, or the expression or activity or level of the same that include administering to the patient (i) a therapeutically effective amount of one or more Trk inhibitors as provided herein and (ii) a therapeutically effective amount of a BRAF inhibitor (e.g., any of the BRAF inhibitors described herein or known in the art).

Provided herein are methods of treating a patient having a Trk-associated cancer (e.g., any of the cancers described herein) that include: identifying a patient having a cancer cell that has dysregulation of a BRAF gene, a BRAF kinase, or the expression or activity or level of the same; and administering to the identified patient (i) a therapeutically effective amount of one or more Trk inhibitors as provided herein and (ii) a therapeutically effective amount of a BRAF inhibitor (e.g., any of the BRAF inhibitors described herein or known in the art).

Provided herein are methods of treating a patient having a Trk-associated cancer (e.g., any of the cancers described herein) that include: administering to a patient a therapeutically effective amount of one or more Trk inhibitors as provided herein (e.g., as a monotherapy) for a first period of time; after the period of time, identifying the patient as having a dysregulation of a BRAF gene, a BRAF kinase, or the expression or activity or level of the same; and administering to the identified patient (i) a therapeutically effective amount of a BRAF inhibitor as a monotherapy, or (ii) a therapeutically effective amount BRAF inhibitor and a therapeutically effective dose of the previously administered Trk inhibitor.

The phrase “dysregulation of a BRAF gene, a BRAF protein, or the expression or activity or level of any of the same” refers to a genetic mutation (e.g., a chromosomal translocation that results in the expression of a fusion protein including a BRAF kinase domain and a fusion partner, a mutation in a BRAF gene that results in the expression of a BRAF protein that includes a deletion of at least one amino acid as compared to a wildtype BRAF protein, a mutation in a BRAF gene that results in the expression of a BRAF protein with one or more point mutations as compared to a wildtype BRAF protein, a mutation in a BRAF gene that results in the expression of a BRAF protein with at least one inserted amino acid as compared to a wildtype BRAF protein, a gene duplication that results in an increased level of BRAF protein in a cell, or a mutation in a regulatory sequence (e.g., a promoter and/or enhancer) that results in an increased level of BRAF protein in a cell), an alternative spliced version of a BRAF mRNA that results in a BRAF protein having a deletion of at least one amino acid in the BRAF protein as compared to the wild-type BRAF protein), or increased expression (e.g., increased levels) of a wildtype BRAF protein in a mammalian cell due to aberrant cell signaling and/or dysregulated autocrine/paracrine signaling (e.g., as compared to a control non-cancerous cell). As another example, a dysregulation of a BRAF gene, a BRAF protein, or expression or activity, or level of any of the same, can be a mutation in a BRAF gene that encodes a BRAF protein that is constitutively active or has increased activity as compared to a protein encoded by a BRAF gene that does not include the mutation. For example, a dysregulation of a BRAF gene, a BRAF protein, or expression or activity, or level of any of the same, can be the result of a gene or chromosome translocation which results in the expression of a fusion protein that contains a first portion of a BRAF protein that includes a functional kinase domain, and a second portion of a partner protein (i.e., that is not BRAF). In some examples, dysregulation of a BRAF gene, a BRAF protein, or expression or activity or level of any of the same can be a result of a gene translocation of one BRAF gene with another non-BRAF gene.

Non-limiting examples of a BRAF inhibitor include vemurafenib (ZELBORAF®), dabrafenib (TAFINLAR®), and encorafenib (BRAFTOVI™), BMS-908662 (XL281), sorafenib, LGX818, PLX3603, RAF265, RO5185426, GSK2118436, ARQ 736, GDC-0879, PLX-4720, AZ304, PLX-8394, HM95573, RO5126766, and LXH254.

Provided herein are methods of treating a patient having a Trk-associated cancer (e.g., any of the cancers described herein) that has dysregulation of a ERK gene, a ERK kinase, or the expression or activity or level of the same that include administering to the patient (i) a therapeutically effective amount of one or more Trk inhibitors as provided herein and (ii) a therapeutically effective amount of a ERK inhibitor (e.g., any of the ERK inhibitors described herein or known in the art).

Provided herein are methods of treating a patient having a Trk-associated cancer (e.g., any of the cancers described herein) that include: identifying a patient having a cancer cell that has dysregulation of a ERK gene, a ERK kinase, or the expression or activity or level of the same; and administering to the identified patient (i) a therapeutically effective amount of one or more Trk inhibitors as provided herein and (ii) a therapeutically effective amount of a ERK inhibitor (e.g., any of the ERK inhibitors described herein or known in the art).

Provided herein are methods of treating a patient having a Trk-associated cancer (e.g., any of the cancers described herein) that include: administering to a patient a therapeutically effective amount of one or more Trk inhibitors as provided herein (e.g., as a monotherapy) for a first period of time; after the period of time, identifying the patient as having a dysregulation of a ERK gene, a ERK kinase, or the expression or activity or level of the same; and administering to the identified patient (i) a therapeutically effective amount of a ERK inhibitor as a monotherapy, or (ii) a therapeutically effective amount ERK inhibitor and a therapeutically effective dose of the previously administered Trk inhibitor.

The phrase “dysregulation of a ERK gene, a ERK protein, or the expression or activity or level of any of the same” refers to a genetic mutation (e.g., a chromosomal translocation that results in the expression of a fusion protein including a ERK kinase domain and a fusion partner, a mutation in a ERK gene that results in the expression of a ERK protein that includes a deletion of at least one amino acid as compared to a wildtype ERK protein, a mutation in a ERK gene that results in the expression of a ERK protein with one or more point mutations as compared to a wildtype ERK protein, a mutation in a ERK gene that results in the expression of a ERK protein with at least one inserted amino acid as compared to a wildtype ERK protein, a gene duplication that results in an increased level of ERK protein in a cell, or a mutation in a regulatory sequence (e.g., a promoter and/or enhancer) that results in an increased level of ERK protein in a cell), an alternative spliced version of a ERK mRNA that results in a ERK protein having a deletion of at least one amino acid in the ERK protein as compared to the wild-type ERK protein), or increased expression (e.g., increased levels) of a wildtype ERK protein in a mammalian cell due to aberrant cell signaling and/or dysregulated autocrine/paracrine signaling (e.g., as compared to a control non-cancerous cell). As another example, a dysregulation of a ERK gene, a ERK protein, or expression or activity, or level of any of the same, can be a mutation in a ERK gene that encodes a ERK protein that is constitutively active or has increased activity as compared to a protein encoded by a ERK gene that does not include the mutation. For example, a dysregulation of a ERK gene, a ERK protein, or expression or activity, or level of any of the same, can be the result of a gene or chromosome translocation which results in the expression of a fusion protein that contains a first portion of a ERK protein that includes a functional kinase domain, and a second portion of a partner protein (i.e., that is not ERK). In some examples, dysregulation of a ERK gene, a ERK protein, or expression or activity or level of any of the same can be a result of a gene translocation of one ERK gene with another non-ERK gene.

Non-limiting examples of a ERK inhibitor include FRI-20 (ON-01060), VTX-11e, 25-OH-D3-3-BE (B3CD, bromoacetoxycalcidiol), FR-180204, AEZ-131 (AEZ S-131), AEZS-136, AZ-13767370, BL-EI-001, LY-3214996, LTT-462, KO-947, KO-947, MK-8353 (SCH900353), SCH772984, ulixertinib (BVD-523), CC-90003, GDC-0994 (RG-7482), ASN007, FR148083, 5-7-Oxozeaenol, 5-iodotubercidin, GDC0994, and ONC201.

Provided herein are methods of treating a patient having a Trk-associated cancer (e.g., any of the cancers described herein) that has dysregulation of a MEK gene, a MEK kinase, or the expression or activity or level of the same that include administering to the patient (i) a therapeutically effective amount of one or more Trk inhibitors as provided herein and (ii) a therapeutically effective amount of a MEK inhibitor (e.g., any of the MEK inhibitors described herein or known in the art).

Provided herein are methods of treating a patient having a Trk-associated cancer (e.g., any of the cancers described herein) that include: identifying a patient having a cancer cell that has dysregulation of a MEK gene, a MEK kinase, or the expression or activity or level of the same; and administering to the identified patient (i) a therapeutically effective amount of one or more Trk inhibitors as provided herein and (ii) a therapeutically effective amount of a MEK inhibitor (e.g., any of the MEK inhibitors described herein or known in the art).

Provided herein are methods of treating a patient having a Trk-associated cancer (e.g., any of the cancers described herein) that include: administering to a patient a therapeutically effective amount of one or more Trk inhibitors as provided herein (e.g., as a monotherapy) for a first period of time; after the period of time, identifying the patient as having a dysregulation of a MEK gene, a MEK kinase, or the expression or activity or level of the same; and administering to the identified patient (i) a therapeutically effective amount of a MEK inhibitor as a monotherapy, or (ii) a therapeutically effective amount MEK inhibitor and a therapeutically effective dose of the previously administered Trk inhibitor.

The phrase “dysregulation of a MEK gene, a MEK protein, or the expression or activity or level of any of the same” refers to a genetic mutation (e.g., a chromosomal translocation that results in the expression of a fusion protein including a MEK kinase domain and a fusion partner, a mutation in a MEK gene that results in the expression of a MEK protein that includes a deletion of at least one amino acid as compared to a wildtype MEK protein, a mutation in a MEK gene that results in the expression of a MEK protein with one or more point mutations as compared to a wildtype MEK protein, a mutation in a MEK gene that results in the expression of a MEK protein with at least one inserted amino acid as compared to a wildtype MEK protein, a gene duplication that results in an increased level of MEK protein in a cell, or a mutation in a regulatory sequence (e.g., a promoter and/or enhancer) that results in an increased level of MEK protein in a cell), an alternative spliced version of a MEK mRNA that results in a MEK protein having a deletion of at least one amino acid in the MEK protein as compared to the wild-type MEK protein), or increased expression (e.g., increased levels) of a wildtype MEK protein in a mammalian cell due to aberrant cell signaling and/or dysregulated autocrine/paracrine signaling (e.g., as compared to a control non-cancerous cell). As another example, a dysregulation of a MEK gene, a MEK protein, or expression or activity, or level of any of the same, can be a mutation in a MEK gene that encodes a MEK protein that is constitutively active or has increased activity as compared to a protein encoded by a MEK gene that does not include the mutation. For example, a dysregulation of a MEK gene, a MEK protein, or expression or activity, or level of any of the same, can be the result of a gene or chromosome translocation which results in the expression of a fusion protein that contains a first portion of a MEK protein that includes a functional kinase domain, and a second portion of a partner protein (i.e., that is not MEK). In some examples, dysregulation of a MEK gene, a MEK protein, or expression or activity or level of any of the same can be a result of a gene translocation of one MEK gene with another non-MEK gene.

Non-limiting examples of a MEK inhibitor include trametinib (MEKINIST®, GSK1120212), cobimetinib (COTELLIC®), binimetinib (MEKTOVI®, MEK162), selumetinib (AZD6244), PD0325901, MSC1936369B, SHR7390, TAK-733, RO5126766, CS3006, WX-554, PD98059, CI1040 (PD184352), and hypothemycin.

Provided herein are methods of treating a patient having a Trk-associated cancer (e.g., any of the cancers described herein) that has dysregulation of a MET gene, a MET kinase, or the expression or activity or level of the same that include administering to the patient (i) a therapeutically effective amount of one or more Trk inhibitors as provided herein and (ii) a therapeutically effective amount of a MET inhibitor (e.g., any of the MET inhibitors described herein or known in the art).

Provided herein are methods of treating a patient having a Trk-associated cancer (e.g., any of the cancers described herein) that include: identifying a patient having a cancer cell that has dysregulation of a MET gene, a MET kinase, or the expression or activity or level of the same; and administering to the identified patient (i) a therapeutically effective amount of one or more Trk inhibitors as provided herein and (ii) a therapeutically effective amount of a MET inhibitor (e.g., any of the MET inhibitors described herein or known in the art).

Provided herein are methods of treating a patient having a Trk-associated cancer (e.g., any of the cancers described herein) that include: administering to a patient a therapeutically effective amount of one or more Trk inhibitors as provided herein (e.g., as a monotherapy) for a first period of time; after the period of time, identifying the patient as having a dysregulation of a MET gene, a MET kinase, or the expression or activity or level of the same; and administering to the identified patient (i) a therapeutically effective amount of a MET inhibitor as a monotherapy, or (ii) a therapeutically effective amount MET inhibitor and a therapeutically effective dose of the previously administered Trk inhibitor.

The phrase “dysregulation of a MET gene, a MET protein, or the expression or activity or level of any of the same” refers to a genetic mutation (e.g., a chromosomal translocation that results in the expression of a fusion protein including a MET kinase domain and a fusion partner, a mutation in a MET gene that results in the expression of a MET protein that includes a deletion of at least one amino acid as compared to a wildtype MET protein, a mutation in a MET gene that results in the expression of a MET protein with one or more point mutations as compared to a wildtype MET protein, a mutation in a MET gene that results in the expression of a MET protein with at least one inserted amino acid as compared to a wildtype MET protein, a gene duplication that results in an increased level of MET protein in a cell, or a mutation in a regulatory sequence (e.g., a promoter and/or enhancer) that results in an increased level of MET protein in a cell), an alternative spliced version of a MET mRNA that results in a MET protein having a deletion of at least one amino acid in the MET protein as compared to the wild-type MET protein), or increased expression (e.g., increased levels) of a wildtype MET protein in a mammalian cell due to aberrant cell signaling and/or dysregulated autocrine/paracrine signaling (e.g., as compared to a control non-cancerous cell). As another example, a dysregulation of a MET gene, a MET protein, or expression or activity, or level of any of the same, can be a mutation in a MET gene that encodes a MET protein that is constitutively active or has increased activity as compared to a protein encoded by a MET gene that does not include the mutation. For example, a dysregulation of a MET gene, a MET protein, or expression or activity, or level of any of the same, can be the result of a gene or chromosome translocation which results in the expression of a fusion protein that contains a first portion of a MET protein that includes a functional kinase domain, and a second portion of a partner protein (i.e., that is not MET). In some examples, dysregulation of a MET gene, a MET protein, or expression or activity or level of any of the same can be a result of a gene translocation of one MET gene with another non-MET gene.

Non-limiting examples of a MET inhibitor include capmatinib (INC280, INCB28060), onartuzumab (MetMAb), Savolitinib, tepotinib (MSC2156119J, EMD1214063), CE-35562, AMG-337, AMG-458, Foretinib, PHA-665725, MK-2461, PF-04217903 and SU11274, SU11274 and PHA-665752, SAIT301, HS-10241, ARGX-111, MSC2156119J, glumetinib (SCC244), EMD 1204831, AZD6094 (savolitinib, volitinib, HMPL-504), PLB1001, ABT-700, AMG 208, INCB028060, AL2846, PF-04217903, rilotumumab (AMG102), ficlatuzumab (AV-299), and TAK701, YYB101, tivantinib (ARQ 197), Golvatinib (E7050), Cabozantinib (XL 184, BMS-907351), Foretinib (GSK1363089), Crizotinib (PF-02341066), MK-2461, BPI-9016M, BPI-9016M, TQ-B3139, MGCD265, MK-8033, ABBV-399, HTI-1066, and JNJ-61186372.

Methods of detecting dysregulation of a kinase gene, a kinase protein, or expression or activity, or level of the same, include, e.g., detection of kinase gene translocations, e.g., using In Situ Hybridization (e.g., Fluorescent In Situ Hybridization (FISH) (e.g., as described in International Application Nos. PCT/US2013/061211 and PCT/US2013/057495, which are incorporated herein by reference)). For example, the use of a kinase specific antibody in combination with the VENTANA OptiView DAB IHC Detection Kit (e.g., using a VENTANA BenchMark ULTRA platform) can be used to detect the level of kinase protein expression (e.g., any of the kinase proteins described herein, e.g., any of the wildtype Trk, BRAF, ERK, MEK, or MET proteins described herein or Trk, BRAF, ERK, MEK, or MET fusion proteins described herein). In-situ hybridization (e.g., fluorescence in situ hybridization) can be used in any of the methods described herein to detect the expression of any of the kinase fusion proteins described herein.

For example, methods of detecting dysregulation of a NTRK gene, a Trk protein, or expression or activity, or level of the same, include, e.g., detection of NTRK gene translocations, e.g., using In Situ Hybridization (e.g., Fluorescent In Situ Hybridization (FISH) (e.g., as described in International Application Nos. PCT/US2013/061211 and PCT/US2013/057495, which are incorporated herein by reference)). For example, the use of pan-TRK (EPR17341) antibody in combination with the VENTANA OptiView DAB IHC Detection Kit (e.g., using a VENTANA BenchMark ULTRA platform) can be used to detect the level of Trk protein expression (e.g., any of the Trk proteins described herein, e.g., any of the wildtype Trk proteins described herein or Trk fusion proteins described herein). In-situ hybridization (e.g., fluorescence in situ hybridization) can be used in any of the methods described herein to detect the expression of any of the Trk fusion proteins described herein.

Also provided are methods of treating a subject identified or diagnosed as having a Trk-associated cancer (e.g., a subject that has been identified or diagnosed as having a Trk-associated cancer through the use of a regulatory agency-approved, e.g., FDA-approved, kit for identifying dysregulation of a NTRK gene, a Trk protein, or expression or activity, or level of the same, in a subject or a biopsy sample from the subject) (e.g., any of the Trk-associated cancers described herein or known in the art) that include administering to the subject a therapeutically effective amount of one or more Trk inhibitors as provided herein and optionally an immunotherapy agent. Also provided is a the use of one or more Trk inhibitors as provided herein and optionally an immunotherapy agent in treating a Trk-associated cancer in a subject identified or diagnosed as having a Trk-associated cancer (e.g., a subject that has been identified or diagnosed as having a Trk-associated cancer through the use of a regulatory agency-approved, e.g., FDA-approved, kit for identifying dysregulation of a NTRK gene, a Trk protein, or expression or activity, or level of the same, in a subject or a biopsy sample from the pediatric subject) (e.g., any of the Trk-associated cancers described herein or known in the art). Also provided is the use of compound of one or more Trk inhibitors as provided herein and optionally an immunotherapy agent for the manufacture of a medicament for treating a Trk-associated cancer in a subject identified or diagnosed as having a Trk-associated cancer (e.g., a subject that has been identified or diagnosed as having a Trk-associated cancer through the use of a regulatory agency-approved, e.g., FDA-approved, kit for identifying dysregulation of a NTRK gene, a Trk protein, or expression or activity, or level of the same, in a subject or a biopsy sample from the subject) (e.g., any of the Trk-associated cancers described herein or known in the art).

Also provided are methods of treating a subject (e.g., a subject suspected of having a Trk-associated cancer, a subject presenting with one or more symptoms of a Trk-associated cancer, or a subject having an elevated risk of developing a Trk-associated cancer) that include performing an assay (e.g., an assay that utilizes next generation sequencing, immunohistochemistry, break apart FISH, or dual-fusion FISH analysis) (e.g., using a regulatory agency-approved, e.g., FDA-approved, kit) on a sample obtained from the subject to determine whether the subject has dysregulation of a NTRK gene, a Trk protein, or expression or activity, or level of the same, and administering (e.g., specifically or selectively administering) a therapeutically effective amount of one or more Trk inhibitors as provided herein and optionally an immunotherapy agent to a subject determined to have dysregulation of a NTRK gene, a Trk protein, or expression or activity, or levels of the same. In some embodiments, the assay is a liquid biopsy. Additional assays, non-limiting assays that may be used in these methods are described herein. Additional assays are also known in the art.

In some embodiments, dysregulation of a Trk gene, a Trk kinase, or the expression or activity or level of any of the same can be identified using a liquid biopsy (variously referred to as a fluid biopsy or fluid phase biopsy). See, e.g., Karachialiou et al., “Real-time liquid biopsies become a reality in cancer treatment”, Ann. Transl. Med., 3(3):36, 2016. Liquid biopsy methods can be used to detect total tumor burden and/or the dysregulation of a Trk gene, a Trk kinase, or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of a Trk gene, a Trk kinase, or the expression or activity or level of any of the same. In some embodiments, liquid biopsies can be used to detect the presence of dysregulation of a Trk gene, a Trk kinase, or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect tumor-derived circulating ribonucleic acid (cRNA). Analysis of cRNA (e.g., using sensitive detection techniques such as, without limitation, next-generation sequencing (NGS), traditional PCR, digital PCR, or microarray analysis) can be used to identify dysregulation of a Trk gene, a Trk kinase, or the expression or activity or level of any of the same. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a Trk gene, a Trk kinase, or the expression or activity or level of any of the same.

In some embodiments, ctDNA derived from a single gene can be detected using a liquid biopsy. In some embodiments, ctDNA derived from a plurality of genes (e.g., 2, 3, 4, 5, 6, 7, 8, 9, 10, 15, 20, 25, 30, 35, 40, 45, 50, 55, 60, 65, 70, 75, 80, 85, 90, 95, 100 or more, or any number of genes in between these numbers) can be detected using a liquid biopsy. In some embodiments, ctDNA derived from a plurality of genes can be detected using any of a variety of commercially-available testing panels (e.g., commercially-available testing panels designed to detect dysregulation of a Trk gene, a Trk kinase, or the expression or activity or level of any of the same). Liquid biopsies can be used to detect dysregulation of a Trk gene, a Trk kinase, or the expression or activity or level of any of the same including, without limitation, point mutations or single nucleotide variants (SNVs), copy number variants (CNVs), genetic fusions (e.g., translocations or rearrangements), insertions, deletions, or any combination thereof. In some embodiments, a liquid biopsy can be used to detect a germline mutation. In some embodiments, a liquid biopsy can be used to detect a somatic mutation. In some embodiments, a liquid biopsy can be used to detect a primary genetic mutation (e.g., a primary mutation or a primary fusion that is associated with initial development of a disease, e.g., cancer). In some embodiments, a liquid biopsy can be used to detect a genetic mutation that develops after development of the primary genetic mutation (e.g., a resistance mutation that arises in response to a treatment administered to a subject). In some embodiments, a dysregulation of a Trk gene, a Trk kinase, or the expression or activity or level of any of the same identified using a liquid biopsy is also present in a cancer cell that is present in the subject (e.g., in a tumor). In some embodiments, any of the types of dysregulation of a Trk gene, a Trk kinase, or the expression or activity or level of any of the same described herein can be detected using a liquid biopsy. In some embodiments, a genetic mutation identified via a liquid biopsy can be used to identify the subject as a candidate for a particular treatment. For example, detection of dysregulation of a Trk gene, a Trk kinase, or the expression or activity or level of any of the same in the subject can indicate that the subject will be responsive to a treatment that includes administration of a one or more Trk inhibitors as provided herein and optionally an immunotherapy agent.

Liquid biopsies can be performed at multiple times during a course of diagnosis, a course of monitoring, and/or a course of treatment to determine one or more clinically relevant parameters including, without limitation, progression of the disease, efficacy of a treatment, or development of resistance mutations after administering a treatment to the subject. For example, a first liquid biopsy can be performed at a first time point and a second liquid biopsy can be performed at a second time point during a course of diagnosis, a course of monitoring, and/or a course of treatment. In some embodiments, the first time point can be a time point prior to diagnosing a subject with a disease (e.g., when the subject is healthy), and the second time point can be a time point after subject has developed the disease (e.g., the second time point can be used to diagnose the subject with the disease). In some embodiments, the first time point can be a time point prior to diagnosing a subject with a disease (e.g., when the subject is healthy), after which the subject is monitored, and the second time point can be a time point after monitoring the subject. In some embodiments, the first time point can be a time point after diagnosing a subject with a disease, after which a treatment is administered to the subject, and the second time point can be a time point after the treatment is administered; in such cases, the second time point can be used to assess the efficacy of the treatment (e.g., if the genetic mutation(s) detected at the first time point are reduced in abundance or are undetectable) or to determine the presence of a resistance mutation that has arisen as a result of the treatment. In some embodiments, a treatment to be administered to a subject can include one or more Trk inhibitors as provided herein and optionally an immunotherapy agent.

In some embodiments provided herein, the sample can be a tissue sample such as a cancer tissue sample, a biopsy sample, a serum sample, a spinal fluid sample, or a urine sample.

Also provided is use of one or more Trk inhibitors as provided herein and optionally an immunotherapy agent in treating a Trk-associated cancer in a subject identified or diagnosed as having a Trk-associated cancer through a step of performing an assay (e.g., an in vitro assay) (e.g., an assay that utilizes next generation sequencing, immunohistochemistry, break apart FISH, or dual-fusion FISH analysis) (e.g., using a regulatory agency-approved, e.g., FDA-approved, kit) on a sample obtained from the subject to determine whether the pediatric subject has dysregulation of a NTRK gene, a Trk protein, or expression or activity, or level of the same, where the presence of dysregulation of a NTRK gene, a Trk protein, or expression or activity, or level of the same, identifies that the subject has a Trk-associated cancer. Some embodiments of any of the methods or uses described herein further include recording in the subject's clinical record (e.g., a computer readable medium) that the subject determined to have dysregulation of a NTRK gene, a Trk protein, or expression or activity, or level of the same, through the performance of the assay, should be administered one or more Trk inhibitors as provided herein and optionally an immunotherapy agent.

In some embodiments of any of the methods or uses described herein, the subject has been identified or diagnosed as having a cancer with dysregulation of a NTRK gene, a Trk protein, or expression or activity, or level of the same (e.g., as determined using a regulatory agency-approved, e.g., FDA-approved, assay or kit). In some embodiments of any of the methods or uses described herein, the subject has a tumor that is positive for dysregulation of a NTRK gene, a Trk protein, or expression or activity, or level of the same (e.g., as determined using a regulatory agency-approved assay or kit). In some embodiments of any of the methods or uses described herein, the subject can be a subject with a tumor(s) that is positive for dysregulation of a NTRK gene, a Trk protein, or expression or activity, or level of the same (e.g., identified as positive using a regulatory agency-approved, e.g., FDA-approved, assay or kit). In some embodiments of any of the methods or uses described herein, the subject can be a subject whose tumors have dysregulation of a NTRK gene, a Trk protein, or expression or activity, or a level of the same (e.g., where the tumor is identified as such using a regulatory agency-approved, e.g., FDA-approved, kit or assay). In some embodiments of any of the methods or uses described herein, the subject is suspected of having a Trk-associated cancer. In some embodiments of any of the methods or uses described herein, the subject has a clinical record indicating that the subject has a tumor that has dysregulation of a NTRK gene, a Trk protein, or expression or activity, or level of the same (and optionally the clinical record indicates that the subject should be treated with any of the compositions provided herein).

Also provided are methods of treating a subject that include administering a therapeutically effective amount of one or more Trk inhibitors as provided herein and optionally an immunotherapy agent to a subject having a clinical record that indicates that the subject has dysregulation of a NTRK gene, a Trk protein, or expression or activity, or level of the same. Also provided is the use of one or more Trk inhibitors as provided herein and optionally an immunotherapy agent for the manufacture of a medicament for treating a Trk-associated cancer in a subject having a clinical record that indicates that the subject has dysregulation of a NTRK gene, a Trk protein, or expression or activity, or level of the same. Also provided is the use of one or more Trk inhibitors as provided herein and optionally an immunotherapy agent for the manufacture of a medicament for treating a Trk-associated cancer in a subject having a clinical record that indicates that the subject has dysregulation of a NTRK gene, a Trk protein, or expression or activity, or level of the same. Some embodiments of these methods and uses can further include: a step of performing an assay (e.g., an in vitro assay) (e.g., an assay that utilizes next generation sequencing, immunohistochemistry, break apart FISH, or dual-fusion FISH analysis) (e.g., using a regulatory agency-approved, e.g., FDA-approved, kit) on a sample obtained from the subject to determine whether the subject has dysregulation of a NTRK gene, a Trk protein, or expression or activity, or level of the same, and recording information in a subject's clinical file (e.g., a computer-readable medium) that the subject has been identified to have dysregulation of a NTRK gene, a Trk protein, or expression or activity, or level of the same.

Also provided are methods (e.g., in vitro methods) of selecting a treatment for a subject that include selecting a treatment including administration of a therapeutically effective amount of one or more Trk inhibitors as provided herein and optionally an immunotherapy agent for a subject identified or diagnosed as having a Trk-associated cancer (e.g., a subject that has been identified or diagnosed as having a Trk-associated cancer through the use of a regulatory agency-approved, e.g., FDA-approved, kit for identifying dysregulation of a NTRK gene, a Trk protein, or expression or activity, or level of the same, in a subject or a biopsy sample from the subject) (e.g., any of the Trk-associated cancers described herein or known in the art). Some embodiments can further include administering the selected treatment to the subject identified or diagnosed as having a Trk-associated cancer. Some embodiments can further include a step of performing an assay (e.g., an in vitro assay) (e.g., an assay that utilizes next generation sequencing, immunohistochemistry, break apart FISH, or dual-fusion FISH analysis) (e.g., using a regulatory agency-approved, e.g., FDA-approved, kit) on a sample obtained from the subject to determine whether the subject has dysregulation of a NTRK gene, a Trk protein, or expression or activity, or level of the same, and identifying or diagnosing a subject determined to have dysregulation of a NTRK gene, a Trk protein, or expression or activity, or level of the same, as having a Trk-associated cancer.

Also provided are methods of selecting a treatment for a subject that include administration of a therapeutically effective amount of one or more Trk inhibitors as provided herein and optionally an immunotherapy agent, wherein the methods include a step of performing an assay (e.g., an in vitro assay) (e.g., an assay that utilizes next generation sequencing, immunohistochemistry, break apart FISH, or dual-fusion FISH analysis) (e.g., using a regulatory agency-approved, e.g., FDA-approved, kit) on a sample obtained from the subject to determine whether the subject has dysregulation of a NTRK gene, a Trk protein, or expression or activity, or level of the same, and identifying or diagnosing a subject determined to have dysregulation of a NTRK gene, a Trk protein, or expression or activity, or level of the same, as having a Trk-associated cancer, and selecting a therapeutic treatment including administration of a therapeutically effective amount of one or more Trk inhibitors as provided herein and optionally an immunotherapy agent for the subject identified or diagnosed as having a Trk-associated cancer. Some embodiments further include administering the selected treatment to the subject identified or diagnosed as having a Trk-associated cancer.

Also provided are methods of selecting a subject for treatment including administration of a therapeutically effective amount of one or more Trk inhibitors as provided herein and optionally an immunotherapy agent that include selecting, identifying, or diagnosing a subject having a Trk-associated cancer, and selecting the subject for treatment including administration of a therapeutically effective amount of one or more Trk inhibitors as provided herein and optionally an immunotherapy agent. In some embodiments, identifying or diagnosing a subject as having a Trk-associated cancer can include a step of performing an assay (e.g., an in vitro assay) (e.g., an assay that utilizes next generation sequencing, immunohistochemistry, break apart FISH, or dual-fusion FISH analysis) (e.g., using a regulatory agency-approved, e.g., FDA-approved, kit) on a sample obtained from the subject to determine whether the subject has dysregulation of a NTRK gene, a Trk protein, or expression or activity, or level of the same, and identifying or diagnosing a subject determined to have dysregulation of a NTRK gene, a Trk protein, or expression or activity, or level of the same, as having a Trk-associated cancer. In some embodiments, the selecting a treatment can be used as part of a clinical study that includes administration of various treatments of a Trk-associated cancer.

In some embodiments of any of the methods or uses described herein, an assay used determine whether the subject has dysregulation of a NTRK gene, a Trk protein, or expression or activity, or level of the same, using a sample (e.g., a biological sample or a biopsy sample (e.g., a paraffin-embedded biopsy sample) from a subject (e.g., a pediatric subject (e.g., an infant, a child, or an adolescent)) suspected of having a Trk-associated cancer, a subject having one or more symptoms of a Trk-associated cancer, and/or a subject that has an increased risk of developing a Trk-associated cancer can include, for example, next generation sequencing, multiplexed assays (e.g., RNA-based multiplexed assays, NANOSTRING nCOUNTER VANTAGE 3D™), immunohistochemistry, fluorescence microscopy, break apart FISH analysis, Southern blotting, Western blotting, FACS analysis, Northern blotting, and PCR-based amplification (e.g., RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of a NTRK gene, a Trk protein, or expression or activity, or levels of the same (see, e.g., the references cited herein).

In some embodiments, the subject (e.g., a pediatric subject, e.g., an infant, child, or adolescent) has been identified or diagnosed as having a cancer with dysregulation of a NTRK gene, a Trk protein, or expression or activity, or level of the same (e.g., as determined using a regulatory agency-approved, e.g., FDA-approved, assay or kit). In some embodiments, the subject (e.g., a pediatric subject, e.g., an infant, child, or adolescent) has a tumor that is positive for dysregulation of a NTRK gene, a Trk protein, or expression or activity, or level of the same (e.g., as determined using a regulatory agency-approved assay or kit). The subject (e.g., a pediatric subject, e.g., an infant, child, or adolescent) can be a subject with a tumor(s) that is positive for dysregulation of a NTRK gene, a Trk protein, or expression or activity, or level of the same (e.g., identified as positive using a regulatory agency-approved, e.g., FDA-approved, assay or kit). The subject (e.g., a pediatric subject, e.g., an infant, child, or adolescent) can be a subject whose tumors have dysregulation of a NTRK gene, a Trk protein, or expression or activity, or a level of the same (e.g., where the tumor is identified as such using a regulatory agency-approved, e.g., FDA-approved, kit or assay). In some embodiments, the subject (e.g., a pediatric subject, e.g., an infant, child, or adolescent) is suspected of having a Trk-associated cancer. In some embodiments, the subject (e.g., a pediatric subject, e.g., an infant, child, or adolescent) has a clinical record (e.g., a computer-readable medium) indicating that the subject has a tumor that has dysregulation of a NTRK gene, a Trk protein, or expression or activity, or level of the same (and optionally the clinical record further indicates that the subject should be treated with any of the compositions provided herein).

In some embodiments, a dose of the one or more Trk inhibitors contains, per unit dosage unit, about 2 mg, about 4 mg, about 6 mg, about 8 mg, about 10 mg, about 12 mg, about 14 mg, about 16 mg, about 18 mg, about 20 mg, about 30 mg, about 40 mg, about 50 mg, about 100 mg, about 150 mg, about 200 mg, about 250 mg, about 300 mg, about 400 mg, or about 500 mg of the one or more Trk inhibitors, independently. The dosages, however, may be varied depending upon the requirement of the patients, the severity of the condition being treated and the compound being employed. In some embodiments, the dosages are administered once daily (QD) or twice daily (BID).

Optimal dosages to be administered may be readily determined by those skilled in the art, and will vary with the mode of administration, the strength of the preparation, the mode of administration, and the advancement of the disease condition. In addition, factors associated with the particular patient being treated, including patient age, weight, diet and time of administration, will result in the need to adjust dosages.

In some embodiments, the compounds provided herein are administered on a continuous 28-day schedule. For example, a single cycle of administration includes 28 days of continuous dosing. Such dosing can be; for example, one daily or twice daily.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

In some embodiments, the patient is refractory to standard therapy (e.g., standard of care). In some embodiments, the patent has no standard therapy option. In some embodiments, the patient relapsed or progressed after standard therapy. In some embodiments, the methods provided herein are useful for treating locally advanced or metastatic solid tumors refractory to standard therapies.

In some embodiments, the methods provided can follow after surgical resection has failed to inhibit progression of the fibrosarcoma in the subject. The methods provided herein can also follow after chemotherapy including administration of at least one of vincristine, actinomycin-D, cyclophosphamide, ifosfamide, etoposide, doxorubicin has failed to inhibit tumor progression in the subject. For example, the methods provided herein can follow after administration of at least one of vincristine, actinomycin-D, and cyclophosphamide has failed to inhibit tumor progression in the subject. The methods provided herein can also follow after administration of at least one of ifosfamide and doxorubicin has failed to inhibit tumor progression in the subject.

In some embodiments, the methods provided can follow after one or more of surgical resection, radiotherapy, and chemotherapy have failed to inhibit progression of a breast cancer in the subject. Non-limiting examples of chemotherapy include administration of at least one of fluorouracil, doxorubicin, cyclophosphamide, gemcitabine, carboplatin, docetaxel, dacarbazine, paclitaxel, anastrozole, letrozole, palbociclib, vinorelbine, ifosfamide, and eribulin. Non-limiting examples of surgical resection include mastectomy. In some embodiments, the chemotherapy that failed to inhibit progression of a breast cancer in the subject was administered in combination with a prophylactic agent. Non-limiting examples of prophylactic agents include mesna and filgrastim. For example, the methods provided herein can follow after administration of mastectomy and palliative radiation has failed to inhibit tumor progression in the subject. The methods provided herein can also follow after administration of one or more of a combination of fluorouracil, doxorubicin, and cyclophosphamide; a combination of carboplatin and docetaxel; a combination of vinorelbine and gemcitabine; a combination of ifosfamide, doxorubicin, dacarbazine, and mesna; a combination of carboplatin and paclitaxel; and surgical resection has failed to inhibit tumor progression in the subject. The methods provided herein can also follow after administration of one or more of a combination of docetaxel and cyclophosphamide; anastrozole; a combination of eribulin and filgrastim; mastectomy; and radiation therapy has failed to inhibit tumor progression in the subject. The methods provided herein can also follow after administration of one or more of surgical resection; adjuvant chemotherapy; and a combination of palbociclib and letrozole has failed to inhibit tumor progression in the subject.

In some embodiments, the method of treating cancer comprises administering to the patient a therapeutically effective amount of a first Trk inhibitor or a pharmaceutically acceptable salt thereof, a second Trk inhibitor or a pharmaceutically acceptable salt thereof, and an immunotherapy agent. In some embodiments, the first Trk inhibitor and second Trk inhibitor are as described herein provided that they are not the same. In some embodiments, the first Trk inhibitor is (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate. In some embodiments, the second Trk inhibitor inhibits a Trk kinase with one or more point mutations. In some embodiments, the second Trk inhibitor is (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one. In some embodiments the first Trk inhibitor is (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, and the second Trk inhibitor is (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one. In some embodiments, the immunotherapy agent is a cellular immunotherapy, an antibody therapy, an antibody-drug conjugate, a toxin, blinatumomab (AMG103) or midostaurin (Rydapt), a cytokine therapy, an immune checkpoint inhibitor, an mRNA-based immunotherapy, bacillus Calmette-Guerin (BCG) therapy, an oncolytic virus therapy, a cancer vaccine, a peptide vaccine, or a DNA-based vaccine as described herein.

In some embodiments, the method of treating cancer comprises administering to the patient a therapeutically effective amount of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, a second Trk inhibitor or a pharmaceutically acceptable salt thereof, and an immunotherapy agent. In some embodiments the second Trk inhibitor is as described herein provided that it is not (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate. In some embodiments, the second Trk inhibitor inhibits a Trk kinase with one or more point mutations. In some embodiments, the second Trk inhibitor is (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one. In some embodiments, the immunotherapy agent is a cellular immunotherapy, an antibody therapy, an antibody-drug conjugate, a toxin, blinatumomab (AMG103) or midostaurin (Rydapt), a cytokine therapy, an immune checkpoint inhibitor, an mRNA-based immunotherapy, bacillus Calmette-Guerin (BCG) therapy, an oncolytic virus therapy, a cancer vaccine, a peptide vaccine, or a DNA-based vaccine as described herein.

In some embodiments, the method of treating cancer comprises administering to the patient a therapeutically effective amount of (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one, a second Trk inhibitor or a pharmaceutically acceptable salt thereof, and an immunotherapy agent. In some embodiments, the second Trk inhibitor is as described herein provided that it is not (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one. In some embodiments, the immunotherapy agent is a cellular immunotherapy, an antibody therapy, an antibody-drug conjugate, a toxin, blinatumomab (AMG103) or midostaurin (Rydapt), a cytokine therapy, an immune checkpoint inhibitor, an mRNA-based immunotherapy, bacillus Calmette-Guerin (BCG) therapy, an oncolytic virus therapy, a cancer vaccine, a peptide vaccine, or a DNA-based vaccine as described herein.

In some embodiments, the method of treating cancer comprises administering to the patient a therapeutically effective amount of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one, and an immunotherapy agent. In some embodiments, the immunotherapy agent is a cellular immunotherapy, an antibody therapy, an antibody-drug conjugate, a toxin, blinatumomab (AMG103) or midostaurin (Rydapt), a cytokine therapy, an immune checkpoint inhibitor, an mRNA-based immunotherapy, bacillus Calmette-Guerin (BCG) therapy, an oncolytic virus therapy, a cancer vaccine, a peptide vaccine, or a DNA-based vaccine as described herein.

In some embodiments, the method of treating cancer comprises administering to the patient a therapeutically effective amount of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate and an immunotherapy agent. In some embodiments, the immunotherapy agent is a cellular immunotherapy, an antibody therapy, an antibody-drug conjugate, a toxin, blinatumomab (AMG103) or midostaurin (Rydapt), a cytokine therapy, an immune checkpoint inhibitor, an mRNA-based immunotherapy, bacillus Calmette-Guerin (BCG) therapy, an oncolytic virus therapy, a cancer vaccine, a peptide vaccine, or a DNA-based vaccine as described herein.

In some embodiments, the method of treating cancer comprises administering to the patient a therapeutically effective amount of (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one and an immunotherapy agent. In some embodiments, the immunotherapy agent is a cellular immunotherapy, an antibody therapy, an antibody-drug conjugate, a toxin, blinatumomab (AMG103) or midostaurin (Rydapt), a cytokine therapy, an immune checkpoint inhibitor, an mRNA-based immunotherapy, bacillus Calmette-Guerin (BCG) therapy, an oncolytic virus therapy, a cancer vaccine, a peptide vaccine, or a DNA-based vaccine as described herein.

In some embodiments, the method of treating cancer comprises administering to the patient a therapeutically effective amount of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, entrectinib, and an immunotherapy agent. In some embodiments, the immunotherapy agent is a cellular immunotherapy, an antibody therapy, an antibody-drug conjugate, a toxin, blinatumomab (AMG103) or midostaurin (Rydapt), a cytokine therapy, an immune checkpoint inhibitor, an mRNA-based immunotherapy, bacillus Calmette-Guerin (BCG) therapy, an oncolytic virus therapy, a cancer vaccine, a peptide vaccine, or a DNA-based vaccine as described herein.

Methods of treating a cancer are provided herein. The term “treating” or “positive response to treatment” means an improvement in the condition of a subject having a cancer, e.g., one or more of a decrease in the size of one or more tumor(s) in a subject, a decrease or no substantial change in the growth rate of one or more tumor(s) in a subject, a decrease in metastasis in a subject, and an increase in the period of remission for a subject (e.g., as compared to the one or more metric(s) in a subject having a similar cancer receiving no treatment or a different treatment, or as compared to the one or more metric(s) in the same subject prior to treatment). Additional metrics for assessing response to a treatment in a subject having a cancer are known in the art.

Non-limiting examples of cancer (e.g., a Trk-associated cancer) include adenocarcinoma, adrenal gland cortical carcinoma, adrenal gland neuroblastoma, anus squamous cell carcinoma, appendix adenocarcinoma, bladder urothelial carcinoma, bile duct adenocarcinoma, biliary tract cancer, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, brain astrocytoma, brain glioblastoma, brain medulloblastoma, brain meningioma, brain oligodendroglioma, breast adenoid cystic carcinoma, breast carcinoma, breast ductal carcinoma in situ, breast invasive ductal carcinoma (e.g., invasive ductal carcinoma (NOS), multifocal invasive ductal carcinoma with secretory features, and invasive ductal carcinoma with secretory features), breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, leukemia lymphocytic, liver cholangiocarcinoma, liver hepatocellular carcinoma, respiratory tract neoplasms, lung adenocarcinoma, lung adenosquamous carcinoma, lung atypical carcinoid, lung carcinosarcoma, lung large cell neuroendocrine carcinoma, lung non-small cell lung carcinoma, lung sarcoma, lung sarcomatoid carcinoma, lung small cell carcinoma, lung small cell undifferentiated carcinoma, lung squamous cell carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, non-Hodgkin's lymphoma, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin adnexal carcinoma, skin basal cell carcinoma, skin melanoma, skin Merkel cell carcinoma, skin squamous cell carcinoma, small intestine adenocarcinoma, small intestine gastrointestinal stromal tumors (GISTs), pan-negative GIST tumors, soft tissue angiosarcoma, soft tissue Ewing sarcoma, soft tissue hemangioendothelioma, soft tissue inflammatory myofibroblastic tumor, soft tissue leiomyosarcoma, soft tissue liposarcoma, soft tissue neuroblastoma, soft tissue paraganglioma, soft tissue perivascular epitheloid cell tumor, soft tissue sarcoma, non-rhabdomyosarcoma soft tissue sarcomas (NRSTS), retroperitoneal congenital sarcoma, soft tissue synovial sarcoma, locally advanced sarcoma, thoracic neoplasms, hepatobiliary cancer, stomach adenocarcinoma, stomach adenocarcinoma diffuse type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma.

Additional examples of cancers (e.g., Trk inhibitor-resistant cancer) include: adrenocortical carcinoma, anal cancer, appendix cancer, atypical teratoid/rhabdoid tumor (e.g., central nervous system atypical teratoid/rhabdoid tumor), B-cell cancer, bile duct cancer, bladder cancer, bone cancer (e.g., osteosarcoma and malignant fibrous histiocytoma), brain cancer (e.g., brain and spinal cord tumor, brain stem glioma, central nervous system embryonal tumors, central nervous system germ cell tumors, craniopharyngioma, and ependymoma), breast cancer, bronchogenic carcinoma, bronchus cancer, cancer of hematological tissues, cancer of the oral cavity or pharynx, carcinoid tumor, cervical cancer, childhood cancers, chordoma, chronic lymphocytic leukemia, chronic myeloproliferative neoplasms, colon cancer, colorectal cancer, cutaneous T-cell lymphoma, ductal carcinoma in situ, embryonal tumor, endometrial cancer, esophageal cancer, esthesioneuroblastoma, extracranial germ cell tumor, extragonadal germ cell tumor, extrahepatic bile duct cancer, eye cancer (e.g., retinoblastoma), fallopian tube cancer, fibrosarcoma, fibrous histiocytoma of bone, gallbladder cancer, gastric cancer, gastrointestinal carcinoid tumor, germ cell tumor, gestational trophoblastic disease, nerve tissue neoplasms, glioblastoma multiforme, glioma (e.g., lower-grade glioma), head and neck cancer, heart cancer, histiocytosis, hypopharyngeal cancer, inflammatory myofibroblastic tumors, intrahepatic cholangiocarcinoma, islet cell tumor, kidney cancer (e.g., renal cell cancer), Langerhans cell histiocytosis, large cell neuroendocrine cancer, laryngeal cancer, leukemia (e.g., acute lymphoblastic leukemia, acute myeloid leukemia, chronic myelogenous leukemia, and hairy cell leukemia), lip cancer, liver cancer, lung cancer, Burkitt lymphoma, Hodgkin's lymphoma, and primary central nervous system lymphoma), medulloblastoma, mesothelioma, mouth cancer, multiple myeloma, myelodysplastic syndromes, nasal cavity and paranasal sinus cancer, nasopharyngeal cancer, neoplasm (e.g., a melanocystic neoplasm), nephroma, neuroblastoma, non-small cell lung cancer, oral cancer, oropharyngeal cancer, ovarian cancer, pancreatic cancer, paraganglioma, parathyroid cancer, glioma (e.g., pediatric gliomas), penile cancer, pharyngeal cancer, pheochromocytoma, pilocytic astrocytoma, pituitary tumor, plasma cell neoplasm, primary peritoneal cancer, prostate cancer, rectum carcinoma, salivary gland cancer, sarcoma (e.g., Ewing sarcoma, rhabdomyosarcoma, uterine sarcoma, and undifferentiated sarcoma), secretory breast carcinoma, Sezary syndrome, skin cancer, small bowel cancer, small cell lung cancer, small intestine cancer, Spitz nevi, Spitz tumors, spitzoid melanoma, stomach cancer, squamous cell carcinoma, squamous neck cancer, testicular cancer, throat cancer, thymoma and thymic carcinoma, thyroid carcinoma, urethral cancer, uterine cancer, uterine endometrical cancer, urinary bladder cancer, vaginal cancer, vulvar cancer, and Wilms tumor.

In some embodiments, the compounds provided herein exhibit brain and/or central nervous system (CNS) penetrance. Such compounds are capable of crossing the blood brain barrier and inhibiting a Trk kinase in the brain and/or other CNS structures. In some embodiments, the one or more Trk inhibitors and optionally an immunotherapy agent provided herein are capable of crossing the blood brain barrier in a therapeutically effective amount. For example, treatment of a patient with cancer (e.g., a Trk-associated cancer such as a Trk-associated brain or CNS cancer) can include administration (e.g., oral administration) of one or more Trk inhibitors and optionally an immunotherapy agent to the patient. In some such embodiments, the one or more Trk inhibitors and optionally an immunotherapy agent provided herein are useful for treating a primary brain tumor or metastatic brain tumor. For example, a Trk-associated primary brain tumor or metastatic brain tumor. In some embodiments, such Trk inhibitors include (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate and (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one.

For example, the one or more Trk inhibitors and optionally an immunotherapy agent provided herein can be used in the treatment of one or more of gliomas such as glioblastoma (also known as glioblastoma multiforme), astrocytomas, oligodendrogliomas, ependymomas, and mixed gliomas, meningiomas, medulloblastomas, gangliogliomas, schwannomas (neurilemmomas), and craniopharyngiomas (see, for example, the tumors listed in Louis, D. N. et al. Acta Neuropathol 131(6), 803-820 (June 2016)). In some embodiments, the brain tumor is a primary brain tumor. In some embodiments, the patient has previously been treated with another anticancer agent, e.g., another Trk inhibitor (e.g., a first or second Trk inhibitor) or a multi-kinase inhibitor. In some embodiments, the brain tumor is a metastatic brain tumor. In some embodiments, the patient has previously been treated with another anticancer agent, e.g., another Trk inhibitor (e.g., a first or second Trk inhibitor) or a multi-kinase inhibitor.

### Definitions

Where a compound disclosed herein has at least one chiral center, the compounds may accordingly exist as enantiomers. Where the compounds possess two chiral centers, the compounds may additionally exist as diastereomers. It is to be understood that all such isomers and mixtures thereof are encompassed within the scope of the present invention. Preferably, wherein the compound is present as the (S,R) isomer, the (S,R) isomer is present at an excess of greater than or equal to about 80%, more preferably at an excess of greater than or equal to about 90%, more preferably still at an excess of greater than or equal to about 95%, more preferably still at an excess of greater than or equal to about 98%, more preferably at an excess of greater than or equal to about 99%.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively. In some embodiments, pharmaceutically acceptable salts may be preferred over the respective free base or free acid because such salts impart greater stability or solubility to the molecule thereby facilitating formulation into a dosage form. Basic compounds are generally capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate),napthalene-2-sulfonate, Ethanedisulfonate, and 2,5-dihydroxybenzoate.

As used herein, unless otherwise noted, the terms “treating,” “treatment,” and the like, shall include the management and care of a subject or patient (preferably mammal, more preferably human) for the purpose of combating a disease, condition, or disorder and includes the administration of a disclosed compound to alleviate the symptoms or complications, or reduce the rate of progression of the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

As used herein, the term “Trk-associated cancer” shall be defined to include cancers associated with or having dysregulation of a NTRK gene, a Trk protein, or expression or activity, or level of the same (e.g., any of types of dysregulation of a NTRK gene, a Trk protein, or expression or activity, or level of the same, described herein). Non-limiting examples of a Trk-associated cancer are described herein.

The term “subject” as used herein, refers to an animal, preferably a mammal, most preferably a human, who has been the object of treatment, observation or experiment. In some embodiments, the subject has experienced and/or exhibited at least one symptom of the disease or disorder to be treated and/or prevented. In some embodiments, a patient is a pediatric patient (i.e. a patient under the age of 21 years at the time of diagnosis or treatment). The term “pediatric” can be further divided into various subpopulations including: neonates (from birth through the first 28 days of life); infants (29 days of age to less than two years of age); children (two years of age to less than 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

The term “Trk” or “Trk protein” includes any of the Trk proteins described herein (e.g., a TrkA, a TrkB, or a TrkC protein).

The term “NTRK gene” includes any of the NTRK genes described herein (e.g., a NTRK1, a NTRK2, or a NTRK3 gene).

The term “wildtype” or “wild-type” describes a nucleic acid (e.g., a NTRK gene or a Trk mRNA) or protein (e.g., a Trk protein) that is found in a subject (e.g., a pediatric subject, e.g., an infant, child, or adolescent) that does not have a Trk-associated cancer (and optionally also does not have an increased risk of developing a Trk-associated cancer or condition and/or is not suspected of having a Trk-associated cancer or condition) or is found in a cell or tissue from a subject (e.g., a pediatric subject, e.g., an infant, child, or adolescent) that typically does not have a Trk-associated cancer or condition (and optionally also does not have an increased risk of developing a Trk-associated cancer or condition and/or is not suspected of having a Trk-associated cancer or condition).

The term “regulatory agency” is a country's agency for the approval of the medical use of pharmaceutical agents with the country. For example, a non-limiting example of a regulatory agency is the U.S. Food and Drug Administration (FDA).

The phrase “dysregulation of a NTRK gene, a Trk protein, or expression or activity, or level of the same” is a genetic mutation (e.g., a NTRK gene translocation that results in the expression of a fusion protein, a deletion in a NTRK gene that results in the expression of a Trk protein that includes a deletion of at least one amino acid as compared to the wild-type Trk protein, or a mutation in a NTRK gene that results in the expression of a Trk protein with one or more point mutations, an alternative spliced version of a Trk mRNA that results in a Trk protein that results in the deletion of at least one amino acid in the Trk protein as compared to the wild-type Trk protein), or a NTRK gene duplication that results in overexpression of a Trk protein) or overexpression of a NTRK gene in a cell, that results in a pathogenic increase in the activity of a kinase domain of a Trk protein (e.g., a constitutively active kinase domain of a Trk protein) in a cell. For example, a dysregulation of a NTRK gene, a Trk protein, or expression or activity, or level of the same, can be a mutation in a NTRK1, NTRK2, or NTRK3 gene that encodes a Trk protein that is constitutively active or has increased activity as compared to a protein encoded by a NTRK1, NTRK2, or NTRK3 gene that does not include the mutation. For example, a dysregulation of a NTRK gene, a Trk protein, or expression or activity, or level of the same, can be the result of a gene translocation which results in the expression of a fusion protein that contains a first portion of TrkA, TrkB, or TrkC that includes a functional kinase domain, and a second portion of a partner protein (i.e., that is not TrkA, TrkB, or TrkC). A gene encoding a fusion protein can include, e.g., the following exons of a wild-type NTRK1 gene: exons 10-19, exons 12-19, exons 12-19, exons 13-19, exons 14-19, or exons 15-19. A gene encoding a fusion protein can include, e.g., the following exons of a wild-type NTRK2 gene: exons 12-21, exons 13-21, exons 15-21, exons 16-21, or exons 17-21. A gene encoding a fusion protein can include, e.g., the following exons of a wild-type NTRK3 gene: exons 17-22 or exons 16-22. Non-limiting examples of fusion proteins that are a result of a NTRK gene translocation are described in Table 2, 5, and 8.

A dysregulation of a NTRK gene, a Trk protein, or expression or activity, or level of the same, can, e.g., include a mutation(s) in a NTRK1, NTRK2, or NTRK3 gene that results in a TrkA, TrkB, or TrkC containing at least one (e.g., two, three, four, or five) point mutations (e.g., one of more of the point mutations listed in Table 3, 4, 6, 7, 9, 10, 11, 12, and 13). A dysregulation of a NTRK gene, a Trk protein, or expression or activity, or level of the same, can be a mutation in a NTRK1, NTRK2, or NTRK3 gene that results in a deletion of one or more contiguous amino acids (e.g., at least two, at least three, at least four, at least 5, at least 6, at least 7, at least 8, at least 9, at least 10, at least 15, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400 amino acids) in the TrkA, TrkB, or TrkC protein (except for the deletion of amino acids in the kinase domain of TrkA, TrkB, or TrkC that would result in inactivation of the kinase domain). In some examples, a dysregulation of a NTRK gene, a Trk protein, or expression or activity, or level of the same, can include an alternate spliced form of a Trk mRNA, e.g., a TrkAIII spliced variant or an alternative spliced form of a TrkA mRNA that results in the production of a TrkA protein that lacks the amino acids encoded by exon 10. In some examples, a dysregulation of a NTRK gene, a Trk protein, or expression or activity, or level of the same, includes an amplification of a NTRK gene (e.g., one, two, three, or four additional copies of the NTRK gene) that can result, e.g., in autocrine or overexpression of a NTRK gene in a cell. The term “overexpression” is a term of art and is used to an increased level of transcription of a gene in a cell as compared to the level of transcription of the gene in a control cell (e.g., a non-cancerous cell of the same cell type).

The term “Trk-associated cancer or tumor” is a cancer that is associated with dysregulation of a NTRK gene, a Trk protein, or expression or activity, or level of the same (e.g., a cancer that is associated with at least one example (e.g., two, three, four, or five examples) of dysregulation of a NTRK gene, a Trk protein, or expression or activity, or level of the same, described herein).

The term “mammal” as used herein, refers to a warm-blooded animal that has or is at risk of developing a disease described herein and includes, but is not limited to, guinea pigs, dogs, cats, rats, mice, hamsters, and primates, including humans.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. For example, a therapeutically effective amount, when administered to a subject in need of such treatment, is sufficient to (i) treat or prevent a particular disease, condition, or disorder which can be treated with an inhibitor of TrkA and/or TrkB, (ii) attenuate, ameliorate, or eliminate one or more symptoms of the particular disease, condition, or disorder, or (iii) prevent or delay the onset of one or more symptoms of the particular disease, condition, or disorder described herein. The amount of one or more Trk inhibitor compounds as provided herein and optionally an immunotherapy agent that will correspond to such a therapeutically effective amount will vary depending upon factors such as the disease condition and its severity, the identity (e.g., weight) of the mammal in need of treatment, but can nevertheless be routinely determined by one skilled in the art. When referring to combinations of compounds, the combination may be “therapeutically effective” even when one or more of the compounds in the combination is administered at a dose that would be sub-therapeutic when the compound is administered alone. Indeed, the combination of compounds, or pharmaceutically acceptable salts or solvates of the foregoing, can be an additive combination, or can be a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon other art-recognized factors, for example, the patient's height, weight, sex, age and medical history.

The term “microsatellite instability” or “MSI”, as defined by the National Cancer Institute (i.e., the NCI Dictionary of Cancer Terms), refers to a change that occurs in the DNA of certain cells (such as tumor cells) in which the number of repeats of microsatellites (short, repeated sequences of DNA) is different than the number of repeats that were in the DNA when it was inherited. The cause of microsatellite instability may be a defect in the ability to repair mistakes made when DNA is copied in the cell. Microsatellites can be indicators of genome instability, especially deficient mismatch repair (dMMR). MSI can have high microsatellite instability (MSI-H) or low microsatellite instability (MSI-L).

In some embodiments, MSI is diagnosed by detecting microsatellite markers, e.g., mononucleotide repeat markers, in a sample or samples from a subject. In some embodiments, immunohistochemistry and/or sequencing is used to detect the microsatellite markers. Non-limiting examples of microsatellite markers include MLH1, PMS2, MSH2, MSH6, BAT-25, BAT26, NR-21, NR24, and MONO-27. In some embodiments, a microsatellite marker with a different number of repeats in a cancerous-tissue sample from a subject compared to the corresponding number of repeats of a microsatellite marker in an adjacent non-cancerous tissue sample from the subject is an abnormal microsatellite marker. In some embodiments, a cancer is determined to have MSI if greater than about 20% of the microsatellite markers are abnormal (e.g., in a sample obtained from the patient). In some embodiments, a cancer is determined to have MSI-H if greater than about 30% of the microsatellite markers analyzed are abnormal (e.g., in a sample obtained from the patient). In some embodiments, a cancer is determined to have MSI-L if about 3.5% to about 30% of the microsatellite markers analyzed are abnormal (e.g., in a sample obtained from the patient). In some embodiments, a cancer is determined to have microsatellite stability (MSS) if less than about 3.5% of the microsatellite markers analyzed are abnormal (e.g., in a sample obtained from the patient). In some embodiments, a Trk-associated cancer is also a cancer with MSI. See, for example, Deihimi et al. Oncotarget, 2017, Vol. 8, (No. 25), pp: 39945-39962, which is hereby incorporated by reference in its entirety.

### Additional Embodiments

Embodiment 1. A method for treating cancer, the method comprising administering to the patient a therapeutically effective amount of a first Trk inhibitor or a pharmaceutically acceptable salt thereof, a second Trk inhibitor or a pharmaceutically acceptable salt thereof, and an immunotherapy agent.
Embodiment 2. The method of embodiment 1, wherein the first Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide); (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate; cabozantinib ((N-(4-((6,7-Dimethoxyquinolin-4-yl)oxy)phenyl)-N′-(4-fluorophenyl)cyclopropane-1,1-dicarboxamide)); dovitinib (4-amino-5-fluoro-3-[6-(4-methylpiperazin-1-yl)-1H-benzimidazol-2-yl]quinolin-2(1H)-one mono 2-hydroxypropanoate hydrate); belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); PLX7486; altiratinib (N-(4-((2-(cyclopropanecarboxamido)pyridin-4-yl)oxy)-2,5-difluorophenyl)-N-(4-fluorophenyl)cyclopropane-1,1-dicarboxamide); AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine); (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one; a (R)-2-phenylpyrrolidine substituted imadazopyridazine; AZD6918; GNF-4256; GTx-186; GNF-5837; AZ623; AG-879; CT327; AR-772; AR-523; AR-786; AR-256; AR-618; AZ-23; CEP-701; CEP-751; PHA-739358; dovitinib; Go 6976; GW441756; MGCD516; ONO-5390556; PHA-848125AC; Regorafenib; Sorafenib; Sunitinib; TSR-011; VM-902A; K252a; a 4-aminopyrazolylpyrimidine; a substituted pyrazolo[1,5-a] pyrimidine compound; BMS-754807; ONO-7579; F17752; ANA-12; ONO-4474; GZ389988; and TPX-0005.
Embodiment 3. The method of any one of embodiments 1-2, wherein the second Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide); (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate; cabozantinib ((N-(4-((6,7-Dimethoxyquinolin-4-yl)oxy)phenyl)-N′-(4-fluorophenyl)cyclopropane-1,1-dicarboxamide)); dovitinib (4-amino-5-fluoro-3-[6-(4-methylpiperazin-1-yl)-1H-benzimidazol-2-yl]quinolin-2(1H)-one mono 2-hydroxypropanoate hydrate); belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); PLX7486; altiratinib (N-(4-((2-(cyclopropanecarboxamido)pyridin-4-yl)oxy)-2,5-difluorophenyl)-N-(4-fluorophenyl)cyclopropane-1,1-dicarboxamide); AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine); (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one; a (R)-2-phenylpyrrolidine substituted imadazopyridazine; AZD6918; GNF-4256; GTx-186; GNF-5837; AZ623; AG-879; CT327; AR-772; AR-523; AR-786; AR-256; AR-618; AZ-23; CEP-701; CEP-751; PHA-739358; dovitinib; Go 6976; GW441756; MGCD516; ONO-5390556; PHA-848125AC; Regorafenib; Sorafenib; Sunitinib; TSR-011; VM-902A; K252a; a 4-aminopyrazolylpyrimidine; a substituted pyrazolo[1,5-a] pyrimidine compound; BMS-754807; ONO-7579; F17752; ANA-12; ONO-4474; GZ389988; and TPX-0005;
provided that the second Trk inhibitor is different than the first Trk inhibitor.
Embodiment 4. The method of any one of embodiments 1-3, wherein the second Trk inhibitor is effective in the presence of a Trk-inhibitor resistance mutation.
Embodiment 5. The method of any one of embodiments 1-4, wherein the Trk inhibitor that is effective in the presence of a Trk-inhibitor resistance mutation is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide); 4-{4-[3-(4-chloro-3-trifluoromethylphenyl)-ureido]-3-fluorophenoxy}-pyridine-2-carboxylic acid methylamide; and (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one.
Embodiment 6. The method of any one of embodiments 1-5, wherein the first Trk inhibitor is (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate.
Embodiment 7. The method of any one of embodiments 1-6, wherein the second Trk inhibitor is (6R,15R)-9-fluoro-15-methyl-2, 11,16,20,21,24-hexaazapentacyclo[16.5.2.0^{2,6}.0^{7,12}.0^{21,25}]pentacosa-1(24),7,9, 11,18(25),19,22-heptaen-17-one.
Embodiment 8. The method of any one of embodiments 1-7, wherein the immunotherapy agent is selected from the group consisting of: a cellular immunotherapy; an antibody therapy; an antibody-drug conjugate; a toxin; blinatumomab (AMG103) or midostaurin (Rydapt); a cytokine therapy; an immune checkpoint inhibitor; an mRNA-based immunotherapy; bacillus Calmette-Guerin (BCG) therapy; an oncolytic virus therapy; a cancer vaccine; a peptide vaccine; and a DNA-based vaccine.
Embodiment 9. The method of any one of embodiments 1-8, wherein the cellular immunotherapy is selected from the group consisting of: adoptive T-cell therapy; dendritic cell therapy; natural killer cell therapy; sipuleucel-T (APC8015); cells that express a chimeric antigen receptor (CAR); CAR-T cell therapy; and tisagenlecleucel.
Embodiment 10. The method of any one of embodiments 1-9, wherein the antibody therapy is selected from the group consisting of: a monoclonal antibody and a conjugated antibody.
Embodiment 11. The method of any one of embodiments 1-10, wherein the antibody therapy is selected from the group consisting of: bevacizumab; trastuzumab; avelumab; rituximab; edrecolomab; daratumuab; olaratumab; ofatumumab; alemtuzumab; cetuximab; oregovomab; pembrolizumab; dinutiximab; obinutuzumab; tremelimumab (CP-675;206); ramucirumab; ublituximab (TG-1101); panitumumab; elotuzumab; avelumab; necitumumab; cirmtuzumab; ibritumomab; isatuximab (SAR650984); nimotuzumab; fresolimumab (GC1008); lirilumab (INN); mogamulizumab; ficlatuzumab (AV-299); denosumab; ganitumab; urelumab; pidilizumab; and amatuximab.
Embodiment 12. The method of any one of embodiments 1-11, wherein the antibody-drug conjugate is selected from the group consisting of: gemtuzumab ozogamicin; inotuzumab ozogamicin; brentuximab vedotin; ado-trastuzumab emtansine (TDM-1); mirvetuximab soravtansine (IMGN853); and anetumab ravtansine.
Embodiment 13. The method of any one of embodiments 1-12, wherein the toxin is denileukin diftitox.
Embodiment 14. The method of any one of embodiments 1-13, wherein the immunotherapy agent is blinatumomab (AMG103).
Embodiment 15. The method of any one of embodiments 1-14, wherein the immunotherapy agent is midostaurin (Rydapt).
Embodiment 16. The method of any one of embodiments 1-15, wherein the cytokine therapy is selected from the group consisting of: an interleukin 2 (IL-2) therapy; an interferon alpha (IFNα) therapy; a granulocyte colony stimulating factor (G-CSF) therapy; an interleukin 12 (IL-12) therapy; an interleukin 15 (IL-15) therapy; an interleukin 7 (IL-7) therapy; and an erythropoietin-alpha (EPO) therapy.
Embodiment 17. The method of any one of embodiments 1-16, wherein the interleukin 2 (IL-2) therapy is aldesleukin.
Embodiment 18. The method of any one of embodiments 1-17, wherein the IFNα therapy is interferon alfa-2b or interferon alfa-2a.
Embodiment 19. The method of any one of embodiments 1-18, wherein the G-CSF therapy is filgrastim.
Embodiment 20. The method of any one of embodiments 1-19, wherein the immune checkpoint inhibitor is selected from the group consisting of: a CTLA-4 inhibitor; a PD-1 inhibitor; and a PD-L1 inhibitor.
Embodiment 21. The method of any one of embodiments 1-20, wherein the CTLA-4 inhibitor is ipilimumab or tremelimumab (CP-675,206).
Embodiment 22. The method of any one of embodiments 1-21, wherein the PD-1 inhibitor is pembrolizumab or nivolumab.
Embodiment 23. The method of any one of embodiments 1-22, wherein the PD-L1 inhibitor is selected from the group consisting of: atezolizumab; avelumab; and durvalumab.
Embodiment 24. The method of any one of embodiments 1-23, wherein the mRNA-based immunotherapy is CV9104.
Embodiment 25. The method of any one of embodiments 1-24, wherein the immunotherapy agent is bacillus Calmette-Guerin (BCG) therapy.
Embodiment 26. The method of any one of embodiments 1-25, wherein the oncolytic virus therapy is talimogene alherparepvec (T-VEC).
Embodiment 27. The method of any one of embodiments 1-26, wherein the cancer vaccine is a human papillomavirus (HPV) vaccine.
Embodiment 28. The method of any one of embodiments 1-27, wherein the human papillomavirus (HPV) vaccine is selected from the group consisting of: a recombinant human papillomavirus vaccine [types 6, 11, 16, and 18]; a recombinant human papillomavirus vaccine [types 6, 11, 16, 18, 31, 33, 45, 52, and 58]; and a recombinant human papillomavirus vaccine [types 16 and 18].
Embodiment 29. The method of any one of embodiments 1-28, wherein the cancer vaccine is a hepatitis B virus (HBV) vaccine.
Embodiment 30. The method of any one of embodiments 1-29, wherein the cancer vaccine is selected from the group consisting of: a combination Hepatitis A and Hepatitis B vaccine; a combination diphtheria, tetanus, pertussis, hepatitis B virus, and poliomyelitis vaccine; dasiprotimut-T; cancer vaccine HSPPC-96; GVAX; ADXS11-001; ALVAC-CEA; rilimogene galvacirepvec/rilimogene glafolivec; CDX-110; CimaVax-EGF; lapuleucel-T (APC8024); GRNVAC1; GRNVAC2; GRN-1201; hepcortespenlisimut-L (Hepko-V5); a dendritic cell vaccine; ICT-107; SCIB1; BMT CTN 1401; PrCa VBIR; PANVAC; a prostate cancer vaccine; DPX-Survivac; and viagenpumatucel-L (HS-110).
Embodiment 31. The method of any one of embodiments 1-30, wherein the peptide vaccine is selected from the group consisting of: nelipepimut-S(E75); IMA901; SurVaxM (SVN53-67); an immunogenic personal neoantigen vaccine; RGSH4K; and NEO-PV-01.
Embodiment 32. The method of any one of embodiments 1-31, wherein the DNA-based vaccine is a mammaglobin-A DNA vaccine.
Embodiment 33. The method of any one of embodiments 1-32, wherein the cancer is a Trk-associated cancer.
Embodiment 34. The method of any one of embodiments 1-33, wherein the Trk-associated cancer is due to oncogenic rearrangements in a NTRK gene selected from the group consisting of: NTRK1, NTRK2, and NTRK3.
Embodiment 35. The method of any one of embodiments 1-34, wherein the Trk-associated cancer has at least one point mutation in a NTRK1 gene that results in the expression of a TrkA protein comprising a mutation at one or more amino acid position(s) selected from the group consisting of: 517, 542, 564, 568, 573, 589, 595, 596, 599, 600, 602, 646, 656, 657, 667, 676, and 679.
Embodiment 36. The method of any one of embodiments 1-35, wherein the Trk-associated cancer has at least one point mutation in a NTRK2 gene that results in the expression of a TrkB protein comprising a mutation at one or more amino acid position(s) selected from the group consisting of: 545, 570, 596, 601, 617, 623, 624, 628, 630, 639, 672, 682, 683, 693, and 702.
Embodiment 37. The method of any one of embodiments 1-36, wherein the Trk-associated cancer has at least one point mutation in a NTRK3 gene that results in the expression of a TrkC protein comprising a mutation at one or more amino acid position(s) selected from the group consisting of: 545, 570, 596, 601, 617, 623, 624, 628, 630, 675, 685, 686, 696, and 705.
Embodiment 38. The method of any one of embodiments 1-37, wherein the cancer is selected from the group consisting of: adenocarcinoma; adrenal gland cortical carcinoma; adrenal gland neuroblastoma; anus squamous cell carcinoma; appendix adenocarcinoma; bladder urothelial carcinoma; bile duct adenocarcinoma; bladder carcinoma; bladder urothelial carcinoma; bone chordoma; bone marrow leukemia lymphocytic chronic; bone marrow leukemia non-lymphocytic acute myelocytic; bone marrow lymph proliferative disease; bone marrow multiple myeloma; bone sarcoma; brain astrocytoma; brain glioblastoma; brain medulloblastoma; brain meningioma; brain oligodendroglioma; breast adenoid cystic carcinoma; breast carcinoma; breast ductal carcinoma in situ; breast invasive ductal carcinoma; breast invasive lobular carcinoma; breast metaplastic carcinoma; cervix neuroendocrine carcinoma; cervix squamous cell carcinoma; colon adenocarcinoma; colon carcinoid tumor; duodenum adenocarcinoma; endometrioid tumor; esophagus adenocarcinoma; eye intraocular melanoma; eye intraocular squamous cell carcinoma; eye lacrimal duct carcinoma; fallopian tube serous carcinoma; gallbladder adenocarcinoma; gallbladder glomus tumor; gastroesophageal junction adenocarcinoma; head and neck adenoid cystic carcinoma; head and neck carcinoma; head and neck neuroblastoma; head and neck squamous cell carcinoma; kidney chromophore carcinoma; kidney medullary carcinoma; kidney renal cell carcinoma; kidney renal papillary carcinoma; kidney sarcomatoid carcinoma; kidney urothelial carcinoma; leukemia lymphocytic; liver cholangiocarcinoma; liver hepatocellular carcinoma; lung adenocarcinoma; lung adenosquamous carcinoma; lung atypical carcinoid; lung carcinosarcoma; lung large cell neuroendocrine carcinoma; lung non-small cell lung carcinoma; lung sarcoma; lung sarcomatoid carcinoma; lung small cell carcinoma; lung small cell undifferentiated carcinoma; lung squamous cell carcinoma; lymph node lymphoma diffuse large B cell; lymph node lymphoma follicular lymphoma; lymph node lymphoma mediastinal B-cell; lymph node lymphoma plasmablastic lung adenocarcinoma; lymphoma follicular lymphoma; non-Hodgkin's lymphoma; nasopharynx and paranasal sinuses undifferentiated carcinoma; ovary carcinoma; ovary carcinosarcoma; ovary clear cell carcinoma; ovary epithelial carcinoma; ovary granulosa cell tumor; ovary serous carcinoma; pancreas carcinoma; pancreas ductal adenocarcinoma; pancreas neuroendocrine carcinoma; peritoneum mesothelioma; peritoneum serous carcinoma; placenta choriocarcinoma; pleura mesothelioma; prostate acinar adenocarcinoma; prostate carcinoma; rectum adenocarcinoma; rectum squamous cell carcinoma; skin adnexal carcinoma; skin basal cell carcinoma; skin melanoma; skin Merkel cell carcinoma; skin squamous cell carcinoma; small intestine adenocarcinoma; small intestine gastrointestinal stromal tumors (GISTs); soft tissue angiosarcoma; soft tissue Ewing sarcoma; soft tissue hemangioendothelioma; soft tissue inflammatory myofibroblastic tumor; soft tissue leiomyosarcoma; soft tissue liposarcoma; soft tissue neuroblastoma; soft tissue paraganglioma; soft tissue perivascular epitheliod cell tumor; soft tissue sarcoma; non-rhabdomyosarcoma soft tissue sarcomas (NRSTS); retroperitoneal congenital sarcoma; soft tissue synovial sarcoma; locally advanced sarcoma; hepatobiliary cancer, stomach adenocarcinoma; stomach adenocarcinoma diffuse type; stomach adenocarcinoma intestinal type; stomach adenocarcinoma intestinal type; stomach leiomyosarcoma; thymus carcinoma; thymus thymoma lymphocytic; thyroid papillary carcinoma; unknown primary adenocarcinoma; unknown primary carcinoma; unknown primary malignant neoplasm; unknown primary melanoma; unknown primary sarcomatoid carcinoma; unknown primary squamous cell carcinoma; unknown undifferentiated neuroendocrine carcinoma; unknown primary undifferentiated small cell carcinoma; uterus carcinosarcoma; uterus endometrial adenocarcinoma; uterus endometrial adenocarcinoma endometrioid; uterus endometrial adenocarcinoma papillary serous; and uterus leiomyosarcoma.
Embodiment 39. The method of any one of embodiments 1-38, wherein the cancer is selected from the group consisting of: adrenocortical carcinoma; anal cancer; appendix cancer; atypical teratoid/rhabdoid tumor (e.g., central nervous system atypical teratoid/rhabdoid tumor); B-cell cancer; bile duct cancer; bladder cancer; bone cancer (e.g., osteosarcoma and malignant fibrous histiocytoma); brain cancer (e.g., brain and spinal cord tumor; brain stem glioma; central nervous system embryonal tumors; central nervous system germ cell tumors; craniopharyngioma; and ependymoma); breast cancer; bronchogenic carcinoma; bronchus cancer; cancer of hematological tissues; cancer of the oral cavity or pharynx; carcinoid tumor; cervical cancer; childhood cancers; chordoma; chronic lymphocytic leukemia; chronic myeloproliferative neoplasms; colon cancer; colorectal cancer; cutaneous T-cell lymphoma; ductal carcinoma in situ; embryonal tumor; endometrial cancer; esophageal cancer; esthesioneuroblastoma; extracranial germ cell tumor; extragonadal germ cell tumor; extrahepatic bile duct cancer; eye cancer (e.g., retinoblastoma); fallopian tube cancer; fibrosarcoma; fibrous histiocytoma of bone; gallbladder cancer; gastric cancer; gastrointestinal carcinoid tumor; germ cell tumor; gestational trophoblastic disease; glioblastoma multiforme; glioma (e.g., lower-grade glioma); head and neck cancer; heart cancer; histiocytosis; hypopharyngeal cancer; inflammatory myofibroblastic tumors; intrahepatic cholangiocarcinoma; islet cell tumor; kidney cancer (e.g., renal cell cancer); Langerhans cell histiocytosis; large cell neuroendocrine cancer; laryngeal cancer; leukemia (e.g., acute lymphoblastic leukemia; acute myeloid leukemia; chronic myelogenous leukemia; and hairy cell leukemia); lip cancer; liver cancer; lung cancer; Burkitt lymphoma; Hodgkin's lymphoma; and primary central nervous system lymphoma); medulloblastoma; mesothelioma; mouth cancer; multiple myeloma; myelodysplastic syndromes; nasal cavity and paranasal sinus cancer; nasopharyngeal cancer; neoplasm (e.g., a melanocystic neoplasm); nephroma; neuroblastoma; non-small cell lung cancer; oral cancer; oropharyngeal cancer; ovarian cancer; pancreatic cancer; paraganglioma; parathyroid cancer; glioma (e.g., pediatric gliomas); penile cancer; pharyngeal cancer; pheochromocytoma; pilocytic astrocytoma; pituitary tumor; plasma cell neoplasm; primary peritoneal cancer; prostate cancer; rectum carcinoma; salivary gland cancer; sarcoma (e.g., Ewing sarcoma; rhabdomyosarcoma; uterine sarcoma; and undifferentiated sarcoma); secretory breast carcinoma; Sezary syndrome; skin cancer; small bowel cancer; small cell lung cancer; small intestine cancer; Spitz nevi; Spitz tumors; spitzoid melanoma; stomach cancer; squamous cell carcinoma; squamous neck cancer; testicular cancer; throat cancer; thymoma and thymic carcinoma; thyroid carcinoma; urethral cancer; uterine cancer; urinary bladder cancer; vaginal cancer; vulvar cancer; and Wilms tumor.
Embodiment 40. A method for treating cancer, the method comprising administering to the patient a therapeutically effective amount of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, a second Trk inhibitor or a pharmaceutically acceptable salt thereof, and an immunotherapy agent.
Embodiment 41. The method of embodiment 40, wherein the second Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide); cabozantinib ((N-(4-((6,7-Dimethoxyquinolin-4-yl)oxy)phenyl)-N′-(4-fluorophenyl)cyclopropane-1,1-dicarboxamide)); dovitinib (4-amino-5-fluoro-3-[6-(4-methylpiperazin-1-yl)-1H-benzimidazol-2-yl]quinolin-2(1H)-one mono 2-hydroxypropanoate hydrate); belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); PLX7486; altiratinib (N-(4-((2-(cyclopropanecarboxamido)pyridin-4-yl)oxy)-2,5-difluorophenyl)-N-(4-fluorophenyl)cyclopropane-1,1-dicarboxamide); AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine); (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one; a (R)-2-phenylpyrrolidine substituted imadazopyridazine; AZD6918; GNF-4256; GTx-186; GNF-5837; AZ623; AG-879; CT327; AR-772; AR-523; AR-786; AR-256; AR-618; AZ-23; CEP-701; CEP-751; PHA-739358; dovitinib; Go 6976; GW441756; MGCD516; ONO-5390556; PHA-848125AC; Regorafenib; Sorafenib; Sunitinib; TSR-011; VM-902A; K252a; a 4-aminopyrazolylpyrimidine; a substituted pyrazolo[1,5-a] pyrimidine compound; BMS-754807; ONO-7579; F17752; ANA-12; ONO-4474; GZ389988; and TPX-0005.
Embodiment 42. The method of any one of embodiments 40-41, wherein the second Trk inhibitor is effective in the presence of a Trk-inhibitor resistance mutation.
Embodiment 43. The method of any one of embodiments 40-42, wherein the Trk inhibitor that is effective in the presence of a Trk-inhibitor resistance mutation is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide); 4-{4-[3-(4-chloro-3-trifluoromethylphenyl)-ureido]-3-fluorophenoxy}-pyridine-2-carboxylic acid methylamide; and (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one.
Embodiment 44. The method of any one of embodiments 40-43, wherein the second Trk inhibitor is (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one.
Embodiment 45. The method of any one of embodiments 40-44, wherein the immunotherapy agent is selected from the group consisting of: a cellular immunotherapy; an antibody therapy; an antibody-drug conjugate; a toxin; blinatumomab (AMG103; Blincyto®) or midostaurin (Rydapt); a cytokine therapy; an immune checkpoint inhibitor; an mRNA-based immunotherapy; bacillus Calmette-Guerin (BCG) therapy; a cancer vaccine; and a peptide vaccine.
Embodiment 46. The method of any one of embodiments 40-45, wherein the cellular immunotherapy is selected from the group consisting of: adoptive T-cell therapy; dendritic cell therapy; natural killer cell therapy; sipuleucel-T (APC8015); cells that express a chimeric antigen receptor (CAR); CAR-T cell therapy; and tisagenlecleucel.
Embodiment 47. The method of any one of embodiments 40-46, wherein the antibody therapy is selected from the group consisting of: a monoclonal antibody and a conjugated antibody.
Embodiment 48. The method of any one of embodiments 40-47, wherein the antibody therapy is selected from the group consisting of: bevacizumab; trastuzumab; avelumab; rituximab; edrecolomab; daratumuab; olaratumab; ofatumumab; alemtuzumab; cetuximab; oregovomab; pembrolizumab; dinutiximab; obinutuzumab; tremelimumab (CP-675;206); ramucirumab; ublituximab (TG-1101); panitumumab; elotuzumab; avelumab; necitumumab; cirmtuzumab; ibritumomab; isatuximab (SAR650984); nimotuzumab; fresolimumab (GC1008); lirilumab (INN); mogamulizumab; ficlatuzumab (AV-299); denosumab; ganitumab; urelumab; pidilizumab; and amatuximab.
Embodiment 49. The method of any one of embodiments 40-48, wherein the antibody-drug conjugate is selected from the group consisting of: gemtuzumab ozogamicin; inotuzumab ozogamicin; brentuximab vedotin; ado-trastuzumab emtansine (TDM-1); mirvetuximab soravtansine (IMGN853); and anetumab ravtansine.
Embodiment 50. The method of any one of embodiments 40-49, wherein the toxin is denileukin diftitox.
Embodiment 51. The method of any one of embodiments 40-50, wherein the immunotherapy agent is blinatumomab (AMG103).
Embodiment 52. The method of any one of embodiments 40-51, wherein the immunotherapy agent is midostaurin (Rydapt).
Embodiment 53. The method of any one of embodiments 40-52, wherein the cytokine therapy is selected from the group consisting of: an interleukin 2 (IL-2) therapy; an interferon alpha (IFNα) therapy; a granulocyte colony stimulating factor (G-CSF) therapy; an interleukin 12 (IL-12) therapy; an interleukin 15 (IL-15) therapy; an interleukin 7 (IL-7) therapy; and an erythropoietin-alpha (EPO) therapy.
Embodiment 54. The method of any one of embodiments 40-53, wherein the interleukin 2 (IL-2) therapy is aldesleukin.
Embodiment 55. The method of any one of embodiments 40-54, wherein the IFNα therapy is interferon alfa-2b or interferon alfa-2a.
Embodiment 56. The method of any one of embodiments 40-55, wherein the G-CSF therapy is filgrastim.
Embodiment 57. The method of any one of embodiments 40-56, wherein the immune checkpoint inhibitor is selected from the group consisting of: a CTLA-4 inhibitor; a PD-1 inhibitor; and a PD-L1 inhibitor.
Embodiment 58. The method of any one of embodiments 40-57, wherein the CTLA-4 inhibitor is ipilimumab or tremelimumab (CP-675,206).
Embodiment 59. The method of any one of embodiments 40-58, wherein the PD-1 inhibitor is pembrolizumab or nivolumab.
Embodiment 60. The method of any one of embodiments 40-59, wherein the PD-L1 inhibitor is atezolizumab; avelumab; and durvalumab.
Embodiment 61. The method of any one of embodiments 40-60, wherein the mRNA-based immunotherapy is CV9104.
Embodiment 62. The method of any one of embodiments 40-61, wherein the immunotherapy agent is bacillus Calmette-Guerin (BCG) therapy.
Embodiment 63. The method of any one of embodiments 40-62, wherein the oncolytic virus therapy is talimogene alherparepvec (T-VEC).
Embodiment 64. The method of any one of embodiments 40-63, wherein the cancer vaccine is a human papillomavirus (HPV) vaccine.
Embodiment 65. The method of any one of embodiments 40-64, wherein the human papillomavirus (HPV) vaccine is selected from the group consisting of: a recombinant human papillomavirus vaccine [types 6, 11, 16, and 18]; a recombinant human papillomavirus vaccine [types 6, 11, 16, 18, 31, 33, 45, 52, and 58]; and a recombinant human papillomavirus vaccine [types 16 and 18].
Embodiment 66. The method of any one of embodiments 40-65, wherein the cancer vaccine is a hepatitis B virus (HBV) vaccine.
Embodiment 67. The method of any one of embodiments 40-66, wherein the cancer vaccine is selected from the group consisting of: a combination Hepatitis A and Hepatitis B vaccine; a combination diphtheria, tetanus, pertussis, hepatitis B virus, and poliomyelitis; dasiprotimut-T; cancer vaccine HSPPC-96; GVAX; ADXS11-001; ALVAC-CEA; rilimogene galvacirepvec/rilimogene glafolivec; CDX-110; CimaVax-EGF, lapuleucel-T (APC8024), GRNVAC1, GRNVAC2, GRN-1201, hepcortespenlisimut-L (Hepko-V5), a dendritic cell vaccine; ICT-107; SCIB1; BMT CTN 1401; PrCa VBIR; PANVAC; a prostate cancer vaccine; DPX-Survivac; and viagenpumatucel-L (HS-110).
Embodiment 68. The method of any one of embodiments 40-67, wherein the peptide vaccine is selected from the group consisting of: nelipepimut-S(E75); IMA901; SurVaxM (SVN53-67); an immunogenic personal neoantigen vaccine; RGSH4K; and NEO-PV-01.
Embodiment 69. The method of any one of embodiments 40-68; wherein the DNA-based vaccine is a mammaglobin-A DNA vaccine.
Embodiment 70. The method of any one of embodiments 40-69 wherein the cancer is a Trk-associated cancer.
Embodiment 71. The method of any one of embodiments 40-70 wherein the Trk-associated cancer is due to oncogenic rearrangements in a NTRK gene selected from the group consisting of: NTRK1, NTRK2, and NTRK3.
Embodiment 72. The method of any one of embodiments 40-71, wherein the Trk-associated cancer has at least one point mutation in a NTRK1 gene that results in the expression of a TrkA protein comprising a mutation at one or more amino acid position(s) selected from the group consisting of: 517, 542, 564, 568, 573, 589, 595, 596, 599, 600, 602, 646, 656, 657, 667, 676, and 679.
Embodiment 73. The method of any one of embodiments 40-72, wherein the Trk-associated cancer has at least one point mutation in a NTRK2 gene that results in the expression of a TrkB protein comprising a mutation at one or more amino acid position(s) selected from the group consisting of: 545, 570, 596, 601, 617, 623, 624, 628, 630, 639, 672, 682, 683, 693, and 702.
Embodiment 74. The method of any one of embodiments 40-73, wherein the Trk-associated cancer has at least one point mutation in a NTRK3 gene that results in the expression of a TrkC protein comprising a mutation at one or more amino acid position(s) selected from the group consisting of: 545, 570, 596, 601, 617, 623, 624, 628, 630, 675, 685, 686, 696, and 705.
Embodiment 75. The method of any one of embodiments 40-74, wherein the cancer is selected from the group consisting of: adenocarcinoma; adrenal gland cortical carcinoma; adrenal gland neuroblastoma; anus squamous cell carcinoma; appendix adenocarcinoma; bladder urothelial carcinoma; bile duct adenocarcinoma; bladder carcinoma; bladder urothelial carcinoma; bone chordoma; bone marrow leukemia lymphocytic chronic; bone marrow leukemia non-lymphocytic acute myelocytic; bone marrow lymph proliferative disease; bone marrow multiple myeloma; bone sarcoma; brain astrocytoma; brain glioblastoma; brain medulloblastoma; brain meningioma; brain oligodendroglioma; breast adenoid cystic carcinoma; breast carcinoma; breast ductal carcinoma in situ; breast invasive ductal carcinoma; breast invasive lobular carcinoma; breast metaplastic carcinoma; cervix neuroendocrine carcinoma; cervix squamous cell carcinoma; colon adenocarcinoma; colon carcinoid tumor; duodenum adenocarcinoma; endometrioid tumor; esophagus adenocarcinoma; eye intraocular melanoma; eye intraocular squamous cell carcinoma; eye lacrimal duct carcinoma; fallopian tube serous carcinoma; gallbladder adenocarcinoma; gallbladder glomus tumor; gastroesophageal junction adenocarcinoma; head and neck adenoid cystic carcinoma; head and neck carcinoma; head and neck neuroblastoma; head and neck squamous cell carcinoma; kidney chromophore carcinoma; kidney medullary carcinoma; kidney renal cell carcinoma; kidney renal papillary carcinoma; kidney sarcomatoid carcinoma; kidney urothelial carcinoma; leukemia lymphocytic; liver cholangiocarcinoma; liver hepatocellular carcinoma; lung adenocarcinoma; lung adenosquamous carcinoma; lung atypical carcinoid; lung carcinosarcoma; lung large cell neuroendocrine carcinoma; lung non-small cell lung carcinoma; lung sarcoma; lung sarcomatoid carcinoma; lung small cell carcinoma; lung small cell undifferentiated carcinoma; lung squamous cell carcinoma; lymph node lymphoma diffuse large B cell; lymph node lymphoma follicular lymphoma; lymph node lymphoma mediastinal B-cell; lymph node lymphoma plasmablastic lung adenocarcinoma; lymphoma follicular lymphoma; non-Hodgkin's lymphoma; nasopharynx and paranasal sinuses undifferentiated carcinoma; ovary carcinoma; ovary carcinosarcoma; ovary clear cell carcinoma; ovary epithelial carcinoma; ovary granulosa cell tumor; ovary serous carcinoma; pancreas carcinoma; pancreas ductal adenocarcinoma; pancreas neuroendocrine carcinoma; peritoneum mesothelioma; peritoneum serous carcinoma; placenta choriocarcinoma; pleura mesothelioma; prostate acinar adenocarcinoma; prostate carcinoma; rectum adenocarcinoma; rectum squamous cell carcinoma; skin adnexal carcinoma; skin basal cell carcinoma; skin melanoma; skin Merkel cell carcinoma; skin squamous cell carcinoma; small intestine adenocarcinoma; small intestine gastrointestinal stromal tumors (GISTs); soft tissue angiosarcoma; soft tissue Ewing sarcoma; soft tissue hemangioendothelioma; soft tissue inflammatory myofibroblastic tumor; soft tissue leiomyosarcoma; soft tissue liposarcoma; soft tissue neuroblastoma; soft tissue paraganglioma; soft tissue perivascular epitheliod cell tumor; soft tissue sarcoma; non-rhabdomyosarcoma soft tissue sarcomas (NRSTS); retroperitoneal congenital sarcoma; soft tissue synovial sarcoma; locally advanced sarcoma; hepatobiliary cancer, stomach adenocarcinoma; stomach adenocarcinoma diffuse type; stomach adenocarcinoma intestinal type; stomach adenocarcinoma intestinal type; stomach leiomyosarcoma; thymus carcinoma; thymus thymoma lymphocytic; thyroid papillary carcinoma; unknown primary adenocarcinoma; unknown primary carcinoma; unknown primary malignant neoplasm; unknown primary melanoma; unknown primary sarcomatoid carcinoma; unknown primary squamous cell carcinoma; unknown undifferentiated neuroendocrine carcinoma; unknown primary undifferentiated small cell carcinoma; uterus carcinosarcoma; uterus endometrial adenocarcinoma; uterus endometrial adenocarcinoma endometrioid; uterus endometrial adenocarcinoma papillary serous; and uterus leiomyosarcoma.
Embodiment 76. The method of any one of embodiments 40-75, wherein the cancer is selected from the group consisting of: adrenocortical carcinoma; anal cancer; appendix cancer; atypical teratoid/rhabdoid tumor (e.g., central nervous system atypical teratoid/rhabdoid tumor); B-cell cancer; bile duct cancer; bladder cancer; bone cancer (e.g., osteosarcoma and malignant fibrous histiocytoma); brain cancer (e.g., brain and spinal cord tumor; brain stem glioma; central nervous system embryonal tumors; central nervous system germ cell tumors; craniopharyngioma; and ependymoma); breast cancer; bronchogenic carcinoma; bronchus cancer; cancer of hematological tissues; cancer of the oral cavity or pharynx; carcinoid tumor; cervical cancer; childhood cancers; chordoma; chronic lymphocytic leukemia; chronic myeloproliferative neoplasms; colon cancer; colorectal cancer; cutaneous T-cell lymphoma; ductal carcinoma in situ; embryonal tumor; endometrial cancer; esophageal cancer; esthesioneuroblastoma; extracranial germ cell tumor; extragonadal germ cell tumor; extrahepatic bile duct cancer; eye cancer (e.g., retinoblastoma); fallopian tube cancer; fibrosarcoma; fibrous histiocytoma of bone; gallbladder cancer; gastric cancer; gastrointestinal carcinoid tumor; germ cell tumor; gestational trophoblastic disease; glioblastoma multiforme; glioma (e.g., lower-grade glioma); head and neck cancer; heart cancer; histiocytosis; hypopharyngeal cancer; inflammatory myofibroblastic tumors; intrahepatic cholangiocarcinoma; islet cell tumor; kidney cancer (e.g., renal cell cancer); Langerhans cell histiocytosis; large cell neuroendocrine cancer; laryngeal cancer; leukemia (e.g., acute lymphoblastic leukemia; acute myeloid leukemia; chronic myelogenous leukemia; and hairy cell leukemia); lip cancer; liver cancer; lung cancer; Burkitt lymphoma; Hodgkin's lymphoma; and primary central nervous system lymphoma); medulloblastoma; mesothelioma; mouth cancer; multiple myeloma; myelodysplastic syndromes; nasal cavity and paranasal sinus cancer; nasopharyngeal cancer; neoplasm (e.g., a melanocystic neoplasm); nephroma; neuroblastoma; non-small cell lung cancer; oral cancer; oropharyngeal cancer; ovarian cancer; pancreatic cancer; paraganglioma; parathyroid cancer; glioma (e.g., pediatric gliomas); penile cancer; pharyngeal cancer; pheochromocytoma; pilocytic astrocytoma; pituitary tumor; plasma cell neoplasm; primary peritoneal cancer; prostate cancer; rectum carcinoma; salivary gland cancer; sarcoma (e.g., Ewing sarcoma; rhabdomyosarcoma; uterine sarcoma; and undifferentiated sarcoma); secretory breast carcinoma; Sezary syndrome; skin cancer; small bowel cancer; small cell lung cancer; small intestine cancer; Spitz nevi; Spitz tumors; spitzoid melanoma; stomach cancer; squamous cell carcinoma; squamous neck cancer; testicular cancer; throat cancer; thymoma and thymic carcinoma; thyroid carcinoma; urethral cancer; uterine cancer; urinary bladder cancer; vaginal cancer; vulvar cancer; and Wilms tumor.
Embodiment 77. A method for treating cancer, the method comprising administering to the patient a therapeutically effective amount of (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one, a second Trk inhibitor, and an immunotherapy agent.
Embodiment 78. The method of embodiment 77, wherein the second Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide); (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate; cabozantinib ((N-(4-((6,7-Dimethoxyquinolin-4-yl)oxy)phenyl)-N′-(4-fluorophenyl)cyclopropane-1,1-dicarboxamide)); dovitinib (4-amino-5-fluoro-3-[6-(4-methylpiperazin-1-yl)-1H-benzimidazol-2-yl]quinolin-2(1H)-one mono 2-hydroxypropanoate hydrate); belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); PLX7486; altiratinib (N-(4-((2-(cyclopropanecarboxamido)pyridin-4-yl)oxy)-2,5-difluorophenyl)-N-(4-fluorophenyl)cyclopropane-1,1-dicarboxamide); AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine); a (R)-2-phenylpyrrolidine substituted imadazopyridazine; AZD6918; GNF-4256; GTx-186; GNF-5837; AZ623; AG-879; CT327; AR-772; AR-523; AR-786; AR-256; AR-618; AZ-23; CEP-701; CEP-751; PHA-739358; dovitinib; Go 6976; GW441756; MGCD516; ONO-5390556; PHA-848125AC; Regorafenib; Sorafenib; Sunitinib; TSR-011; VM-902A; K252a; a 4-aminopyrazolylpyrimidine; a substituted pyrazolo[1,5-a]pyrimidine compound; BMS-754807; ONO-7579; F17752; ANA-12; ONO-4474; GZ389988; and TPX-0005.
Embodiment 79. The method of any one of embodiments 77-78, wherein the second Trk inhibitor is (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate.
Embodiment 80. The method of any one of embodiments 77-79, wherein the immunotherapy agent is selected from the group consisting of: a cellular immunotherapy; an antibody therapy; an antibody-drug conjugate; a toxin; blinatumomab (AMG103) or midostaurin (Rydapt); a cytokine therapy; an immune checkpoint inhibitor; an mRNA-based immunotherapy; bacillus Calmette-Guerin (BCG) therapy; an oncolytic virus therapy; a cancer vaccine; a peptide vaccine; and a DNA-based vaccine.
Embodiment 81. The method of any one of embodiments 77-80, wherein the cellular immunotherapy is selected from the group consisting of: adoptive T-cell therapy; dendritic cell therapy; natural killer cell therapy; sipuleucel-T (APC8015); cells that express a chimeric antigen receptor (CAR); CAR-T cell therapy; and tisagenlecleucel.
Embodiment 82. The method of any one of embodiments 77-81 wherein the antibody therapy is selected from the group consisting of: a monoclonal antibody and a conjugated antibody.
Embodiment 83. The method of any one of embodiments 77-82, wherein the antibody therapy is selected from the group consisting of: bevacizumab; trastuzumab; avelumab; rituximab; edrecolomab; daratumuab; olaratumab; ofatumumab; alemtuzumab; cetuximab; oregovomab; pembrolizumab; dinutiximab; obinutuzumab; tremelimumab (CP-675;206); ramucirumab; ublituximab (TG-1101); panitumumab; elotuzumab; avelumab; necitumumab; cirmtuzumab; ibritumomab; isatuximab (SAR650984); nimotuzumab; fresolimumab (GC1008); lirilumab (INN); mogamulizumab; ficlatuzumab (AV-299); denosumab; ganitumab; urelumab; pidilizumab; and amatuximab.
Embodiment 84. The method of any one of embodiments 77-83, wherein the antibody-drug conjugate is selected from the group consisting of: gemtuzumab ozogamicin; inotuzumab ozogamicin; brentuximab vedotin; ado-trastuzumab emtansine (TDM-1); mirvetuximab soravtansine (IMGN853); and anetumab ravtansine.
Embodiment 85. The method of any one of embodiments 77-84, wherein the toxin is denileukin diftitox.
Embodiment 86. The method of any one of embodiments 77-85, wherein the immunotherapy agent is blinatumomab (AMG103).
Embodiment 87. The method of any one of embodiments 77-86, wherein the immunotherapy agent is midostaurin (Rydapt).
Embodiment 88. The method of any one of embodiments 77-87, wherein the cytokine therapy is selected from the group consisting of: an interleukin 2 (IL-2) therapy; an interferon alpha (IFNα) therapy; a granulocyte colony stimulating factor (G-CSF) therapy; an interleukin 12 (IL-12) therapy; an interleukin 15 (IL-15) therapy; an interleukin 7 (IL-7) therapy; and an erythropoietin-alpha (EPO) therapy.
Embodiment 89. The method of any one of embodiments 77-88, wherein the interleukin 2 (IL-2) therapy is aldesleukin.
Embodiment 90. The method of any one of embodiments 77-89, wherein the IFNα therapy is interferon alfa-2b or interferon alfa-2a.
Embodiment 91. The method of any one of embodiments 77-90, wherein the G-CSF therapy is filgrastim.
Embodiment 92. The method of any one of embodiments 77-91, wherein the immune checkpoint inhibitor is selected from the group consisting of: a CTLA-4 inhibitor; a PD-1 inhibitor; and a PD-L1 inhibitor.
Embodiment 93. The method of any one of embodiments 77-92, wherein the CTLA-4 inhibitor is ipilimumab or tremelimumab (CP-675,206).
Embodiment 94. The method of any one of embodiments 77-93, wherein the PD-1 inhibitor is pembrolizumab or nivolumab.
Embodiment 95. The method of any one of embodiments 77-94, wherein the PD-L1 inhibitor is selected from the group consisting of: atezolizumab; avelumab; and durvalumab.
Embodiment 96. The method of any one of embodiments 77-95, wherein the mRNA-based immunotherapy is CV9104.
Embodiment 97. The method of any one of embodiments 77-96, wherein the immunotherapy agent is bacillus Calmette-Guerin (BCG) therapy.
Embodiment 98. The method of any one of embodiments 77-98, wherein the oncolytic virus therapy is talimogene alherparepvec (T-VEC).
Embodiment 99. The method of any one of embodiments 77-98, wherein the cancer vaccine is a human papillomavirus (HPV) vaccine.
Embodiment 100. The method of any one of embodiments 77-99, wherein the human papillomavirus (HPV) vaccine is selected from the group consisting of: a recombinant human papillomavirus vaccine [types 6, 11, 16, and 18]; a recombinant human papillomavirus vaccine [types 6, 11, 16, 18, 31, 33, 45, 52, and 58]; and a recombinant human papillomavirus vaccine [types 16 and 18].
Embodiment 101. The method of any one of embodiments 77-100, wherein the cancer vaccine is a hepatitis B virus (HBV) vaccine.
Embodiment 102. The method of any one of embodiments 77-101, wherein the cancer vaccine is selected from the group consisting of: a combination Hepatitis A and Hepatitis B vaccine; a combination diphtheria, tetanus, pertussis, hepatitis B virus, and poliomyelitis vaccine; dasiprotimut-T; cancer vaccine HSPPC-96; GVAX; ADXS11-001; ALVAC-CEA; rilimogene galvacirepvec/rilimogene glafolivec; CDX-110; CimaVax-EGF; lapuleucel-T (APC8024); GRNVAC1; GRNVAC2; GRN-1201; hepcortespenlisimut-L (Hepko-V5); a dendritic cell vaccine; ICT-107; SCIB1; BMT CTN 1401; PrCa VBIR; PANVAC; a prostate cancer vaccine; DPX-Survivac; and viagenpumatucel-L (HS-110).
Embodiment 103. The method of any one of embodiments 77-102, wherein the peptide vaccine is selected from the group consisting of: nelipepimut-S(E75); IMA901; SurVaxM (SVN53-67); an immunogenic personal neoantigen vaccine; RGSH4K; and NEO-PV-01.
Embodiment 104. The method of any one of embodiments 77-103, wherein the DNA-based vaccine is a mammaglobin-A DNA vaccine.
Embodiment 105. The method of any one of embodiments 77-104, wherein the cancer is a Trk-associated cancer.
Embodiment 106. The method of any one of embodiments 77-105, wherein the Trk-associated cancer is due to oncogenic rearrangements in a NTRK gene selected from the group consisting of: NTRK1, NTRK2, and NTRK3.
Embodiment 107. The method of any one of embodiments 77-106, wherein the Trk-associated cancer has at least one point mutation in a NTRK1 gene that results in the expression of a TrkA protein comprising a mutation at one or more amino acid position(s) selected from the group consisting of: 517, 542, 564, 568, 573, 589, 595, 596, 599, 600, 602, 646, 656, 657, 667, 676, and 679.
Embodiment 108. The method of any one of embodiments 77-107, wherein the Trk-associated cancer has at least one point mutation in a NTRK2 gene that results in the expression of a TrkB protein comprising a mutation at one or more amino acid position(s) selected from the group consisting of: 545, 570, 596, 601, 617, 623, 624, 628, 630, 639, 672, 682, 683, 693, and 702.
Embodiment 109. The method of any one of embodiments 77-108, wherein the Trk-associated cancer has at least one point mutation in a NTRK3 gene that results in the expression of a TrkC protein comprising a mutation at one or more amino acid position(s) selected from the group consisting of: 545, 570, 596, 601, 617, 623, 624, 628, 630, 675, 685, 686, 696, and 705.
Embodiment 110. The method of any one of embodiments 77-109, wherein the cancer is selected from the group consisting of: adenocarcinoma; adrenal gland cortical carcinoma; adrenal gland neuroblastoma; anus squamous cell carcinoma; appendix adenocarcinoma; bladder urothelial carcinoma; bile duct adenocarcinoma; bladder carcinoma; bladder urothelial carcinoma; bone chordoma; bone marrow leukemia lymphocytic chronic; bone marrow leukemia non-lymphocytic acute myelocytic; bone marrow lymph proliferative disease; bone marrow multiple myeloma; bone sarcoma; brain astrocytoma; brain glioblastoma; brain medulloblastoma; brain meningioma; brain oligodendroglioma; breast adenoid cystic carcinoma; breast carcinoma; breast ductal carcinoma in situ; breast invasive ductal carcinoma; breast invasive lobular carcinoma; breast metaplastic carcinoma; cervix neuroendocrine carcinoma; cervix squamous cell carcinoma; colon adenocarcinoma; colon carcinoid tumor; duodenum adenocarcinoma; endometrioid tumor; esophagus adenocarcinoma; eye intraocular melanoma; eye intraocular squamous cell carcinoma; eye lacrimal duct carcinoma; fallopian tube serous carcinoma; gallbladder adenocarcinoma; gallbladder glomus tumor; gastroesophageal junction adenocarcinoma; head and neck adenoid cystic carcinoma; head and neck carcinoma; head and neck neuroblastoma; head and neck squamous cell carcinoma; kidney chromophore carcinoma; kidney medullary carcinoma; kidney renal cell carcinoma; kidney renal papillary carcinoma; kidney sarcomatoid carcinoma; kidney urothelial carcinoma; leukemia lymphocytic; liver cholangiocarcinoma; liver hepatocellular carcinoma; lung adenocarcinoma; lung adenosquamous carcinoma; lung atypical carcinoid; lung carcinosarcoma; lung large cell neuroendocrine carcinoma; lung non-small cell lung carcinoma; lung sarcoma; lung sarcomatoid carcinoma; lung small cell carcinoma; lung small cell undifferentiated carcinoma; lung squamous cell carcinoma; lymph node lymphoma diffuse large B cell; lymph node lymphoma follicular lymphoma; lymph node lymphoma mediastinal B-cell; lymph node lymphoma plasmablastic lung adenocarcinoma; lymphoma follicular lymphoma; non-Hodgkin's lymphoma; nasopharynx and paranasal sinuses undifferentiated carcinoma; ovary carcinoma; ovary carcinosarcoma; ovary clear cell carcinoma; ovary epithelial carcinoma; ovary granulosa cell tumor; ovary serous carcinoma; pancreas carcinoma; pancreas ductal adenocarcinoma; pancreas neuroendocrine carcinoma; peritoneum mesothelioma; peritoneum serous carcinoma; placenta choriocarcinoma; pleura mesothelioma; prostate acinar adenocarcinoma; prostate carcinoma; rectum adenocarcinoma; rectum squamous cell carcinoma; skin adnexal carcinoma; skin basal cell carcinoma; skin melanoma; skin Merkel cell carcinoma; skin squamous cell carcinoma; small intestine adenocarcinoma; small intestine gastrointestinal stromal tumors (GISTs); soft tissue angiosarcoma; soft tissue Ewing sarcoma; soft tissue hemangioendothelioma; soft tissue inflammatory myofibroblastic tumor; soft tissue leiomyosarcoma; soft tissue liposarcoma; soft tissue neuroblastoma; soft tissue paraganglioma; soft tissue perivascular epitheliod cell tumor; soft tissue sarcoma; non-rhabdomyosarcoma soft tissue sarcomas (NRSTS); retroperitoneal congenital sarcoma; soft tissue synovial sarcoma; hepatobiliary cancer; locally advanced sarcoma; stomach adenocarcinoma; stomach adenocarcinoma diffuse type; stomach adenocarcinoma intestinal type; stomach adenocarcinoma intestinal type; stomach leiomyosarcoma; thymus carcinoma; thymus thymoma lymphocytic; thyroid papillary carcinoma; unknown primary adenocarcinoma; unknown primary carcinoma; unknown primary malignant neoplasm; unknown primary melanoma; unknown primary sarcomatoid carcinoma; unknown primary squamous cell carcinoma; unknown undifferentiated neuroendocrine carcinoma; unknown primary undifferentiated small cell carcinoma; uterus carcinosarcoma; uterus endometrial adenocarcinoma; uterus endometrial adenocarcinoma endometrioid; uterus endometrial adenocarcinoma papillary serous; and uterus leiomyosarcoma.
Embodiment 111. The method of any one of embodiments 77-110, wherein the cancer is selected from the group consisting of: adrenocortical carcinoma; anal cancer; appendix cancer; atypical teratoid/rhabdoid tumor (e.g., central nervous system atypical teratoid/rhabdoid tumor); B-cell cancer; bile duct cancer; bladder cancer; bone cancer (e.g., osteosarcoma and malignant fibrous histiocytoma); brain cancer (e.g., brain and spinal cord tumor; brain stem glioma; central nervous system embryonal tumors; central nervous system germ cell tumors; craniopharyngioma; and ependymoma); breast cancer; bronchogenic carcinoma; bronchus cancer; cancer of hematological tissues; cancer of the oral cavity or pharynx; carcinoid tumor; cervical cancer; childhood cancers; chordoma; chronic lymphocytic leukemia; chronic myeloproliferative neoplasms; colon cancer; colorectal cancer; cutaneous T-cell lymphoma; ductal carcinoma in situ; embryonal tumor; endometrial cancer; esophageal cancer; esthesioneuroblastoma; extracranial germ cell tumor; extragonadal germ cell tumor; extrahepatic bile duct cancer; eye cancer (e.g., retinoblastoma); fallopian tube cancer; fibrosarcoma; fibrous histiocytoma of bone; gallbladder cancer; gastric cancer; gastrointestinal carcinoid tumor; germ cell tumor; gestational trophoblastic disease; glioblastoma multiforme; glioma (e.g., lower-grade glioma); head and neck cancer; heart cancer; histiocytosis; hypopharyngeal cancer; inflammatory myofibroblastic tumors; intrahepatic cholangiocarcinoma; islet cell tumor; kidney cancer (e.g., renal cell cancer); Langerhans cell histiocytosis; large cell neuroendocrine cancer; laryngeal cancer; leukemia (e.g., acute lymphoblastic leukemia; acute myeloid leukemia; chronic myelogenous leukemia; and hairy cell leukemia); lip cancer; liver cancer; lung cancer; Burkitt lymphoma; Hodgkin's lymphoma; and primary central nervous system lymphoma); medulloblastoma; mesothelioma; mouth cancer; multiple myeloma; myelodysplastic syndromes; nasal cavity and paranasal sinus cancer; nasopharyngeal cancer; neoplasm (e.g., a melanocystic neoplasm); nephroma; neuroblastoma; non-small cell lung cancer; oral cancer; oropharyngeal cancer; ovarian cancer; pancreatic cancer; paraganglioma; parathyroid cancer; glioma (e.g., pediatric gliomas); penile cancer; pharyngeal cancer; pheochromocytoma; pilocytic astrocytoma; pituitary tumor; plasma cell neoplasm; primary peritoneal cancer; prostate cancer; rectum carcinoma; salivary gland cancer; sarcoma (e.g., Ewing sarcoma; rhabdomyosarcoma; uterine sarcoma; and undifferentiated sarcoma); secretory breast carcinoma; Sezary syndrome; skin cancer; small bowel cancer; small cell lung cancer; small intestine cancer; Spitz nevi; Spitz tumors; spitzoid melanoma; stomach cancer; squamous cell carcinoma; squamous neck cancer; testicular cancer; throat cancer; thymoma and thymic carcinoma; thyroid carcinoma; urethral cancer; uterine cancer; urinary bladder cancer; vaginal cancer; vulvar cancer; and Wilms tumor.
Embodiment 112. A method for treating cancer, the method comprising administering to the patient a therapeutically effective amount of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate, (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one, and an immunotherapy agent.
Embodiment 113. The method of embodiment 112, wherein the immunotherapy agent is selected from the group consisting of: a cellular immunotherapy; an antibody therapy; an antibody-drug conjugate; a toxin; blinatumomab (AMG103) or midostaurin (Rydapt); a cytokine therapy; an immune checkpoint inhibitor; an mRNA-based immunotherapy; bacillus Calmette-Guerin (BCG) therapy; an oncolytic virus therapy; a cancer vaccine; a peptide vaccine; and a DNA-based vaccine.
Embodiment 114. The method of any one of embodiments 112-113, wherein the cellular immunotherapy is selected from the group consisting of: adoptive T-cell therapy; dendritic cell therapy; natural killer cell therapy; sipuleucel-T (APC8015); cells that express a chimeric antigen receptor (CAR); CAR-T cell therapy; and tisagenlecleucel.
Embodiment 115. The method of any one of embodiments 112-114, wherein the antibody therapy is selected from the group consisting of: a monoclonal antibody and a conjugated antibody.
Embodiment 116. The method of any one of embodiments 112-115, wherein the antibody therapy is selected from the group consisting of: bevacizumab; trastuzumab; avelumab; rituximab; edrecolomab; daratumuab; olaratumab; ofatumumab; alemtuzumab; cetuximab; oregovomab; pembrolizumab; dinutiximab; obinutuzumab; tremelimumab (CP-675;206); ramucirumab; ublituximab (TG-1101); panitumumab; elotuzumab; avelumab; necitumumab; cirmtuzumab; ibritumomab; isatuximab (SAR650984); nimotuzumab; fresolimumab (GC1008); lirilumab (INN); mogamulizumab; ficlatuzumab (AV-299); denosumab; ganitumab; urelumab; pidilizumab; and amatuximab.
Embodiment 117. The method of any one of embodiments 112-116, wherein the antibody-drug conjugate is selected from the group consisting of: gemtuzumab ozogamicin; inotuzumab ozogamicin; brentuximab vedotin; ado-trastuzumab emtansine (TDM-1); mirvetuximab soravtansine (IMGN853); and anetumab ravtansine.
Embodiment 118. The method of any one of embodiments 112-117, wherein the toxin is denileukin diftitox.
Embodiment 119. The method of any one of embodiments 112-118, wherein the immunotherapy agent is blinatumomab (AMG103).
Embodiment 120. The method of any one of embodiments 112-119, wherein the immunotherapy agent is midostaurin (Rydapt).
Embodiment 121. The method of any one of embodiments 112-120, wherein the cytokine therapy is selected from the group consisting of: an interleukin 2 (IL-2) therapy; an interferon alpha (IFNα) therapy; a granulocyte colony stimulating factor (G-CSF) therapy; an interleukin 12 (IL-12) therapy; an interleukin 15 (IL-15) therapy; an interleukin 7 (IL-7) therapy; and an erythropoietin-alpha (EPO) therapy.
Embodiment 122. The method of any one of embodiments 112-121, wherein the interleukin 2 (IL-2) therapy is aldesleukin.
Embodiment 123. The method of any one of embodiments 112-122, wherein the IFNα therapy is interferon alfa-2b or interferon alfa-2a.
Embodiment 124. The method of any one of embodiments 112-123, wherein the G-CSF therapy is filgrastim.
Embodiment 125. The method of any one of embodiments 112-124, wherein the immune checkpoint inhibitor is selected from the group consisting of: a CTLA-4 inhibitor; a PD-1 inhibitor; and a PD-L1 inhibitor.
Embodiment 126. The method of any one of embodiments 112-125, wherein the CTLA-4 inhibitor is ipilimumab or tremelimumab (CP-675,206).
Embodiment 127. The method of any one of embodiments 112-126, wherein the PD-1 inhibitor is pembrolizumab or nivolumab.
Embodiment 128. The method of any one of embodiments 112-127, wherein the PD-L1 inhibitor is selected from the group consisting of: atezolizumab; avelumab; and durvalumab.
Embodiment 129. The method of any one of embodiments 112-128, wherein the mRNA-based immunotherapy is CV9104.
Embodiment 130. The method of any one of embodiments 112-129, wherein the immunotherapy agent is bacillus Calmette-Guerin (BCG) therapy.
Embodiment 131. The method of any one of embodiments 112-130, wherein the oncolytic virus therapy is talimogene alherparepvec (T-VEC).
Embodiment 132. The method of any one of embodiments 112-131, wherein the cancer vaccine is a human papillomavirus (HPV) vaccine.
Embodiment 133. The method of any one of embodiments 112-132, wherein the human papillomavirus (HPV) vaccine is selected from the group consisting of: a recombinant human papillomavirus vaccine [types 6, 11, 16, and 18]; a recombinant human papillomavirus vaccine [types 6, 11, 16, 18, 31, 33, 45, 52, and 58]; and a recombinant human papillomavirus vaccine [types 16 and 18].
Embodiment 134. The method of any one of embodiments 112-133, wherein the cancer vaccine is a hepatitis B virus (HBV) vaccine.
Embodiment 135. The method of any one of embodiments 112-134, wherein the cancer vaccine is selected from the group consisting of: a combination Hepatitis A and Hepatitis B vaccine; a combination diphtheria, tetanus, pertussis, hepatitis B virus, and poliomyelitis vaccine; dasiprotimut-T; cancer vaccine HSPPC-96; GVAX; ADXS11-001; ALVAC-CEA; rilimogene galvacirepvec/rilimogene glafolivec; CDX-110; CimaVax-EGF; lapuleucel-T (APC8024); GRNVAC1; GRNVAC2; GRN-1201; hepcortespenlisimut-L (Hepko-V5); a dendritic cell vaccine; ICT-107; SCIB1; BMT CTN 1401; PrCa VBIR; PANVAC; a prostate cancer vaccine; DPX-Survivac; and viagenpumatucel-L (HS-110).
Embodiment 136. The method of any one of embodiments 112-135, wherein the peptide vaccine is selected from the group consisting of: nelipepimut-S(E75); IMA901; SurVaxM (SVN53-67); an immunogenic personal neoantigen vaccine; RGSH4K; and NEO-PV-01.
Embodiment 137. The method of any one of embodiments 112-136, wherein the DNA-based vaccine is a mammaglobin-A DNA vaccine.
Embodiment 138. The method of any one of embodiments 112-137, wherein the cancer is a Trk-associated cancer.
Embodiment 139. The method of any one of embodiments 112-138, wherein the Trk-associated cancer is due to oncogenic rearrangements in a NTRK gene selected from the group consisting of: NTRK1, NTRK2, and NTRK3.
Embodiment 140. The method of any one of embodiments 112-139, wherein the Trk-associated cancer has at least one point mutation in a NTRK1 gene that results in the expression of a TrkA protein comprising a mutation at one or more amino acid position(s) selected from the group consisting of: 517, 542, 564, 568, 573, 589, 595, 596, 599, 600, 602, 646, 656, 657, 667, 676, and 679.
Embodiment 141. The method of any one of embodiments 112-140, wherein the Trk-associated cancer has at least one point mutation in a NTRK2 gene that results in the expression of a TrkB protein comprising a mutation at one or more amino acid position(s) selected from the group consisting of: 545, 570, 596, 601, 617, 623, 624, 628, 630, 639, 672, 682, 683, 693, and 702.
Embodiment 142. The method of any one of embodiments 112-141, wherein the Trk-associated cancer has at least one point mutation in a NTRK3 gene that results in the expression of a TrkC protein comprising a mutation at one or more amino acid position(s) selected from the group consisting of: 545, 570, 596, 601, 617, 623, 624, 628, 630, 675, 685, 686, 696, and 705.
Embodiment 143. The method of any one of embodiments 112-142, wherein the cancer is selected from the group consisting of: adenocarcinoma; adrenal gland cortical carcinoma; adrenal gland neuroblastoma; anus squamous cell carcinoma; appendix adenocarcinoma; bladder urothelial carcinoma; bile duct adenocarcinoma; bladder carcinoma; bladder urothelial carcinoma; bone chordoma; bone marrow leukemia lymphocytic chronic; bone marrow leukemia non-lymphocytic acute myelocytic; bone marrow lymph proliferative disease; bone marrow multiple myeloma; bone sarcoma; brain astrocytoma; brain glioblastoma; brain medulloblastoma; brain meningioma; brain oligodendroglioma; breast adenoid cystic carcinoma; breast carcinoma; breast ductal carcinoma in situ; breast invasive ductal carcinoma; breast invasive lobular carcinoma; breast metaplastic carcinoma; cervix neuroendocrine carcinoma; cervix squamous cell carcinoma; colon adenocarcinoma; colon carcinoid tumor; duodenum adenocarcinoma; endometrioid tumor; esophagus adenocarcinoma; eye intraocular melanoma; eye intraocular squamous cell carcinoma; eye lacrimal duct carcinoma; fallopian tube serous carcinoma; gallbladder adenocarcinoma; gallbladder glomus tumor; gastroesophageal junction adenocarcinoma; head and neck adenoid cystic carcinoma; head and neck carcinoma; head and neck neuroblastoma; head and neck squamous cell carcinoma; kidney chromophore carcinoma; kidney medullary carcinoma; kidney renal cell carcinoma; kidney renal papillary carcinoma; kidney sarcomatoid carcinoma; kidney urothelial carcinoma; leukemia lymphocytic; liver cholangiocarcinoma; liver hepatocellular carcinoma; lung adenocarcinoma; lung adenosquamous carcinoma; lung atypical carcinoid; lung carcinosarcoma; lung large cell neuroendocrine carcinoma; lung non-small cell lung carcinoma; lung sarcoma; lung sarcomatoid carcinoma; lung small cell carcinoma; lung small cell undifferentiated carcinoma; lung squamous cell carcinoma; lymph node lymphoma diffuse large B cell; lymph node lymphoma follicular lymphoma; lymph node lymphoma mediastinal B-cell; lymph node lymphoma plasmablastic lung adenocarcinoma; lymphoma follicular lymphoma; non-Hodgkin's lymphoma; nasopharynx and paranasal sinuses undifferentiated carcinoma; ovary carcinoma; ovary carcinosarcoma; ovary clear cell carcinoma; ovary epithelial carcinoma; ovary granulosa cell tumor; ovary serous carcinoma; pancreas carcinoma; pancreas ductal adenocarcinoma; pancreas neuroendocrine carcinoma; peritoneum mesothelioma; peritoneum serous carcinoma; placenta choriocarcinoma; pleura mesothelioma; prostate acinar adenocarcinoma; prostate carcinoma; rectum adenocarcinoma; rectum squamous cell carcinoma; skin adnexal carcinoma; skin basal cell carcinoma; skin melanoma; skin Merkel cell carcinoma; skin squamous cell carcinoma; small intestine adenocarcinoma; small intestine gastrointestinal stromal tumors (GISTs); soft tissue angiosarcoma; soft tissue Ewing sarcoma; soft tissue hemangioendothelioma; soft tissue inflammatory myofibroblastic tumor; soft tissue leiomyosarcoma; soft tissue liposarcoma; soft tissue neuroblastoma; soft tissue paraganglioma; soft tissue perivascular epitheliod cell tumor; soft tissue sarcoma; non-rhabdomyosarcoma soft tissue sarcomas (NRSTS); retroperitoneal congenital sarcoma; soft tissue synovial sarcoma; locally advanced sarcoma; hepatobiliary cancer; stomach adenocarcinoma; stomach adenocarcinoma diffuse type; stomach adenocarcinoma intestinal type; stomach adenocarcinoma intestinal type; stomach leiomyosarcoma; thymus carcinoma; thymus thymoma lymphocytic; thyroid papillary carcinoma; unknown primary adenocarcinoma; unknown primary carcinoma; unknown primary malignant neoplasm; unknown primary melanoma; unknown primary sarcomatoid carcinoma; unknown primary squamous cell carcinoma; unknown undifferentiated neuroendocrine carcinoma; unknown primary undifferentiated small cell carcinoma; uterus carcinosarcoma; uterus endometrial adenocarcinoma; uterus endometrial adenocarcinoma endometrioid; uterus endometrial adenocarcinoma papillary serous; and uterus leiomyosarcoma.
Embodiment 144. The method of any one of embodiments 112-143, wherein the cancer is selected from the group consisting of: adrenocortical carcinoma; anal cancer; appendix cancer; atypical teratoid/rhabdoid tumor (e.g., central nervous system atypical teratoid/rhabdoid tumor); B-cell cancer; bile duct cancer; bladder cancer; bone cancer (e.g., osteosarcoma and malignant fibrous histiocytoma); brain cancer (e.g., brain and spinal cord tumor; brain stem glioma; central nervous system embryonal tumors; central nervous system germ cell tumors; craniopharyngioma; and ependymoma); breast cancer; bronchogenic carcinoma; bronchus cancer; cancer of hematological tissues; cancer of the oral cavity or pharynx; carcinoid tumor; cervical cancer; childhood cancers; chordoma; chronic lymphocytic leukemia; chronic myeloproliferative neoplasms; colon cancer; colorectal cancer; cutaneous T-cell lymphoma; ductal carcinoma in situ; embryonal tumor; endometrial cancer; esophageal cancer; esthesioneuroblastoma; extracranial germ cell tumor; extragonadal germ cell tumor; extrahepatic bile duct cancer; eye cancer (e.g., retinoblastoma); fallopian tube cancer; fibrosarcoma; fibrous histiocytoma of bone; gallbladder cancer; gastric cancer; gastrointestinal carcinoid tumor; germ cell tumor; gestational trophoblastic disease; glioblastoma multiforme; glioma (e.g., lower-grade glioma); head and neck cancer; heart cancer; histiocytosis; hypopharyngeal cancer; inflammatory myofibroblastic tumors; intrahepatic cholangiocarcinoma; islet cell tumor; kidney cancer (e.g., renal cell cancer); Langerhans cell histiocytosis; large cell neuroendocrine cancer; laryngeal cancer; leukemia (e.g., acute lymphoblastic leukemia; acute myeloid leukemia; chronic myelogenous leukemia; and hairy cell leukemia); lip cancer; liver cancer; lung cancer; Burkitt lymphoma; Hodgkin's lymphoma; and primary central nervous system lymphoma); medulloblastoma; mesothelioma; mouth cancer; multiple myeloma; myelodysplastic syndromes; nasal cavity and paranasal sinus cancer; nasopharyngeal cancer; neoplasm (e.g., a melanocystic neoplasm); nephroma; neuroblastoma; non-small cell lung cancer; oral cancer; oropharyngeal cancer; ovarian cancer; pancreatic cancer; paraganglioma; parathyroid cancer; glioma (e.g., pediatric gliomas); penile cancer; pharyngeal cancer; pheochromocytoma; pilocytic astrocytoma; pituitary tumor; plasma cell neoplasm; primary peritoneal cancer; prostate cancer; rectum carcinoma; salivary gland cancer; sarcoma (e.g., Ewing sarcoma; rhabdomyosarcoma; uterine sarcoma; and undifferentiated sarcoma); secretory breast carcinoma; Sezary syndrome; skin cancer; small bowel cancer; small cell lung cancer; small intestine cancer; Spitz nevi; Spitz tumors; spitzoid melanoma; stomach cancer; squamous cell carcinoma; squamous neck cancer; testicular cancer; throat cancer; thymoma and thymic carcinoma; thyroid carcinoma; urethral cancer; uterine cancer; urinary bladder cancer; vaginal cancer; vulvar cancer; and Wilms tumor.
Embodiment 145. A method for treating cancer, the method comprising administering to the patient a therapeutically effective amount of (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate and an immunotherapy agent.
Embodiment 146. The method of embodiment 145, wherein the immunotherapy agent is selected from the group consisting of: a cellular immunotherapy; an antibody therapy; an antibody-drug conjugate; a toxin; blinatumomab (AMG103) or midostaurin (Rydapt); a cytokine therapy; an immune checkpoint inhibitor; an mRNA-based immunotherapy; bacillus Calmette-Guerin (BCG) therapy; an oncolytic virus therapy; a cancer vaccine; a peptide vaccine; and a DNA-based vaccine.
Embodiment 147. The method of any one of embodiments 145-146, wherein the cellular immunotherapy is selected from the group consisting of: adoptive T-cell therapy; dendritic cell therapy; natural killer cell therapy; sipuleucel-T (APC8015); cells that express a chimeric antigen receptor (CAR); CAR-T cell therapy; and tisagenlecleucel.
Embodiment 148. The method of any one of embodiments 145-147, wherein the antibody therapy is selected from the group consisting of: a monoclonal antibody and a conjugated antibody.
Embodiment 149. The method of any one of embodiments 145-148, wherein the antibody therapy is selected from the group consisting of: bevacizumab; trastuzumab; avelumab; rituximab; edrecolomab; daratumuab; olaratumab; ofatumumab; alemtuzumab; cetuximab; oregovomab; pembrolizumab; dinutiximab; obinutuzumab; tremelimumab (CP-675;206); ramucirumab; ublituximab (TG-1101); panitumumab; elotuzumab; avelumab; necitumumab; cirmtuzumab; ibritumomab; isatuximab (SAR650984); nimotuzumab; fresolimumab (GC1008); lirilumab (INN); mogamulizumab; ficlatuzumab (AV-299); denosumab; ganitumab; urelumab; pidilizumab; and amatuximab.
Embodiment 150. The method of any one of embodiments 145-149, wherein the antibody-drug conjugate is selected from the group consisting of: gemtuzumab ozogamicin; inotuzumab ozogamicin; brentuximab vedotin; ado-trastuzumab emtansine (TDM-1); mirvetuximab soravtansine (IMGN853); and anetumab ravtansine.
Embodiment 151. The method of any one of embodiments 145-150, wherein the toxin is denileukin diftitox.
Embodiment 152. The method of any one of embodiments 145-151, wherein the immunotherapy agent is blinatumomab (AMG103).
Embodiment 153. The method of any one of embodiments 145-152, wherein the immunotherapy agent is midostaurin (Rydapt).
Embodiment 154. The method of any one of embodiments 145-153, wherein the cytokine therapy is selected from the group consisting of: an interleukin 2 (IL-2) therapy; an interferon alpha (IFNα) therapy; a granulocyte colony stimulating factor (G-CSF) therapy; an interleukin 12 (IL-12) therapy; an interleukin 15 (IL-15) therapy; an interleukin 7 (IL-7) therapy; and an erythropoietin-alpha (EPO) therapy.
Embodiment 155. The method of any one of embodiments 145-154, wherein the interleukin 2 (IL-2) therapy is aldesleukin.
Embodiment 156. The method of any one of embodiments 145-155, wherein the IFNα therapy is interferon alfa-2b or interferon alfa-2a.
Embodiment 157. The method of any one of embodiments 145-156, wherein the G-CSF therapy is filgrastim.
Embodiment 158. The method of any one of embodiments 145-157, wherein the immune checkpoint inhibitor is selected from the group consisting of: a CTLA-4 inhibitor; a PD-1 inhibitor; and a PD-L1 inhibitor.
Embodiment 159. The method of any one of embodiments 145-158, wherein the CTLA-4 inhibitor is ipilimumab or tremelimumab (CP-675,206).
Embodiment 160. The method of any one of embodiments 145-159, wherein the PD-1 inhibitor is pembrolizumab or nivolumab.
Embodiment 161. The method of any one of embodiments 145-160, wherein the PD-L1 inhibitor is selected from the group consisting of: atezolizumab; avelumab; and durvalumab.
Embodiment 162. The method of any one of embodiments 145-161, wherein the mRNA-based immunotherapy is CV9104.
Embodiment 163. The method of any one of embodiments 145-162, wherein the immunotherapy agent is bacillus Calmette-Guerin (BCG) therapy.
Embodiment 164. The method of any one of embodiments 145-163, wherein the oncolytic virus therapy is talimogene alherparepvec (T-VEC).
Embodiment 165. The method of any one of embodiments 145-164, wherein the cancer vaccine is a human papillomavirus (HPV) vaccine.
Embodiment 166. The method of any one of embodiments 145-165, wherein the human papillomavirus (HPV) vaccine is selected from the group consisting of: a recombinant human papillomavirus vaccine [types 6, 11, 16, and 18]; a recombinant human papillomavirus vaccine [types 6, 11, 16, 18, 31, 33, 45, 52, and 58]; and a recombinant human papillomavirus vaccine [types 16 and 18].
Embodiment 167. The method of any one of embodiments 145-166, wherein the cancer vaccine is a hepatitis B virus (HBV) vaccine.
Embodiment 168. The method of any one of embodiments 145-167, wherein the cancer vaccine is selected from the group consisting of: a combination Hepatitis A and Hepatitis B vaccine; a combination diphtheria, tetanus, pertussis, hepatitis B virus, and poliomyelitis vaccine; dasiprotimut-T; cancer vaccine HSPPC-96; GVAX; ADXS11-001; ALVAC-CEA; rilimogene galvacirepvec/rilimogene glafolivec; CDX-110; CimaVax-EGF; lapuleucel-T (APC8024); GRNVAC1; GRNVAC2; GRN-1201; hepcortespenlisimut-L (Hepko-V5); a dendritic cell vaccine; ICT-107; SCIB1; BMT CTN 1401; PrCa VBIR; PANVAC; a prostate cancer vaccine; DPX-Survivac; and viagenpumatucel-L (HS-110).
Embodiment 169. The method of any one of embodiments 145-168, wherein the peptide vaccine is selected from the group consisting of: nelipepimut-S(E75); IMA901; SurVaxM (SVN53-67); an immunogenic personal neoantigen vaccine; RGSH4K; and NEO-PV-01.
Embodiment 170. The method of any one of embodiments 145-169, wherein the DNA-based vaccine is a mammaglobin-A DNA vaccine.
Embodiment 171. The method of any one of embodiments 145-170, wherein the cancer is a Trk-associated cancer.
Embodiment 172. The method of any one of embodiments 145-171, wherein the Trk-associated cancer is due to oncogenic rearrangements in a NTRK gene selected from the group consisting of: NTRK1, NTRK2, and NTRK3.
Embodiment 173. The method of any one of embodiments 145-172, wherein the Trk-associated cancer has at least one point mutation in a NTRK1 gene that results in the expression of a TrkA protein comprising a mutation at one or more amino acid position(s) selected from the group consisting of: 517, 542, 564, 568, 573, 589, 595, 596, 599, 600, 602, 646, 656, 657, 667, 676, and 679.
Embodiment 174. The method of any one of embodiments 145-173, wherein the Trk-associated cancer has at least one point mutation in a NTRK2 gene that results in the expression of a TrkB protein comprising a mutation at one or more amino acid position(s) selected from the group consisting of: 545, 570, 596, 601, 617, 623, 624, 628, 630, 639, 672, 682, 683, 693, and 702.
Embodiment 175. The method of any one of embodiments 145-174, wherein the Trk-associated cancer has at least one point mutation in a NTRK3 gene that results in the expression of a TrkC protein comprising a mutation at one or more amino acid position(s) selected from the group consisting of: 545, 570, 596, 601, 617, 623, 624, 628, 630, 675, 685, 686, 696, and 705.
Embodiment 176. The method of any one of embodiments 145-175, wherein the cancer is selected from the group consisting of: adenocarcinoma; adrenal gland cortical carcinoma; adrenal gland neuroblastoma; anus squamous cell carcinoma; appendix adenocarcinoma; bladder urothelial carcinoma; bile duct adenocarcinoma; bladder carcinoma; bladder urothelial carcinoma; bone chordoma; bone marrow leukemia lymphocytic chronic; bone marrow leukemia non-lymphocytic acute myelocytic; bone marrow lymph proliferative disease; bone marrow multiple myeloma; bone sarcoma; brain astrocytoma; brain glioblastoma; brain medulloblastoma; brain meningioma; brain oligodendroglioma; breast adenoid cystic carcinoma; breast carcinoma; breast ductal carcinoma in situ; breast invasive ductal carcinoma; breast invasive lobular carcinoma; breast metaplastic carcinoma; cervix neuroendocrine carcinoma; cervix squamous cell carcinoma; colon adenocarcinoma; colon carcinoid tumor; duodenum adenocarcinoma; endometrioid tumor; esophagus adenocarcinoma; eye intraocular melanoma; eye intraocular squamous cell carcinoma; eye lacrimal duct carcinoma; fallopian tube serous carcinoma; gallbladder adenocarcinoma; gallbladder glomus tumor; gastroesophageal junction adenocarcinoma; head and neck adenoid cystic carcinoma; head and neck carcinoma; head and neck neuroblastoma; head and neck squamous cell carcinoma; kidney chromophore carcinoma; kidney medullary carcinoma; kidney renal cell carcinoma; kidney renal papillary carcinoma; kidney sarcomatoid carcinoma; kidney urothelial carcinoma; leukemia lymphocytic; liver cholangiocarcinoma; liver hepatocellular carcinoma; lung adenocarcinoma; lung adenosquamous carcinoma; lung atypical carcinoid; lung carcinosarcoma; lung large cell neuroendocrine carcinoma; lung non-small cell lung carcinoma; lung sarcoma; lung sarcomatoid carcinoma; lung small cell carcinoma; lung small cell undifferentiated carcinoma; lung squamous cell carcinoma; lymph node lymphoma diffuse large B cell; lymph node lymphoma follicular lymphoma; lymph node lymphoma mediastinal B-cell; lymph node lymphoma plasmablastic lung adenocarcinoma; lymphoma follicular lymphoma; non-Hodgkin's lymphoma; nasopharynx and paranasal sinuses undifferentiated carcinoma; ovary carcinoma; ovary carcinosarcoma; ovary clear cell carcinoma; ovary epithelial carcinoma; ovary granulosa cell tumor; ovary serous carcinoma; pancreas carcinoma; pancreas ductal adenocarcinoma; pancreas neuroendocrine carcinoma; peritoneum mesothelioma; peritoneum serous carcinoma; placenta choriocarcinoma; pleura mesothelioma; prostate acinar adenocarcinoma; prostate carcinoma; rectum adenocarcinoma; rectum squamous cell carcinoma; skin adnexal carcinoma; skin basal cell carcinoma; skin melanoma; skin Merkel cell carcinoma; skin squamous cell carcinoma; small intestine adenocarcinoma; small intestine gastrointestinal stromal tumors (GISTs); soft tissue angiosarcoma; soft tissue Ewing sarcoma; soft tissue hemangioendothelioma; soft tissue inflammatory myofibroblastic tumor; soft tissue leiomyosarcoma; soft tissue liposarcoma; soft tissue neuroblastoma; soft tissue paraganglioma; soft tissue perivascular epitheliod cell tumor; soft tissue sarcoma; non-rhabdomyosarcoma soft tissue sarcomas (NRSTS); retroperitoneal congenital sarcoma; soft tissue synovial sarcoma; locally advanced sarcoma; hepatobiliary cancer; stomach adenocarcinoma; stomach adenocarcinoma diffuse type; stomach adenocarcinoma intestinal type; stomach adenocarcinoma intestinal type; stomach leiomyosarcoma; thymus carcinoma; thymus thymoma lymphocytic; thyroid papillary carcinoma; unknown primary adenocarcinoma; unknown primary carcinoma; unknown primary malignant neoplasm; unknown primary melanoma; unknown primary sarcomatoid carcinoma; unknown primary squamous cell carcinoma; unknown undifferentiated neuroendocrine carcinoma; unknown primary undifferentiated small cell carcinoma; uterus carcinosarcoma; uterus endometrial adenocarcinoma; uterus endometrial adenocarcinoma endometrioid; uterus endometrial adenocarcinoma papillary serous; and uterus leiomyosarcoma.
Embodiment 177. The method of any one of embodiments 145-176, wherein the cancer is selected from the group consisting of: adrenocortical carcinoma; anal cancer; appendix cancer; atypical teratoid/rhabdoid tumor (e.g., central nervous system atypical teratoid/rhabdoid tumor); B-cell cancer; bile duct cancer; bladder cancer; bone cancer (e.g., osteosarcoma and malignant fibrous histiocytoma); brain cancer (e.g., brain and spinal cord tumor; brain stem glioma; central nervous system embryonal tumors; central nervous system germ cell tumors; craniopharyngioma; and ependymoma); breast cancer; bronchogenic carcinoma; bronchus cancer; cancer of hematological tissues; cancer of the oral cavity or pharynx; carcinoid tumor; cervical cancer; childhood cancers; chordoma; chronic lymphocytic leukemia; chronic myeloproliferative neoplasms; colon cancer; colorectal cancer; cutaneous T-cell lymphoma; ductal carcinoma in situ; embryonal tumor; endometrial cancer; esophageal cancer; esthesioneuroblastoma; extracranial germ cell tumor; extragonadal germ cell tumor; extrahepatic bile duct cancer; eye cancer (e.g., retinoblastoma); fallopian tube cancer; fibrosarcoma; fibrous histiocytoma of bone; gallbladder cancer; gastric cancer; gastrointestinal carcinoid tumor; germ cell tumor; gestational trophoblastic disease; glioblastoma multiforme; glioma (e.g., lower-grade glioma); head and neck cancer; heart cancer; histiocytosis; hypopharyngeal cancer; inflammatory myofibroblastic tumors; intrahepatic cholangiocarcinoma; islet cell tumor; kidney cancer (e.g., renal cell cancer); Langerhans cell histiocytosis; large cell neuroendocrine cancer; laryngeal cancer; leukemia (e.g., acute lymphoblastic leukemia; acute myeloid leukemia; chronic myelogenous leukemia; and hairy cell leukemia); lip cancer; liver cancer; lung cancer; Burkitt lymphoma; Hodgkin's lymphoma; and primary central nervous system lymphoma); medulloblastoma; mesothelioma; mouth cancer; multiple myeloma; myelodysplastic syndromes; nasal cavity and paranasal sinus cancer; nasopharyngeal cancer; neoplasm (e.g., a melanocystic neoplasm); nephroma; neuroblastoma; non-small cell lung cancer; oral cancer; oropharyngeal cancer; ovarian cancer; pancreatic cancer; paraganglioma; parathyroid cancer; glioma (e.g., pediatric gliomas); penile cancer; pharyngeal cancer; pheochromocytoma; pilocytic astrocytoma; pituitary tumor; plasma cell neoplasm; primary peritoneal cancer; prostate cancer; rectum carcinoma; salivary gland cancer; sarcoma (e.g., Ewing sarcoma; rhabdomyosarcoma; uterine sarcoma; and undifferentiated sarcoma); secretory breast carcinoma; Sezary syndrome; skin cancer; small bowel cancer; small cell lung cancer; small intestine cancer; Spitz nevi; Spitz tumors; spitzoid melanoma; stomach cancer; squamous cell carcinoma; squamous neck cancer; testicular cancer; throat cancer; thymoma and thymic carcinoma; thyroid carcinoma; urethral cancer; uterine cancer; urinary bladder cancer; vaginal cancer; vulvar cancer; and Wilms tumor.
Embodiment 178. A method for treating cancer, the method comprising administering to the patient a therapeutically effective amount of (6R,15R)-9-fluoro-15-methyl-2,11,16,20,21,24-hexaazapentacyclo[16.5.2.0^{2,6}.0^{7,12}.0^{21,25}]pentacosa-1(24),7,9, 11,18(25),19,22-heptaen-17-one and an immunotherapy agent.
Embodiment 179. The method of embodiment 178, wherein the immunotherapy agent is selected from the group consisting of: a cellular immunotherapy; an antibody therapy; an antibody-drug conjugate; a toxin; blinatumomab (AMG103) or midostaurin (Rydapt); a cytokine therapy; an immune checkpoint inhibitor; an mRNA-based immunotherapy; bacillus Calmette-Guerin (BCG) therapy; an oncolytic virus therapy; a cancer vaccine; a peptide vaccine; and a DNA-based vaccine.
Embodiment 180. The method of any one of embodiments 178-179, wherein the cellular immunotherapy is selected from the group consisting of: adoptive T-cell therapy; dendritic cell therapy; natural killer cell therapy; sipuleucel-T (APC8015); cells that express a chimeric antigen receptor (CAR); CAR-T cell therapy; and tisagenlecleucel.
Embodiment 181. The method of any one of embodiments 178-180, wherein the antibody therapy is selected from the group consisting of: a monoclonal antibody and a conjugated antibody.
Embodiment 182. The method of any one of embodiments 178-181, wherein the antibody therapy is selected from the group consisting of: bevacizumab; trastuzumab; avelumab; rituximab; edrecolomab; daratumuab; olaratumab; ofatumumab; alemtuzumab; cetuximab; oregovomab; pembrolizumab; dinutiximab; obinutuzumab; tremelimumab (CP-675;206); ramucirumab; ublituximab (TG-1101); panitumumab; elotuzumab; avelumab; necitumumab; cirmtuzumab; ibritumomab; isatuximab (SAR650984); nimotuzumab; fresolimumab (GC1008); lirilumab (INN); mogamulizumab; ficlatuzumab (AV-299); denosumab; ganitumab; urelumab; pidilizumab; and amatuximab.
Embodiment 183. The method of any one of embodiments 178-182, wherein the antibody-drug conjugate is selected from the group consisting of: gemtuzumab ozogamicin; inotuzumab ozogamicin; brentuximab vedotin; ado-trastuzumab emtansine (TDM-1); mirvetuximab soravtansine (IMGN853); and anetumab ravtansine.
Embodiment 184. The method of any one of embodiments 178-183, wherein the toxin is denileukin diftitox.
Embodiment 185. The method of any one of embodiments 178-184, wherein the immunotherapy agent is blinatumomab (AMG103).
Embodiment 186. The method of any one of embodiments 178-185, wherein the immunotherapy agent is midostaurin (Rydapt).
Embodiment 187. The method of any one of embodiments 178-186, wherein the cytokine therapy is selected from the group consisting of: an interleukin 2 (IL-2) therapy; an interferon alpha (IFNα) therapy; a granulocyte colony stimulating factor (G-CSF) therapy; an interleukin 12 (IL-12) therapy; an interleukin 15 (IL-15) therapy; an interleukin 7 (IL-7) therapy; and an erythropoietin-alpha (EPO) therapy.
Embodiment 188. The method of any one of embodiments 178-187, wherein the interleukin 2 (IL-2) therapy is aldesleukin.
Embodiment 189. The method of any one of embodiments 178-188, wherein the IFNα therapy is interferon alfa-2b or interferon alfa-2a.
Embodiment 190. The method of any one of embodiments 178-189, wherein the G-CSF therapy is filgrastim.
Embodiment 191. The method of any one of embodiments 178-190, wherein the immune checkpoint inhibitor is selected from the group consisting of: a CTLA-4 inhibitor; a PD-1 inhibitor; and a PD-L1 inhibitor.
Embodiment 192. The method of any one of embodiments 178-191, wherein the CTLA-4 inhibitor is ipilimumab or tremelimumab (CP-675,206).
Embodiment 193. The method of any one of embodiments 178-192, wherein the PD-1 inhibitor is pembrolizumab or nivolumab.
Embodiment 194. The method of any one of embodiments 178-193, wherein the PD-L1 inhibitor is selected from the group consisting of: atezolizumab; avelumab; and durvalumab.
Embodiment 195. The method of any one of embodiments 178-194, wherein the mRNA-based immunotherapy is CV9104.
Embodiment 196. The method of any one of embodiments 178-195, wherein the immunotherapy agent is bacillus Calmette-Guerin (BCG) therapy.
Embodiment 197. The method of any one of embodiments 178-196, wherein the oncolytic virus therapy is talimogene alherparepvec (T-VEC).
Embodiment 198. The method of any one of embodiments 178-197, wherein the cancer vaccine is a human papillomavirus (HPV) vaccine.
Embodiment 199. The method of any one of embodiments 178-198, wherein the human papillomavirus (HPV) vaccine is selected from the group consisting of: a recombinant human papillomavirus vaccine [types 6, 11, 16, and 18]; a recombinant human papillomavirus vaccine [types 6, 11, 16, 18, 31, 33, 45, 52, and 58]; and a recombinant human papillomavirus vaccine [types 16 and 18].
Embodiment 200. The method of any one of embodiments 178-199, wherein the cancer vaccine is a hepatitis B virus (HBV) vaccine.
Embodiment 201. The method of any one of embodiments 178-200, wherein the cancer vaccine is selected from the group consisting of: a combination Hepatitis A and Hepatitis B vaccine; a combination diphtheria, tetanus, pertussis, hepatitis B virus, and poliomyelitis vaccine; dasiprotimut-T; cancer vaccine HSPPC-96; GVAX; ADXS11-001; ALVAC-CEA; rilimogene galvacirepvec/rilimogene glafolivec; CDX-110; CimaVax-EGF; lapuleucel-T (APC8024); GRNVAC1; GRNVAC2; GRN-1201; hepcortespenlisimut-L (Hepko-V5); a dendritic cell vaccine; ICT-107; SCIB1; BMT CTN 1401; PrCa VBIR; PANVAC; a prostate cancer vaccine; DPX-Survivac; and viagenpumatucel-L (HS-110).
Embodiment 202. The method of any one of embodiments 178-201, wherein the peptide vaccine is selected from the group consisting of: nelipepimut-S(E75); IMA901; SurVaxM (SVN53-67); an immunogenic personal neoantigen vaccine; RGSH4K; and NEO-PV-01.
Embodiment 203. The method of any one of embodiments 178-202, wherein the DNA-based vaccine is a mammaglobin-A DNA vaccine.
Embodiment 204. The method of any one of embodiments 178-203, wherein the cancer is a Trk-associated cancer.
Embodiment 205. The method of any one of embodiments 178-204, wherein the Trk-associated cancer is due to oncogenic rearrangements in a NTRK gene selected from the group consisting of: NTRK1, NTRK2, and NTRK3.
Embodiment 206. The method of any one of embodiments 178-205, wherein the Trk-associated cancer has at least one point mutation in a NTRK1 gene that results in the expression of a TrkA protein comprising a mutation at one or more amino acid position(s) selected from the group consisting of: 517, 542, 564, 568, 573, 589, 595, 596, 599, 600, 602, 646, 656, 657, 667, 676, and 679.
Embodiment 207. The method of any one of embodiments 178-206, wherein the Trk-associated cancer has at least one point mutation in a NTRK2 gene that results in the expression of a TrkB protein comprising a mutation at one or more amino acid position(s) selected from the group consisting of: 545, 570, 596, 601, 617, 623, 624, 628, 630, 639, 672, 682, 683, 693, and 702.
Embodiment 208. The method of any one of embodiments 178-207, wherein the Trk-associated cancer has at least one point mutation in a NTRK3 gene that results in the expression of a TrkC protein comprising a mutation at one or more amino acid position(s) selected from the group consisting of: 545, 570, 596, 601, 617, 623, 624, 628, 630, 675, 685, 686, 696, and 705.
Embodiment 209. The method of any one of embodiments 178-208, wherein the cancer is selected from the group consisting of: adenocarcinoma; adrenal gland cortical carcinoma; adrenal gland neuroblastoma; anus squamous cell carcinoma; appendix adenocarcinoma; bladder urothelial carcinoma; bile duct adenocarcinoma; bladder carcinoma; bladder urothelial carcinoma; bone chordoma; bone marrow leukemia lymphocytic chronic; bone marrow leukemia non-lymphocytic acute myelocytic; bone marrow lymph proliferative disease; bone marrow multiple myeloma; bone sarcoma; brain astrocytoma; brain glioblastoma; brain medulloblastoma; brain meningioma; brain oligodendroglioma; breast adenoid cystic carcinoma; breast carcinoma; breast ductal carcinoma in situ; breast invasive ductal carcinoma; breast invasive lobular carcinoma; breast metaplastic carcinoma; cervix neuroendocrine carcinoma; cervix squamous cell carcinoma; colon adenocarcinoma; colon carcinoid tumor; duodenum adenocarcinoma; endometrioid tumor; esophagus adenocarcinoma; eye intraocular melanoma; eye intraocular squamous cell carcinoma; eye lacrimal duct carcinoma; fallopian tube serous carcinoma; gallbladder adenocarcinoma; gallbladder glomus tumor; gastroesophageal junction adenocarcinoma; head and neck adenoid cystic carcinoma; head and neck carcinoma; head and neck neuroblastoma; head and neck squamous cell carcinoma; kidney chromophore carcinoma; kidney medullary carcinoma; kidney renal cell carcinoma; kidney renal papillary carcinoma; kidney sarcomatoid carcinoma; kidney urothelial carcinoma; leukemia lymphocytic; liver cholangiocarcinoma; liver hepatocellular carcinoma; lung adenocarcinoma; lung adenosquamous carcinoma; lung atypical carcinoid; lung carcinosarcoma; lung large cell neuroendocrine carcinoma; lung non-small cell lung carcinoma; lung sarcoma; lung sarcomatoid carcinoma; lung small cell carcinoma; lung small cell undifferentiated carcinoma; lung squamous cell carcinoma; lymph node lymphoma diffuse large B cell; lymph node lymphoma follicular lymphoma; lymph node lymphoma mediastinal B-cell; lymph node lymphoma plasmablastic lung adenocarcinoma; lymphoma follicular lymphoma; non-Hodgkin's lymphoma; nasopharynx and paranasal sinuses undifferentiated carcinoma; ovary carcinoma; ovary carcinosarcoma; ovary clear cell carcinoma; ovary epithelial carcinoma; ovary granulosa cell tumor; ovary serous carcinoma; pancreas carcinoma; pancreas ductal adenocarcinoma; pancreas neuroendocrine carcinoma; peritoneum mesothelioma; peritoneum serous carcinoma; placenta choriocarcinoma; pleura mesothelioma; prostate acinar adenocarcinoma; prostate carcinoma; rectum adenocarcinoma; rectum squamous cell carcinoma; skin adnexal carcinoma; skin basal cell carcinoma; skin melanoma; skin Merkel cell carcinoma; skin squamous cell carcinoma; small intestine adenocarcinoma; small intestine gastrointestinal stromal tumors (GISTs); soft tissue angiosarcoma; soft tissue Ewing sarcoma; soft tissue hemangioendothelioma; soft tissue inflammatory myofibroblastic tumor; soft tissue leiomyosarcoma; soft tissue liposarcoma; soft tissue neuroblastoma; soft tissue paraganglioma; soft tissue perivascular epitheliod cell tumor; soft tissue sarcoma; non-rhabdomyosarcoma soft tissue sarcomas (NRSTS); retroperitoneal congenital sarcoma; soft tissue synovial sarcoma; locally advanced sarcoma; hepatobiliary cancer; stomach adenocarcinoma; stomach adenocarcinoma diffuse type; stomach adenocarcinoma intestinal type; stomach adenocarcinoma intestinal type; stomach leiomyosarcoma; thymus carcinoma; thymus thymoma lymphocytic; thyroid papillary carcinoma; unknown primary adenocarcinoma; unknown primary carcinoma; unknown primary malignant neoplasm; unknown primary melanoma; unknown primary sarcomatoid carcinoma; unknown primary squamous cell carcinoma; unknown undifferentiated neuroendocrine carcinoma; unknown primary undifferentiated small cell carcinoma; uterus carcinosarcoma; uterus endometrial adenocarcinoma; uterus endometrial adenocarcinoma endometrioid; uterus endometrial adenocarcinoma papillary serous; and uterus leiomyosarcoma.
Embodiment 210. The method of any one of embodiments 178-209, wherein the cancer is selected from the group consisting of: adrenocortical carcinoma; anal cancer; appendix cancer; atypical teratoid/rhabdoid tumor (e.g., central nervous system atypical teratoid/rhabdoid tumor); B-cell cancer; bile duct cancer; bladder cancer; bone cancer (e.g., osteosarcoma and malignant fibrous histiocytoma); brain cancer (e.g., brain and spinal cord tumor; brain stem glioma; central nervous system embryonal tumors; central nervous system germ cell tumors; craniopharyngioma; and ependymoma); breast cancer; bronchogenic carcinoma; bronchus cancer; cancer of hematological tissues; cancer of the oral cavity or pharynx; carcinoid tumor; cervical cancer; childhood cancers; chordoma; chronic lymphocytic leukemia; chronic myeloproliferative neoplasms; colon cancer; colorectal cancer; cutaneous T-cell lymphoma; ductal carcinoma in situ; embryonal tumor; endometrial cancer; esophageal cancer; esthesioneuroblastoma; extracranial germ cell tumor; extragonadal germ cell tumor; extrahepatic bile duct cancer; eye cancer (e.g., retinoblastoma); fallopian tube cancer; fibrosarcoma; fibrous histiocytoma of bone; gallbladder cancer; gastric cancer; gastrointestinal carcinoid tumor; germ cell tumor; gestational trophoblastic disease; glioblastoma multiforme; glioma (e.g., lower-grade glioma); head and neck cancer; heart cancer; histiocytosis; hypopharyngeal cancer; inflammatory myofibroblastic tumors; intrahepatic cholangiocarcinoma; islet cell tumor; kidney cancer (e.g., renal cell cancer); Langerhans cell histiocytosis; large cell neuroendocrine cancer; laryngeal cancer; leukemia (e.g., acute lymphoblastic leukemia; acute myeloid leukemia; chronic myelogenous leukemia; and hairy cell leukemia); lip cancer; liver cancer; lung cancer; Burkitt lymphoma; Hodgkin's lymphoma; and primary central nervous system lymphoma); medulloblastoma; mesothelioma; mouth cancer; multiple myeloma; myelodysplastic syndromes; nasal cavity and paranasal sinus cancer; nasopharyngeal cancer; neoplasm (e.g., a melanocystic neoplasm); nephroma; neuroblastoma; non-small cell lung cancer; oral cancer; oropharyngeal cancer; ovarian cancer; pancreatic cancer; paraganglioma; parathyroid cancer; glioma (e.g., pediatric gliomas); penile cancer; pharyngeal cancer; pheochromocytoma; pilocytic astrocytoma; pituitary tumor; plasma cell neoplasm; primary peritoneal cancer; prostate cancer; rectum carcinoma; salivary gland cancer; sarcoma (e.g., Ewing sarcoma; rhabdomyosarcoma; uterine sarcoma; and undifferentiated sarcoma); secretory breast carcinoma; Sezary syndrome; skin cancer; small bowel cancer; small cell lung cancer; small intestine cancer; Spitz nevi; Spitz tumors; spitzoid melanoma; stomach cancer; squamous cell carcinoma; squamous neck cancer; testicular cancer; throat cancer; thymoma and thymic carcinoma; thyroid carcinoma; urethral cancer; uterine cancer; urinary bladder cancer; vaginal cancer; vulvar cancer; and Wilms tumor.
Embodiment 211. A method for treating MSI-H cancer, the method comprising:
(a) detecting one or more point mutations in an NTRK gene selected from the group consisting of: NTRK1, NTRK2, and NTRK3; and
(b) administering a Trk inhibitor, or a pharmaceutically acceptable salt thereof.
Embodiment 212. The method of embodiment 211, wherein the MSI-H cancer is colorectal cancer.
Embodiment 213. A method for treating cancer, the method comprising:
(a) detecting high microsatellite instability;
(b) detecting one or more point mutations in an NTRK gene selected from the group consisting of: NTRK1, NTRK2, and NTRK3; and
(c) administering a Trk inhibitor, or a pharmaceutically acceptable salt thereof.
Embodiment 214. The method of embodiment 213, wherein the cancer is colorectal cancer.
Embodiment 215. The method of any one of embodiments 211-214, wherein the one or more point mutations in an NTRK1 gene results in the expression of a TrkA protein comprising a mutation at one or more amino acid position(s) selected from the group consisting of: 613 and 699.
Embodiment 216. The method of any one of embodiments 211-215, wherein the one or more point mutations in a NTRK2 gene results in the expression of a TrkB protein comprising a mutation at one or more amino acid position(s) selected from the group consisting of: 716, 675, and 662.
Embodiment 217. The method of any one of embodiments 211-216, wherein the one or more point mutations in a NTRK3 gene results in the expression of a TrkC protein comprising a mutation at one or more amino acid position(s) selected from the group consisting of: 678 and 745.
Embodiment 218. The method of any one of embodiments 211-217, wherein the Trk inhibitor is selected from the group consisting of: (R)—N-(5-(2-(2,5-difluorophenyl)pyrrolidin-1-yl)pyrazolo[1,5-a]pyrimidin-3-yl)-3-hydroxyazetidine-1-carboxamide;
N-(5-(2-(3-fluorophenyl)-2-methylpyrrolidin-1-yl)pyrazolo[1,5-a]pyrimidin-3-yl)-3-hydroxyazetidine-1-carboxamide;
(R)-1-(5-(2-(2,5-difluorophenyl)pyrrolidin-1-yl)pyrazolo[1,5-a]pyrimidin-3-yl)-3-phenylurea;
(R)—N-(5-(2-(2-(difluoromethyl)-5-fluorophenyl)pyrrolidin-1-yl)pyrazolo[1,5-a]pyrimidin-3-yl)-3-hydroxyazetidine-1-carboxamide;
(R)—N-(5-(2-(3-fluorophenyl)pyrrolidin-1-yl)pyrazolo[1,5-a]pyrimidin-3-yl)-1-methyl-6-oxo-1,6-dihydropyridazine-3-carboxamide;
(S)—N-(5-((R)-2-(2,5-difluorophenyl)pyrrolidin-1-yl)pyrazolo[1,5-a]pyrimidin-3-yl)-3-hydroxypyrrolidine-1-carboxamide;
(3R,4R)—N-(5-((R)-2-(2,5-difluorophenyl)pyrrolidin-1-yl)pyrazolo[1,5-a]pyrimidin-3-yl)-3,4-dihydroxypyrrolidine-1-carboxamide;
(S)—N-(5-((R)-2-(2-chloro-5-fluorophenyl)pyrrolidin-1-yl)pyrazolo[1,5-a]pyrimidin-3-yl)-3-methylpiperazine-1-carboxamide;
(R)—N-(5-(2-(2,5-difluorophenyl)pyrrolidin-1-yl)pyrazolo[1,5-a]pyrimidin-3-yl)-3-hydroxy-3-methylazetidine-1-carboxamide;
(R)—N-(5-(2-(3-fluorophenyl)pyrrolidin-1-yl)pyrazolo[1,5-a]pyrimidin-3-yl)-3-hydroxyazetidine-1-carboxamide;
(R)-1-(4-chlorophenyl)-3-(5-(2-(2,5-difluorophenyl)pyrrolidin-1-yl)pyrazolo[1,5-a]pyrimidin-3-yl)urea;
(6R)-9-fluoro-15-hydroxy-13-oxa-2,11,17,21,22,25-hexaazapentacyclo[17.5.2.0^{2,6}.0^{7,12}.0^{22,26}] hexacosa-1(25),7,9,11,19(26),20,23-heptaen-18-one;
(6R,15R)-9-fluoro-15-hydroxy-13-oxa-2,11,17,21,22,25-hexaazapentacyclo-[17.5.2.0^{2,6}.0^{7,12}.0^{22,26}] hexacosa-1(25),7,9,11,19(26),20,23-heptaen-18-one;
(6R)-9-fluoro-13-oxa-2,11,16,20,21,24-hexaazapentacyclo[16.5.2.0^{2,6}.0^{7,12}.0^{21,25}]pentacosa-1(24),7, 9,11,18(25),19,22-heptaen-17-one;
(6R)-9-fluoro-13-oxa-2,11,18,22,23,26-hexaazapentacyclo[18.5.2.0^{2,6}.0^{7,12}.0^{23,27}]heptacosa-1(26),7,9,11,20(27),21,24-heptaen-19-one;
(6R)-9-fluoro-2,11,13,16,20,21,24-heptaazapentacyclo[16.5.2.0^{2,6}.0^{7,12}.0^{21,25}]pentacosa-1(24),7,9,11,18(25),19,22-heptaen-17-one;
(6R)-9-fluoro-2,11,13,17,21,22,25-heptaazapentacyclo[17.5.2.0^{2,6}.0^{7,12}.0^{22,26}]hexacosa-1(25),7,9,11,19(26),20,23-heptaen-18-one;
(6R)-9-fluoro-17-methyl-13-oxa-2,11,17,21,22,25-hexaazapentacyclo[17.5.2.0^{2,6}.0^{7,12}.0^{22,26}] hexacosa-1(25),7,9,11,19(26),20,23-heptaen-18-one;
(6R)-9,15,15-trifluoro-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one;
(6R)-9-fluoro-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R,15R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R)-9-fluoro-15,15-dimethyl-13-oxa-2,11,17,21,22,25-hexaazapentacyclo [17.5.2.0^{2,6}.0^{7,12}.0^{22,26}] hexacosa-1(25),7,9,11,19(26),20,23-heptaen-18-one; and
(6R)-9-fluoro-15,15-dimethyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
entrectinib (N-[5-(3,5-difluoro-benzyl)-1H-indazol-3-yl]-4-(4-methylpiperazin-1-yl)-2-(tetrahydro-pyran-4-ylamino)-benzamide); and

### TPX-0005;

or a pharmaceutically acceptable salt thereof.
Embodiment 219. The method of any one of embodiments 211-218, wherein the Trk inhibitor is (S)—N-(5-((R)-2-(2,5-difluorophenyl)pyrrolidin-1-yl)pyrazolo[1,5-a]pyrimidin-3-yl)-3-hydroxypyrrolidine-1-carboxamide, or a pharmaceutically acceptable salt thereof.
Embodiment 220. The method of any one of embodiments 211-218, wherein the Trk inhibitor is (6R,15R)-9-fluoro-15-methyl-2,11,16,20,21,24-hexaazapentacyclo[16.5.2.02,6.07,12.021,25]pentacosa-1(24),7,9,11,18(25),19,22-heptaen-17-one, or a pharmaceutically acceptable salt thereof.
Embodiment 221. The method of any one of embodiments 211-220, wherein the method further comprises administering an immunotherapy agent.
Embodiment 222. The method of any one of embodiments 211-221, wherein the immunotherapy agent is selected from the group consisting of: a cellular immunotherapy; an antibody therapy; an antibody-drug conjugate; a toxin; blinatumomab (AMG103) or midostaurin (Rydapt); a cytokine therapy; an immune checkpoint inhibitor; an mRNA-based immunotherapy; bacillus Calmette-Guerin (BCG) therapy; an oncolytic virus therapy; a cancer vaccine; a peptide vaccine; and a DNA-based vaccine.
Embodiment 223. The method of any one of embodiments 211-222, wherein the cellular immunotherapy is selected from the group consisting of: adoptive T-cell therapy; dendritic cell therapy; natural killer cell therapy; sipuleucel-T (APC8015); cells that express a chimeric antigen receptor (CAR); CAR-T cell therapy; and tisagenlecleucel.
Embodiment 224. The method of any one of embodiments 211-223, wherein the antibody therapy is selected from the group consisting of: a monoclonal antibody and a conjugated antibody.
Embodiment 225. The method of any one of embodiments 211-224, wherein the antibody therapy is selected from the group consisting of: bevacizumab; trastuzumab; avelumab; rituximab; edrecolomab; daratumuab; olaratumab; ofatumumab; alemtuzumab; cetuximab; oregovomab; pembrolizumab; dinutiximab; obinutuzumab; tremelimumab (CP-675;206); ramucirumab; ublituximab (TG-1101); panitumumab; elotuzumab; avelumab; necitumumab; cirmtuzumab; ibritumomab; isatuximab (SAR650984); nimotuzumab; fresolimumab (GC1008); lirilumab (INN); mogamulizumab; ficlatuzumab (AV-299); denosumab; ganitumab; urelumab; pidilizumab; and amatuximab.
Embodiment 226. The method of any one of embodiments 211-225, wherein the antibody-drug conjugate is selected from the group consisting of: gemtuzumab ozogamicin; inotuzumab ozogamicin; brentuximab vedotin; ado-trastuzumab emtansine (TDM-1); mirvetuximab soravtansine (IMGN853); and anetumab ravtansine.
Embodiment 227. The method of any one of embodiments 211-226, wherein the toxin is denileukin diftitox.
Embodiment 228. The method of any one of embodiments 211-227, wherein the immunotherapy agent is blinatumomab (AMG103).
Embodiment 229. The method of any one of embodiments 211-228, wherein the immunotherapy agent is midostaurin (Rydapt).
Embodiment 230. The method of any one of embodiments 211-229, wherein the cytokine therapy is selected from the group consisting of: an interleukin 2 (IL-2) therapy; an interferon alpha (IFNα) therapy; a granulocyte colony stimulating factor (G-CSF) therapy; an interleukin 12 (IL-12) therapy; an interleukin 15 (IL-15) therapy; an interleukin 7 (IL-7) therapy; and an erythropoietin-alpha (EPO) therapy.
Embodiment 231. The method of any one of embodiments 211-230, wherein the interleukin 2 (IL-2) therapy is aldesleukin.
Embodiment 232. The method of any one of embodiments 211-231, wherein the IFNα therapy is interferon alfa-2b or interferon alfa-2a.
Embodiment 233. The method of any one of embodiments 211-232, wherein the G-CSF therapy is filgrastim.
Embodiment 234. The method of any one of embodiments 211-233, wherein the immune checkpoint inhibitor is selected from the group consisting of: a CTLA-4 inhibitor; a PD-1 inhibitor; and a PD-L1 inhibitor.
Embodiment 235. The method of any one of embodiments 211-234, wherein the CTLA-4 inhibitor is ipilimumab or tremelimumab (CP-675,206).
Embodiment 236. The method of any one of embodiments 211-235, wherein the PD-1 inhibitor is pembrolizumab or nivolumab.
Embodiment 237. The method of any one of embodiments 211-236, wherein the PD-1 inhibitor is pembrolizumab.
Embodiment 238. The method of any one of embodiments 211-237, wherein the PD-L1 inhibitor is selected from the group consisting of: atezolizumab; avelumab; and durvalumab.
Embodiment 239. The method of any one of embodiments 211-238, wherein the mRNA-based immunotherapy is CV9104.
Embodiment 240. The method of any one of embodiments 211-239, wherein the immunotherapy agent is bacillus Calmette-Guerin (BCG) therapy.
Embodiment 241. The method of any one of embodiments 211-240, wherein the oncolytic virus therapy is talimogene alherparepvec (T-VEC).
Embodiment 242. The method of any one of embodiments 211-241, wherein the cancer vaccine is a human papillomavirus (HPV) vaccine.
Embodiment 243. The method of any one of embodiments 211-242, wherein the human papillomavirus (HPV) vaccine is selected from the group consisting of: a recombinant human papillomavirus vaccine [types 6, 11, 16, and 18]; a recombinant human papillomavirus vaccine [types 6, 11, 16, 18, 31, 33, 45, 52, and 58]; and a recombinant human papillomavirus vaccine [types 16 and 18].
Embodiment 244. The method of any one of embodiments 211-243, wherein the cancer vaccine is a hepatitis B virus (HBV) vaccine.
Embodiment 245. The method of any one of embodiments 211-244, wherein the cancer vaccine is selected from the group consisting of: a combination Hepatitis A and Hepatitis B vaccine; a combination diphtheria, tetanus, pertussis, hepatitis B virus, and poliomyelitis vaccine; dasiprotimut-T; cancer vaccine HSPPC-96; GVAX; ADXS11-001; ALVAC-CEA; rilimogene galvacirepvec/rilimogene glafolivec; CDX-110; CimaVax-EGF; lapuleucel-T (APC8024); GRNVAC1; GRNVAC2; GRN-1201; hepcortespenlisimut-L (Hepko-V5); a dendritic cell vaccine; ICT-107; SCIB1; BMT CTN 1401; PrCa VBIR; PANVAC; a prostate cancer vaccine; DPX-Survivac; and viagenpumatucel-L (HS-110).
Embodiment 246. The method of any one of embodiments 211-245, wherein the peptide vaccine is selected from the group consisting of: nelipepimut-S(E75); IMA901; SurVaxM (SVN53-67); an immunogenic personal neoantigen vaccine; RGSH4K; and NEO-PV-01.
Embodiment 247. The method of any one of embodiments 211-246, wherein the DNA-based vaccine is a mammaglobin-A DNA vaccine.
Embodiment 248. A method for treating a MSI-H cancer, the method comprising:
(a) detecting one or more point mutations in an NTRK gene selected from the group consisting of: NTRK1, NTRK2, and NTRK3; and
(b) administering a Trk inhibitor, or a pharmaceutically acceptable salt thereof, and a PD-1 inhibitor.
Embodiment 249. The method of embodiment 248, wherein the MSI-H cancer is colorectal cancer.
Embodiment 250. A method for treating cancer, the method comprising:
(a) detecting high microsatellite instability;
(b) detecting one or more point mutations in an NTRK gene selected from the group consisting of: NTRK1, NTRK2, and NTRK3; and
(c) administering a Trk inhibitor, or a pharmaceutically acceptable salt thereof, and a PD-1 inhibitor.
Embodiment 251. The method of embodiment 250, wherein the cancer is colorectal cancer.
Embodiment 252. The method of any one of embodiments 248-251, wherein the one or more point mutations in an NTRK1 gene results in the expression of a TrkA protein comprising a mutation at one or more amino acid position(s) selected from the group consisting of: 613 and 699.
Embodiment 253. The method of any one of embodiments 248-252, wherein the one or more point mutations in a NTRK2 gene results in the expression of a TrkB protein comprising a mutation at one or more amino acid position(s) selected from the group consisting of: 716, 675, and 662.
Embodiment 254. The method of any one of embodiments 248-253, wherein the one or more point mutations in a NTRK3 gene results in the expression of a TrkC protein comprising a mutation at one or more amino acid position(s) selected from the group consisting of: 678 and 745.
Embodiment 255. The method of any one of embodiments 248-254, wherein the Trk inhibitor is selected from the group consisting of:
(R)—N-(5-(2-(2,5-difluorophenyl)pyrrolidin-1-yl)pyrazolo[1,5-a]pyrimidin-3-yl)-3-hydroxyazetidine-1-carboxamide;
N-(5-(2-(3-fluorophenyl)-2-methylpyrrolidin-1-yl)pyrazolo[1,5-a]pyrimidin-3-yl)-3-hydroxyazetidine-1-carboxamide;
(R)-1-(5-(2-(2,5-difluorophenyl)pyrrolidin-1-yl)pyrazolo[1,5-a]pyrimidin-3-yl)-3-phenylurea;
(R)—N-(5-(2-(2-(difluoromethyl)-5-fluorophenyl)pyrrolidin-1-yl)pyrazolo[1,5-a]pyrimidin-3-yl)-3-hydroxyazetidine-1-carboxamide;
(R)—N-(5-(2-(3-fluorophenyl)pyrrolidin-1-yl)pyrazolo[1,5-a]pyrimidin-3-yl)-1-methyl-6-oxo-1,6-dihydropyridazine-3-carboxamide;
(S)—N-(5-((R)-2-(2,5-difluorophenyl)pyrrolidin-1-yl)pyrazolo[1,5-a]pyrimidin-3-yl)-3-hydroxypyrrolidine-1-carboxamide;
(3R,4R)—N-(5-((R)-2-(2,5-difluorophenyl)pyrrolidin-1-yl)pyrazolo[1,5-a]pyrimidin-3-yl)-3,4-dihydroxypyrrolidine-1-carboxamide;
(S)—N-(5-((R)-2-(2-chloro-5-fluorophenyl)pyrrolidin-1-yl)pyrazolo[1,5-a]pyrimidin-3-yl)-3-methylpiperazine-1-carboxamide;
(R)—N-(5-(2-(2,5-difluorophenyl)pyrrolidin-1-yl)pyrazolo[1,5-a]pyrimidin-3-yl)-3-hydroxy-3-methylazetidine-1-carboxamide;
(R)—N-(5-(2-(3-fluorophenyl)pyrrolidin-1-yl)pyrazolo[1,5-a]pyrimidin-3-yl)-3-hydroxyazetidine-1-carboxamide;
(R)-1-(4-chlorophenyl)-3-(5-(2-(2,5-difluorophenyl)pyrrolidin-1-yl)pyrazolo[1,5-a]pyrimidin-3-yl)urea;
(6R)-9-fluoro-15-hydroxy-13-oxa-2,11,17,21,22,25-hexaazapentacyclo[17.5.2.0^{2,6}.0^{7,12}.0^{22,26}] hexacosa-1(25),7,9,11,19(26),20,23-heptaen-18-one;
(6R,15R)-9-fluoro-15-hydroxy-13-oxa-2,11,17,21,22,25-hexaazapentacyclo-[17.5.2.0^{2,6}.0^{7,12}.0^{22,26}] hexacosa-1(25),7,9,11,19(26),20,23-heptaen-18-one;
(6R)-9-fluoro-13-oxa-2,11,16,20,21,24-hexaazapentacyclo[16.5.2.0^{2,6}.0^{7,12}.0^{21,25}]pentacosa-1(24),7, 9,11,18(25),19,22-heptaen-17-one;
(6R)-9-fluoro-13-oxa-2,11,18,22,23,26-hexaazapentacyclo[18.5.2.0^{2,6}.0^{7,12}.0^{23,27}]heptacosa-1(26),7,9,11,20(27),21,24-heptaen-19-one;
(6R)-9-fluoro-2,11,13,16,20,21,24-heptaazapentacyclo[16.5.2.0^{2,6}.0^{7,12}.0^{21,25}]pentacosa-1(24),7,9,11,18(25),19,22-heptaen-17-one;
(6R)-9-fluoro-2,11,13,17,21,22,25-heptaazapentacyclo[17.5.2.0^{2,6}.0^{7,12}.0^{22,26}]hexacosa-1(25),7,9,11,19(26),20,23-heptaen-18-one;
(6R)-9-fluoro-17-methyl-13-oxa-2,11,17,21,22,25-hexaazapentacyclo[17.5.2.0^{2,6}.0^{7,12}.0^{22,26}] hexacosa-1(25),7,9,11,19(26),20,23-heptaen-18-one;
(6R)-9,15,15-trifluoro-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one;
(6R)-9-fluoro-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R,15R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R)-9-fluoro-15,15-dimethyl-13-oxa-2,11,17,21,22,25-hexaazapentacyclo [17.5.2.0^{2,6}.0^{7,12}.0^{22,26}] hexacosa-1(25),7,9,11,19(26),20,23-heptaen-18-one; and
(6R)-9-fluoro-15,15-dimethyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
entrectinib (N-[5-(3,5-difluoro-benzyl)-1H-indazol-3-yl]-4-(4-methylpiperazin-1-yl)-2-(tetrahydro-pyran-4-ylamino)-benzamide); and

### TPX-0005;

or a pharmaceutically acceptable salt thereof.
Embodiment 256. The method of any one of embodiments 248-255, wherein the Trk inhibitor is (S)—N-(5-((R)-2-(2,5-difluorophenyl)pyrrolidin-1-yl)pyrazolo[1,5-a]pyrimidin-3-yl)-3-hydroxypyrrolidine-1-carboxamide, or a pharmaceutically acceptable salt thereof.
Embodiment 257. The method of any one of embodiments 248-255, wherein the Trk inhibitor is (6R,15R)-9-fluoro-15-methyl-2,11,16,20,21,24-hexaazapentacyclo[16.5.2.02,6.07,12.021,25]pentacosa-1(24),7,9,11,18(25),19,22-heptaen-17-one, or a pharmaceutically acceptable salt thereof.
Embodiment 258. The method of any one of embodiments 248-257, wherein the PD-1 inhibitor is pembrolizumab or nivolumab.
Embodiment 259. The method of any one of embodiments 248-258, wherein the PD-1 inhibitor is pembrolizumab.
Embodiment 260. The method of any one of embodiments 248-259, wherein the Trk inhibitor is (S)—N-(5-((R)-2-(2,5-difluorophenyl)pyrrolidin-1-yl)pyrazolo[1,5-a]pyrimidin-3-yl)-3-hydroxypyrrolidine-1-carboxamide, or a pharmaceutically acceptable salt thereof, and the PD-1 inhibitor is pembrolizumab.
Embodiment 261. The method of any one of embodiments 248-259, wherein the Trk inhibitor is (6R,15R)-9-fluoro-15-methyl-2,11,16,20,21,24-hexaazapentacyclo[16.5.2.02,6.07,12.021,25]pentacosa-1(24),7,9,11,18(25),19,22-heptaen-17-one, or a pharmaceutically acceptable salt thereof, the PD-1 inhibitor is pembrolizumab.
Embodiment 262. A method for treating a Trk-associated cancer in a subject in need thereof, wherein the cancer has at least one fusion selected from the group consisting of: RFWD2-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, AMOTL2-TrkA, MEF2D-TrkA, L7a-TrkA, ZBTB7B-TrkA, TRIM63-TrkA, DDR2-TrkA, GON4L-TrkA, PDE4DIP-TrkA, NTRK1-P2RY8, CTRC-TrkA, VANGL2-TrkA, DAB2IP-TrkB, TrkB-TERT, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, STRN-TrkB, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-NTRK3, AKAP13-NTRK3, NTRK3-LOXL2, NTRK3-PEAK1, ZNF710-TrkC, TPM4-TrkC, and LMNA-TrkC, the method comprising administering a compound selected from the group consisting of:
(R)—N-(5-(2-(2,5-difluorophenyl)pyrrolidin-1-yl)pyrazolo[1,5-a]pyrimidin-3-yl)-3-hydroxyazetidine-1-carboxamide;
N-(5-(2-(3-fluorophenyl)-2-methylpyrrolidin-1-yl)pyrazolo[1,5-a]pyrimidin-3-yl)-3-hydroxyazetidine-1-carboxamide;
(R)-1-(5-(2-(2,5-difluorophenyl)pyrrolidin-1-yl)pyrazolo[1,5-a]pyrimidin-3-yl)-3-phenylurea;
(R)—N-(5-(2-(2-(difluoromethyl)-5-fluorophenyl)pyrrolidin-1-yl)pyrazolo[1,5-a]pyrimidin-3-yl)-3-hydroxyazetidine-1-carboxamide;
(R)—N-(5-(2-(3-fluorophenyl)pyrrolidin-1-yl)pyrazolo[1,5-a]pyrimidin-3-yl)-1-methyl-6-oxo-1,6-dihydropyridazine-3-carboxamide;
(S)—N-(5-((R)-2-(2,5-difluorophenyl)pyrrolidin-1-yl)pyrazolo[1,5-a]pyrimidin-3-yl)-3-hydroxypyrrolidine-1-carboxamide;
(3R,4R)—N-(5-((R)-2-(2,5-difluorophenyl)pyrrolidin-1-yl)pyrazolo[1,5-a]pyrimidin-3-yl)-3,4-dihydroxypyrrolidine-1-carboxamide;
(S)—N-(5-((R)-2-(2-chloro-5-fluorophenyl)pyrrolidin-1-yl)pyrazolo[1,5-a]pyrimidin-3-yl)-3-methylpiperazine-1-carboxamide;
(R)—N-(5-(2-(2,5-difluorophenyl)pyrrolidin-1-yl)pyrazolo[1,5-a]pyrimidin-3-yl)-3-hydroxy-3-methylazetidine-1-carboxamide;
(R)—N-(5-(2-(3-fluorophenyl)pyrrolidin-1-yl)pyrazolo[1,5-a]pyrimidin-3-yl)-3-hydroxyazetidine-1-carboxamide;
(R)-1-(4-chlorophenyl)-3-(5-(2-(2,5-difluorophenyl)pyrrolidin-1-yl)pyrazolo[1,5-a]pyrimidin-3-yl)urea;
(6R)-9-fluoro-15-hydroxy-13-oxa-2,11,17,21,22,25-hexaazapentacyclo[17.5.2.0^{2,6}.0^{7,12}.0^{22,26}] hexacosa-1(25),7,9,11,19(26),20,23-heptaen-18-one;
(6R,15R)-9-fluoro-15-hydroxy-13-oxa-2,11,17,21,22,25-hexaazapentacyclo-[17.5.2.0^{2,6}.0^{7,12}.0^{22,26}] hexacosa-1(25),7,9,11,19(26),20,23-heptaen-18-one;
(6R)-9-fluoro-13-oxa-2,11,16,20,21,24-hexaazapentacyclo[16.5.2.0^{2,6}.0^{7,12}.0^{21,25}]pentacosa-1(24),7, 9,11,18(25),19,22-heptaen-17-one;
(6R)-9-fluoro-13-oxa-2,11,18,22,23,26-hexaazapentacyclo[18.5.2.0^{2,6}.0^{7,12}.0^{23,27}]heptacosa-1(26),7,9,11,20(27),21,24-heptaen-19-one;
(6R)-9-fluoro-2,11,13,16,20,21,24-heptaazapentacyclo[16.5.2.0^{2,6}.0^{7,12}.0^{21,25}]pentacosa-1(24),7,9,11,18(25),19,22-heptaen-17-one;
(6R)-9-fluoro-2,11,13,17,21,22,25-heptaazapentacyclo[17.5.2.0^{2,6}.0^{7,12}.0^{22,26}]hexacosa-1(25),7,9,11,19(26),20,23-heptaen-18-one;
(6R)-9-fluoro-17-methyl-13-oxa-2,11,17,21,22,25-hexaazapentacyclo[17.5.2.0^{2,6}.0^{7,12}.0^{22,26}] hexacosa-1(25),7,9,11,19(26),20,23-heptaen-18-one;
(6R)-9,15,15-trifluoro-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one;
(6R)-9-fluoro-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R,15R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
(6R)-9-fluoro-15,15-dimethyl-13-oxa-2,11,17,21,22,25-hexaazapentacyclo [17.5.2.0^{2,6}.0^{7,12}.0^{22,26}] hexacosa-1(25),7,9,11,19(26),20,23-heptaen-18-one; and
(6R)-9-fluoro-15,15-dimethyl-2,11,16,20,21,24-hexaazapentacyclo[16.5.2.0^{2,6}.0^{7,12}.0^{21,25}]pentacosa-1(24),7,9,11,18(25),19,22-heptaen-17-one;
entrectinib (N-[5-(3,5-difluoro-benzyl)-1H-indazol-3-yl]-4-(4-methylpiperazin-1-yl)-2-(tetrahydro-pyran-4-ylamino)-benzamide); and TPX-0005;
   or a pharmaceutically acceptable salt thereof.
   Embodiment 263. The method of embodiment 262, wherein the compound is ((R)—N-(5-(2-(2,5-difluorophenyl)pyrrolidin-1-yl)pyrazolo[1,5-a]pyrimidin-3-yl)-3-hydroxyazetidine-1-carboxamide, or a pharmaceutically acceptable salt thereof.
   Embodiment 264. The method of embodiment 262, wherein the compound is N-(5-(2-(3-fluorophenyl)-2-methylpyrrolidin-1-yl)pyrazolo[1,5-a]pyrimidin-3-yl)-3-hydroxyazetidine-1-carboxamide, or a pharmaceutically acceptable salt thereof.
   Embodiment 265. The method of embodiment 262, wherein the compound is (R)-1-(5-(2-(2, 5-difluorophenyl)pyrrolidin-1-yl)pyrazolo[1,5-a]pyrimidin-3-yl)-3-phenylurea, or a pharmaceutically acceptable salt thereof.
   Embodiment 266. The method of embodiment 262, wherein the compound is (R)—N-(5-(2-(2-(difluoromethyl)-5-fluorophenyl)pyrrolidin-1-yl)pyrazolo[1,5-a]pyrimidin-3-yl)-3-hydroxyazetidine-1-carboxamide, or a pharmaceutically acceptable salt thereof.
   Embodiment 267. The method of embodiment 262, wherein the compound is ((R)—N-(5-(2-(3-fluorophenyl)pyrrolidin-1-yl)pyrazolo[1,5-a]pyrimidin-3-yl)-1-methyl-6-oxo-1,6-dihydropyridazine-3-carboxamide, or a pharmaceutically acceptable salt thereof.
   Embodiment 268. The method of embodiment 262, wherein the compound is (S)—N-(5-((R)-2-(2,5-difluorophenyl)pyrrolidin-1-yl)pyrazolo[1,5-a]pyrimidin-3-yl)-3-hydroxypyrrolidine-1-carboxamide, or a pharmaceutically acceptable salt thereof.
   Embodiment 269. The method of embodiment 262, wherein the compound is (3R,4R)—N-(5-((R)-2-(2,5-difluorophenyl)pyrrolidin-1-yl)pyrazolo[1,5-a]pyrimidin-3-yl)-3,4-dihydroxypyrrolidine-1-carboxamide, or a pharmaceutically acceptable salt thereof.
   Embodiment 270. The method of embodiment 262, wherein the compound is (S)—N-(5-((R)-2-(2-chloro-5-fluorophenyl)pyrrolidin-1-yl)pyrazolo[1,5-a]pyrimidin-3-yl)-3-methylpiperazine-1-carboxamide, or a pharmaceutically acceptable salt thereof.
   Embodiment 271. The method of embodiment 262, wherein the compound is (R)—N-(5-(2-(2,5-difluorophenyl)pyrrolidin-1-yl)pyrazolo[1,5-a]pyrimidin-3-yl)-3-hydroxy-3-methylazetidine-1-carboxamide, or a pharmaceutically acceptable salt thereof.
   Embodiment 272. The method of embodiment 262, wherein the compound is (R)—N-(5-(2-(3-fluorophenyl)pyrrolidin-1-yl)pyrazolo[1,5-a]pyrimidin-3-yl)-3-hydroxyazetidine-1-carboxamide, or a pharmaceutically acceptable salt thereof.
   Embodiment 273. The method of embodiment 262, wherein the compound is (R)-1-(4-chlorophenyl)-3-(5-(2-(2, 5-difluorophenyl)pyrrolidin-1-yl)pyrazolo[1,5-a]pyrimidin-3-yl)urea, or a pharmaceutically acceptable salt thereof.
   Embodiment 274. The method of embodiment 262, wherein the compound is (6R)-9-fluoro-15-hydroxy-13-oxa-2,11,17,21,22,25-hexaazapentacyclo[17.5.2.0^{2,6}.0^{7,12}.0^{22,26}] hexacosa-1(25),7,9,11,19(26),20,23-heptaen-18-one, or a pharmaceutically acceptable salt thereof.
   Embodiment 275. The method of embodiment 262, wherein the compound is (6R,15R)-9-fluoro-15-hydroxy-13-oxa-2,11,17,21,22,25-hexaazapentacyclo-[17.5.2.0^{2,6}.0^{7,12}.0^{22,26}] hexacosa-1(25),7,9,11,19(26),20,23-heptaen-18-one, or a pharmaceutically acceptable salt thereof.
   Embodiment 276. The method of embodiment 262, wherein the compound is (6R)-9-fluoro-13-oxa-2,11,16,20,21,24-hexaazapentacyclo[16.5.2.0^{2,6}.0^{7,12}.0^{21,25}]pentacosa-1(24),7, 9,11,18(25),19,22-heptaen-17-one, or a pharmaceutically acceptable salt thereof.
   Embodiment 277. The method of embodiment 262, wherein the compound is (6R)-9-fluoro-13-oxa-2,11,18,22,23,26-hexaazapentacyclo[18.5.2.0^{2,6}.0^{7,12}.0^{23,27}]heptacosa-1(26),7,9,11,20(27),21,24-heptaen-19-one, or a pharmaceutically acceptable salt thereof.
   Embodiment 278. The method of embodiment 262, wherein the compound is (6R)-9-fluoro-2, 11,13,16,20,21,24-heptaazapentacyclo[16.5.2.0^{2,6}.0^{7,12}.0^{21,25}]pentacosa-1(24),7,9,11,18(25),19,22-heptaen-17-one, or a pharmaceutically acceptable salt thereof.
   Embodiment 279. The method of embodiment 262, wherein the compound is (6R)-9-fluoro-2,11,13,17,21,22,25-heptaazapentacyclo[17.5.2.0^{2,6}.0^{7,12}.0^{22,26}]hexacosa-1(25),7,9,11,19(26),20,23-heptaen-18-one, or a pharmaceutically acceptable salt thereof.
   Embodiment 280. The method of embodiment 262, wherein the compound is (6R)-9-fluoro-17-methyl-13-oxa-2,11,17,21,22,25-hexaazapentacyclo[17.5.2.0^{2,6}.0^{7,12}.0^{22,26}] hexacosa-1(25),7,9,11,19(26),20,23-heptaen-18-one, or a pharmaceutically acceptable salt thereof.
   Embodiment 281. The method of embodiment 262, wherein the compound is (6R)-9,15,15-trifluoro-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one, or a pharmaceutically acceptable salt thereof.
   Embodiment 282. The method of embodiment 262, wherein the compound is (6R)-9-fluoro-2, 11,16,20,21,24-hexaazapentacyclo[16.5.2.0^{2,6}.0^{7,12}.0^{21,25}]pentacosa-1(24),7,9,11,18(25),19,22-heptaen-17-one, or a pharmaceutically acceptable salt thereof,
   Embodiment 283. The method of embodiment 262, wherein the compound is (6R)-9-fluoro-15-meth 1-2,11,16,20,21,24-hexaazapentacyclo[16.5.2.0^{2,6}.0^{7,12}.0^{21,25}]pentacosa-1(24),7,9,11,18(25),19,22-heptaen-17-one, or a pharmaceutically acceptable salt thereof.
   Embodiment 284. The method of embodiment 262, wherein the compound is (6R,15R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one, or a pharmaceutically acceptable salt thereof.
   Embodiment 285. The method of embodiment 262, wherein the compound is (6R)-9-fluoro-15,15-dimethyl-13-oxa-2,11,17,21,22,25-hexaazapentacyclo [17.5.2.0^{2,6}.0^{7,12}.0^{22,26}] hexacosa-1(25),7,9,11,19(26),20,23-heptaen-18-one, or a pharmaceutically acceptable salt thereof.
   Embodiment 286. The method of embodiment 262, wherein the compound is (6R)-9-fluoro-15,15-dimethyl-2,11,16,20,21,24-hexaazapentacyclo[16.5.2.0^{2,6}.0^{7,12}.0^{21,25}]pentacosa-1(24),7,9,11,18(25),19,22-heptaen-17-one, or a pharmaceutically acceptable salt thereof.
   Embodiment 287. The method of embodiment 262, wherein the compound is entrectinib (N-[5-(3,5-difluoro-benzyl)-1H-indazol-3-yl]-4-(4-methylpiperazin-1-yl)-2-(tetrahydro-pyran-4-ylamino)-benzamide), or a pharmaceutically acceptable salt thereof.
   Embodiment 288. The method of embodiment 262, wherein the compound is TPX-0005, or a pharmaceutically acceptable salt thereof.
   Embodiment 289. The method of any one of embodiments 262-288, wherein the Trk-associated cancer is one or more of large cell neuroendocrine cancer, sarcoma, breast cancer, non-small cell lung cancer, uterus carcinoma, papillary thyroid carcinoma, pancreatic adenocarcinoma, lower grade glioma, papillary thyroid cancer, bladder urothelial carcinoma, a non-spitzoid metastasizing melanoma, soft tissue sarcoma (myopericytoma), pancreatic cancer colorectal cancer, thyroid cancer, anaplastic astrocytoma, glioblastoma, astrocytoma, ganglioma, ganglioglioma, complex glioneuronal tumor, malignant epithelioid glioneuronal tumor, melanoma, oligoastrocytoma, salivary gland cancer, fibrosarcoma (e.g., pediatric fibrosarcoma or infantile fibrosarcoma), glioblastoma, colon cancer, mesenchymal tumor, congenital mesoblastic nephroma, pancreatic adenocarcinoma, soft tissue sarcoma, soft tissue solitary fibrous tumor, cervical squamous cell carcinoma, melanocytic tumor (e.g., spitz tumor), melanoma, spitz tumor, renal metanephric adenoma, thyroid carcinoma, multiple myeloma, colon adenocarcinoma, lower grade glioma, and soft tissue sarcoma.
   Embodiment 290. A method for treating a Trk-associated cancer in a subject in need thereof, wherein the cancer has a fusion selected from the group consisting of: TP53-TrkA, CD74-TrkA, TFG-TrkA, NFASC-TrkA, BCAN-TrkA, MPRIP-TrkA, TPR-TrkA, RFWD2-TrkA, IRF2BP2-TrkA, SQSTM1-TrkA, SSBP2-TrkA, RABGAP1L-TrkA, C18ORF8-TrkA, RNF213-TrkA, TBC1D22A-TrkA, C200RF112-TrkA, DNER-TrkA, ARHGEF2-TrkA, CHTOP-TrkA, PPL-TrkA, PLEKHA6-TrkA, PEAR1-TrkA, MRPL24-TrkA, MDM4-TrkA, LRRC71-TrkA, GRIPAP1-TrkA, TAF-TrkA, EPS15-TrkA, DYNC2H1-TrkA, CEL-TrkA, EPHB2-TrkA, TGF-TrkA, NELL1-TrkA, EPL4-TrkA, CTNND2-TrkA, TCEANC2-TrkA, SCYL3-TrkA, AMOTL2-TrkA, MEF2D-TrkA, L7a-TrkA, ZBTB7B-TrkA, TRIM63-TrkA, DDR2-TrkA, GON4L-TrkA, PDE4DIP-TrkA, NTRK1-P2RY8, CTRC-TrkA, VANGL2-TrkA, ETV6-TrkA, NACC2-TrkB, AFAP1-TrkB, PAN3-TrkB, SQSTM1-TrkB, TRIM24-TrkB, VCL-TrkB, AGBL4-TrkB, DAB2IP-TrkB, TrkB-TERT, NOS1AP-TrkB, GKAP1-TrkB, KCTD8-TrkB, TBC1D2-TrkB, VCAN-TrkB, SLMAP-TrkB, TLE4-TrkB, STRN3-TrkB, WNK2-TrkB, TrkB-BEND5, TrkB-TRAF2, Nav1-TrkB, STRN-TrkB, RIP13-TrkB, BTBD1-TrkC, LYN-TrkC, RBPMS-TrkC, EML4-TrkC, TrkC-HOMER2, TFG-TrkC, FAT1-TrkC, MYO5A-TrkC, MYH9-TrkC, KANK1-TrkC, SQSTM1-TrkC, UBE2R2-TrkC, HNRNPA2B1-TrkC, VPS18-NTRK3, AKAP13-NTRK3, NTRK3-LOXL2, NTRK3-PEAK1, ZNF710-TrkC, TPM4-TrkC, LMNA-TrkC, the method comprising administering a compound selected from the group consisting of:
(R)—N-(5-(2-(2,5-difluorophenyl)pyrrolidin-1-yl)pyrazolo[1,5-a]pyrimidin-3-yl)-3-hydroxyazetidine-1-carboxamide;
N-(5-(2-(3-fluorophenyl)-2-methylpyrrolidin-1-yl)pyrazolo[1,5-a]pyrimidin-3-yl)-3-hydroxyazetidine-1-carboxamide;
(R)-1-(5-(2-(2,5-difluorophenyl)pyrrolidin-1-yl)pyrazolo[1,5-a]pyrimidin-3-yl)-3-phenylurea;
(R)—N-(5-(2-(2-(difluoromethyl)-5-fluorophenyl)pyrrolidin-1-yl)pyrazolo[1,5-a]pyrimidin-3-yl)-3-hydroxyazetidine-1-carboxamide;
(R)—N-(5-(2-(3-fluorophenyl)pyrrolidin-1-yl)pyrazolo[1,5-a]pyrimidin-3-yl)-1-methyl-6-oxo-1,6-dihydropyridazine-3-carboxamide;
(S)—N-(5-((R)-2-(2,5-difluorophenyl)pyrrolidin-1-yl)pyrazolo[1,5-a]pyrimidin-3-yl)-3-hydroxypyrrolidine-1-carboxamide;
(3R,4R)—N-(5-((R)-2-(2,5-difluorophenyl)pyrrolidin-1-yl)pyrazolo[1,5-a]pyrimidin-3-yl)-3,4-dihydroxypyrrolidine-1-carboxamide;
(S)—N-(5-((R)-2-(2-chloro-5-fluorophenyl)pyrrolidin-1-yl)pyrazolo[1,5-a]pyrimidin-3-yl)-3-methylpiperazine-1-carboxamide;
(R)—N-(5-(2-(2,5-difluorophenyl)pyrrolidin-1-yl)pyrazolo[1,5-a]pyrimidin-3-yl)-3-hydroxy-3-methylazetidine-1-carboxamide;
(R)—N-(5-(2-(3-fluorophenyl)pyrrolidin-1-yl)pyrazolo[1,5-a]pyrimidin-3-yl)-3-hydroxyazetidine-1-carboxamide;
(R)-1-(4-chlorophenyl)-3-(5-(2-(2,5-difluorophenyl)pyrrolidin-1-yl)pyrazolo[1,5-a]pyrimidin-3-yl)urea;
(6R)-9-fluoro-15-hydroxy-13-oxa-2,11,17,21,22,25-hexaazapentacyclo[17.5.2.02,6.07,12.022,26] hexacosa-1(25),7,9,11,19(26),20,23-heptaen-18-one;
(6R,15R)-9-fluoro-15-hydroxy-13-oxa-2,11,17,21,22,25-hexaazapentacyclo-[17.5.2.02,6.07,12.022,26] hexacosa-1(25),7,9,11,19(26),20,23-heptaen-18-one;
(6R)-9-fluoro-13-oxa-2,11,16,20,21,24-hexaazapentacyclo[16.5.2.02,6.07,12.021,25]pentacosa-1(24),7, 9,11,18(25),19,22-heptaen-17-one;
(6R)-9-fluoro-13-oxa-2,11,18,22,23,26-hexaazapentacyclo[18.5.2.02,6.07,12.023,27]heptacosa-1(26),7,9,11,20(27),21,24-heptaen-19-one;
(6R)-9-fluoro-2,11,13,16,20,21,24-heptaazapentacyclo[16.5.2.02,6.07,12.021,25]pentacosa-1(24),7,9,11,18(25),19,22-heptaen-17-one;
(6R)-9-fluoro-2,11,13,17,21,22,25-heptaazapentacyclo[17.5.2.02,6.07,12.022,26]hexacosa-1(25),7,9,11,19(26),20,23-heptaen-18-one;
(6R)-9-fluoro-17-methyl-13-oxa-2,11,17,21,22,25-hexaazapentacyclo[17.5.2.02,6.07,12.022,26] hexacosa-1(25),7,9,11,19(26),20,23-heptaen-18-one;
(6R)-9,15,15-trifluoro-13-oxa-2,11,17,21,22,25-hexaazapentacyclo[17.5.2.02,6.07,12.022,26]hexacosa-1(25),7,9,11,19(26),20,23-heptaen-18-one;
(6R)-9-fluoro-2,11,16,20,21,24-hexaazapentacyclo[16.5.2.02,6.07,12.021,25]pentacosa-1(24),7,9,11,18(25),19,22-heptaen-17-one;
(6R)-9-fluoro-15-methyl-2,11,16,20,21,24-hexaazapentacyclo[16.5.2.02,6.07,12.021,25]pentacosa-1(24),7,9,11,18(25),19,22-heptaen-17-one;
(6R,15R)-9-fluoro-15-methyl-2,11,16,20,21,24-hexaazapentacyclo[16.5.2.02,6.07,12.021,25]pentacosa-1(24),7,9,11,18(25),19,22-heptaen-17-one;
(6R)-9-fluoro-15,15-dimethyl-13-oxa-2,11,17,21,22,25-hexaazapentacyclo [17.5.2.02,6.07,12.022,26] hexacosa-1(25),7,9,11,19(26),20,23-heptaen-18-one; and
(6R)-9-fluoro-15,15-dimethyl-2,11,16,20,21,24-hexaazapentacyclo[16.5.2.02,6.07,12.021,25]pentacosa-1(24),7,9,11,18(25),19,22-heptaen-17-one; and

### TPX-0005;

or a pharmaceutically acceptable salt thereof.

Embodiment 291. The method of embodiment 290, wherein the compound is ((R)—N-(5-(2-(2,5-difluorophenyl)pyrrolidin-1-yl)pyrazolo[1,5-a]pyrimidin-3-yl)-3-hydroxyazetidine-1-carboxamide, or a pharmaceutically acceptable salt thereof.
Embodiment 292. The method of embodiment 290, wherein the compound is N-(5-(2-(3-fluorophenyl)-2-methylpyrrolidin-1-yl)pyrazolo[1,5-a]pyrimidin-3-yl)-3-hydroxyazetidine-1-carboxamide, or a pharmaceutically acceptable salt thereof.
Embodiment 293. The method of embodiment 290, wherein the compound is (R)-1-(5-(2-(2,5-difluorophenyl)pyrrolidin-1-yl)pyrazolo[1,5-a]pyrimidin-3-yl)-3-phenylurea, or a pharmaceutically acceptable salt thereof.
Embodiment 294. The method of embodiment 290, wherein the compound is (R)—N-(5-(2-(2-(difluoromethyl)-5-fluorophenyl)pyrrolidin-1-yl)pyrazolo[1,5-a]pyrimidin-3-yl)-3-hydroxyazetidine-1-carboxamide, or a pharmaceutically acceptable salt thereof.
Embodiment 295. The method of embodiment 290, wherein the compound is ((R)—N-(5-(2-(3-fluorophenyl)pyrrolidin-1-yl)pyrazolo[1,5-a]pyrimidin-3-yl)-1-methyl-6-oxo-1,6-dihydropyridazine-3-carboxamide, or a pharmaceutically acceptable salt thereof.
Embodiment 296. The method of embodiment 290, wherein the compound is (S)—N-(5-((R)-2-(2,5-difluorophenyl)pyrrolidin-1-yl)pyrazolo[1,5-a]pyrimidin-3-yl)-3-hydroxypyrrolidine-1-carboxamide, or a pharmaceutically acceptable salt thereof.
Embodiment 297. The method of embodiment 290, wherein the compound is (3R,4R)—N-(5-((R)-2-(2,5-difluorophenyl)pyrrolidin-1-yl)pyrazolo[1,5-a]pyrimidin-3-yl)-3,4-dihydroxypyrrolidine-1-carboxamide, or a pharmaceutically acceptable salt thereof.
Embodiment 298. The method of embodiment 290, wherein the compound is (S)—N-(5-((R)-2-(2-chloro-5-fluorophenyl)pyrrolidin-1-yl)pyrazolo[1,5-a]pyrimidin-3-yl)-3-methylpiperazine-1-carboxamide, or a pharmaceutically acceptable salt thereof.
Embodiment 299. The method of embodiment 290, wherein the compound is (R)—N-(5-(2-(2,5-difluorophenyl)pyrrolidin-1-yl)pyrazolo[1,5-a]pyrimidin-3-yl)-3-hydroxy-3-methylazetidine-1-carboxamide, or a pharmaceutically acceptable salt thereof.
Embodiment 300. The method of embodiment 290, wherein the compound is (R)—N-(5-(2-(3-fluorophenyl)pyrrolidin-1-yl)pyrazolo[1,5-a]pyrimidin-3-yl)-3-hydroxyazetidine-1-carboxamide, or a pharmaceutically acceptable salt thereof.
Embodiment 301. The method of embodiment 290, wherein the compound is (R)-1-(4-chlorophenyl)-3-(5-(2-(2, 5-difluorophenyl)pyrrolidin-1-yl)pyrazolo[1,5-a]pyrimidin-3-yl)urea, or a pharmaceutically acceptable salt thereof.
Embodiment 302. The method of embodiment 290, wherein the compound is (6R)-9-fluoro-15-hydroxy-13-oxa-2,11,17,21,22,25-hexaazapentacyclo[17.5.2.02,6.07,12.022,26] hexacosa-1(25),7,9,11,19(26),20,23-heptaen-18-one, or a pharmaceutically acceptable salt thereof.
Embodiment 303. The method of embodiment 290, wherein the compound is (6R,15R)-9-fluoro-15-hydroxy-13-oxa-2,11,17,21,22,25-hexaazapentacyclo-[17.5.2.02,6.07,12.022,26] hexacosa-1(25),7,9,11,19(26),20,23-heptaen-18-one, or a pharmaceutically acceptable salt thereof.
Embodiment 304. The method of embodiment 290, wherein the compound is (6R)-9-fluoro-13-oxa-2,11,16,20,21,24-hexaazapentacyclo[16.5.2.02,6.07,12.021,25]pentacosa-1(24),7, 9,11,18(25),19,22-heptaen-17-one, or a pharmaceutically acceptable salt thereof.
Embodiment 305. The method of embodiment 290, wherein the compound is (6R)-9-fluoro-13-oxa-2,11,18,22,23,26-hexaazapentacyclo[18.5.2.02,6.07,12.023,2]heptacosa-1(26),7,9,11,20(27),21,24-heptaen-19-one, or a pharmaceutically acceptable salt thereof.
Embodiment 306. The method of embodiment 290, wherein the compound is (6R)-9-fluoro-2,11,13,16,20,21,24-heptaazapentacyclo[16.5.2.02,6.07,12.021,25]pentacosa-1(24),7,9,11,18(25),19,22-heptaen-17-one, or a pharmaceutically acceptable salt thereof.
Embodiment 307. The method of embodiment 290, wherein the compound is (6R)-9-fluoro-2,11,13,17,21,22,25-heptaazapentacyclo[17.5.2.02,6.07,12.022,26]hexacosa-1(25),7,9,11,19(26),20,23-heptaen-18-one, or a pharmaceutically acceptable salt thereof.
Embodiment 308. The method of embodiment 290, wherein the compound is (6R)-9-fluoro-17-methyl-13-oxa-2,11,17,21,22,25-hexaazapentacyclo[17.5.2.02,6.07,12.022,26] hexacosa-1(25),7,9,11,19(26),20,23-heptaen-18-one, or a pharmaceutically acceptable salt thereof.
Embodiment 309. The method of embodiment 290, wherein the compound is (6R)-9,15,15-trifluoro-13-oxa-2,11,17,21,22,25-hexaazapentacyclo[17.5.2.02,6.07,12.022,26]hexacosa-1(25),7,9,11,19(26),20,23-heptaen-18-one, or a pharmaceutically acceptable salt thereof.
Embodiment 310. The method of embodiment 290, wherein the compound is (6R)-9-fluoro-2,11,16,20,21,24-hexaazapentacyclo[16.5.2.02,6.07,12.021,25]pentacosa-1(24),7,9,11,18(25),19,22-heptaen-17-one, or a pharmaceutically acceptable salt thereof.
Embodiment 311. The method of embodiment 290, wherein the compound is (6R)-9-fluoro-15-methyl-2, 11,16,20,21,24-hexaazapentacyclo[16.5.2.02,6.07,12.021,25]pentacosa-1(24),7,9,11,18(25),19,22-heptaen-17-one, or a pharmaceutically acceptable salt thereof.
Embodiment 312. The method of embodiment 290, wherein the compound is (6R,15R)-9-fluoro-15-methyl-2,11,16,20,21,24-hexaazapentacyclo[16.5.2.02,6.07,12.021,25]pentacosa-1(24),7,9,11,18(25),19,22-heptaen-17-one, or a pharmaceutically acceptable salt thereof.
Embodiment 313. The method of embodiment 290, wherein the compound is (6R)-9-fluoro-15,15-dimethyl-13-oxa-2,11,17,21,22,25-hexaazapentacyclo [17.5.2.02,6.07,12.022,26] hexacosa-1(25),7,9,11,19(26),20,23-heptaen-18-one, or a pharmaceutically acceptable salt thereof.
Embodiment 314. The method of embodiment 290, wherein the compound is (6R)-9-fluoro-15,15-dimethyl-2,11,16,20,21,24-hexaazapentacyclo[16.5.2.02,6.07,12.021,25]pentacosa-1(24),7,9,11,18(25),19,22-heptaen-17-one, or a pharmaceutically acceptable salt thereof.
Embodiment 315. The method of embodiment 290, wherein the compound is TPX-0005, or a pharmaceutically acceptable salt thereof.
Embodiment 316. The method of any one of embodiments 290-315, wherein the Trk-associated cancer is one or more of a spitzoid neoplasm, lung adenocarcinoma, papillary thyroid carcinoma, histiocytic neoplasms, glioma (e.g., glioblastoma, glioblastoma multiforme, high-grade glioma, glioneuronal tumor, pediatric high-grade glioma, pilocytic astrocytoma, lower grade glioma, pilocytic astrocytoma with anaplasia, anaplastic astrocytoma, astrocytoma, ganglioma, ganglioglioma, complex glioneuronal tumor, malignant epithelioid glioneuronal tumor, oligoastrocytoma), colorectal cancer, an LPF-like neural tumor, spindle cell uterine sarcoma, myofibroma/myofibromatosis, dendritic cell neoplasm, large cell neuroendrocine cancer, non-small cell lung cancer, thyroid cancer (e.g., thyroid carcinoma, papillary thyroid cancer, thyroid gland carcinoma, post-chernobyl radiation-induced thyroid cancer, sporadic pediatric differentiated thyroid carcinoma), soft tissue fibrosarcoma, non-small cell lung cancer, intrahepatic cholangiocarcinoma, sarcoma, breast cancer, uterus carcinoma, pancreatic adenocarcinoma, bladder urothelial carcinoma, a non-spitzoid metastasizing melanoma, soft tissue sarcoma, pancreatic cancer, head and neck squamous cell carcinoma, melanoma, salivary gland cancer, uterine spindle cell sarcoma, fibrosarcoma (e.g., pediatric fibrosarcoma or infantile fibrosarcoma,), mesenchymal tumor, congenital mesoblastic nephroma, soft tissue solitary fibrous tumor, cervical squamous cell carcinoma, melanocytic tumor (e.g., spitz tumor), renal metanephric adenoma, multiple myeloma, and colon adenocarcinoma.
Embodiment 317. The method of any one of embodiments 262-316, wherein the method further comprises administering an immunotherapy agent.
Embodiment 318. The method of any one of embodiments 262-317, wherein the immunotherapy agent is selected from the group consisting of: a cellular immunotherapy; an antibody therapy; an antibody-drug conjugate; a toxin; blinatumomab (AMG103) or midostaurin (Rydapt); a cytokine therapy; an immune checkpoint inhibitor; an mRNA-based immunotherapy; bacillus Calmette-Guerin (BCG) therapy; an oncolytic virus therapy; a cancer vaccine; a peptide vaccine; and a DNA-based vaccine.
Embodiment 319. The method of any one of embodiments 262-318, wherein the cellular immunotherapy is selected from the group consisting of: adoptive T-cell therapy; dendritic cell therapy; natural killer cell therapy; sipuleucel-T (APC8015); cells that express a chimeric antigen receptor (CAR); CAR-T cell therapy; and tisagenlecleucel.
Embodiment 320. The method of any one of embodiments 262-319, wherein the antibody therapy is selected from the group consisting of: a monoclonal antibody and a conjugated antibody.
Embodiment 321. The method of any one of embodiments 262-320, wherein the antibody therapy is selected from the group consisting of: bevacizumab; trastuzumab; avelumab; rituximab; edrecolomab; daratumuab; olaratumab; ofatumumab; alemtuzumab; cetuximab; oregovomab; pembrolizumab; dinutiximab; obinutuzumab; tremelimumab (CP-675;206); ramucirumab; ublituximab (TG-1101); panitumumab; elotuzumab; avelumab; necitumumab; cirmtuzumab; ibritumomab; isatuximab (SAR650984); nimotuzumab; fresolimumab (GC1008); lirilumab (INN); mogamulizumab; ficlatuzumab (AV-299); denosumab; ganitumab; urelumab; pidilizumab; and amatuximab.
Embodiment 322. The method of any one of embodiments 262-321, wherein the antibody-drug conjugate is selected from the group consisting of: gemtuzumab ozogamicin; inotuzumab ozogamicin; brentuximab vedotin; ado-trastuzumab emtansine (TDM-1); mirvetuximab soravtansine (IMGN853); and anetumab ravtansine.
Embodiment 323. The method of any one of embodiments 262-322, wherein the toxin is denileukin diftitox.
Embodiment 324. The method of any one of embodiments 262-323, wherein the immunotherapy agent is blinatumomab (AMG103).
Embodiment 325. The method of any one of embodiments 262-324, wherein the immunotherapy agent is midostaurin (Rydapt).
Embodiment 326. The method of any one of embodiments 262-325, wherein the cytokine therapy is selected from the group consisting of: an interleukin 2 (IL-2) therapy; an interferon alpha (IFNα) therapy; a granulocyte colony stimulating factor (G-CSF) therapy; an interleukin 12 (IL-12) therapy; an interleukin 15 (IL-15) therapy; an interleukin 7 (IL-7) therapy; and an erythropoietin-alpha (EPO) therapy.
Embodiment 327. The method of any one of embodiments 262-326, wherein the interleukin 2 (IL-2) therapy is aldesleukin.
Embodiment 328. The method of any one of embodiments 262-327, wherein the IFNα therapy is interferon alfa-2b or interferon alfa-2a.
Embodiment 329. The method of any one of embodiments 262-328, wherein the G-CSF therapy is filgrastim.
Embodiment 330. The method of any one of embodiments 262-329, wherein the immune checkpoint inhibitor is selected from the group consisting of: a CTLA-4 inhibitor; a PD-1 inhibitor; and a PD-L1 inhibitor.
Embodiment 331. The method of any one of embodiments 262-330, wherein the CTLA-4 inhibitor is ipilimumab or tremelimumab (CP-675,206).
Embodiment 332. The method of any one of embodiments 262-331, wherein the PD-1 inhibitor is pembrolizumab or nivolumab.
Embodiment 333. The method of any one of embodiments 262-332, wherein the PD-L1 inhibitor is selected from the group consisting of: atezolizumab; avelumab; and durvalumab.
Embodiment 334. The method of any one of embodiments 262-333, wherein the mRNA-based immunotherapy is CV9104.
Embodiment 335. The method of any one of embodiments 262-334, wherein the immunotherapy agent is bacillus Calmette-Guerin (BCG) therapy.
Embodiment 336. The method of any one of embodiments 262-335, wherein the oncolytic virus therapy is talimogene alherparepvec (T-VEC).
Embodiment 337. The method of any one of embodiments 262-336, wherein the cancer vaccine is a human papillomavirus (HPV) vaccine.
Embodiment 338. The method of any one of embodiments 262-337, wherein the human papillomavirus (HPV) vaccine is selected from the group consisting of: a recombinant human papillomavirus vaccine [types 6, 11, 16, and 18]; a recombinant human papillomavirus vaccine [types 6, 11, 16, 18, 31, 33, 45, 52, and 58]; and a recombinant human papillomavirus vaccine [types 16 and 18].
Embodiment 339. The method of any one of embodiments 262-338, wherein the cancer vaccine is a hepatitis B virus (HBV) vaccine.
Embodiment 340. The method of any one of embodiments 262-339, wherein the cancer vaccine is selected from the group consisting of: a combination Hepatitis A and Hepatitis B vaccine; a combination diphtheria, tetanus, pertussis, hepatitis B virus, and poliomyelitis vaccine; dasiprotimut-T; cancer vaccine HSPPC-96; GVAX; ADXS11-001; ALVAC-CEA; rilimogene galvacirepvec/rilimogene glafolivec; CDX-110; CimaVax-EGF; lapuleucel-T (APC8024); GRNVAC1; GRNVAC2; GRN-1201; hepcortespenlisimut-L (Hepko-V5); a dendritic cell vaccine; ICT-107; SCIB1; BMT CTN 1401; PrCa VBIR; PANVAC; a prostate cancer vaccine; DPX-Survivac; and viagenpumatucel-L (HS-110).
Embodiment 341. The method of any one of embodiments 262-340, wherein the peptide vaccine is selected from the group consisting of: nelipepimut-S(E75); IMA901; SurVaxM (SVN53-67); an immunogenic personal neoantigen vaccine; RGSH4K; and NEO-PV-01.
Embodiment 342. The method of any one of embodiments 262-341, wherein the DNA-based vaccine is a mammaglobin-A DNA vaccine.

As used herein, the term “composition” is intended to encompass a product comprising the specified ingredients in the specified amounts, as well as any product which results, directly or indirectly, from combinations of the specified ingredients in the specified amounts.

In some embodiments, the term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any range therein.

A number of embodiments of the invention have been described. Nevertheless, it will be understood that various modifications may be made without departing from the spirit and scope of the invention. For example, other Trk inhibitors that are not specifically described herein may be used. Accordingly, other embodiments are within the scope of the following claims.

1. A method for treating cancer, the method comprising administering to the patient a therapeutically effective amount of a first Trk inhibitor or a pharmaceutically acceptable salt thereof, a second Trk inhibitor or a pharmaceutically acceptable salt thereof, and an immunotherapy agent.

2. The method of claim 1, wherein the first Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide); (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate; cabozantinib ((N-(4-(6,7-Dimethoxyquinolin-4-yl)oxy)phenyl)-N′-(4-fluorophenyl)cyclopropane-1,1-dicarboxamide)); dovitinib (4-amino-5-fluoro-3-[6-(4-methylpiperazin-1-yl)-1H-benzimidazol-2-yl]quinolin-2(1H)-one mono 2-hydroxypropanoate hydrate); belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); PLX7486; altiratinib (N-(4-((2-(cyclopropanecarboxamido)pyridin-4-yl)oxy)-2,5-difluorophenyl)-N-(4-fluorophenyl)cyclopropane-1,1-dicarboxamide); AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine); (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one; a (R)-2-phenylpyrrolidine substituted imadazopyridazine; AZD6918; GNF-4256; GTx-186; GNF-5837; AZ623; AG-879; CT327; AR-772; AR-523; AR-786; AR-256; AR-618; AZ-23; CEP-701; CEP-751; PHA-739358; dovitinib; Gö 6976; GW441756; MGCD516; ONO-5390556; PHA-848125AC; Regorafenib; Sorafenib; Sunitinib; TSR-011; VM-902A; K252a; a 4-aminopyrazolylpyrimidine; a substituted pyrazolo[1,5-a]pyrimidine compound; BMS-754807; ONO-7579; F17752; ANA-12; ONO-4474; GZ389988; and TPX-0005.

3. The method of claim 1, wherein the first Trk inhibitor is (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate or (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one.

4. The method of claim 1, wherein the second Trk inhibitor is selected from the group consisting of: entrectinib (N-[5-(3,5-difluoro-benzyl)-1H-indazol-3-yl]-4-(4-methylpiperazin-1-yl)-2-(tetrahydro-pyran-4-ylamino)-benzamide); (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate; cabozantinib ((N-(4-((6,7-Dimethoxyquinolin-4-yl)oxy)phenyl)-N′-(4-fluorophenyl)cyclopropane-1,1-dicarboxamide)); dovitinib (4-amino-5-fluoro-3-[6-(4-methylpiperazin-1-yl)-1H-benzimidazol-2-yl]quinolin-2(1H)-one mono 2-hydroxypropanoate hydrate); belizatinib (4-fluoro-N-(6-((4-(2-hydroxypropan-2-yl)piperidin-1-yl)methyl)-1-((1s,4s)-4-(isopropylcarbamoyl)cyclohexyl)-1H-benzo[d]imidazol-2-yl)benzamide); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); PLX7486; altiratinib (N-(4-((2-(cyclopropanecarboxamido)pyridin-4-yl)oxy)-2,5-difluorophenyl)-N-(4-fluorophenyl)cyclopropane-1,1-dicarboxamide); AZD7451 ((S)—N-(1-(5-fluoropyrimidin-2-yl)ethyl)-3-(5-isopropoxy-1H-pyrazol-3-yl)-3H-imidazo[4,5-b]pyridin-5-amine); (6R,15R)-9-fluoro-15-methyl-2,11,16,20,21,24-hexaazapentacyclo[16.5.2.02,6.07,12.021,25]pentacosa-1(24),7,9, 11,18(25),19,22-heptaen-17-one; a (R)-2-phenylpyrrolidine substituted imadazopyridazine; AZD6918; GNF-4256; GTx-186; GNF-5837; AZ623; AG-879; CT327; AR-772; AR-523; AR-786; AR-256; AR-618; AZ-23; CEP-701; CEP-751; PHA-739358; dovitinib; Gö 6976; GW441756; MGCD516; ONO-5390556; PHA-848125AC; Regorafenib; Sorafenib; Sunitinib; TSR-011; VM-902A; K252a; a 4-aminopyrazolylpyrimidine; a substituted pyrazolo[1,5-a]pyrimidine compound; BMS-754807; ONO-7579; F17752; ANA-12; ONO-4474; GZ389988; and TPX-0005;
provided that the second Trk inhibitor is different than the first Trk inhibitor.

5. The method of claim 1, wherein the second Trk inhibitor is (S)—N-(5-((R)-2-(2,5-difluorophenyl)pyrrolidin-1-yl)pyrazolo[1,5-a]pyrimidin-3-yl)-3-hydroxypyrrolidine-1-carboxamide sulfate.

6. The method of claim 1, wherein the first Trk inhibitor is (6R,15R)-9-fluoro-15-methyl-2, 11,16,20,21,24-hexaazapentacyclo[16.5.2.02,6.07,12.021,25]pentacosa-1(24),7,9, 11,18(25),19,22-heptaen-17-one and the second Trk inhibitor is (S)—N-(5-((R)-2-(2,5-difluorophenyl)pyrrolidin-1-yl)pyrazolo[1, 5-a]pyrimidin-3-yl)-3-hydroxypyrrolidine-1-carboxamide sulfate.

7. The method of claim 1, wherein the cancer is a Trk-associated cancer.

8. The method of claim 7, wherein the Trk-associated cancer is due to oncogenic rearrangements in a NTRK gene selected from the group consisting of: NTRK1, NTRK2, and NTRK3.

9. The method of claim 1, wherein the cancer is selected from the group consisting of: adrenocortical carcinoma; anal cancer; appendix cancer; atypical teratoid/rhabdoid tumor (e.g., central nervous system atypical teratoid/rhabdoid tumor); B-cell cancer; bile duct cancer; bladder cancer; bone cancer (e.g., osteosarcoma and malignant fibrous histiocytoma); brain cancer (e.g., brain and spinal cord tumor; brain stem glioma; central nervous system embryonal tumors; central nervous system germ cell tumors; craniopharyngioma; and ependymoma); breast cancer; bronchogenic carcinoma; bronchus cancer; cancer of hematological tissues; cancer of the oral cavity or pharynx; carcinoid tumor; cervical cancer; childhood cancers; chordoma; chronic lymphocytic leukemia; chronic myeloproliferative neoplasms; colon cancer; colorectal cancer; cutaneous T-cell lymphoma; ductal carcinoma in situ; embryonal tumor; endometrial cancer; esophageal cancer; esthesioneuroblastoma; extracranial germ cell tumor; extragonadal germ cell tumor; extrahepatic bile duct cancer; eye cancer (e.g., retinoblastoma); fallopian tube cancer; fibrosarcoma; fibrous histiocytoma of bone; gallbladder cancer; gastric cancer; gastrointestinal carcinoid tumor; germ cell tumor; gestational trophoblastic disease; glioblastoma multiforme; glioma (e.g., lower-grade glioma); head and neck cancer; heart cancer; histiocytosis; hypopharyngeal cancer; inflammatory myofibroblastic tumors; intrahepatic cholangiocarcinoma; islet cell tumor; kidney cancer (e.g., renal cell cancer); Langerhans cell histiocytosis; large cell neuroendocrine cancer; laryngeal cancer; leukemia (e.g., acute lymphoblastic leukemia; acute myeloid leukemia; chronic myelogenous leukemia; and hairy cell leukemia); lip cancer; liver cancer; lung cancer; Burkitt lymphoma; Hodgkin's lymphoma; and primary central nervous system lymphoma); medulloblastoma; mesothelioma; mouth cancer; multiple myeloma; myelodysplastic syndromes; nasal cavity and paranasal sinus cancer; nasopharyngeal cancer; neoplasm (e.g., a melanocystic neoplasm); nephroma; neuroblastoma; non-small cell lung cancer; oral cancer; oropharyngeal cancer; ovarian cancer; pancreatic cancer; paraganglioma; parathyroid cancer; glioma (e.g., pediatric gliomas); penile cancer; pharyngeal cancer; pheochromocytoma; pilocytic astrocytoma; pituitary tumor; plasma cell neoplasm; primary peritoneal cancer; prostate cancer; rectum carcinoma; salivary gland cancer; sarcoma (e.g., Ewing sarcoma; rhabdomyosarcoma; uterine sarcoma; and undifferentiated sarcoma); secretory breast carcinoma; Sezary syndrome; skin cancer; small bowel cancer; small cell lung cancer; small intestine cancer; Spitz nevi; Spitz tumors; spitzoid melanoma; stomach cancer; squamous cell carcinoma; squamous neck cancer; testicular cancer; throat cancer; thymoma and thymic carcinoma; thyroid carcinoma; urethral cancer; uterine cancer; urinary bladder cancer; vaginal cancer; vulvar cancer; and Wilms tumor.

10. The method of claim 1, wherein the first Trk inhibitor and the second Trk inhibitor are administered simultaneously, separately, or sequentially to treat cancer.
